(19) United States
(12) Patent Application Publication
(10) Pub. No.: US 2022/0223793 A1
(43) Pub. Date: Jul. 14, 2022

(54) ORGANIC LIGHT EMITTING DIODE AND ORGANIC LIGHT EMITTING DEVICE INCLUDING THE SAME

(71) Applicant: LG DISPLAY CO., LTD., Seoul (KR)

(72) Inventors: Dae Won RYU, Paju-si (KR); In Bum SONG, Paju-si (KR); Seung Hee YOON, Paju-si (KR); Sang Beom KIM, Paju-si (KR)

(73) Assignee: LG DISPLAY CO., LTD., Seoul (KR)

(21) Appl. No.: 17/614,863

(22) PCT Filed: Dec. 23, 2020
(86) PCT No.: PCT/KR2020/018950

§ 371 Date: Nov. 29, 2021

(30) Foreign Application Priority Data
Dec. 30, 2019 (KR) 10-2019-0178653

(51) Int. Cl. H01L 51/00; C07C 15/28; C09K 11/06; C07D 307/91

(52) U.S. Cl. CPC H01L 51/0058; C07C 15/28; C09K 11/06; H01L 51/5012; H01L 51/0052; H01L 51/0073; H01L 51/0054; C07D 307/91

(57) ABSTRACT

The present disclosure relates to an OLED that includes a first electrode; a second electrode facing the first electrode; and a first emitting material layer including a first host being an anthracene derivative and a first dopant being a pyrene derivative and positioned between the first and second electrodes, wherein at least one of an anthracene core of the first host and a pyrene core of the first dopant is deuterated.

### CROSS-REFERENCE TO RELATED APPLICATIONS

This application is a U.S. National Phase of PCT/KR2020/018950, filed Dec. 23, 2020, which claims priority to Korean Patent Application No. 10-2019-0178653 filed in the Republic of Korea on Dec. 30, 2019, the entire contents of all of these applications being expressly incorporated by reference into the present application.

### TECHNICAL FIELD

The present disclosure relates to an organic light emitting diode (OLED), and more specifically, to an OLED having enhanced emitting efficiency and lifespan and an organic light emitting device including the same.

### BACKGROUND ART

As requests for a flat panel display device having a small occupied area have been increased, an organic light emitting display device including an OLED has been research and development.

The OLED emits light by injecting electrons from a cathode as an electron injection electrode and holes from an anode as a hole injection electrode into an emitting material layer (EML), combining the electrons with the holes, generating an exciton, and transforming the exciton from an excited state to a ground state. A flexible substrate, for example, a plastic substrate, can be used as a base substrate where elements are formed. In addition, the organic light emitting display device can be operated at a voltage (e.g., 10V or below) lower than a voltage required to operate other display devices. Moreover, the organic light emitting display device has advantages in the power consumption and the color sense.

The OLED includes a first electrode as an anode over a substrate, a second electrode, which is spaced apart from and faces the first electrode, and an organic emitting layer therebetween.

For example, the organic light emitting display device can include a red pixel region, a green pixel region and a blue pixel region, and the OLED can be formed in each of the red, green and blue pixel regions.

However, the OLED in the blue pixel does not provide sufficient emitting efficiency and lifespan such that the organic light emitting display device has a limitation in the emitting efficiency and the lifespan.

### DISCLOSURE

### Technical Problem

Accordingly, the present disclosure is directed to an OLED and an organic light emitting device including the OLED that substantially obviate one or more of the problems due to the limitations and disadvantages of the related art.

An object of the present disclosure is to provide an OLED having enhanced emitting efficiency and lifespan and an organic light emitting device including the same.

Additional features and advantages of the disclosure will be set forth in the description which follows, and in part will be apparent from the description, or can be learned by practice of the disclosure. The objectives and other advantages of the disclosure will be realized and attained by the structure particularly pointed out in the written description and claims hereof as well as the appended drawings.

### Technical Solution

According to an aspect, the present disclosure provides an OLED that includes a first electrode; a second electrode facing the first electrode; and a first emitting material layer including a first host being an anthracene derivative and a first dopant being a pyrene derivative and positioned between the first and second electrodes, wherein at least one of an anthracene core of the first host and a pyrene core of the first dopant is deuterated.

As an example, all of the hydrogen atoms in at least one of the anthracene derivative and the pyrene derivative are deuterated.

As an example, at least one of an anthracene core of the anthracene derivative and a pyrene core of the pyrene derivative is deuterated.

The OLED can include a single emitting part or a tandem structure of a multiple emitting parts.

The tandem-structured OLED can emit blue color or white color light.

According to another aspect, the present disclosure provides an organic light emitting device comprising the OLED, as described above.

For example, the organic light emitting device can be an organic light emitting display device or a lightening device.

It is to be understood that both the foregoing general description and the following detailed description are examples and are explanatory and are intended to provide further explanation of the disclosure as claimed.

### Advantageous Effects

An emitting material layer of an OLED of the present disclosure includes a host of an anthracene derivative and a dopant of a pyrene derivative, and at least one of an anthracene core of the anthracene derivative and a pyrene core of the pyrene derivative is deuterated. As a result, an emitting efficiency and a lifespan of the OLED and an organic light emitting device including the OLED are improved with minimizing production cost increase.

### DESCRIPTION OF DRAWINGS

The accompanying drawings, which are included to provide a further understanding of the disclosure, are incorporated in and constitute a part of this specification, illustrate implementations of the disclosure and together with the description serve to explain the principles of embodiments of the disclosure.

FIG. 1 is a schematic circuit diagram illustrating an organic light emitting display device of the present disclosure.

FIG. 2 is a schematic cross-sectional view illustrating an organic light emitting display device according to a first embodiment of the present disclosure.

FIG. 3 is a schematic cross-sectional view illustrating an OLED having a single emitting part for the organic light emitting display device according to the first embodiment of the present disclosure.

FIG. 4 is a schematic cross-sectional view illustrating an OLED having a tandem structure of two emitting parts according to the first embodiment of the present disclosure.

FIG. 5 is a schematic cross-sectional view illustrating an organic light emitting display device according to a second embodiment of the present disclosure.

FIG. 6 is a schematic cross-sectional view illustrating an OLED for the organic light emitting display device according to the second embodiment of the present disclosure.

FIG. 7 is a schematic cross-sectional view illustrating an organic light emitting display device according to a third embodiment of the present disclosure.

### DETAILED DESCRIPTION OF THE EMBODIMENTS

Reference will now be made in detail to aspects of the disclosure, examples of which are illustrated in the accompanying drawings.

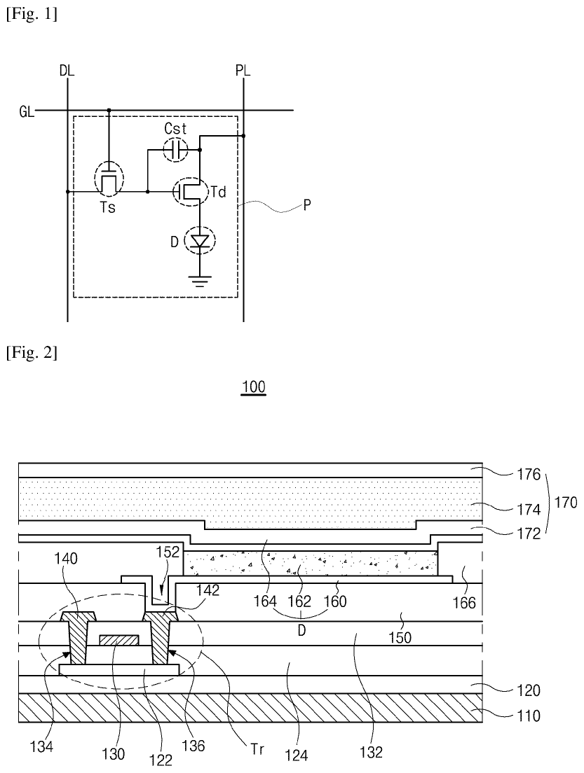

FIG. 1 is a schematic circuit diagram illustrating an organic light emitting display device of the present disclosure.

As illustrated in FIG. 1, a gate line GL and a data line DL, which cross each other to define a pixel (pixel region) P, and a power line PL are formed in an organic light emitting display device. A switching thin film transistor (TFT) Ts, a driving TFT Td, a storage capacitor Cst and an OLED D are formed in the pixel region P. The pixel region P can include a red pixel, a green pixel and a blue pixel.

The switching thin film transistor Ts is connected to the gate line GL and the data line DL, and the driving thin film transistor Td and the storage capacitor Cst are connected between the switching thin film transistor Ts and the power line PL. The OLED D is connected to the driving thin film transistor Td. When the switching thin film transistor Ts is turned on by the gate signal applied through the gate line GL, the data signal applied through the data line DL is applied a gate electrode of the driving thin film transistor Td and one electrode of the storage capacitor Cst through the switching thin film transistor Ts.

The driving thin film transistor Td is turned on by the data signal applied into the gate electrode so that a current proportional to the data signal is supplied from the power line PL to the OLED D through the driving thin film transistor Tr. The OLED D emits light having a luminance proportional to the current flowing through the driving thin film transistor Td. In this case, the storage capacitor Cst is charge with a voltage proportional to the data signal so that the voltage of the gate electrode in the driving thin film transistor Td is kept constant during one frame. Therefore, the organic light emitting display device can display a desired image.

FIG. 2 is a schematic cross-sectional view illustrating an organic light emitting display device according to a first embodiment of the present disclosure.

As illustrated in FIG. 2, the organic light emitting display device 100 includes a substrate 110, a TFT Tr and an OLED D connected to the TFT Tr. For example, the organic light emitting display device 100 can include a red pixel, a green pixel and a blue pixel, and the OLED D can be formed in each of the red, green and blue pixels. Namely, the OLEDs D emitting red light, green light and blue light can be provided in the red, green and blue pixels, respectively.

The substrate 110 can be a glass substrate or a plastic substrate. For example, the substrate 110 can be a polyimide substrate.

A buffer layer 120 is formed on the substrate, and the TFT Tr is formed on the buffer layer 120. The buffer layer 120 can be omitted.

A semiconductor layer 122 is formed on the buffer layer 120. The semiconductor layer 122 can include an oxide semiconductor material or polycrystalline silicon.

When the semiconductor layer 122 includes the oxide semiconductor material, a light-shielding pattern can be formed under the semiconductor layer 122. The light to the semiconductor layer 122 is shielded or blocked by the light-shielding pattern such that thermal degradation of the semiconductor layer 122 can be prevented. On the other hand, when the semiconductor layer 122 includes polycrystalline silicon, impurities can be doped into both sides of the semiconductor layer 122.

A gate insulating layer 124 is formed on the semiconductor layer 122. The gate insulating layer 124 can be formed of an inorganic insulating material such as silicon oxide or silicon nitride.

A gate electrode 130, which is formed of a conductive material, e.g., metal, is formed on the gate insulating layer 124 to correspond to a center of the semiconductor layer 122.

In FIG. 2, the gate insulating layer 124 is formed on an entire surface of the substrate 110. Alternatively, the gate insulating layer 124 can be patterned to have the same shape as the gate electrode 130.

An interlayer insulating layer 132, which is formed of an insulating material, is formed on the gate electrode 130. The interlayer insulating layer 132 can be formed of an inorganic insulating material, e.g., silicon oxide or silicon nitride, or an organic insulating material, e.g., benzocyclobutene or photo-acryl.

The interlayer insulating layer 132 includes first and second contact holes 134 and 136 exposing both sides of the semiconductor layer 122. The first and second contact holes 134 and 136 are positioned at both sides of the gate electrode 130 to be spaced apart from the gate electrode 130.

The first and second contact holes 134 and 136 are formed through the gate insulating layer 124. Alternatively, when the gate insulating layer 124 is patterned to have the same shape as the gate electrode 130, the first and second contact holes 134 and 136 is formed only through the interlayer insulating layer 132.

A source electrode 140 and a drain electrode 142, which are formed of a conductive material, e.g., metal, are formed on the interlayer insulating layer 132.

The source electrode 140 and the drain electrode 142 are spaced apart from each other with respect to the gate electrode 130 and respectively contact both sides of the semiconductor layer 122 through the first and second contact holes 134 and 136.

The semiconductor layer 122, the gate electrode 130, the source electrode 140 and the drain electrode 142 constitute the TFT Tr. The TFT Tr serves as a driving element. Namely, the TFT Tr can correspond to the driving TFT Td (of FIG. 1).

In the TFT Tr, the gate electrode 130, the source electrode 140, and the drain electrode 142 are positioned over the semiconductor layer 122. Namely, the TFT Tr has a coplanar structure.

Alternatively, in the TFT Tr, the gate electrode can be positioned under the semiconductor layer, and the source and drain electrodes can be positioned over the semiconductor layer such that the TFT Tr can have an inverted staggered structure. In this instance, the semiconductor layer can include amorphous silicon.

The gate line and the data line cross each other to define the pixel, and the switching TFT is formed to be connected to the gate and data lines. The switching TFT is connected to the TFT Tr as the driving element.

In addition, the power line, which can be formed to be parallel to and spaced apart from one of the gate and data lines, and the storage capacitor for maintaining the voltage of the gate electrode of the TFT Tr in one frame can be further formed.

A passivation layer 150, which includes a drain contact hole 152 exposing the drain electrode 142 of the TFT Tr, is formed to cover the TFT Tr.

A first electrode 160, which is connected to the drain electrode 142 of the TFT Tr through the drain contact hole 152, is separately formed in each pixel. The first electrode 160 can be an anode and can be formed of a conductive material having a relatively high work function. For example, the first electrode 160 can be formed of a transparent conductive material such as indium-tin-oxide (ITO) or indium-zinc-oxide (IZO).

When the OLED device 100 is operated in a top-emission type, a reflection electrode or a reflection layer can be formed under the first electrode 160. For example, the reflection electrode or the reflection layer can be formed of aluminum-palladium-copper (APC) alloy.

A bank layer 166 is formed on the passivation layer 150 to cover an edge of the first electrode 160. Namely, the bank layer 166 is positioned at a boundary of the pixel and exposes a center of the first electrode 160 in the pixel.

An organic emitting layer 162 is formed on the first electrode 160. The organic emitting layer 162 can have a single-layered structure of an emitting material layer including an emitting material. To increase an emitting efficiency of the OLED D and/or the organic light emitting display device 100, the organic emitting layer 162 can have a multi-layered structure.

The organic emitting layer 162 is separated in each of the red, green and blue pixels. As illustrated below, the organic emitting layer 162 in the blue pixel includes a host of an anthracene derivative and a dopant of a pyrene derivative, and at least one of an anthracene core of the anthracene derivative and a pyrene core of the pyrene derivative is deuterated. As a result, the emitting efficiency and the lifespan of the OLED D in the blue pixel are improved.

A second electrode 164 is formed over the substrate 110 where the organic emitting layer 162 is formed. The second electrode 164 covers an entire surface of the display area and can be formed of a conductive material having a relatively low work function to serve as a cathode. For example, the second electrode 164 can be formed of aluminum (Al), magnesium (Mg), silver (Ag), Al—Mg alloy (AlMg) or Mg—Ag alloy (MgAg).

The first electrode 160, the organic emitting layer 162 and the second electrode 164 constitute the OLED D.

An encapsulation film 170 is formed on the second electrode 164 to prevent penetration of moisture into the OLED D. The encapsulation film 170 includes a first inorganic insulating layer 172, an organic insulating layer 174 and a second inorganic insulating layer 176 sequentially stacked, but it is not limited thereto. The encapsulation film 170 can be omitted.

A polarization plate for reducing an ambient light reflection can be disposed over the top-emission type OLED D. For example, the polarization plate can be a circular polarization plate.

In addition, a cover window can be attached to the encapsulation film 170 or the polarization plate. In this instance, the substrate 110 and the cover window have a flexible property such that a flexible display device can be provided.

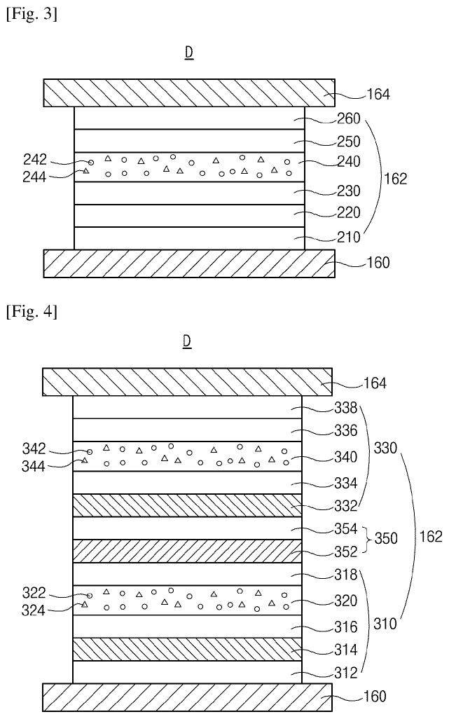

FIG. 3 is a schematic cross-sectional view illustrating an OLED having a single emitting unit for the organic light emitting display device according to the first embodiment of the present disclosure.

As illustrated in FIG. 3, the OLED D includes the first and second electrodes 160 and 164, which face each other, and the organic emitting layer 162 therebetween. The organic emitting layer 162 includes an emitting material layer (EML) 240 between the first and second electrodes 160 and 164.

The first electrode 160 can be formed of a conductive material having a relatively high work function to serve as an anode. The second electrode 164 can be formed of a conductive material having a relatively low work function to serve as a cathode. One of the first and second electrodes 160 and 164 is a transparent electrode (or a semi-transparent electrode), and the other one of the first and second electrodes 160 and 164 is a reflective electrode.

The organic emitting layer 162 can further include an electron blocking layer (EBL) 230 between the first electrode 160 and the EML 240 and a hole blocking layer (HBL) 250 between the EML 240 and the second electrode 164.

In addition, the organic emitting layer 162 can further include a hole transporting layer (HTL) 220 between the first electrode 160 and the EBL 230.

Moreover, the organic emitting layer 162 can further include a hole injection layer (HIL) 210 between the first electrode 160 and the HTL 220 and an electron injection layer (EIL) 260 between the second electrode 164 and the HBL 250.

In the OLED D of the present disclosure, the HBL 250 can include a hole blocking material of an azine derivative. The hole blocking material has an electron transporting property such that an electron transporting layer can be omitted. The HBL 250 directly contacts the EIL 260. Alternatively, the HBL can directly contact the second electrode without the EIL 260. However, an electron transporting layer can be formed between the HBL 250 and the EIL 260.

The organic emitting layer 162, e.g., the EML 240, includes the host 242 of an anthracene derivative, the dopant 244 of a pyrene derivative and provides blue emission. In this case, at least one of an anthracene core of the anthracene derivative 242 and a pyrene core of the pyrene derivative 244 is deuterated.

In the EML 240, when the anthracene core of the host 242 is deuterated (e.g., “core-deuterated anthracene derivative”), the dopant 244 can be non-deuterated (e.g., “non-deuterated pyrene derivative”) or all of the pyrene core and a substituent of the dopant 244 can be deuterated (e.g., “wholly-deuterated pyrene derivative”). Alternatively, the pyrene core of the dopant 244 except the substituent can be deuterated (e.g., “core-deuterated pyrene derivative”), or the substituent of the dopant 244 except the pyrene core can be deuterated (e.g., “substituent-deuterated pyrene derivative”).

The anthracene derivative as the host 242, in which the anthracene core is deuterated, can be represented by Formula 1:

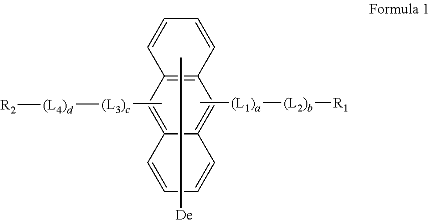

In Formula 1, each of R₁ and R₂ is independently C₆˜C₃₀ aryl group or C₅˜C₃₀ heteroaryl group, and each of L₁, L₂, L₃ and L₄ is independently C₆˜C₃₀ arylene group, each of a, b, c and d is an integer of 0 or 1, and e is an integer of 1 to 8.

Namely, in the core-deuterated anthracene derivative as the host 242, the anthracene moiety as the core is substituted by deuterium (D), and the substituent except the anthracene moiety is not deuterated.

For example, each of R₁ and R₂ can be selected from the group consisting of phenyl, naphthyl, dimethylfluorenyl, dibenzofuranyl, dibenzothiophenyl, phenanthrenyl, and carbazolyl. The dimethylfluorenyl, dibenzofuranyl, dibenzothiophenyl, phenanthrenyl, and carbazolyl can be substituted by C₆˜C₃₀ aryl group, e.g., phenyl or naphthyl. Each of L₁, L₂, L₃ and L₄ can be phenylene or naphthylene. At least one of a, b, c and d can be 0, and e can be 8.

In an exemplary embodiment, the host 242 can be a compound being one of the followings in Formula 2:

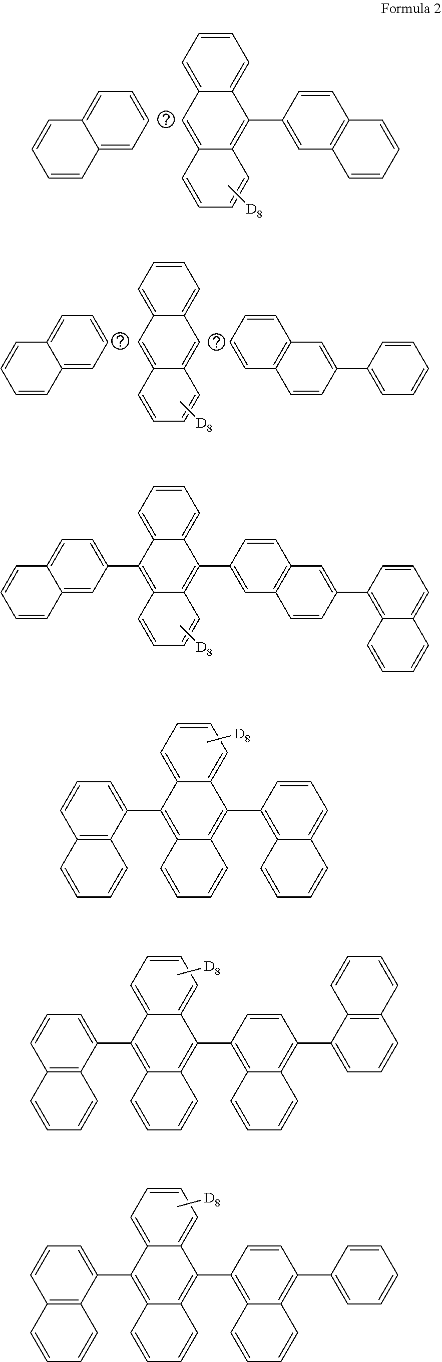

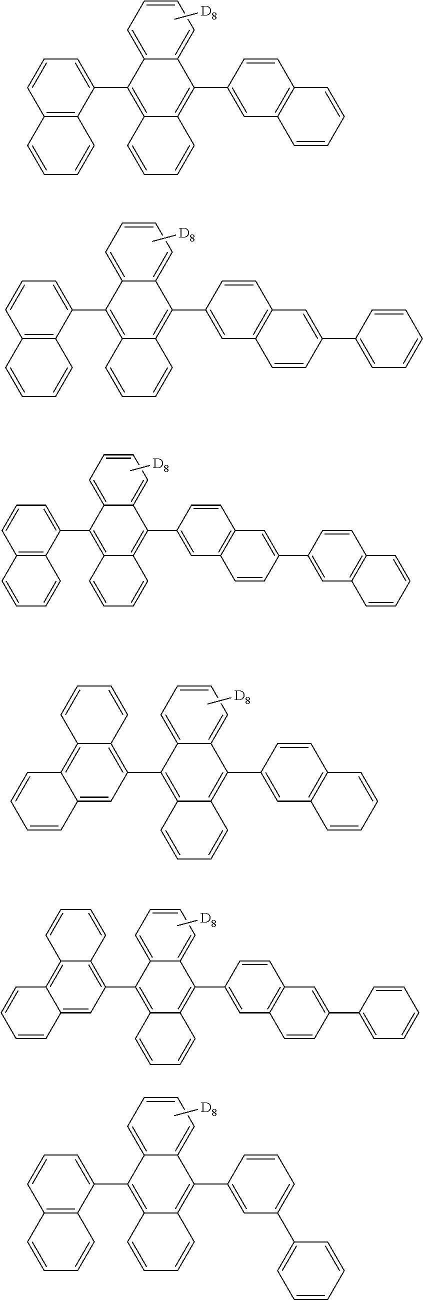

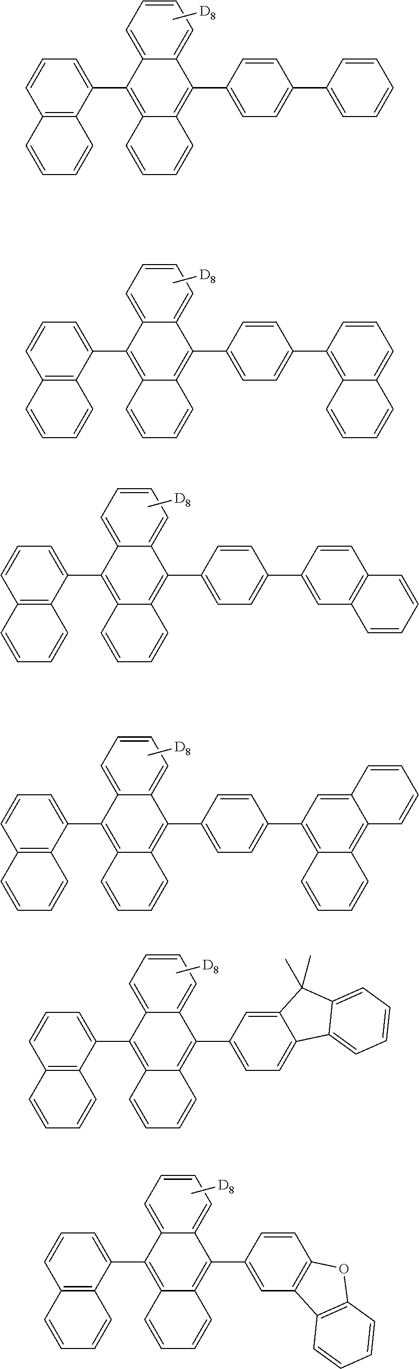

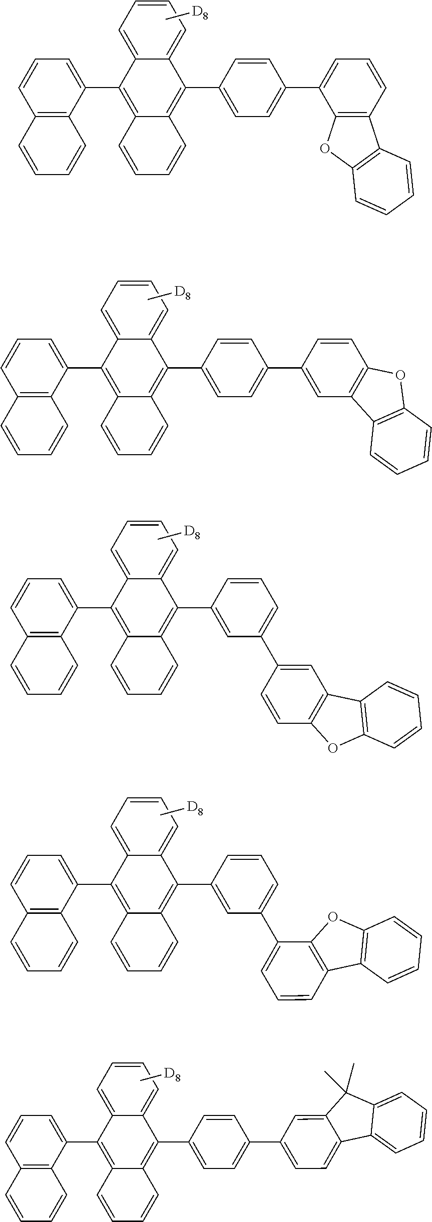

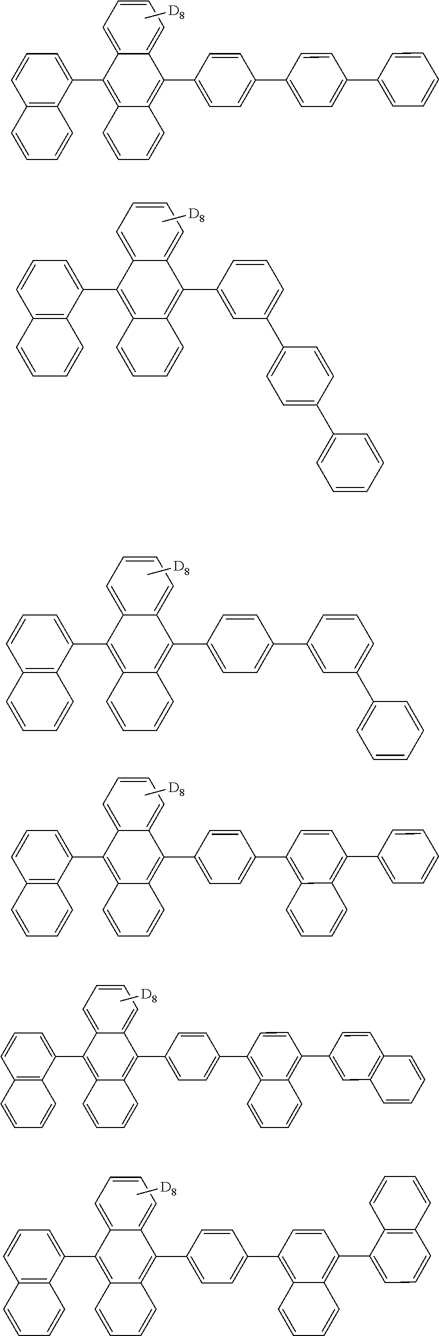

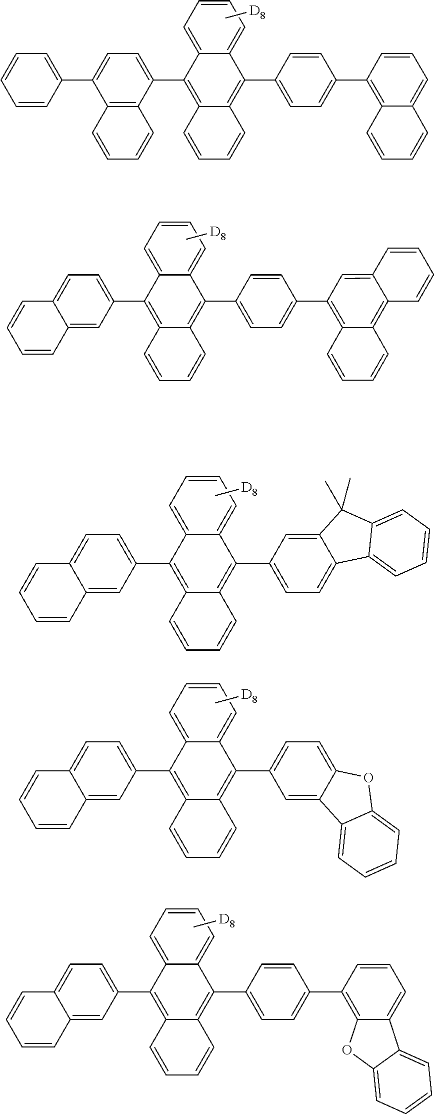

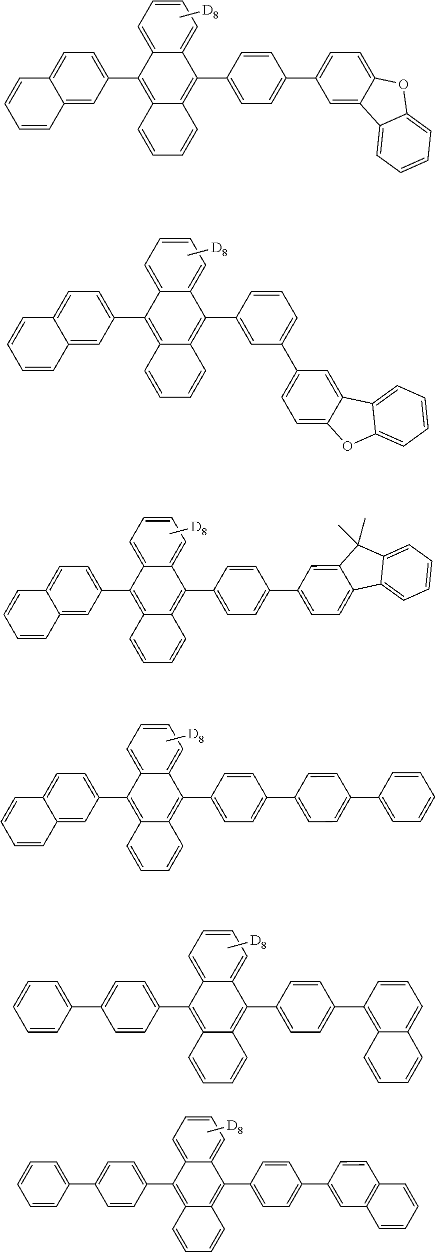

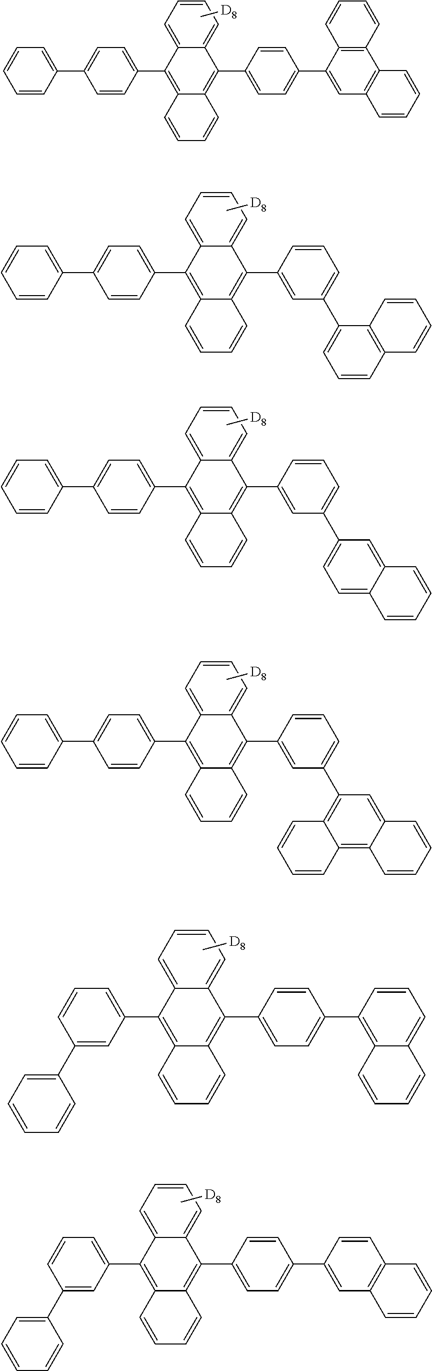

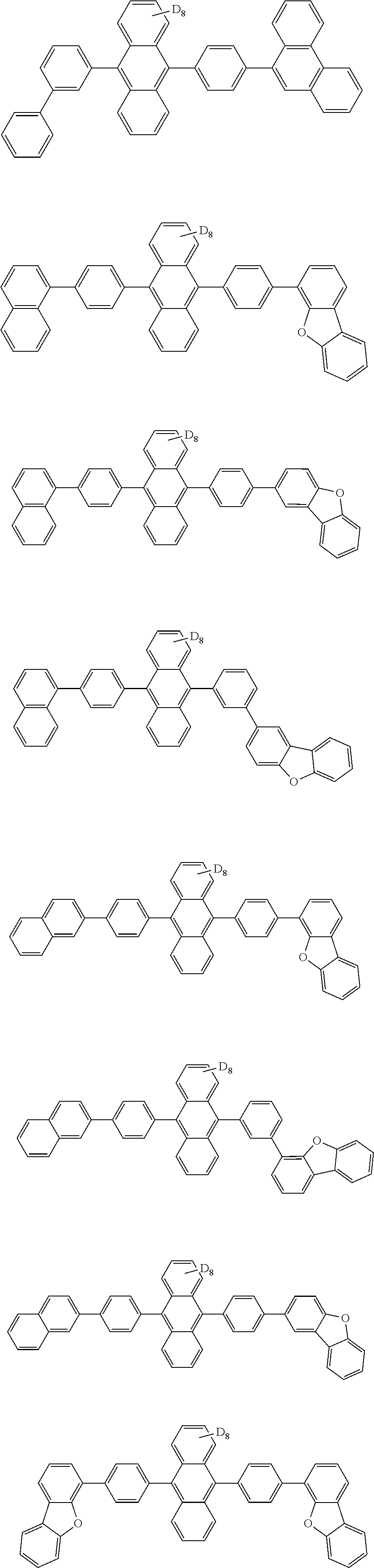

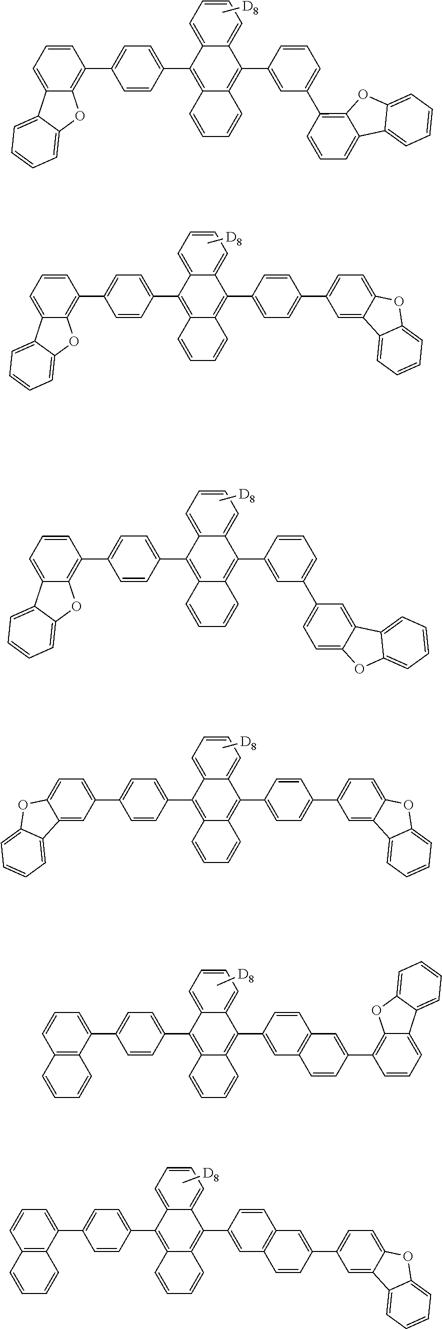

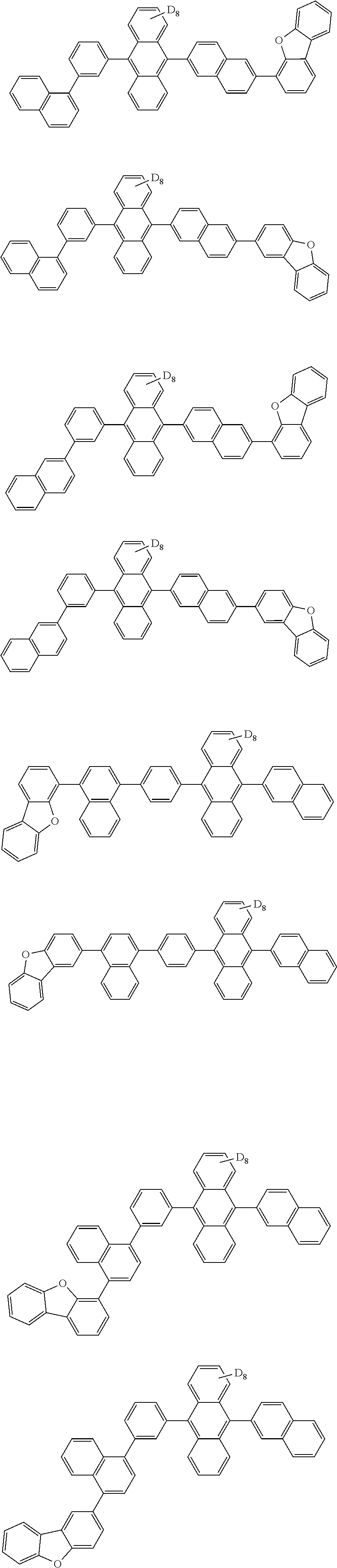

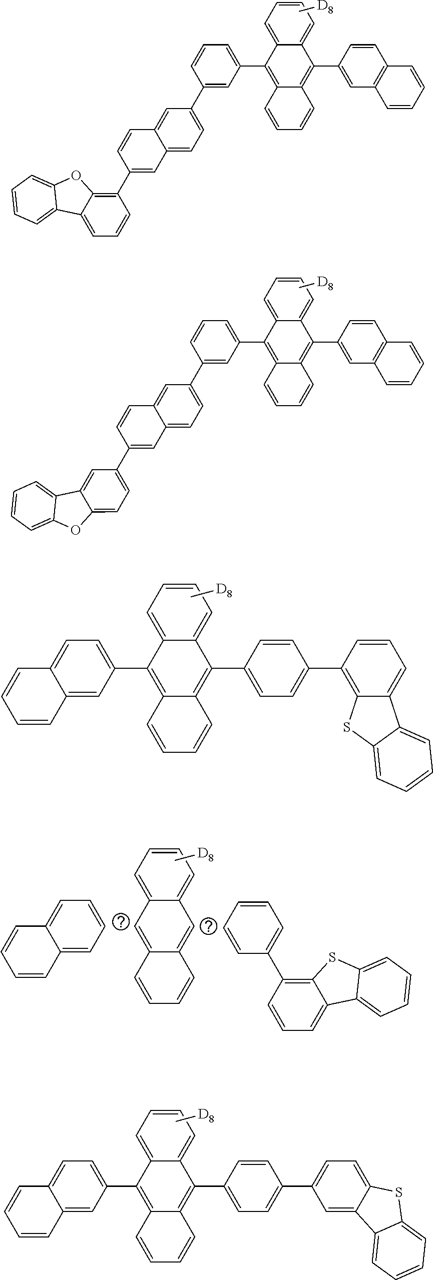

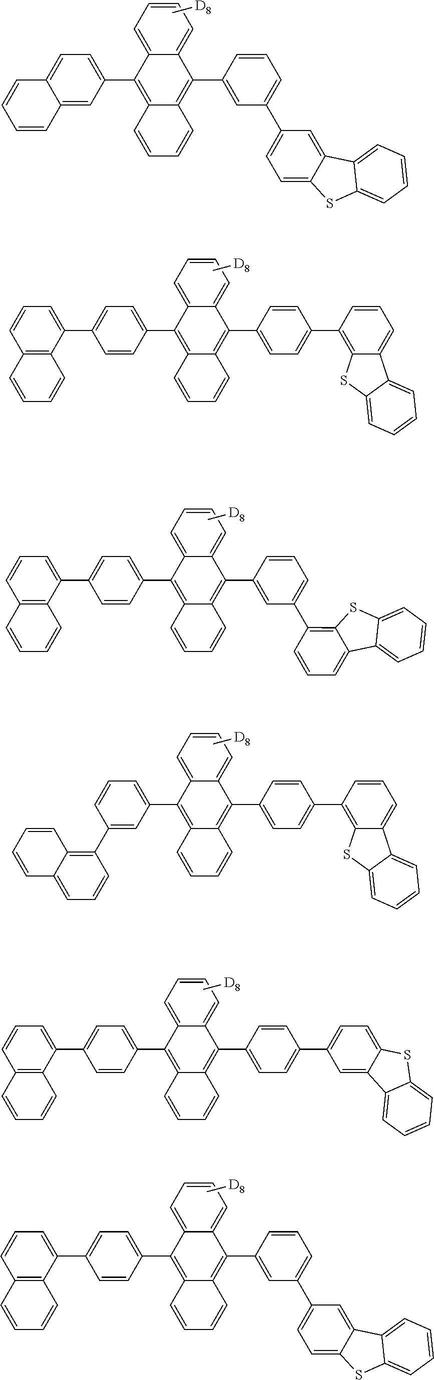

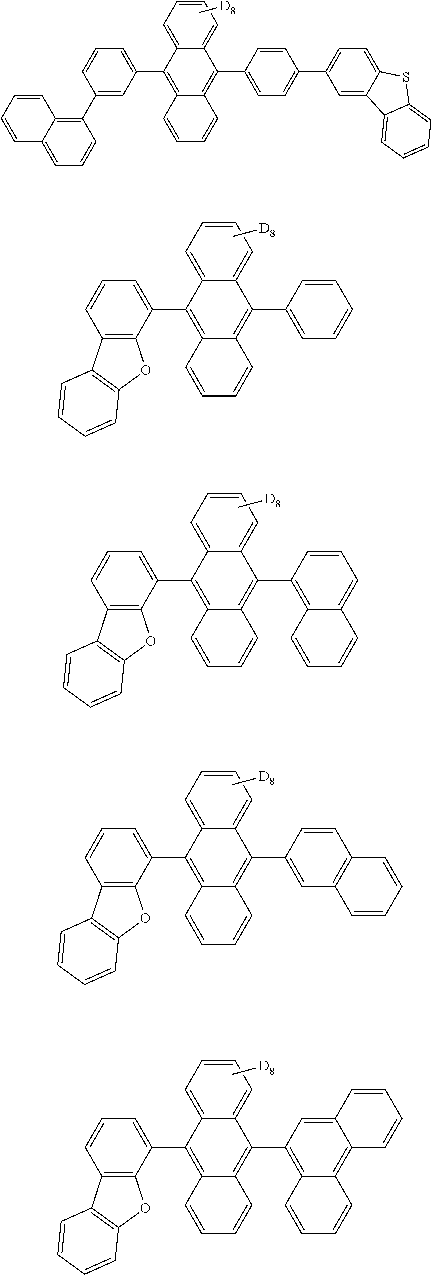

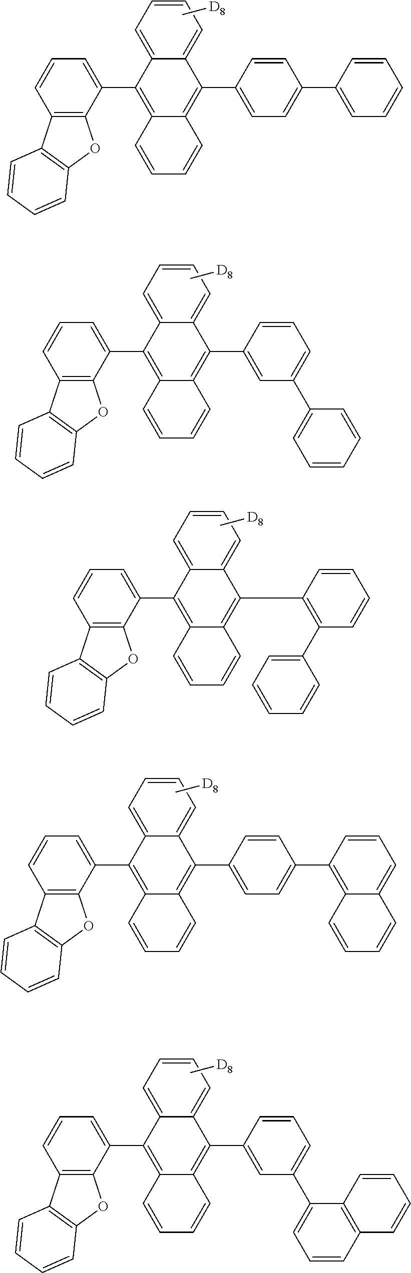

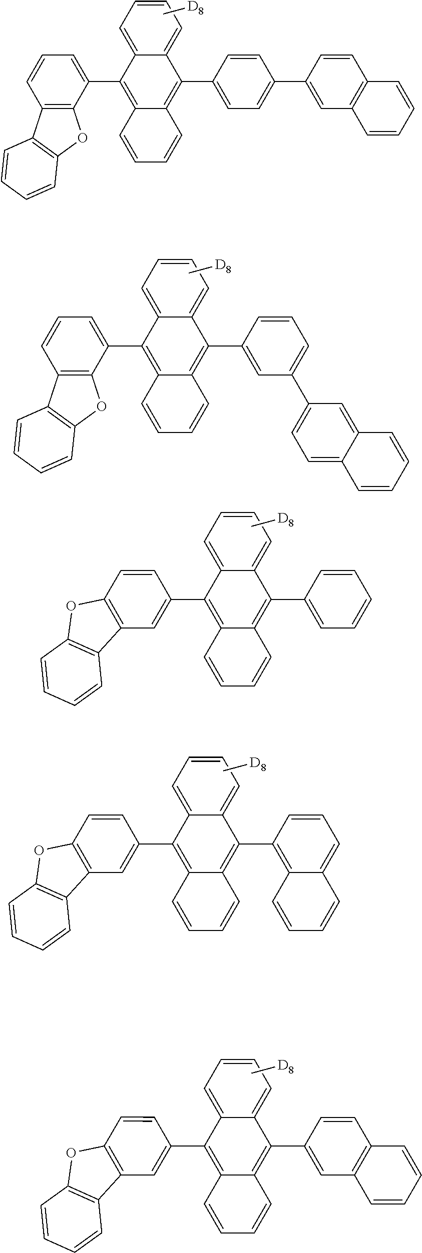

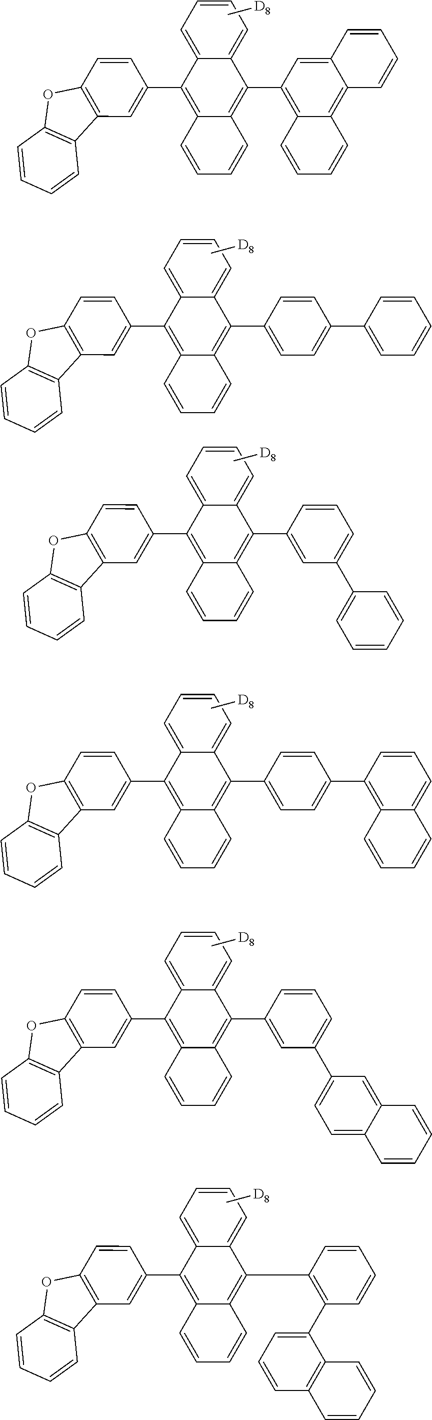

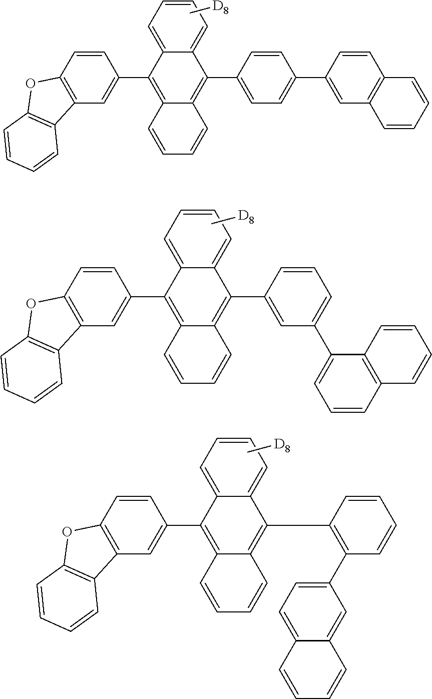

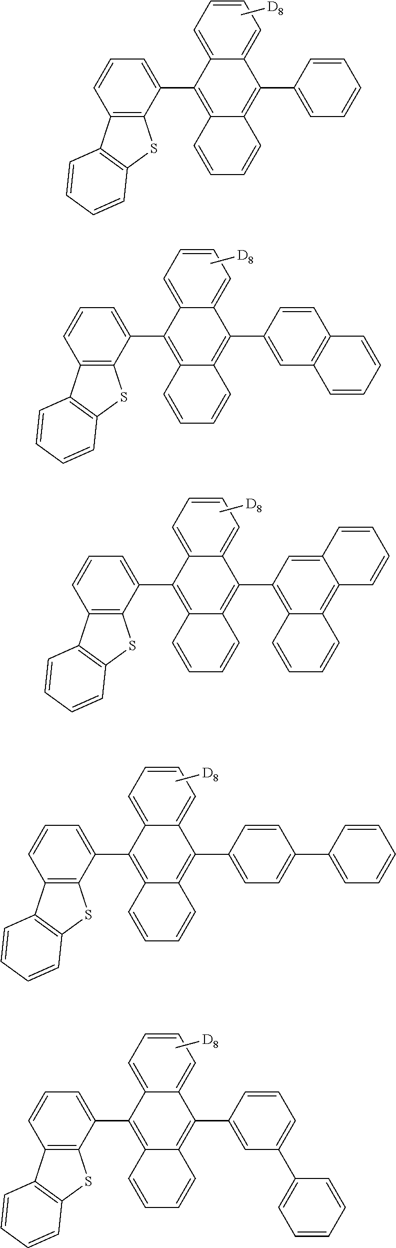

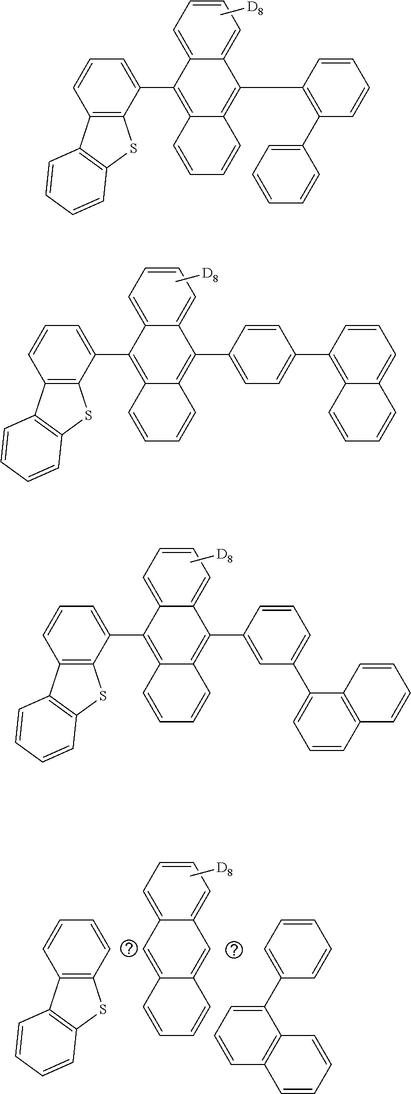

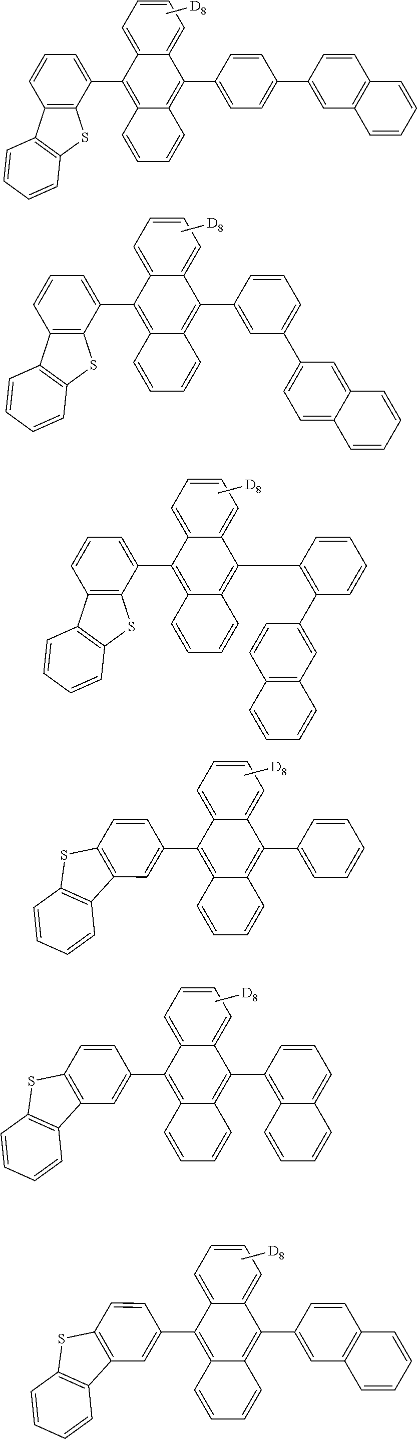

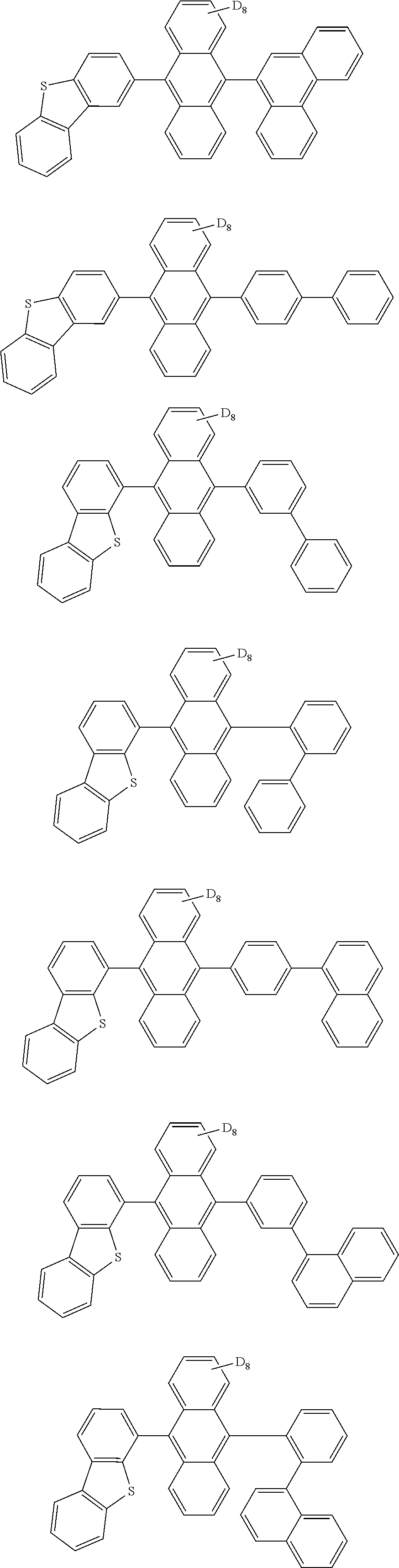

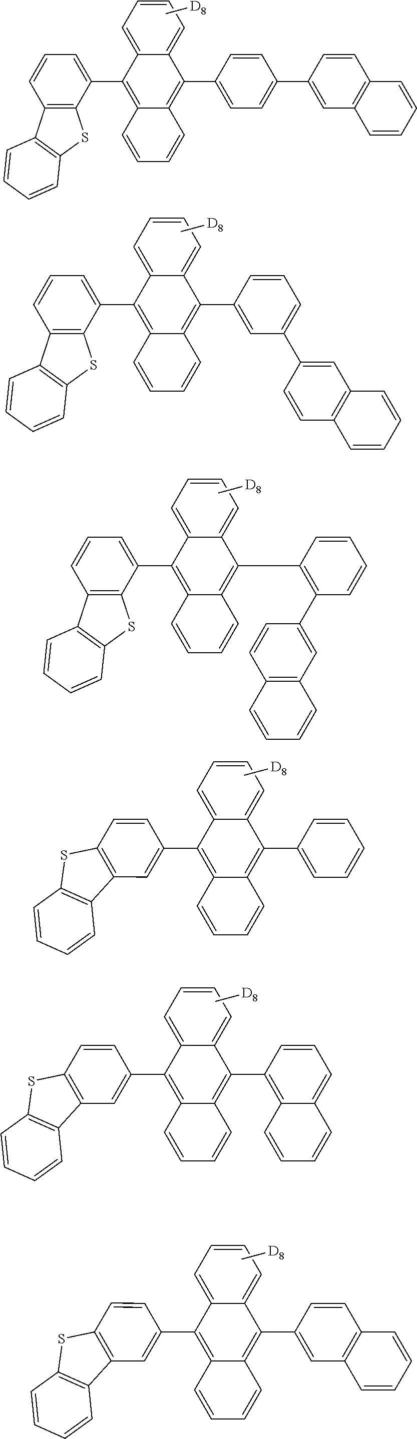

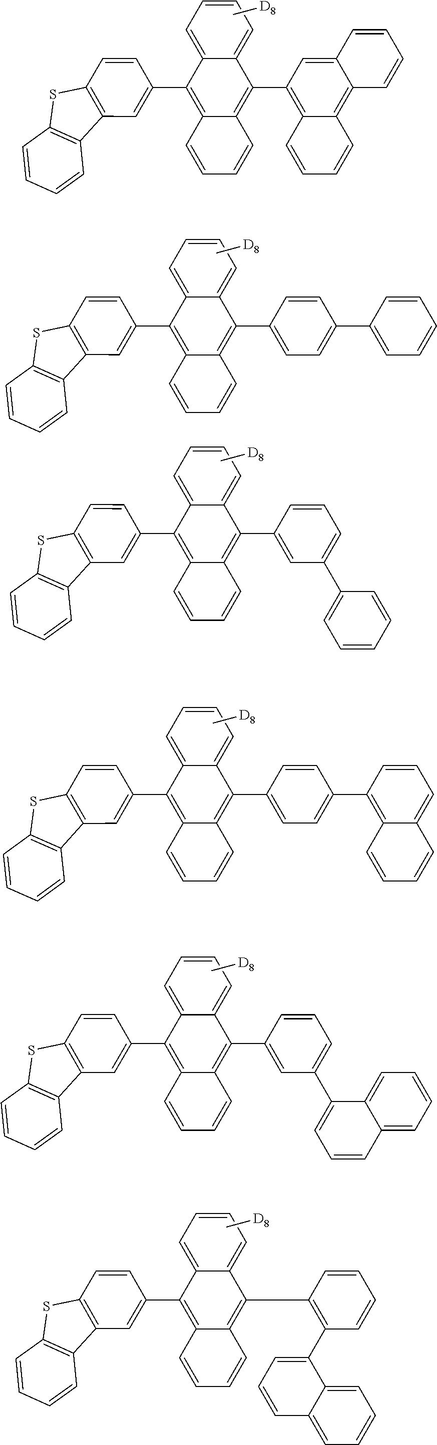

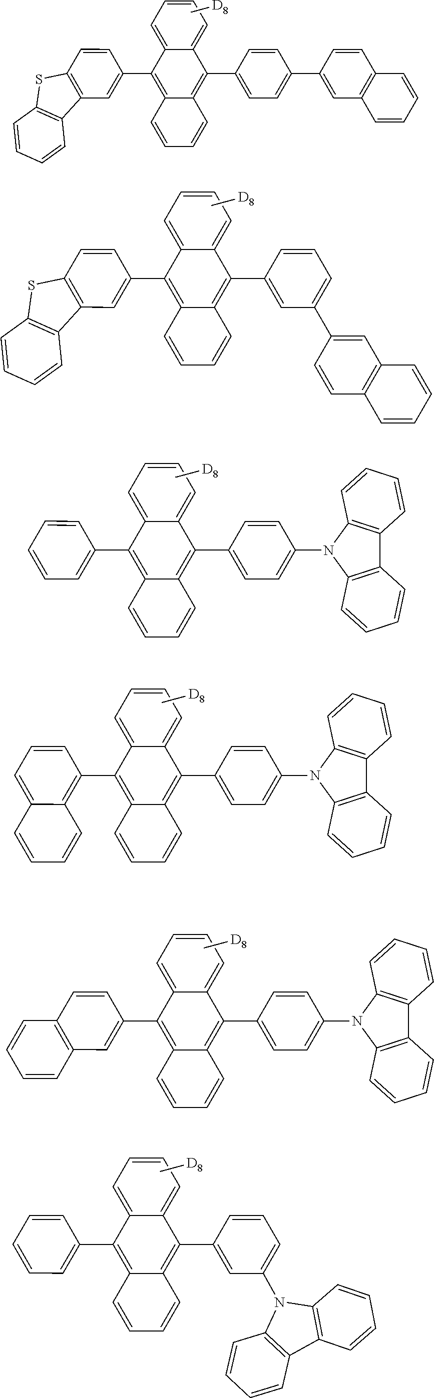

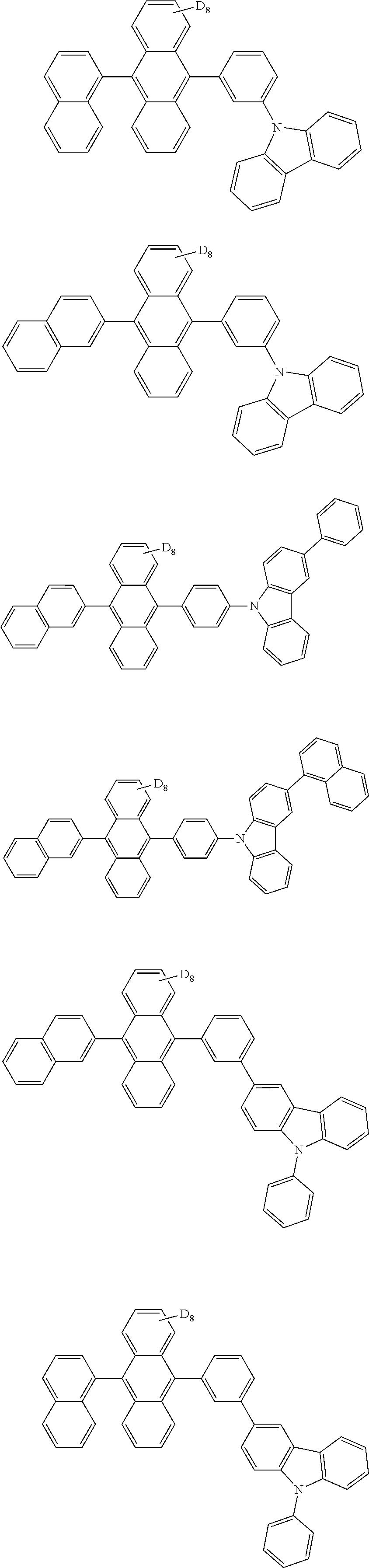

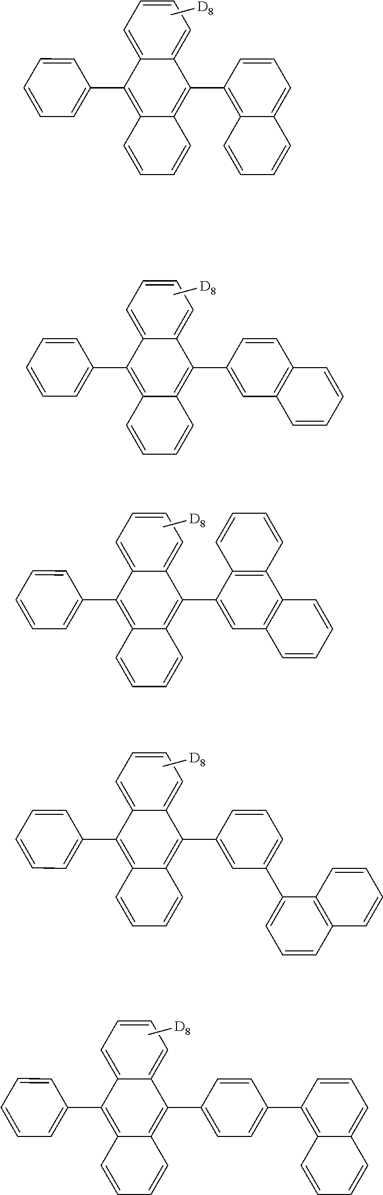

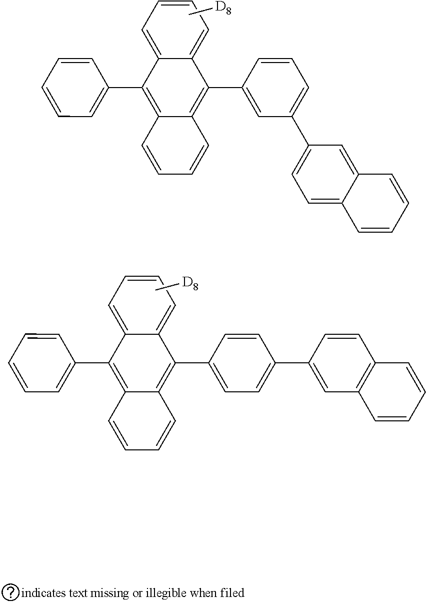

On the other hand, in the EML 240, when the pyrene core of the dopant 244 is deuterated (e.g., “core-deuterated pyrene derivative”), the host 242 can be non-deuterated (e.g., “non-deuterated anthracene derivative”) or all of the anthracene core and a substituent of the host 242 can be deuterated (e.g., “wholly-deuterated anthracene derivative”). Alternatively, the anthracene core of the host 242 except the substituent can be deuterated (e.g., “core-deuterated anthracene derivative”), or the substituent of the host 242 except the anthracene core can be deuterated (e.g., “substituent-deuterated anthracene derivative”).

The pyrene derivative as the dopant 244, in which the pyrene core is deuterated, can be represented by Formula 3:

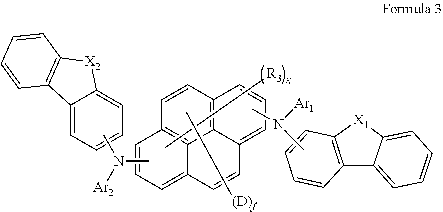

In Formula 3, each of X₁ and X₂ is independently O or S, each of Ar₁ and Ar₂ is independently C₆˜C₃₀ aryl group or C₅˜C₃₀ heteroaryl group, and R₃ is C₁˜C₁₀ alkyl group or C₁˜C₁₀ cycloalkyl group. In addition, f is an integer of 1 to 8, g is an integer of 0 to 2, and a summation off and g is 8 or less.

Namely, in the core-deuterated pyrene derivative as the dopant 244, the pyrene moiety as the core is substituted by deuterium (D), and the substituent except the pyrene moiety is not deuterated.

For example, each of Ar₁ and Ar₂ can be selected from the group consisting of phenyl, dibenzofuranyl, dibenzothiophenyl, dimethylfluorenyl, pyridyl, and quinolinyl and can be substituted by C₁˜C₁₀ alkyl group or C₁˜C₁₀ cycloalkyl group, trimethylsilyl, or trifluoromethyl. In addition, R₃ can be methyl, ethyl, propyl, butyl, heptyl, cyclopentyl, cyclobutyl, or cyclopropyl.

In an exemplary embodiment, the dopant 244 of Formula 3 can be a compound being one of the followings in Formula 4:

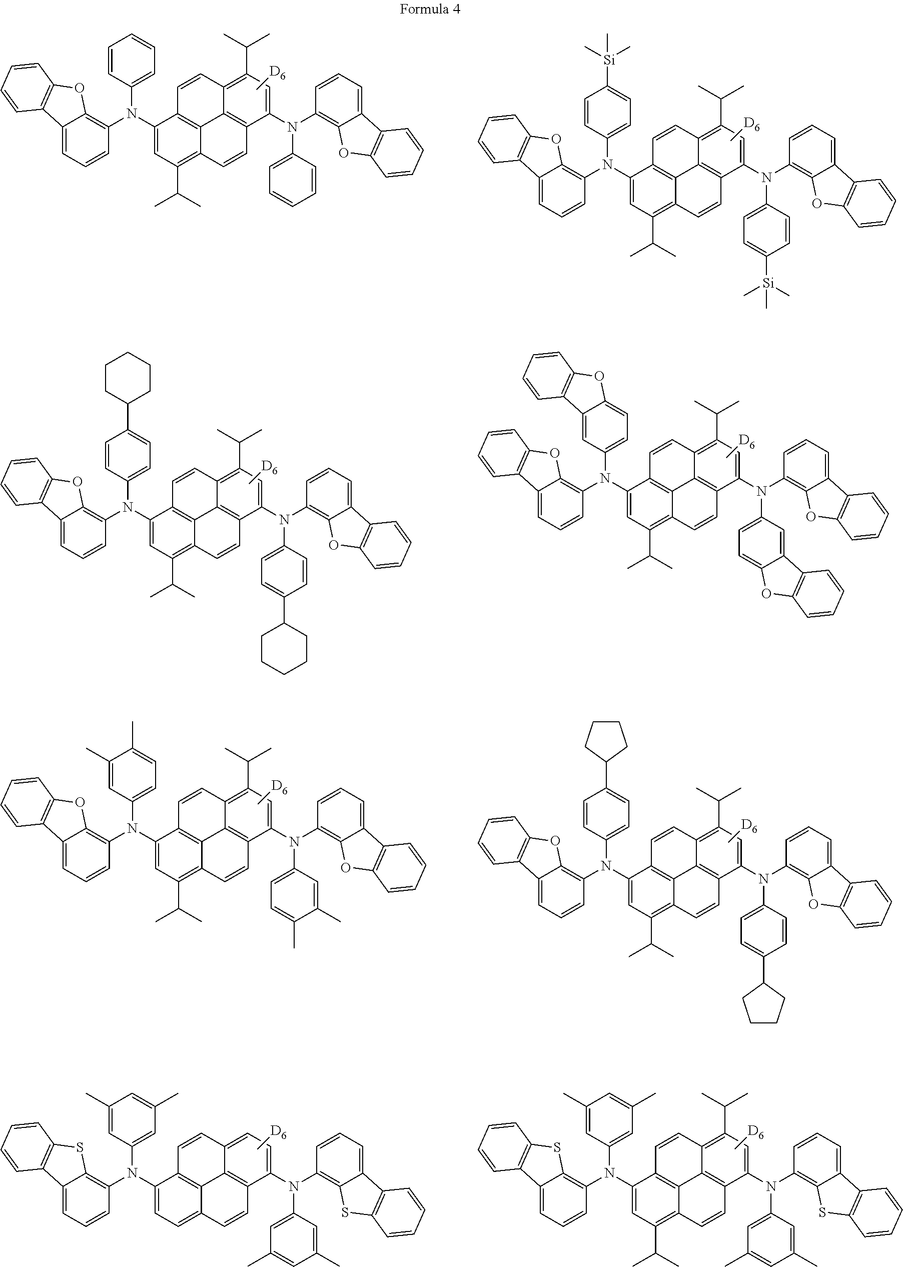

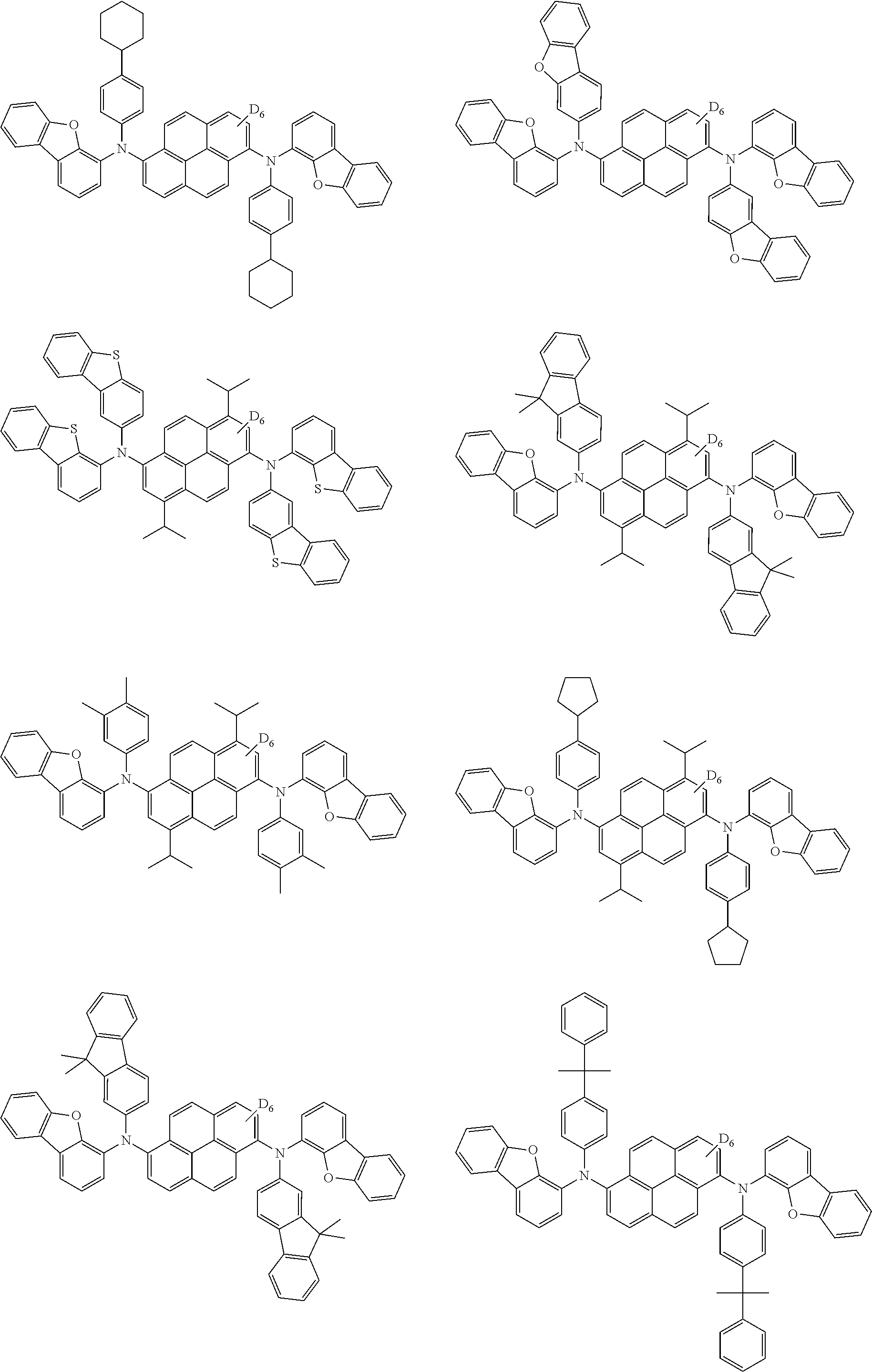

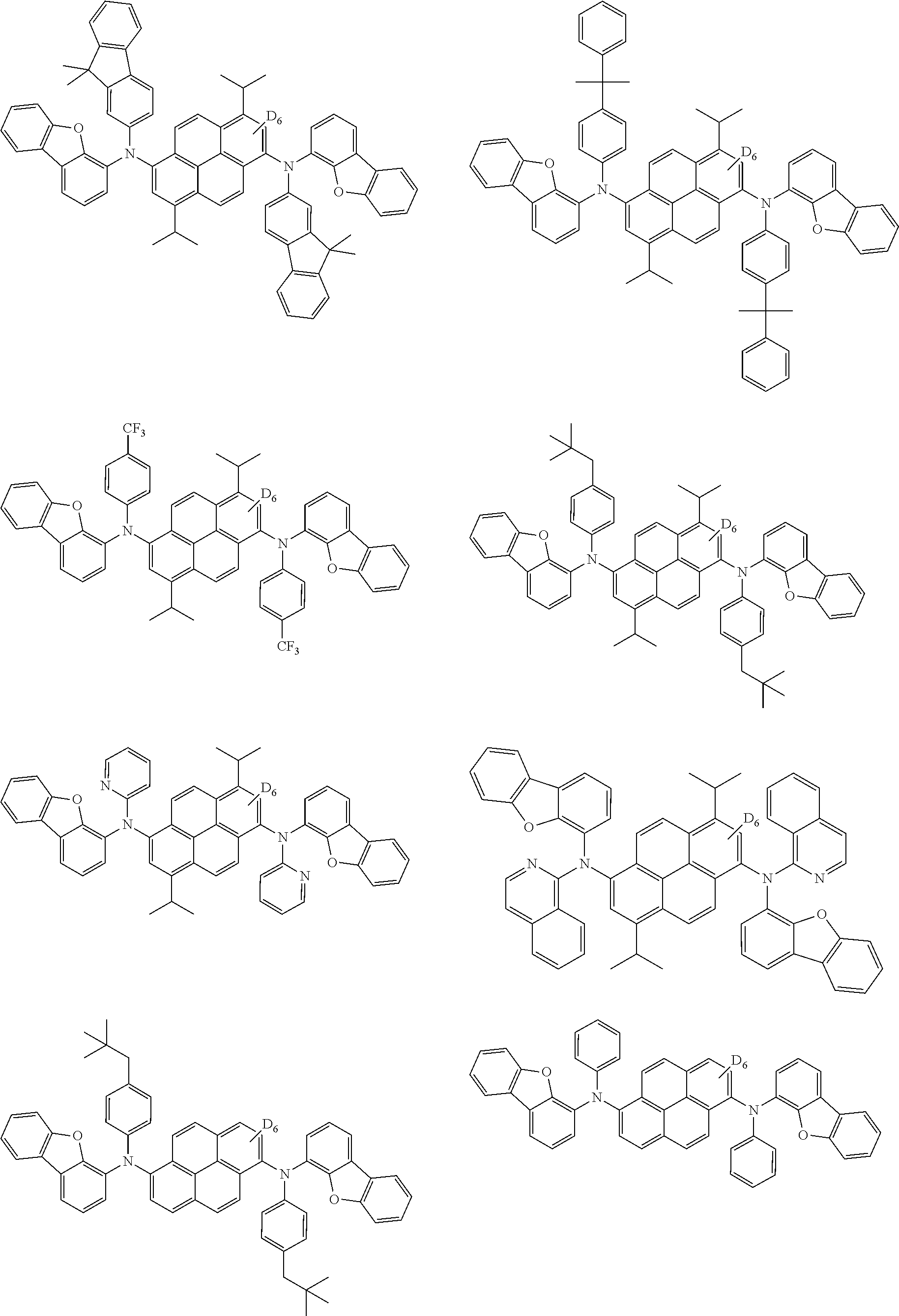

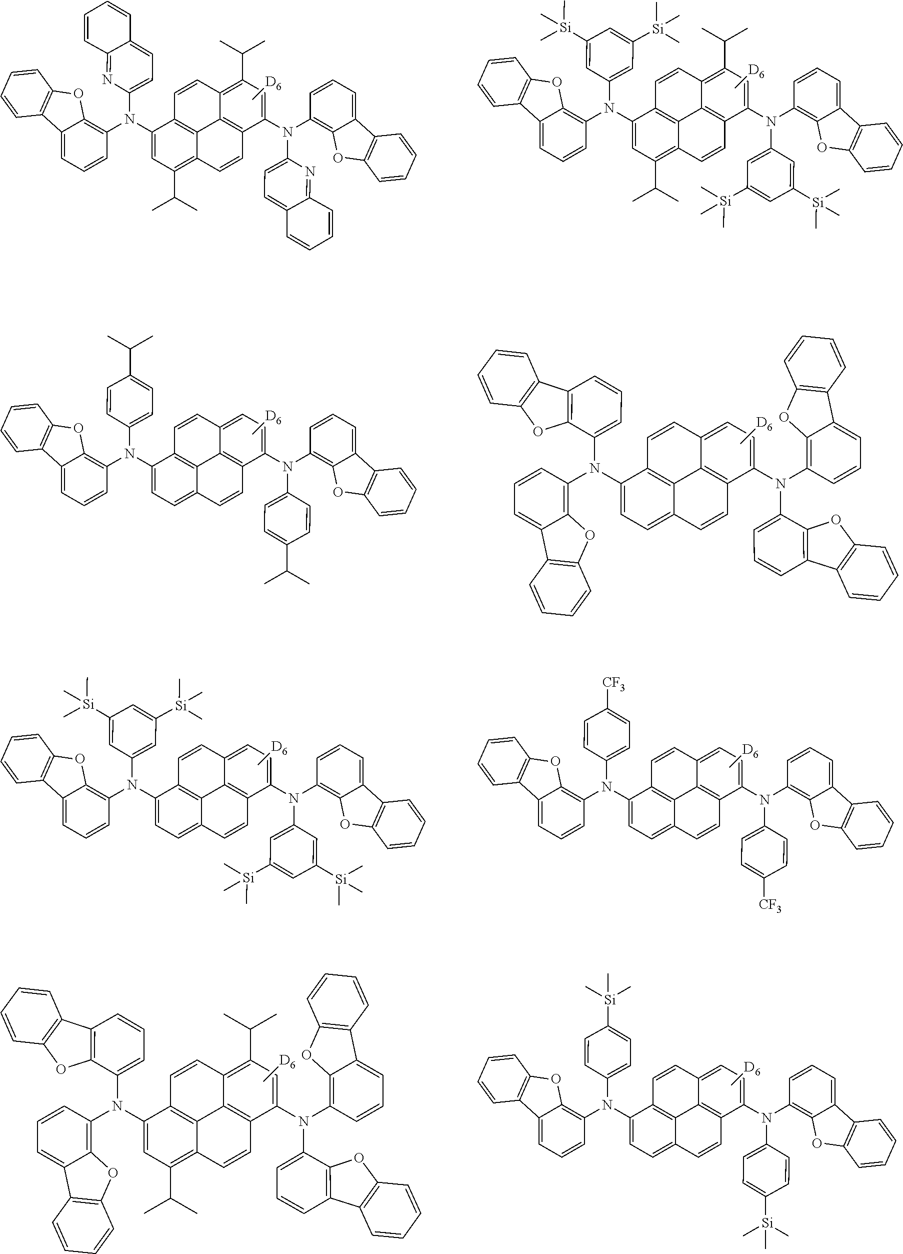

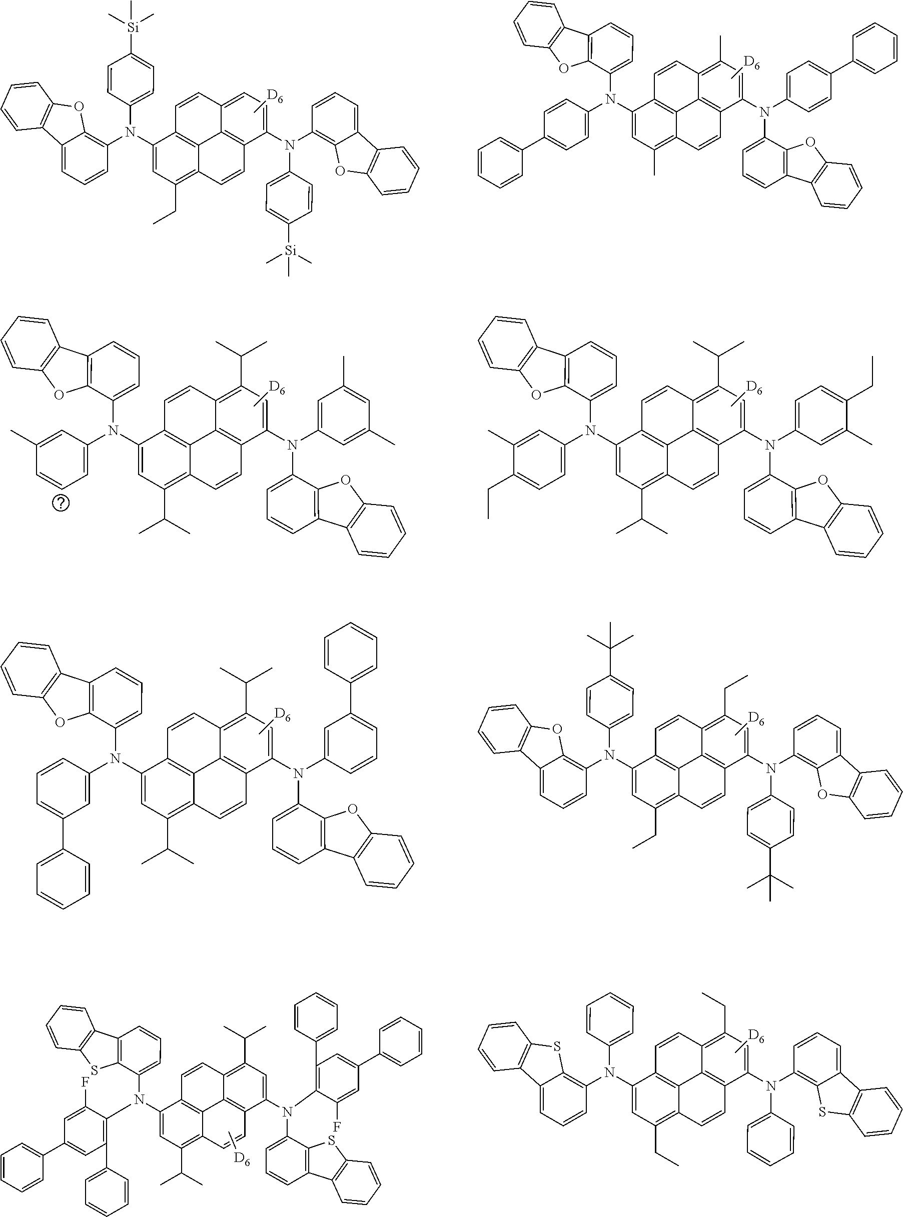

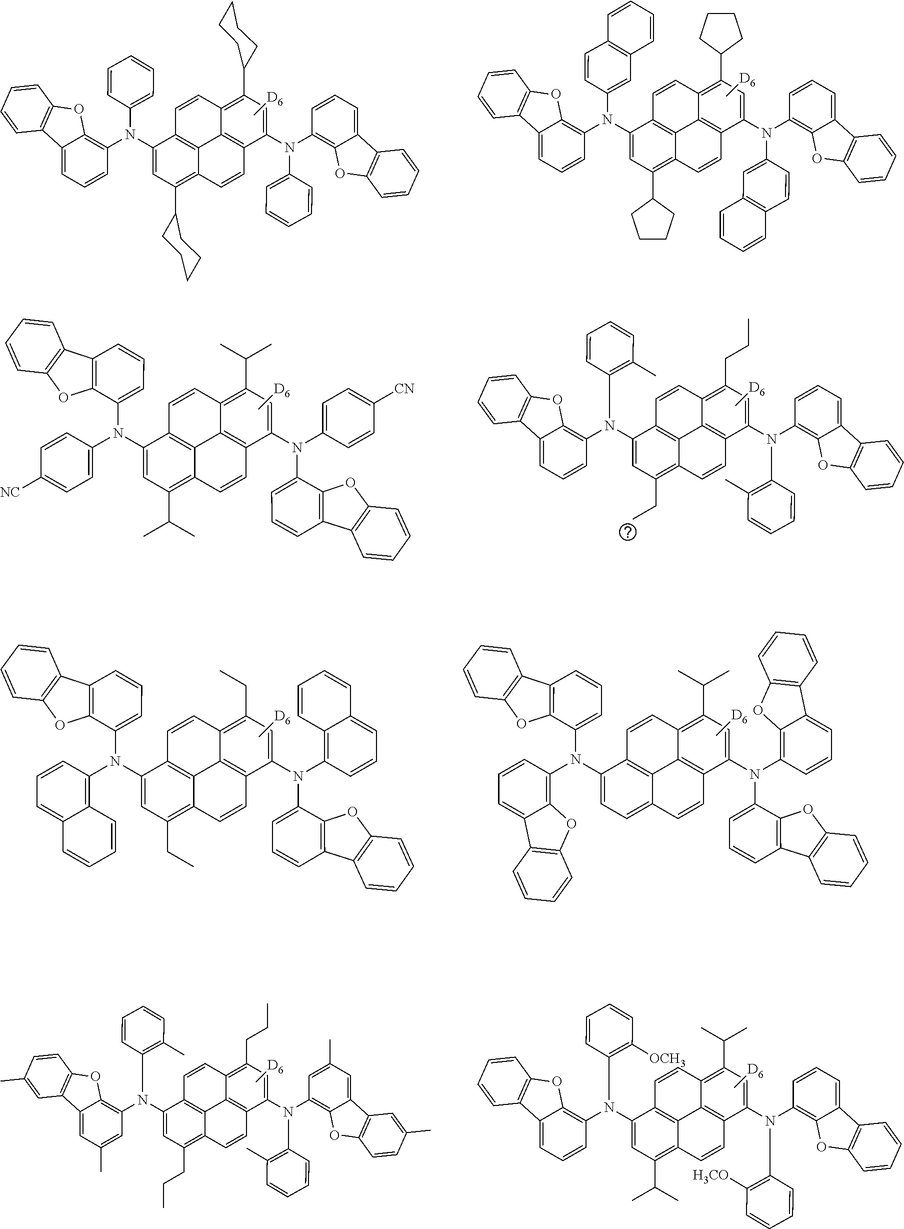

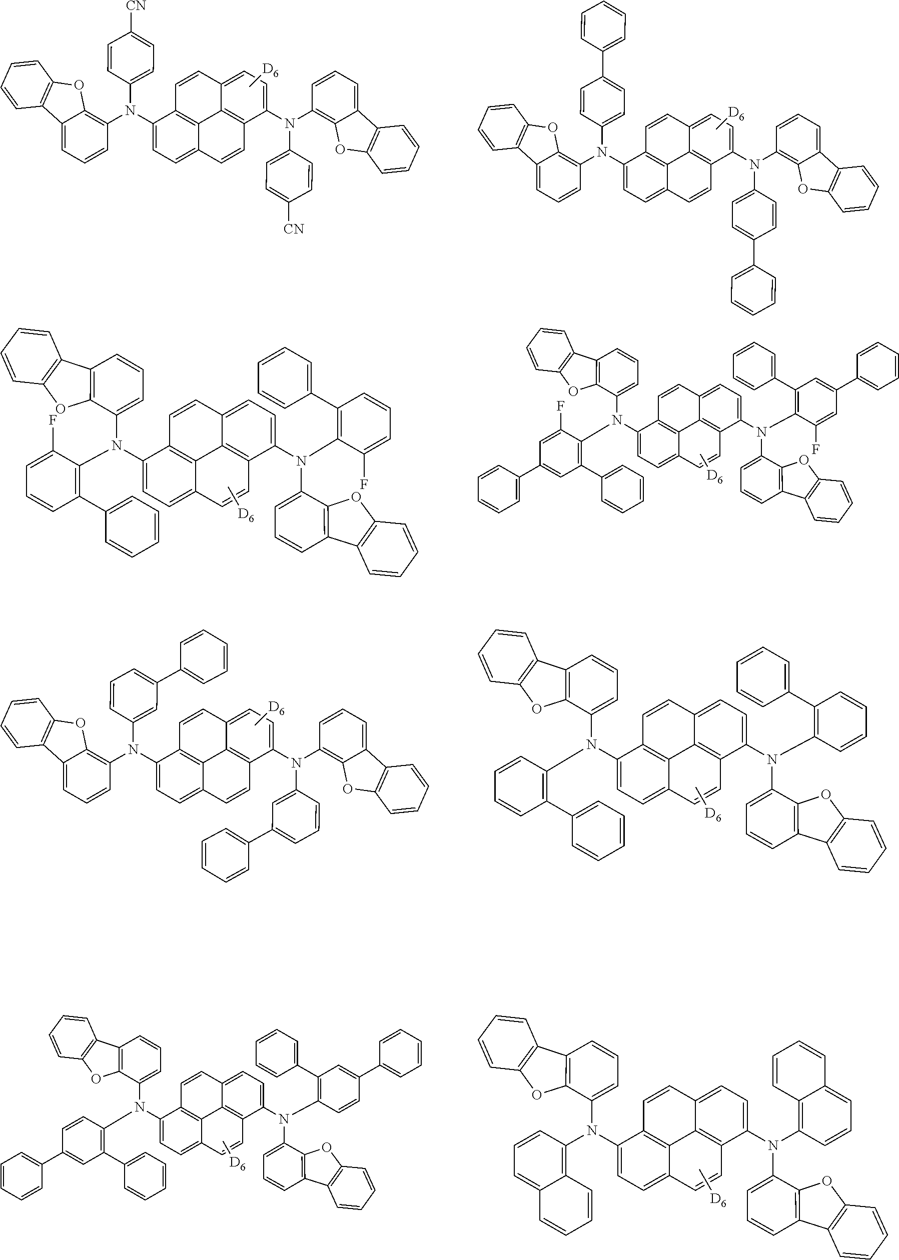

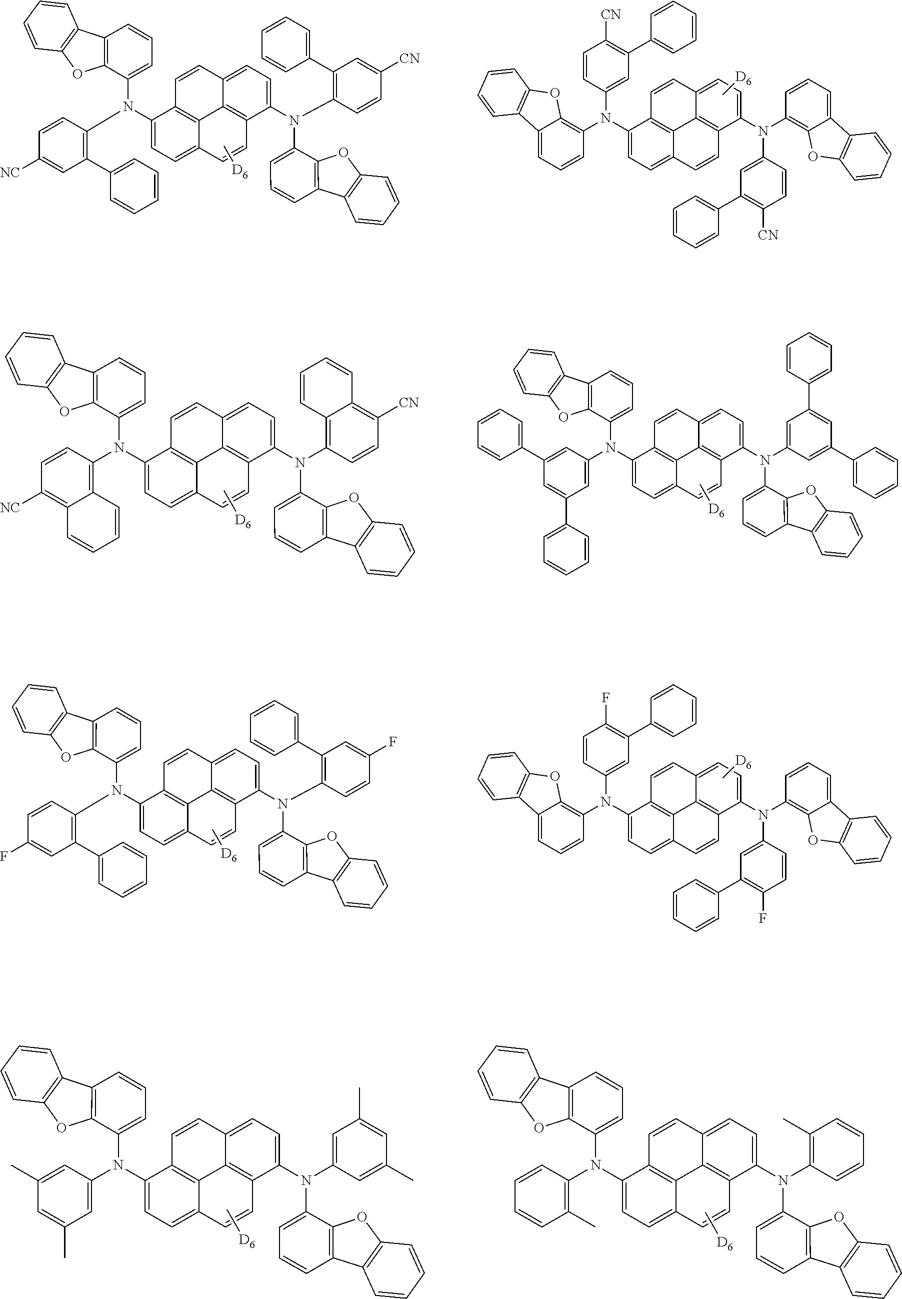

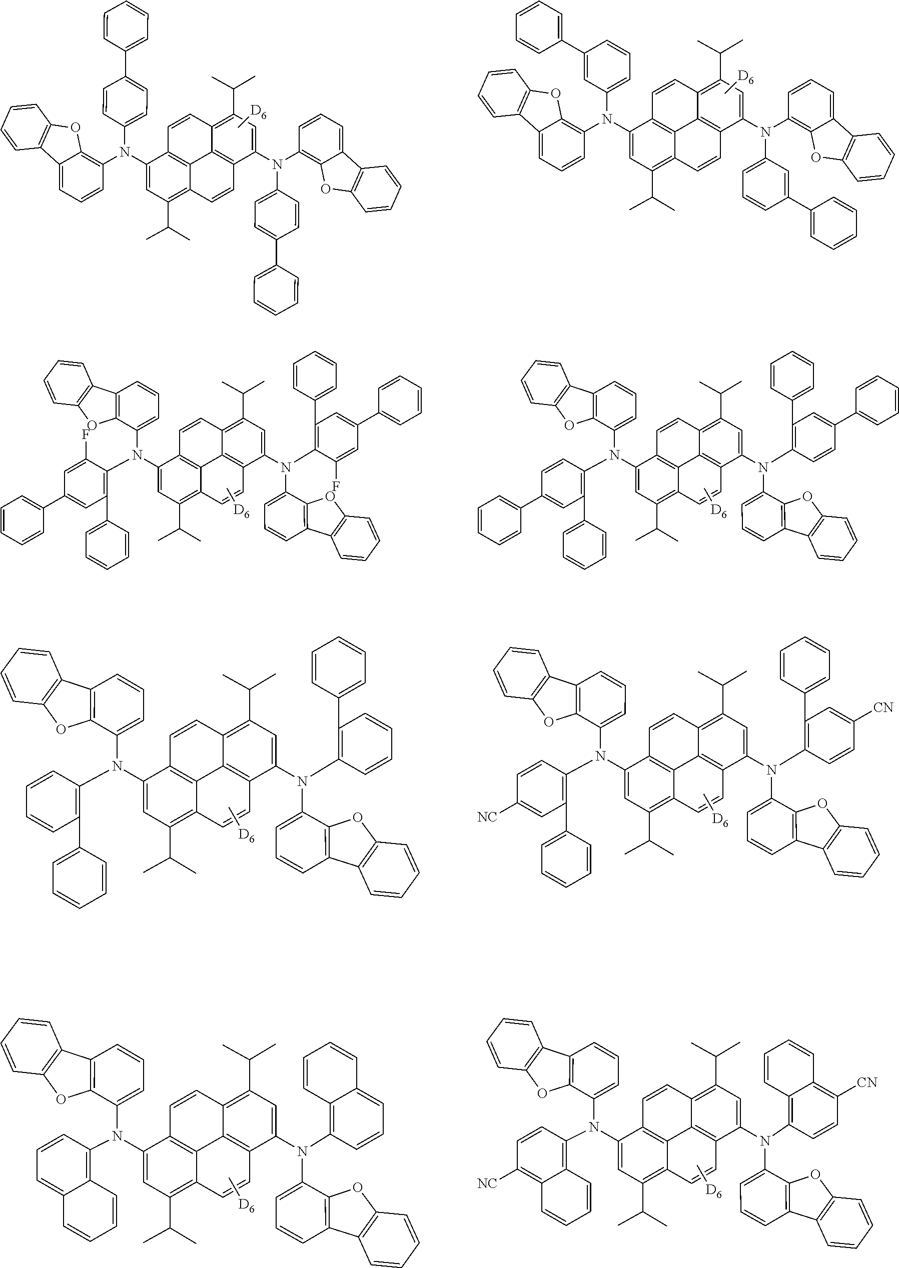

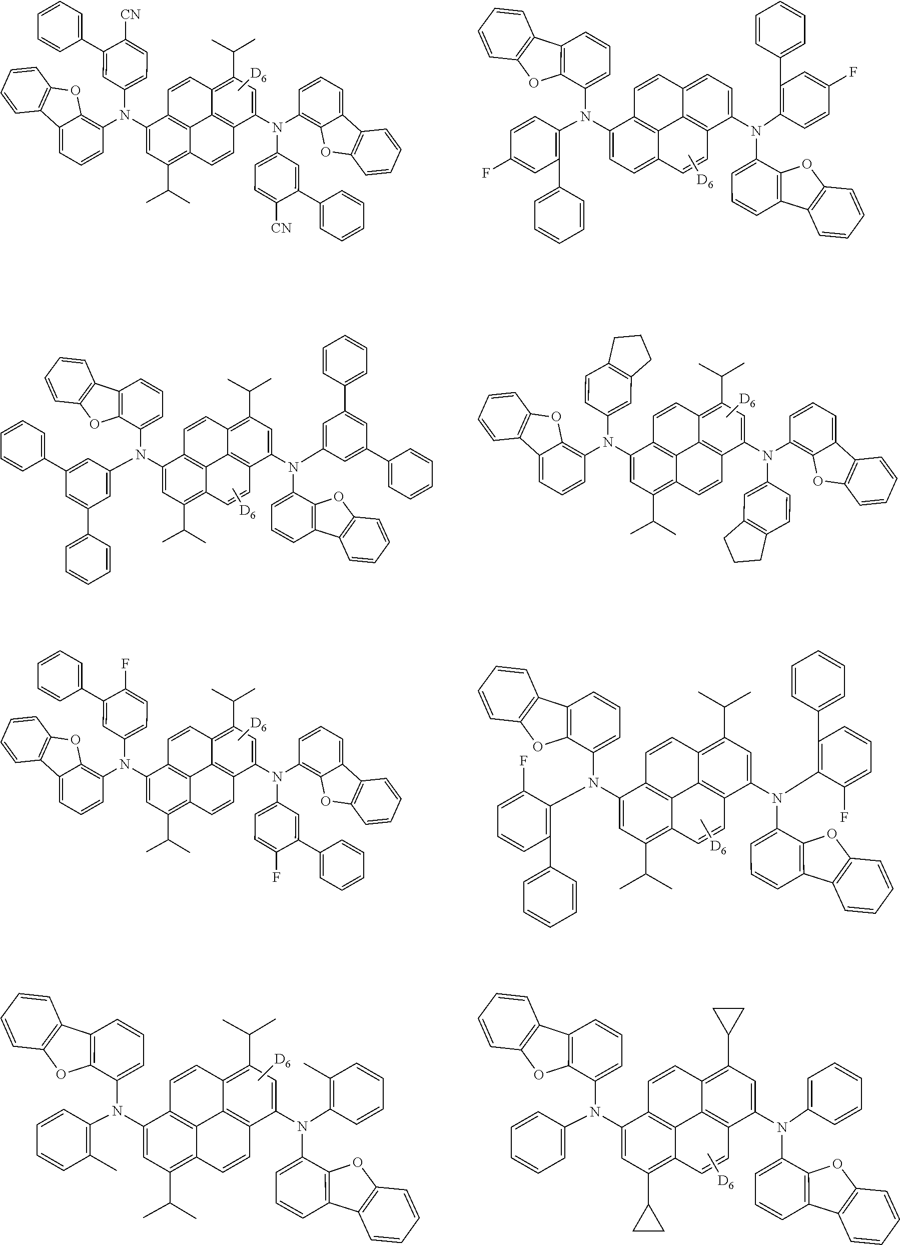

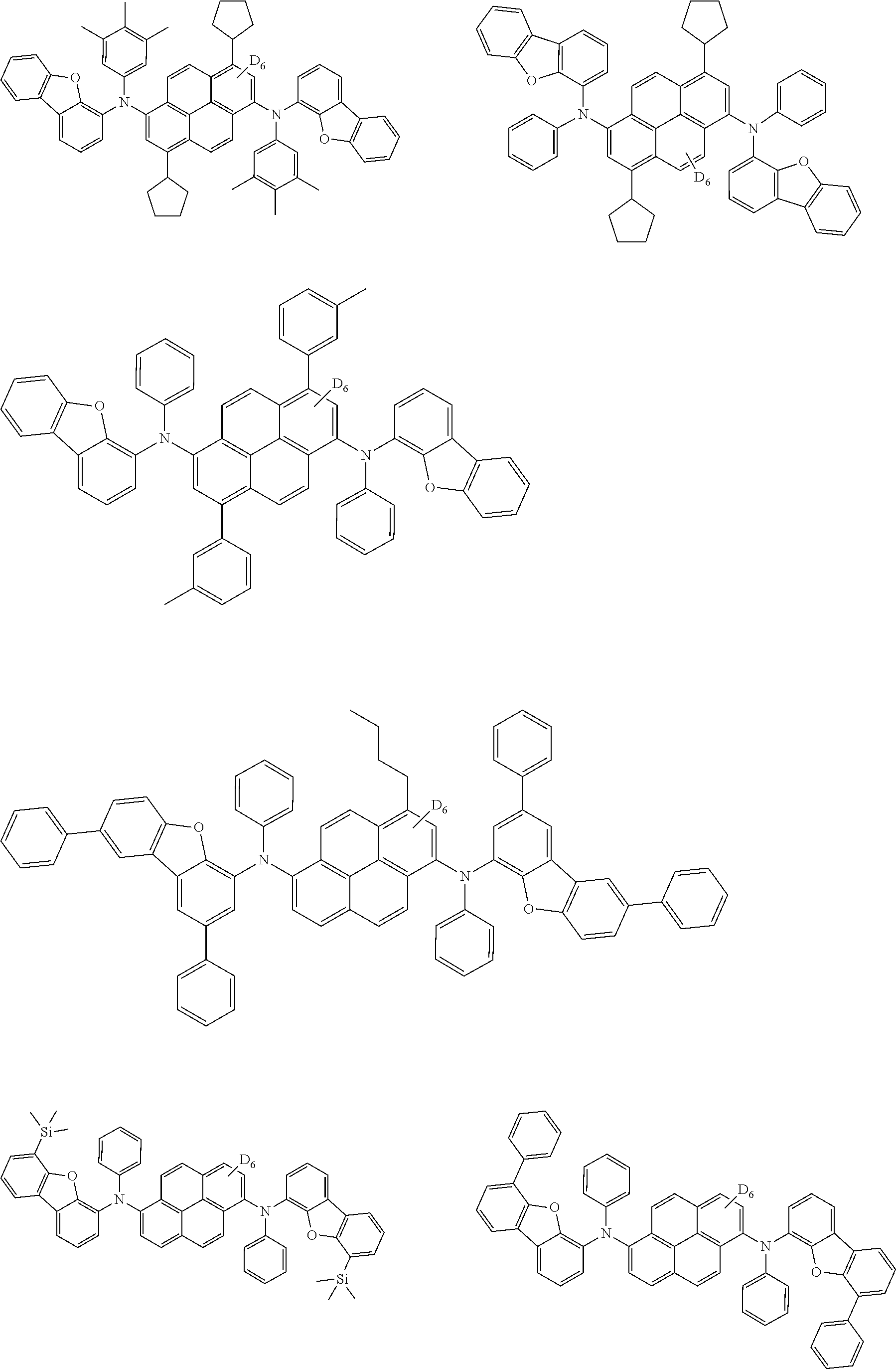

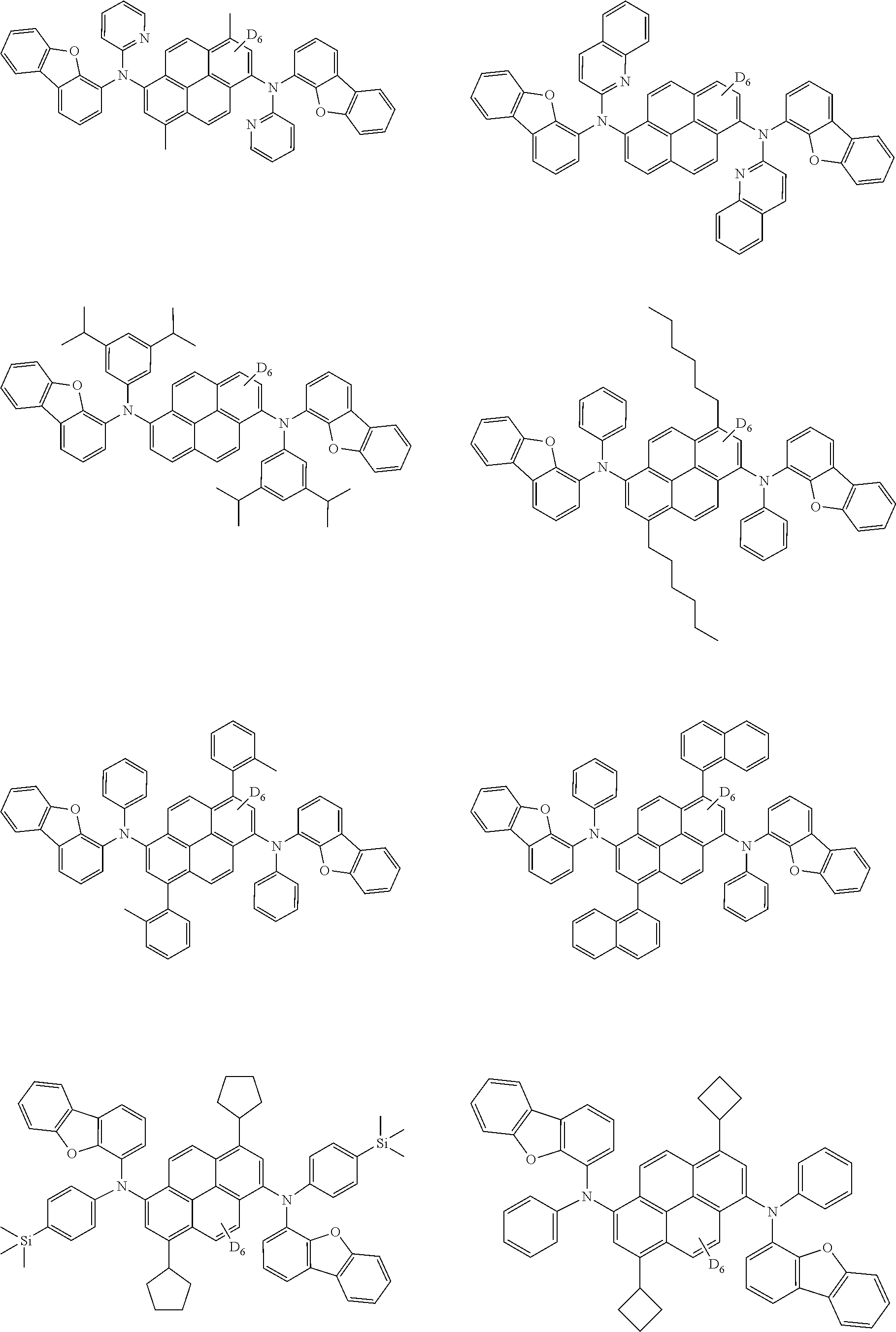

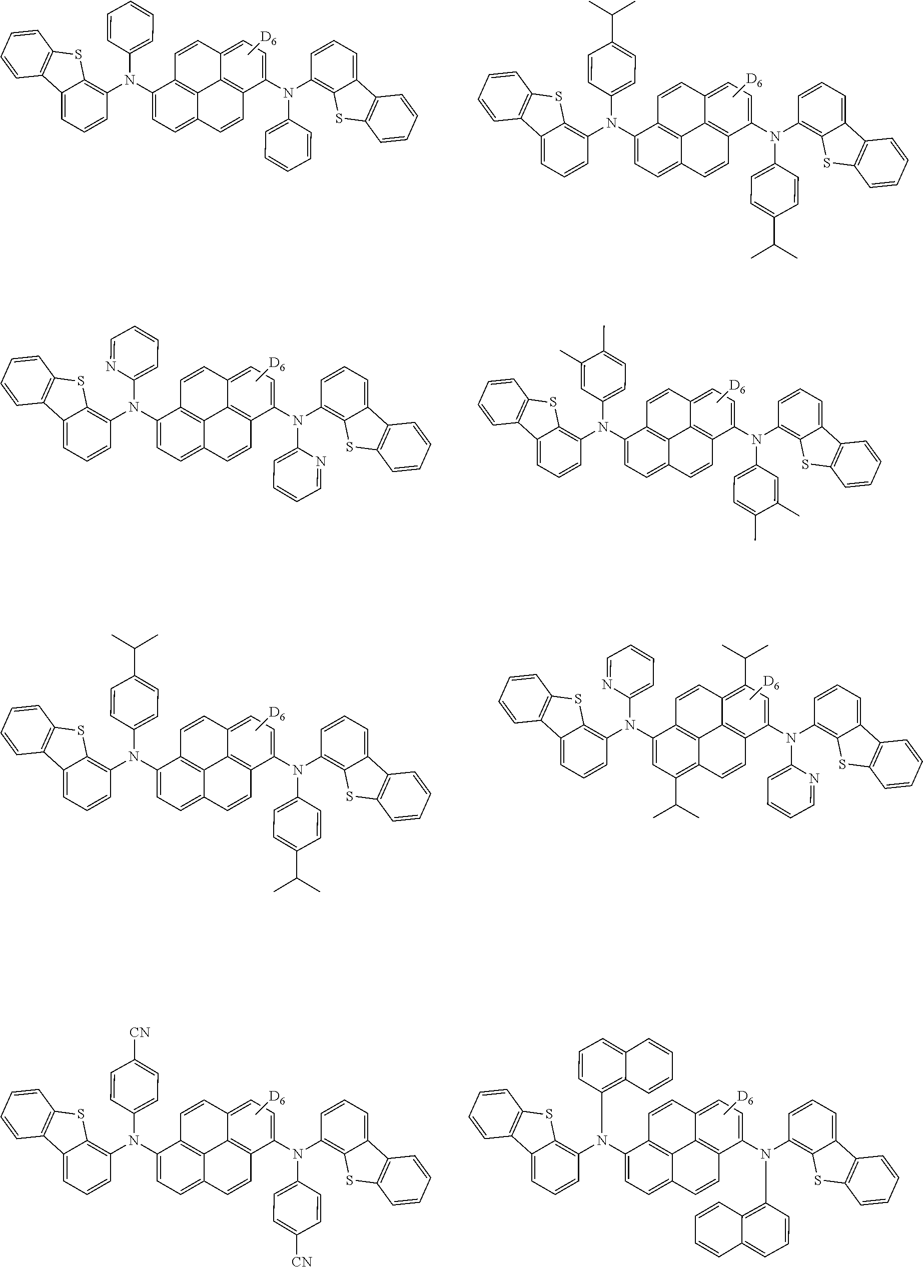

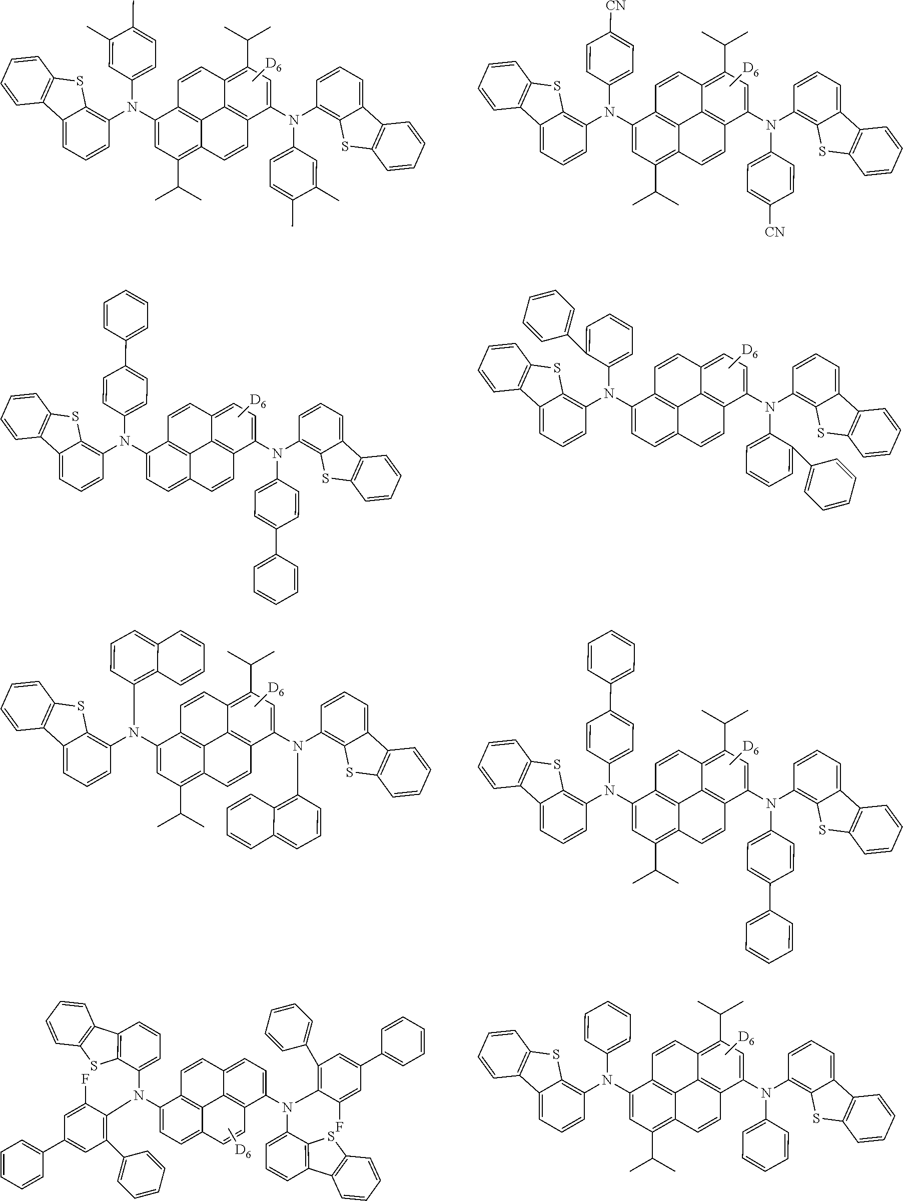

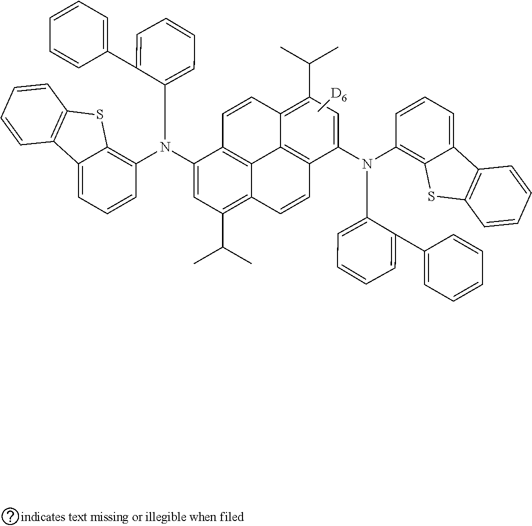

For example, when the host 242 is a compound of Formula 1, the dopant 244 can be a compound of one of Formula 3 and Formulas 5-1 to 5-3.

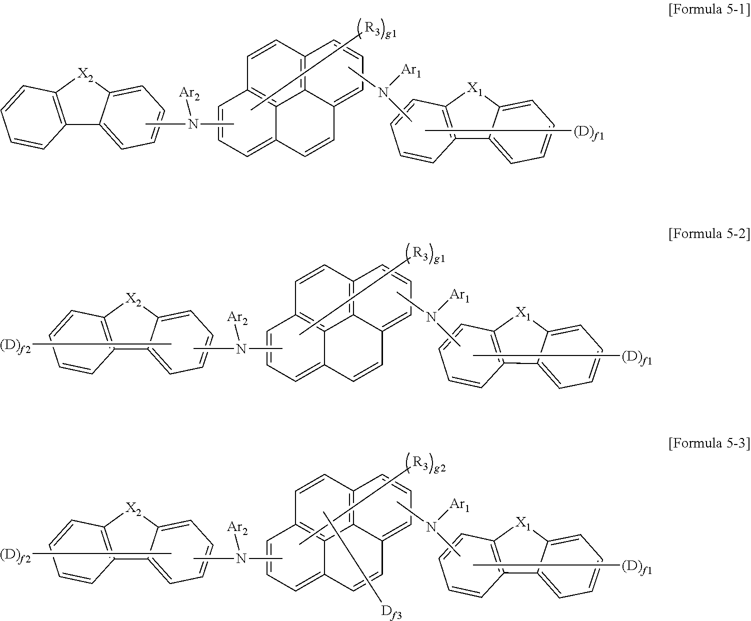

In Formulas 5-1 to 5-3, each of X₁ and X₂ is independently O or S, each of Ar₁ and Ar₂ is independently C₆˜C₃₀ aryl group or C₅˜C₃₀ heteroaryl group, and R₃ is C₁˜C₁₀ alkyl group or C₁˜C₁₀ cycloalkyl group. In addition, each of f1 and f2 is independently an integer of 1 to 7, and g1 is an integer of 0 to 8. In Formula 5-3, f3 is an integer of 1 to 8, g2 is an integer of 0 to 2, and a summation of f3 and g2 is 8. In addition, a part or all of hydrogen atoms of Ar₁ and Ar₂ can be substituted by D.

When the dopant 244 is a compound of Formula 3, the host 242 is one of a compound of Formula 1, a compound of Formula 1, in which at least one of L1, L2, L3, L4, R1 and R2 is deuterated, and a compound of Formula 1, in which the anthracene core is not deuterated (e=0) and at least one of L1, L2, L3, L4, R1 and R2 is deuterated.

In the EML 240 of the OLED D, the host 242 can have a weight % of about 70 to 99.9, and the dopant 244 can have a weight % of about 0.1 to 30. To provide sufficient emitting efficiency and lifespan, a weight % of the dopant 244 can be about 0.1 to 10, preferably about 1 to 5.

As mentioned above, the EML 240 of the OLED D includes the host 242 of the anthracene derivative, the dopant 244 of the pyrene derivative, and at least one of an anthracene core of the anthracene derivative 242 and a pyrene core of the pyrene derivative 244 is deuterated. As a result, the OLED D and the organic light emitting display device 100 have advantages in the emitting efficiency and the lifespan.

### Synthesis of the Host

### 1. Synthesis of the Compound Host1D

### (1) Compound H-1

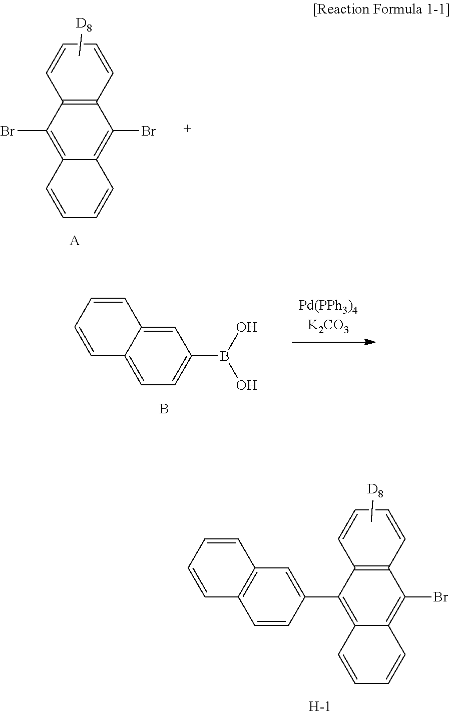

The compound A (11.90 mmol) and and the compound B (13.12 mmol) were dissolved in toluene (100 mL), Pd(PPh₃)₄ (0.59 mmol) and 2M K₂CO₃ (24 mL) were slowly added into the mixture. The mixture was reacted for 48 hours. After cooling, the temperature is set to the room temperature, and the solvent was removed under the reduced pressure. The reaction mixture was extracted with chloroform. The extracted solution was washed twice with sodium chloride supersaturated solution and water, and then the organic layer was collected and dried over anhydrous magnesium sulfate. Thereafter, the solvent was evaporated to obtain a crude product, and the column chromatography using silica gel was performed to the crude product to obtain the compound H-1. (2.27 g, 57%)

### (2) Compound Host1D

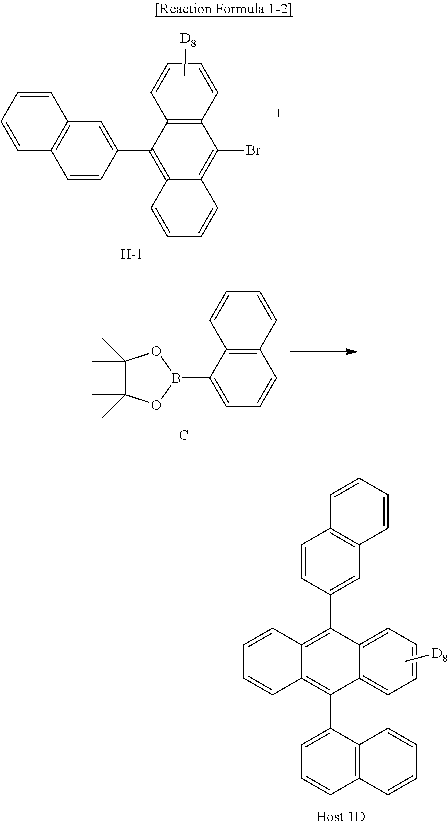

The compound H-1 (5.23 mmol), the compound C (5.74 mmol), tris(dibenzylideneacetone)dipalladium(0) (0.26 mmol) and toluene (50 mL) were added to the flask (250 mL) in a glove box. After the reaction flask was removed from the drying box, degassed aqueous sodium carbonate (2M, 20 mL) was added to the mixture. The mixture was stirred and heated at 90° C. overnight. The reaction was monitored by high-performance liquid chromatography (HPLC). After cooling to the room temperature, the organic layer was separated. The aqueous layer was washed twice with dichloromethane (DCM), and the organic layer was concentrated by rotary evaporation to obtain a gray powder. The compound Host1D was obtained by performing purification using neutral alumina, precipitation using hexane, and column chromatography using silica gel. (2.00 g, 89%)

### 2. Synthesis of the Compound Host2D

### (1) Compound H-2

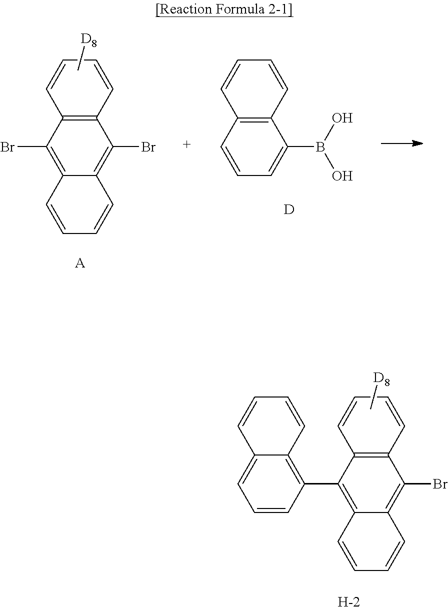

In the synthesis of the compound H-1, the compound D was used instead of the compound B to obtain the compound H-2.

### (2) Compound Host2D

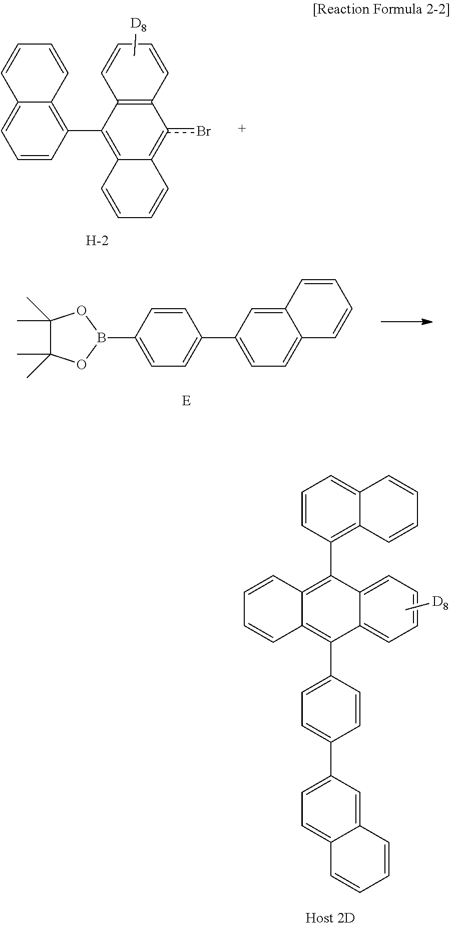

The compound H-2 (5.23 mmol), the compound E (5.74 mmol), tris(dibenzylideneacetone)dipalladium(0) (0.26 mmol) and toluene (50 mL) were added to the flask (250 mL) in a glove box. After the reaction flask was removed from the drying box, degassed aqueous sodium carbonate (2M, 20 mL) was added to the mixture. The mixture was stirred and heated at 90° C. overnight. The reaction was monitored by HPLC. After cooling to the room temperature, the organic layer was separated. The aqueous layer was washed twice with DCM, and the organic layer was concentrated by rotary evaporation to obtain a gray powder. The compound Host2D was obtained by performing purification using neutral alumina, precipitation using hexane, and column chromatography using silica gel. (2.28 g, 86%)

### 3. Synthesis of the Compound Host3D

### (1) Compound H-3

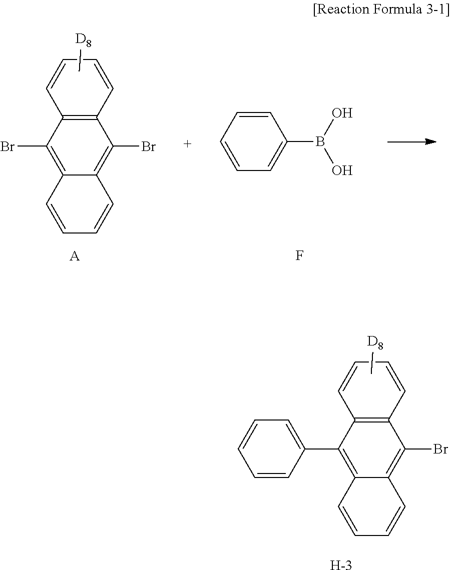

In the synthesis of the compound H-1, the compound F was used instead of the compound B to obtain the compound H-3.

### (2) Compound Host3D

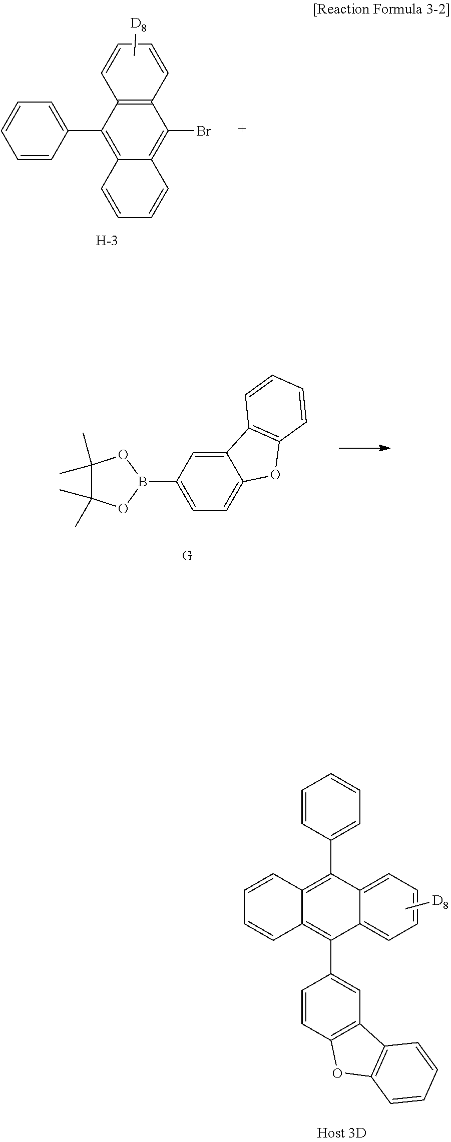

The compound H-3 (5.23 mmol), the compound G (5.74 mmol), tris(dibenzylideneacetone)dipalladium(0) (0.26 mmol) and toluene (50 mL) were added to the flask (250 mL) in a glove box. After the reaction flask was removed from the drying box, degassed aqueous sodium carbonate (2M, 20 mL) was added to the mixture. The mixture was stirred and heated at 90° C. overnight. The reaction was monitored by HPLC. After cooling to the room temperature, the organic layer was separated. The aqueous layer was washed twice with DCM, and the organic layer was concentrated by rotary evaporation to obtain a gray powder. The compound Host3D was obtained by performing purification using neutral alumina, precipitation using hexane, and column chromatography using silica gel. (1.71 g, 78%)

### 4. Synthesis of the Compound Host4D

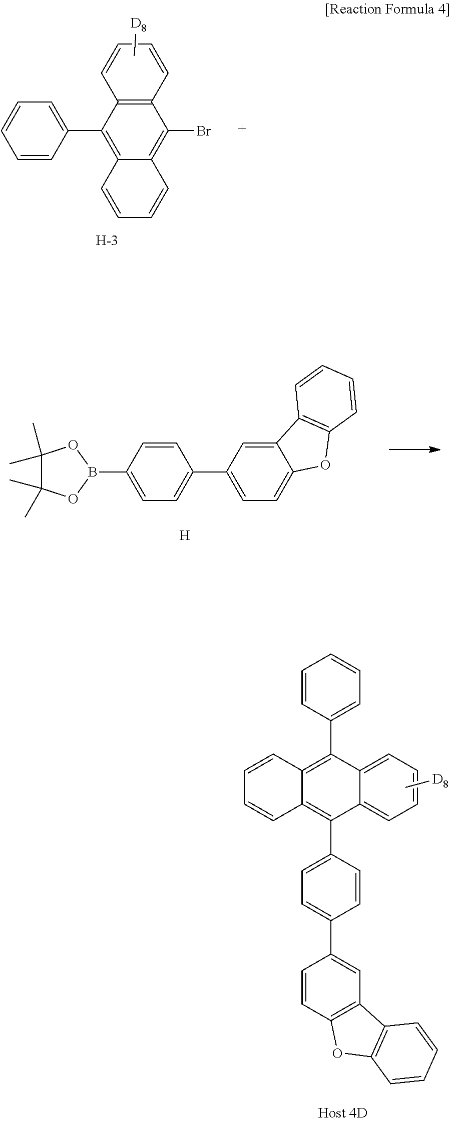

The compound H-3 (5.23 mmol), the compound H (5.74 mmol), tris(dibenzylideneacetone)dipalladium(0) (0.26 mmol) and toluene (50 mL) were added to the flask (250 mL) in a glove box. After the reaction flask was removed from the drying box, degassed aqueous sodium carbonate (2M, 20 mL) was added to the mixture. The mixture was stirred and heated at 90° C. overnight. The reaction was monitored by HPLC. After cooling to the room temperature, the organic layer was separated. The aqueous layer was washed twice with DCM, and the organic layer was concentrated by rotary evaporation to obtain a gray powder. The compound Host4D was obtained by performing purification using neutral alumina, precipitation using hexane, and column chromatography using silica gel. (1.75 g, 67%)

### Synthesis of the Dopant

### 1. Synthesis of the Compound Dopant1D

### (1) Compound D-1

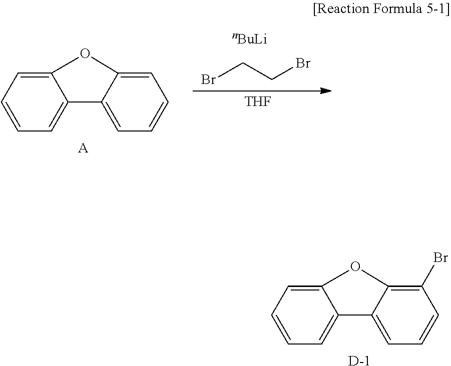

Under argon conditions, dibenzofuran (30.0 g) and dehydrated tetrahydrofuran (THF, 300 mL) were added to a distillation flask (1000 mL). The mixture was cooled to −65° C., and n-butyllithium hexane solution (1.65 M, 120 mL) was added. The mixture was slowly heated up and reacted at the room temperature for 3 hours. After the mixture was cooled to −65° C. again, 1,2-dibromoethane (23.1 mL) was added. The mixture was slowly heated up and reacted at the room temperature for 3 hours. 2N hydrochloric acid and ethyl acetate were added into the mixture for separation and extraction, and the organic layer was washed with water and saturated brine and dried over sodium sulfate. The crude product obtained by concentration was purified by silica gel chromatography using methylene chloride, and the obtained solid was dried under reduced pressure to obtain the compound D-1. (43.0 g)

### (2) Compound D-2

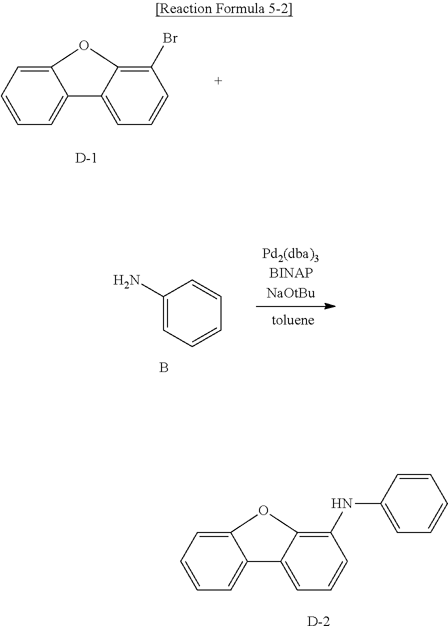

Under argon conditions, the compound D-1 (11.7 g), the compound B (10.7 mL), tris(dibenzylideneacetone)dipalladium(0) (Pd2(dba)₃, 0.26 mmol), 2,2′-bis(diphenylphosphino)-1,1′-binapthyl (BINAP, 0.87 g), sodium tert-butoxide (9.1 g), and dehydrated toluene (131 mL) were added to a distillation flask (300 mL) and reacted at 85° C. for 6 hours. After cooling, the reaction solution was filtered through celite. The obtained crude product was purified by silica gel chromatography using n-hexane and methylene chloride (volume ratio=3:1), and the obtained solid was dried under reduced pressure to obtain compound D-2. (10.0 g)

### (3) Compound Dopant1D

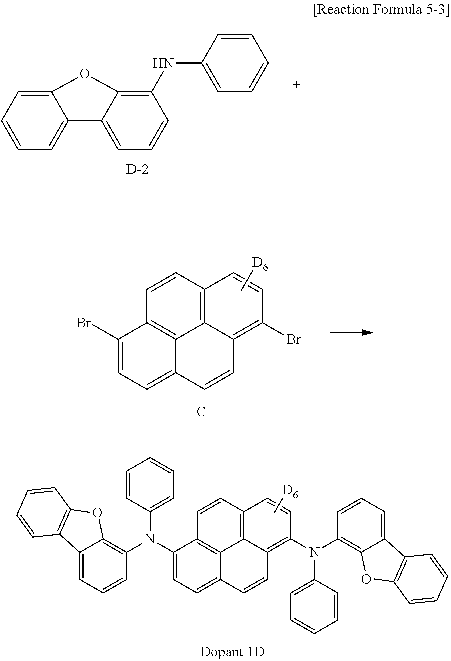

Under argon conditions, the compound D-2 (8.6 g), the compound C (4.8 g), sodium tert-butoxide (2.5 g), palladium(II)acetate (Pd(OAc)₂, 150 mg), tri-tert-butylphosphine (135 mg), and dehydrated toluene (90 mL) were added into a distillation flask (300 mL) and reacted at 85° C. for 7 hours. The reaction solution was filtered, and the obtained crude product was purified by silica gel chromatography using toluene. The obtained solid was recrystallized using toluene and dried under reduced pressure to obtain the compound Dopant1D. (8.3 g)

### 2. Synthesis of the Compound Dopant2D

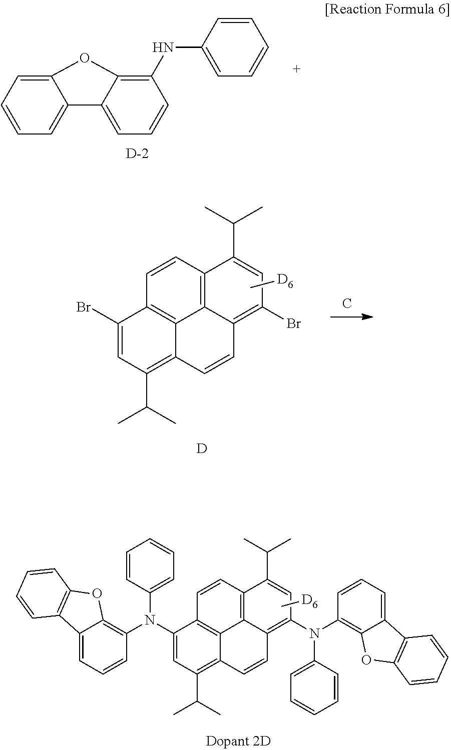

In the synthesis of the compound Dopant1D, the compound D was used instead of the compound C to obtain the compound Dopant2D.

[Organic Light Emitting Diode]

The anode (ITO, 0.5 mm), the HIL (Formula 6 (97 wt %) and Formula 7 (3 wt %), 100 Å), the HTL (Formula 6, 1000 Å), the EBL (Formula 8, 100 Å), the EML (host (98 wt %) and dopant (2 wt %), 200 Å), the HBL (Formula 9, 100 Å), the EIL (Formula 10 (98 wt %) and Li (2 wt %), 200 Å) and the cathode (Al, 500 Å) was sequentially deposited, and an encapsulation film was formed on the cathode using UV epoxy resin and moisture getter to form the OLED.

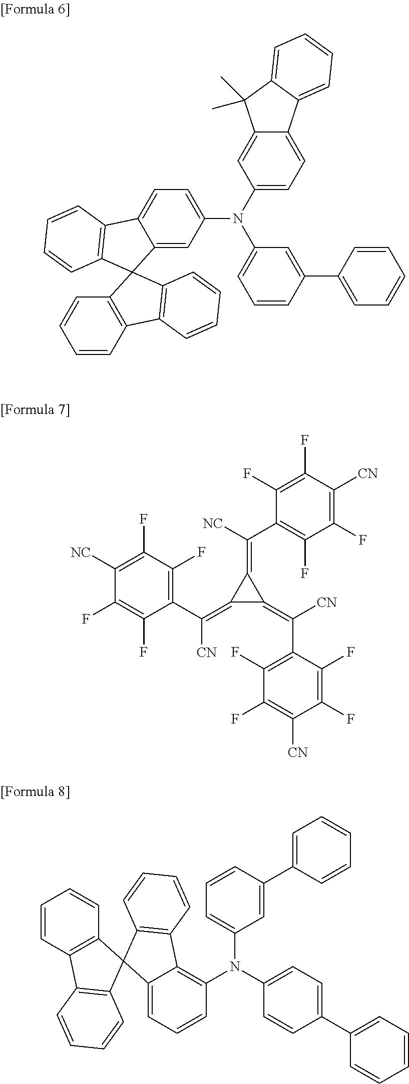 [SMILES: CC1(C)c2ccccc2-c2ccc(N(c3cccc(-c4ccccc4)c3)c3ccc4c(c3)C3(c5ccccc5-c5ccccc53)c3ccccc3-4)cc21 ; N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; c1ccc(-c2ccc(N(c3cccc(-c4ccccc4)c3)c3cccc4c3-c3ccccc3C43c4ccccc4-c4ccccc43)cc2)cc1]

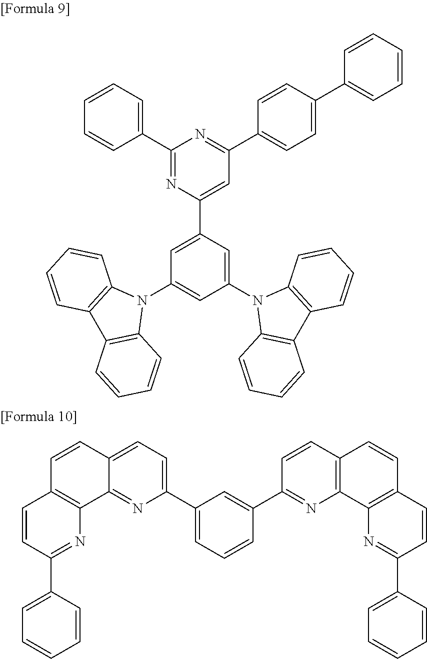 [SMILES: c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; c1ccc(-c2ccc3ccc4ccc(-c5cccc(-c6ccc7ccc8ccc(-c9ccccc9)nc8c7n6)c5)nc4c3n2)cc1]

### 1. Comparative Examples

### (1) Comparative Examples 1 to 4 (Ref1 to Ref4)

The compound “Dopant1” in Formula 11 is used as the dopant, and the compounds “Host1”, “Host2”, “Host3”, and “Host4” in Formula 12 are used as the host, respectively, to form the EML.

### (2) Comparative Examples 5 to 8 (Ref5 to Ref8)

The compound “Dopant2” in Formula 11 is used as the dopant, and the compounds “Host1”, “Host2”, “Host3”, and “Host4” in Formula 12 are used as the host, respectively, to form the EML.

### 2. Examples

### (1) Examples 1 to 4 (Ex1 to Ex4)

The compound “Dopant1” in Formula 11 is used as the dopant, and the compound “Host1D”, and the compounds “Host1D-A”, “Host1D-P1”, and “Host1D-P2” in Formula 12 are used as the host, respectively, to form the EML.

### (2) Examples 5 to 9 (Ex5 to Ex9)

The compound “Dopant1D” is used as the dopant, and the compounds “Host1”, “Host1D”, “Host1D-A”, “Host1D-P1”, and “Host1D-P2” are used as the host, respectively, to form the EML.

### (3) Examples 10 to 14 (Ex10 to Ex14)

The compound “Dopant1D-A” in Formula 11 is used as the dopant, and the compounds “Host1”, “Host1D”, “Host1D-A”, “Host1D-P1”, and “Host1D-P2” are used as the host, respectively, to form the EML.

### (4) Examples 15 to 18 (Ex15 to Ex18)

The compound “Dopant1” in Formula 11 is used as the dopant, and the compounds “Host2D”, “Host2D-A”, “Host2D-P1”, and “Host2D-P2” are used as the host, respectively, to form the EML.

### (5) Examples 19 to 23 (Ex19 to Ex23)

The compound “Dopant1D” is used as the dopant, and the compounds “Host2”, “Host2D”, “Host2D-A”, “Host2D-P1”, and “Host2D-P2” are used as the host, respectively, to form the EML.

### (6) Examples 24 to 28 (Ex24 to Ex28)

The compound “Dopant1D-A” in Formula 11 is used as the dopant, and the compounds “Host2”, “Host2D”, “Host2D-A”, “Host2D-P1”, and “Host2D-P2” are used as the host, respectively, to form the EML.

### (7) Examples 29 to 32 (Ex29 to Ex32)

The compound “Dopant1” in Formula 11 is used as the dopant, and the compound “Host3D”, and the compounds “Host3D-A”, “Host3D-P1”, and “Host3D-P2” in Formula 12 are used as the host, respectively, to form the EML.

### (8) Examples 33 to 37 (Ex33 to Ex37)

The compound “Dopant1D” is used as the dopant, and the compounds “Host3”, “Host3D”, “Host3D-A”, “Host3D-P1”, and “Host3D-P2” are used as the host, respectively, to form the EML.

### (9) Examples 38 to 42 (Ex38 to Ex42)

The compound “Dopant1D-A” in Formula 11 is used as the dopant, and the compounds “Host3”, “Host3D”, “Host3D-A”, “Host3D-P1”, and “Host3D-P2” are used as the host, respectively, to form the EML.

### (10) Examples 43 to 46 (Ex43 to Ex46)

The compound “Dopant1” in Formula 11 is used as the dopant, and the compounds “Host4D”, “Host4D-A”, “Host4D-P1”, and “Host4D-P2” are used as the host, respectively, to form the EML.

### (11) Examples 47 to 51 (Ex47 to Ex51)

The compound “Dopant1D” is used as the dopant, and the compounds “Host4”, “Host4D”, “Host4D-A”, “Host4D-P1”, and “Host4D-P2” are used as the host, respectively, to form the EML.

### (12) Examples 52 to 56 (Ex52 to Ex56)

The compound “Dopant1D-A” in Formula 11 is used as the dopant, and the compounds “Host4”, “Host4D”, “Host4D-A”, “Host4D-P1”, and “Host4D-P2” are used as the host, respectively, to form the EML.

### (13) Examples 57 to 60 (Ex57 to Ex60)

The compound “Dopant2” in Formula 11 is used as the dopant, and the compound “Host1D”, and the compounds “Host1 D-A”, “Host1 D-P1”, and “Host1 D-P2” in Formula 12 are used as the host, respectively, to form the EML.

### (14) Examples 61 to 65 (Ex61 to Ex65)

The compound “Dopant2D” is used as the dopant, and the compounds “Host1”, “Host1D”, “Host1D-A”, “Host1D-P1”, and “Host1D-P2” are used as the host, respectively, to form the EML.

### (15) Examples 66 to 70 (Ex66 to Ex70)

The compound “Dopant2D-A” in Formula 11 is used as the dopant, and the compounds “Host1”, “Host1D”, “Host1D-A”, “Host1D-P1”, and “Host1D-P2” are used as the host, respectively, to form the EML.

### (16) Examples 71 to 74 (Ex71 to Ex74)

The compound “Dopant2” in Formula 11 is used as the dopant, and the compounds “Host2D”, “Host2D-A”, “Host2D-P1”, and “Host2D-P2” are used as the host, respectively, to form the EML.

### (17) Examples 75 to 79 (Ex75 to Ex79)

The compound “Dopant2D” is used as the dopant, and the compounds “Host2”, “Host2D”, “Host2D-A”, “Host2D-P1”, and “Host2D-P2” are used as the host, respectively, to form the EML.

### (18) Examples 80 to 84 (Ex80 to Ex84)

The compound “Dopant2D-A” in Formula 11 is used as the dopant, and the compounds “Host2”, “Host2D”, “Host2D-A”, “Host2D-P1”, and “Host2D-P2” are used as the host, respectively, to form the EML.

### (19) Examples 85 to 88 (Ex85 to Ex88)

The compound “Dopant2” in Formula 11 is used as the dopant, and the compound “Host3D”, and the compounds “Host3D-A”, “Host3D-P1”, and “Host3D-P2” in Formula 12 are used as the host, respectively, to form the EML.

### (20) Examples 89 to 93 (Ex89 to Ex93)

The compound “Dopant2D” is used as the dopant, and the compounds “Host3”, “Host3D”, “Host3D-A”, “Host3D-P1”, and “Host3D-P2” are used as the host, respectively, to form the EML.

### (21) Examples 94 to 98 (Ex94 to Ex98)

The compound “Dopant2D-A” in Formula 11 is used as the dopant, and the compounds “Host3”, “Host3D”, “Host3D-A”, “Host3D-P1”, and “Host3D-P2” are used as the host, respectively, to form the EML.

### (22) Examples 99 to 102 (Ex99 to Ex102)

The compound “Dopant2” in Formula 11 is used as the dopant, and the compounds “Host4D”, “Host4D-A”, “Host4D-P1”, and “Host4D-P2” are used as the host, respectively, to form the EML.

### (23) Examples 103 to 107 (Ex103 to Ex107)

The compound “Dopant2D” is used as the dopant, and the compounds “Host4”, “Host4D”, “Host4D-A”, “Host4D-P1”, and “Host4D-P2” are used as the host, respectively, to form the EML.

### (24) Examples 108 to 112 (Ex108 to Ex112)

The compound “Dopant2D-A” in Formula 11 is used as the dopant, and the compounds “Host4”, “Host4D”, “Host4D-A”, “Host4D-P1”, and “Host4D-P2” are used as the host, respectively, to form the EML.

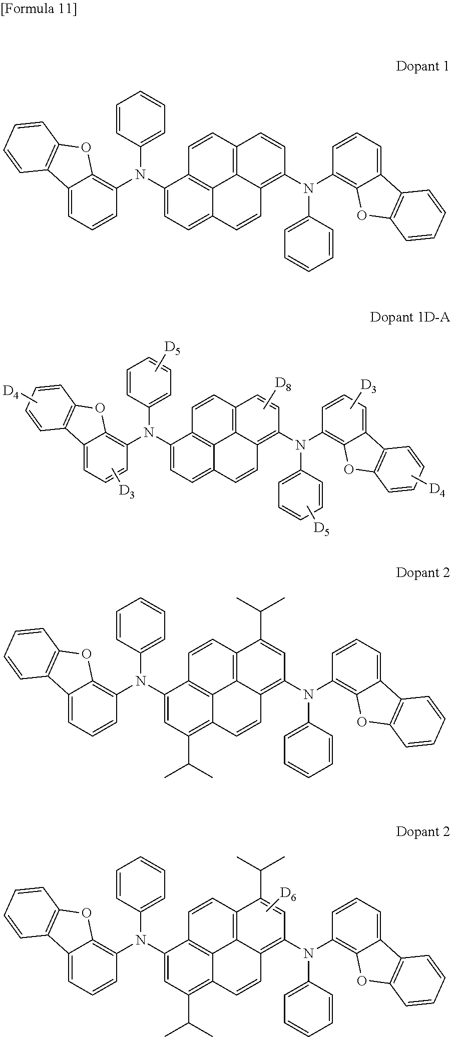

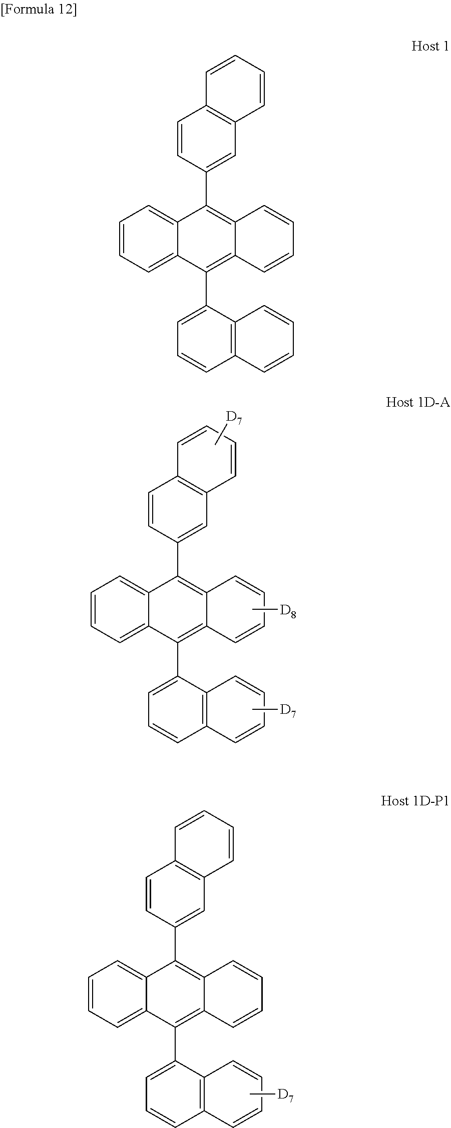

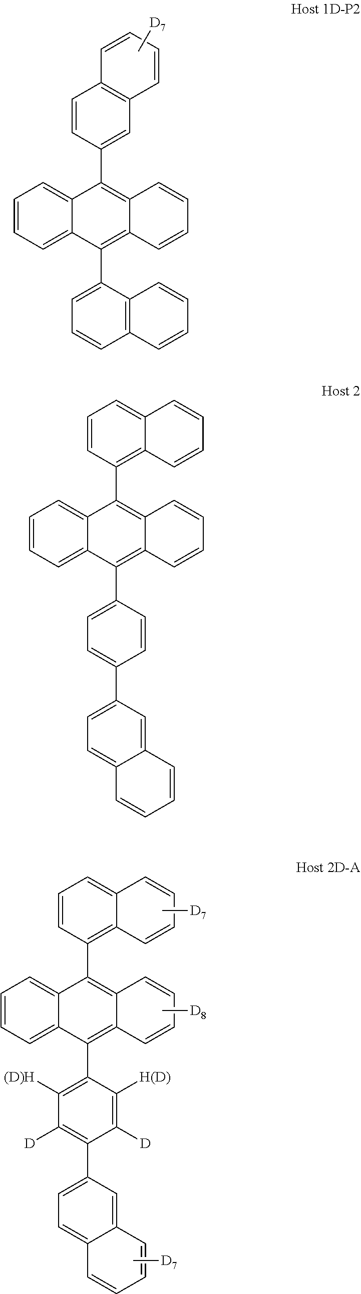

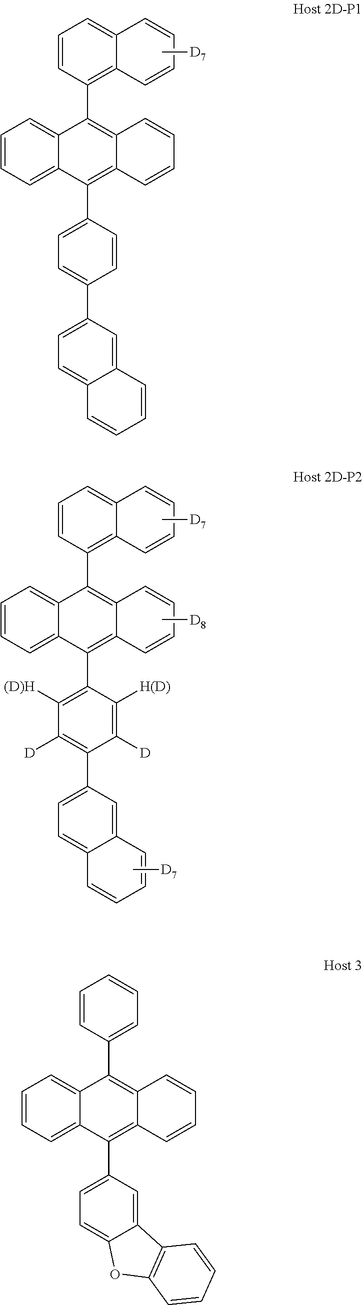

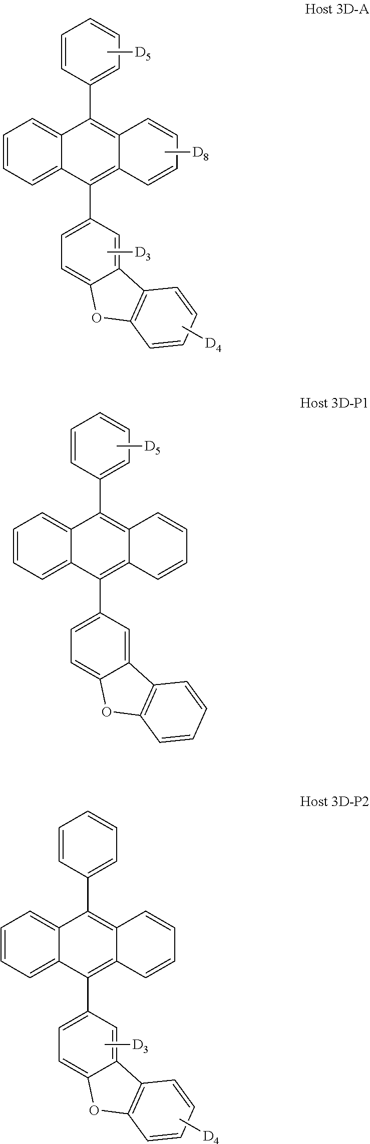

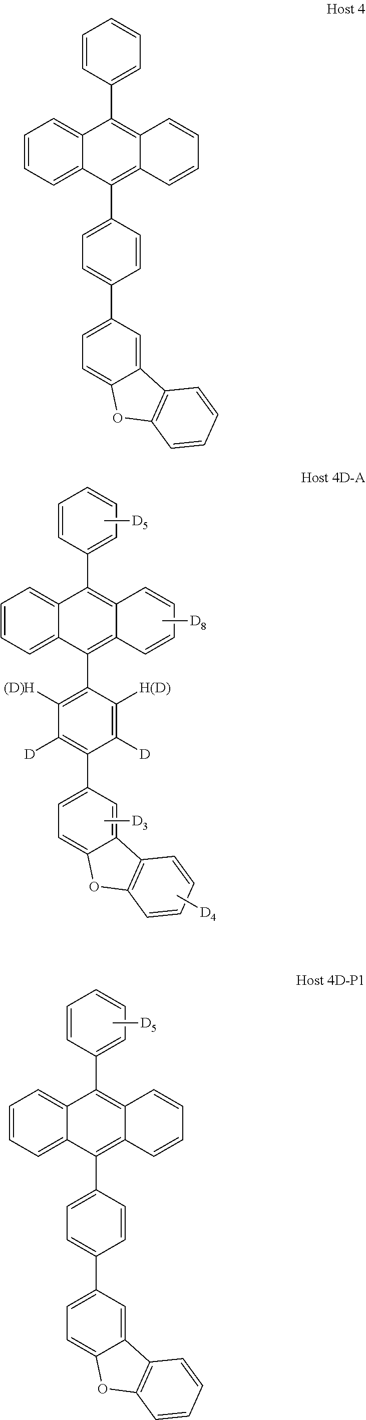

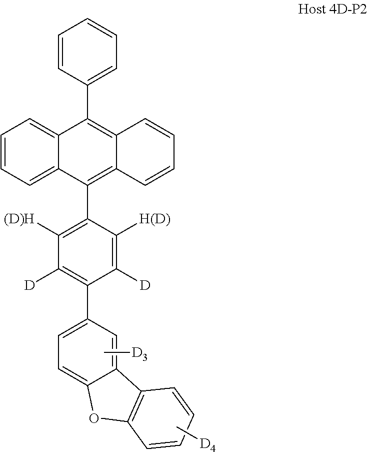

The properties, i.e., voltage (V), efficiency (cd/A), color coordinate (CIE), FWHM and lifespan (T95), of the OLEDs manufactured in Comparative Examples 1 to 8 and Examples 1 to 112 are measured and listed in Tables 1 to 8.

TABLE 1

| | EML | | V | cd/A | CIE (x, y) | | T95 [hr] |
|---|---|---|---|---|---|---|---|
| Ref 1 | Dopant 1 | Host 1 | 3.83 | 6.62 | 0.1382 | 0.1019 | 321 |
| Ex 1 | Dopant 1 | Host 1D | 3.84 | 6.60 | 0.1393 | 0.1019 | 549 |
| Ex 2 | Dopant 1 | Host 1D-A | 3.82 | 6.61 | 0.1384 | 0.1018 | 562 |
| Ex 3 | Dopant 1 | Host 1D-P1 | 3.83 | 6.60 | 0.1381 | 0.1020 | 320 |
| Ex 4 | Dopant 1 | Host 1D-P2 | 3.84 | 6.62 | 0.1385 | 0.1019 | 321 |
| Ex 5 | Dopant 1D | Host 1 | 3.83 | 6.61 | 0.1390 | 0.1018 | 417 |
| Ex 6 | Dopant 1D | Host 1D | 3.83 | 6.61 | 0.1392 | 0.1018 | 704 |
| Ex 7 | Dopant 1D | Host 1D-A | 3.84 | 6.60 | 0.1390 | 0.1019 | 730 |
| Ex 8 | Dopant 1D | Host 1D-P1 | 3.82 | 6.61 | 0.1391 | 0.1020 | 417 |
| Ex 9 | Dopant 1D | Host 1D-P2 | 3.83 | 6.63 | 0.1388 | 0.1021 | 418 |
| Ex 10 | Dopant 1D-A | Host 1 | 3.82 | 6.62 | 0.1386 | 0.1018 | 433 |
| Ex 11 | Dopant 1D-A | Host 1D | 3.84 | 6.61 | 0.1391 | 0.1018 | 747 |
| Ex 12 | Dopant 1D-A | Host 1D-A | 3.83 | 6.61 | 0.1385 | 0.1018 | 762 |
| Ex 13 | Dopant 1D-A | Host 1D-P1 | 3.83 | 6.60 | 0.1387 | 0.1019 | 430 |
| Ex 14 | Dopant 1D-A | Host 1D-P2 | 3.84 | 6.61 | 0.1386 | 0.1019 | 433 |

TABLE 2

| | EML | | V | cd/A | CIE (x, y) | | T95 [hr] |
|---|---|---|---|---|---|---|---|
| Ref 2. | Dopant 1 | Host 2 | 3.64 | 6.84 | 0.1383 | 0.1019 | 322 |
| Ex 15. | Dopant 1 | Host 2D | 3.63 | 6.84 | 0.1392 | 0.1020 | 554 |
| Ex 16. | Dopant 1 | Host 2D-A | 3.63 | 6.83 | 0.1390 | 0.1018 | 566 |
| Ex 17. | Dopant 1 | Host 2D-P1 | 3.64 | 6.82 | 0.1391 | 0.1018 | 322 |
| Ex 18. | Dopant 1 | Host 2D-P2 | 3.62 | 6.86 | 0.1392 | 0.1019 | 323 |
| Ex 19. | Dopant 1D | Host 2 | 3.63 | 6.85 | 0.1392 | 0.1019 | 422 |
| Ex 20. | Dopant 1D | Host 2D | 3.64 | 6.84 | 0.1394 | 0.1018 | 713 |
| Ex 21. | Dopant 1D | Host 2D-A | 3.65 | 6.84 | 0.1389 | 0.1020 | 734 |
| Ex 22. | Dopant 1D | Host 2D-P1 | 3.62 | 6.83 | 0.1392 | 0.1022 | 422 |
| Ex 23. | Dopant 1D | Host 2D-P2 | 3.63 | 6.83 | 0.1393 | 0.1018 | 422 |
| Ex 24. | Dopant 1D-A | Host 2 | 3.63 | 6.85 | 0.1386 | 0.1021 | 438 |
| Ex 25. | Dopant 1D-A | Host 2D | 3.63 | 6.84 | 0.1394 | 0.1017 | 753 |
| Ex 26. | Dopant 1D-A | Host 2D-A | 3.64 | 6.82 | 0.1387 | 0.1019 | 771 |
| Ex 27. | Dopant 1D-A | Host 2D-P1 | 3.63 | 6.83 | 0.1392 | 0.1018 | 440 |
| Ex 28. | Dopant 1D-A | Host 2D-P2 | 3.64 | 6.84 | 0.1392 | 0.1019 | 438 |

TABLE 3

| | EML | | V | cd/A | CIE (x, y) | | T95 [hr] |
|---|---|---|---|---|---|---|---|
| Ref 3. | Dopant 1 | Host 3 | 3.54 | 6.54 | 0.1393 | 0.1032 | 282 |
| Ex 29. | Dopant 1 | Host 3D | 3.52 | 6.55 | 0.1390 | 0.1035 | 482 |
| Ex 30. | Dopant 1 | Host 3D-A | 3.50 | 6.50 | 0.1389 | 0.1025 | 495 |
| Ex 31. | Dopant 1 | Host 3D-P1 | 3.52 | 6.52 | 0.1390 | 0.1030 | 282 |
| Ex 32. | Dopant 1 | Host 3D-P2 | 3.54 | 6.52 | 0.1391 | 0.1031 | 281 |
| Ex 33. | Dopant 1D | Host 3 | 3.54 | 6.53 | 0.1392 | 0.1033 | 375 |
| Ex 34. | Dopant 1D | Host 3D | 3.53 | 6.53 | 0.1391 | 0.1033 | 631 |
| Ex 35. | Dopant 1D | Host 3D-A | 3.55 | 6.55 | 0.1393 | 0.1028 | 656 |
| Ex 36. | Dopant 1D | Host 3D-P1 | 3.50 | 6.51 | 0.1390 | 0.1028 | 374 |
| Ex 37. | Dopant 1D | Host 3D-P2 | 3.56 | 6.50 | 0.1391 | 0.1032 | 375 |
| Ex 38. | Dopant 1D-A | Host 3 | 3.52 | 6.56 | 0.1388 | 0.1031 | 381 |
| Ex 39. | Dopant 1D-A | Host 3D | 3.52 | 6.54 | 0.1392 | 0.1032 | 681 |
| Ex 40. | Dopant 1D-A | Host 3D-A | 3.53 | 6.54 | 0.1390 | 0.1032 | 686 |
| Ex 41. | Dopant 1D-A | Host 3D-P1 | 3.52 | 6.53 | 0.1392 | 0.1030 | 381 |
| Ex 42. | Dopant 1D-A | Host 3D-P2 | 3.54 | 6.51 | 0.1391 | 0.1031 | 381 |

TABLE 4

| | EML | | V | cd/A | CIE (x, y) | | T95 [hr] |
|---|---|---|---|---|---|---|---|
| Ref 4. | Dopant 1 | Host 4 | 3.59 | 6.60 | 0.1393 | 0.1029 | 290 |
| Ex 43. | Dopant 1 | Host 4D | 3.60 | 6.62 | 0.1393 | 0.1030 | 502 |
| Ex 44. | Dopant 1 | Host 4D-A | 3.58 | 6.57 | 0.1380 | 0.1024 | 516 |
| Ex 45. | Dopant 1 | Host 4D-P1 | 3.62 | 6.65 | 0.1391 | 0.1029 | 291 |
| Ex 46. | Dopant 1 | Host 4D-P2 | 3.60 | 6.60 | 0.1398 | 0.1035 | 291 |
| Ex 47. | Dopant 1D | Host 4 | 3.59 | 6.60 | 0.1391 | 0.1030 | 383 |
| Ex 48. | Dopant 1D | Host 4D | 3.60 | 6.61 | 0.1390 | 0.1030 | 654 |
| Ex 49. | Dopant 1D | Host 4D-A | 3.59 | 6.61 | 0.1395 | 0.1035 | 678 |
| Ex 50. | Dopant 1D | Host 4D-P1 | 3.59 | 6.54 | 0.1392 | 0.1032 | 383 |
| Ex 51. | Dopant 1D | Host 4D-P2 | 3.57 | 6.58 | 0.1382 | 0.1030 | 381 |
| Ex 52. | Dopant 1D-A | Host 4 | 3.59 | 6.60 | 0.1390 | 0.1032 | 392 |
| Ex 53. | Dopant 1D-A | Host 4D | 3.60 | 6.60 | 0.1390 | 0.1031 | 690 |
| Ex 54. | Dopant 1D-A | Host 4D-A | 3.64 | 6.67 | 0.1388 | 0.1033 | 706 |
| Ex 55. | Dopant 1D-A | Host 4D-P1 | 3.63 | 6.60 | 0.1390 | 0.1030 | 392 |
| Ex 56. | Dopant 1D-A | Host 4D-P2 | 6.62 | 6.58 | 0.1391 | 0.1027 | 392 |

TABLE 5

| | EML | | V | cd/A | CIE (x, y) | | T95 [hr] |
|---|---|---|---|---|---|---|---|
| Ref 5. | Dopant 2 | Host 1 | 3.75 | 6.73 | 0.1380 | 0.1010 | 385 |
| Ex 57. | Dopant 2 | Host 1D | 3.75 | 6.73 | 0.1381 | 0.1010 | 658 |
| Ex 58. | Dopant 2 | Host 1D-A | 3.70 | 6.71 | 0.1382 | 0.1015 | 670 |
| Ex 59. | Dopant 2 | Host 1D-P1 | 3.75 | 6.72 | 0.1382 | 0.1009 | 385 |
| Ex 60. | Dopant 2 | Host 1D-P2 | 3.72 | 6.70 | 0.1381 | 0.1012 | 385 |
| Ex 61. | Dopant 2D | Host 1 | 3.76 | 6.72 | 0.1382 | 0.1008 | 500 |
| Ex 62. | Dopant 2D | Host 1D | 3.76 | 6.71 | 0.1382 | 0.1012 | 839 |
| Ex 63. | Dopant 2D | Host 1D-A | 3.71 | 6.80 | 0.1378 | 0.1013 | 877 |
| Ex 64. | Dopant 2D | Host 1D-P1 | 3.74 | 6.72 | 0.1382 | 0.1007 | 500 |
| Ex 65. | Dopant 2D | Host 1D-P2 | 3.75 | 6.68 | 0.1381 | 0.1014 | 501 |
| Ex 66. | Dopant 2D-A | Host 1 | 3.78 | 6.70 | 0.1378 | 0.1013 | 524 |
| Ex 67. | Dopant 2D-A | Host 1D | 3.77 | 6.70 | 0.1382 | 0.1013 | 880 |
| Ex 68. | Dopant 2D-A | Host 1D-A | 3.71 | 6.72 | 0.1383 | 0.1010 | 901 |
| Ex 69. | Dopant 2D-A | Host 1D-P1 | 3.72 | 6.71 | 0.1382 | 0.1011 | 525 |
| Ex 70. | Dopant 2D-A | Host 1D-P2 | 3.75 | 6.66 | 0.1380 | 0.1012 | 525 |

TABLE 6

| | EML | | V | cd/A | CIE (x, y) | | T95 [hr] |
|---|---|---|---|---|---|---|---|
| Ref 6. | Dopant 2 | Host 2 | 3.60 | 6.91 | 0.1381 | 0.1021 | 385 |
| Ex 71. | Dopant 2 | Host 2D | 3.60 | 6.92 | 0.1383 | 0.1023 | 660 |
| Ex 72. | Dopant 2 | Host 2D-A | 3.55 | 6.85 | 0.1381 | 0.1022 | 674 |
| Ex 73. | Dopant 2 | Host 2D-P1 | 3.58 | 6.90 | 0.1382 | 0.1019 | 384 |
| Ex 74. | Dopant 2 | Host 2D-P2 | 3.58 | 6.88 | 0.1382 | 0.1022 | 386 |
| Ex 75. | Dopant 2D | Host 2 | 3.59 | 6.91 | 0.1380 | 0.1024 | 502 |
| Ex 76. | Dopant 2D | Host 2D | 3.60 | 6.90 | 0.1381 | 0.1023 | 845 |
| Ex 77. | Dopant 2D | Host 2D-A | 3.58 | 6.92 | 0.1377 | 0.1022 | 879 |
| Ex 78. | Dopant 2D | Host 2D-P1 | 3.62 | 6.84 | 0.1382 | 0.1019 | 502 |
| Ex 79. | Dopant 2D | Host 2D-P2 | 3.56 | 6.87 | 0.1383 | 0.1020 | 501 |
| Ex 80. | Dopant 2D-A | Host 2 | 3.55 | 6.90 | 0.1380 | 0.1020 | 520 |
| Ex 81. | Dopant 2D-A | Host 2D | 3.61 | 6.91 | 0.1381 | 0.1023 | 899 |
| Ex 82. | Dopant 2D-A | Host 2D-A | 3.62 | 6.88 | 0.1382 | 0.1021 | 920 |
| Ex 83. | Dopant 2D-A | Host 2D-P1 | 3.55 | 6.84 | 0.1383 | 0.1022 | 520 |
| Ex 84. | Dopant 2D-A | Host 2D-P2 | 3.57 | 6.85 | 0.1381 | 0.1019 | 522 |

TABLE 7

| | EML | | V | cd/A | CIE (x, y) | | T95 [hr] |
|---|---|---|---|---|---|---|---|
| Ref 7. | Dopant 2 | Host 3 | 3.52 | 6.68 | 0.1386 | 0.1033 | 338 |
| Ex 85. | Dopant 2 | Host 3D | 3.53 | 6.66 | 0.1381 | 0.1032 | 585 |
| Ex 86. | Dopant 2 | Host 3D-A | 3.51 | 6.65 | 0.1381 | 0.1033 | 599 |
| Ex 87. | Dopant 2 | Host 3D-P1 | 3.51 | 6.61 | 0.1382 | 0.1031 | 338 |
| Ex 88. | Dopant 2 | Host 3D-P2 | 3.52 | 6.68 | 0.1384 | 0.1033 | 338 |
| Ex 89. | Dopant 2D | Host 3 | 3.52 | 6.67 | 0.1382 | 0.1032 | 412 |
| Ex 90. | Dopant 2D | Host 3D | 3.51 | 6.69 | 0.1385 | 0.1032 | 737 |
| Ex 91. | Dopant 2D | Host 3D-A | 3.50 | 6.66 | 0.1382 | 0.1029 | 748 |
| Ex 92. | Dopant 2D | Host 3D-P1 | 3.55 | 6.68 | 0.1388 | 0.1033 | 410 |
| Ex 93. | Dopant 2D | Host 3D-P2 | 3.51 | 6.65 | 0.1385 | 0.1031 | 414 |
| Ex 94. | Dopant 2D-A | Host 3 | 3.52 | 6.69 | 0.1381 | 0.1031 | 456 |
| Ex 95. | Dopant 2D-A | Host 3D | 3.51 | 6.69 | 0.1384 | 0.1031 | 774 |
| Ex 96. | Dopant 2D-A | Host 3D-A | 3.53 | 6.68 | 0.1381 | 0.1032 | 812 |
| Ex 97. | Dopant 2D-A | Host 3D-P1 | 3.51 | 6.62 | 0.1384 | 0.1033 | 455 |
| Ex 98. | Dopant 2D-A | Host 3D-P2 | 3.50 | 6.67 | 0.1384 | 0.1033 | 456 |

TABLE 8

| | EML | | V | cd/A | CIE (x, y) | | T95 [hr] |
|---|---|---|---|---|---|---|---|
| Ref 8. | Dopant 2 | Host 4 | 3.54 | 6.70 | 0.1382 | 0.1031 | 351 |
| Ex 99. | Dopant 2 | Host 4D | 3.54 | 6.73 | 0.1381 | 0.1031 | 600 |
| Ex 100. | Dopant 2 | Host 4D-A | 3.55 | 6.69 | 0.1380 | 0.1033 | 610 |
| Ex 101. | Dopant 2 | Host 4D-P1 | 3.51 | 6.68 | 0.1381 | 0.1032 | 351 |
| Ex 102. | Dopant 2 | Host 4D-P2 | 3.50 | 6.68 | 0.1385 | 0.1031 | 351 |
| Ex 103. | Dopant 2D | Host 4 | 3.53 | 6.72 | 0.1387 | 0.1030 | 431 |
| Ex 104. | Dopant 2D | Host 4D | 3.53 | 6.70 | 0.1383 | 0.1032 | 764 |
| Ex 105. | Dopant 2D | Host 4D-A | 3.53 | 6.72 | 0.1382 | 0.1032 | 790 |
| Ex 106. | Dopant 2D | Host 4D-P1 | 3.52 | 6.71 | 0.1378 | 0.1033 | 433 |
| Ex 107. | Dopant 2D | Host 4D-P2 | 3.51 | 6.70 | 0.1382 | 0.1030 | 435 |
| Ex 108. | Dopant 2D-A | Host 4 | 3.54 | 6.68 | 0.1383 | 0.1032 | 473 |
| Ex 109. | Dopant 2D-A | Host 4D | 3.53 | 6.71 | 0.1383 | 0.1032 | 800 |
| Ex 110. | Dopant 2D-A | Host 4D-A | 3.51 | 6.70 | 0.1381 | 0.1030 | 828 |
| Ex 111. | Dopant 2D-A | Host 4D-P1 | 3.50 | 6.68 | 0.1380 | 0.1033 | 473 |
| Ex 112. | Dopant 2D-A | Host 4D-P2 | 3.54 | 6.69 | 0.1382 | 0.1031 | 473 |

As shown in Tables 1 to 8, the lifespan of the OLED in Examples 1, 2, 6, 7, 11, 12, 15, 16, 20, 21, 25, 26, 29, 30, 34, 35, 39, 40, 43, 44, 48, 49, 53, 54, 57, 58, 62, 63, 67, 68, 71, 72, 76, 77, 81, 82, 85, 86, 90, 91, 95, 96, 99, 100, 104, 105, 109 and 110, which uses an anthracene derivative including the deuterated anthracene core as the host, is significantly increased.

On the other hand, in comparison to the OLED in Examples 2, 7, 12, 16, 21, 26, 30, 35, 40, 44, 49, 54, 58, 63, 68, 72, 77, 82, 86, 91, 96, 100, 105 and 110, which uses the wholly-deuterated anthracene derivative as the host, the lifespan of the OLED in Examples 1, 6, 11, 15, 20, 25, 29, 34, 39, 43, 48, 53, 57, 62, 67, 71, 76, 81, 85, 90, 95, 99, 104 and 109, which uses the core-deuterated anthracene derivative as the host, is slightly short. However, the OLED in Examples 1, 6, 11, 15, 20, 25, 29, 34, 39, 43, 48, 53, 57, 62, 67, 71, 76, 81, 85, 90, 95, 99, 104 and 109 provides sufficient lifespan increase with low ratio of deuterium, which is expensive. Namely, the OLED in Examples 1, 6, 11, 15, 20, 25, 29, 34, 39, 43, 48, 53, 57, 62, 67, 71, 76, 81, 85, 90, 95, 99, 104 and 109 has enhanced emitting efficiency and lifespan with minimizing production cost increase.

In addition, the lifespan of the OLED in Examples 5 to 14, 19 to 28, 33 to 42, 47 to 56, 61 to 70, 75 to 84, 89 to 98 and 103 to 112, which uses a pyrene derivative including the deuterated pyrene core as the dopant, is significantly increased.

On the other hand, in comparison to the OLED in Examples 10 to 14, 24 to 28, 38 to 42, 52 to 56, 66 to 70, 80 to 84, 94 to 98, 108 to 112, which uses the wholly-deuterated pyrene derivative as the dopant, the lifespan of the OLED in Examples 5 to 9, 19 to 23, 33 to 37, 47 to 51, 61 to 65, 75 to 79, 89 to 93 and 103 to 107, which uses the core-deuterated pyrene derivative as the dopant, is slightly short. However, the OLED in Examples 5 to 9, 19 to 23, 33 to 37, 47 to 51, 61 to 65, 75 to 79, 89 to 93 and 103 to 107 provides sufficient lifespan increase with low ratio of deuterium, which is expensive.

In the OLED D of the present disclosure, the EML 240 includes the host of the anthracene derivative and the dopant of the pyrene derivative, and at least one of the anthracene core of the anthracene derivative and the pyrene core of the pyrene derivative is deuterated. As a result, the OLED D and the organic light emitting display device 100 have advantages in the emitting efficiency and the lifespan.

FIG. 4 is a schematic cross-sectional view illustrating an OLED having a tandem structure of two emitting units according to the first embodiment of the present disclosure.

As shown in FIG. 4, the OLED D includes the first and second electrodes 160 and 164 facing each other and the organic emitting layer 162 between the first and second electrodes 160 and 164. The organic emitting layer 162 includes a first emitting part 310 including a first EML 320, a second emitting part 330 including a second EML 340 and a charge generation layer (CGL) 350 between the first and second emitting parts 310 and 330. Namely, the OLED D in FIG. 4 and the OLED D in FIG. 3 have a difference in the organic emitting layer 162.

The first electrode 160 can be formed of a conductive material having a relatively high work function to serve as an anode for injecting a hole into the organic emitting layer 162. The second electrode 164 can be formed of a conductive material having a relatively low work function to serve as a cathode for injecting an electron into the organic emitting layer 162. The first electrode 160 can be formed of ITO or IZO, and the second electrode 164 can be formed of Al, Mg, Ag, AlMg or MgAg.

The CGL 350 is positioned between the first and second emitting parts 310 and 330, and the first emitting part 310, the CGL 350 and the second emitting part 330 are sequentially stacked on the first electrode 160. Namely, the first emitting part 310 is positioned between the first electrode 160 and the CGL 350, and the second emitting part 330 is positioned between the second electrode 164 and the CGL 350.

The first emitting part 310 includes a first EML 320. In addition, the first emitting part 310 can further include a first EBL 316 between the first electrode 160 and the first EML 320 and a first HBL 318 between the first EML 320 and the CGL 350.

In addition, the first emitting part 310 can further include a first HTL 314 between the first electrode 160 and the first EBL 316 and an HIL 312 between the first electrode 160 and the first HTL 314.

The first EML 320 includes a host 322, which is an anthracene derivative, and a dopant 324, which is a pyrene derivative, and at least one of an anthracene core of the anthracene derivative and a pyrene core of the pyrene derivative is deuterated. The first EML 320 provides a blue emission.

For example, when the anthracene core of the host 322 is deuterated (e.g., “core-deuterated anthracene derivative”), the dopant 324 can be non-deuterated (e.g., “non-deuterated pyrene derivative”) or all of the pyrene core and a substituent of the dopant 324 can be deuterated (e.g., “wholly-deuterated pyrene derivative”). Alternatively, the pyrene core of the dopant 324 except the substituent can be deuterated (e.g., “core-deuterated pyrene derivative”), or the substituent of the dopant 324 except the pyrene core can be deuterated (e.g., “substituent-deuterated pyrene derivative”).

Alternatively, in the first EML 320, when the pyrene core of the dopant 324 is deuterated (e.g., “core-deuterated pyrene derivative”), the host 322 can be non-deuterated (e.g., “non-deuterated anthracene derivative”) or all of the anthracene core and a substituent of the host 322 can be deuterated (e.g., “wholly-deuterated anthracene derivative”). Alternatively, the anthracene core of the host 322 except the substituent can be deuterated (e.g., “core-deuterated anthracene derivative”), or the substituent of the host 322 except the anthracene core can be deuterated (e.g., “substituent-deuterated anthracene derivative”).

In the first EML 320, the host 322 can have a weight % of about 70 to 99.9, and the dopant 324 can have a weight % of about 0.1 to 30. To provide sufficient emitting efficiency and lifespan, a weight % of the dopant 324 can be about 0.1 to 10, preferably about 1 to 5.

The second emitting part 330 includes the second EML 340. In addition, the second emitting part 330 can further include a second EBL 334 between the CGL 350 and the second EML 340 and a second HBL 336 between the second EML 340 and the second electrode 164.

In addition, the second emitting part 330 can further include a second HTL 332 between the CGL 350 and the second EBL 334 and an EIL 338 between the second HBL 336 and the second electrode 164.

The second EML 340 includes a host 342, which is an anthracene derivative, a dopant 344, which is a pyrene derivative, and at least one of an anthracene core of the anthracene derivative and a pyrene core of the pyrene derivative is deuterated. The second EML 340 provides a blue emission.

For example, when the anthracene core of the host 342 is deuterated (e.g., “core-deuterated anthracene derivative”), the dopant 344 can be non-deuterated (e.g., “non-deuterated pyrene derivative”) or all of the pyrene core and a substituent of the dopant 344 can be deuterated (e.g., “wholly-deuterated pyrene derivative”). Alternatively, the pyrene core of the dopant 344 except the substituent can be deuterated (e.g., “core-deuterated pyrene derivative”), or the substituent of the dopant 344 except the pyrene core can be deuterated (e.g., “substituent-deuterated pyrene derivative”).

Alternatively, in the second EML 340, when the pyrene core of the dopant 344 is deuterated (e.g., “core-deuterated pyrene derivative”), the host 342 can be non-deuterated (e.g., “non-deuterated anthracene derivative”) or all of the anthracene core and a substituent of the host 342 can be deuterated (e.g., “wholly-deuterated anthracene derivative”). Alternatively, the anthracene core of the host 342 except the substituent can be deuterated (e.g., “core-deuterated anthracene derivative”), or the substituent of the host 342 except the anthracene core can be deuterated (e.g., “substituent-deuterated anthracene derivative”).

In the second EML 340, the host 342 can have a weight % of about 70 to 99.9, and the dopant 344 can have a weight % of about 0.1 to 30. To provide sufficient emitting efficiency and lifespan, a weight % of the dopant 344 can be about 0.1 to 10, preferably about 1 to 5.

The host 342 of the second EML 340 can be same as or different from the host 322 of the first EML 320, and the dopant 344 of the second EML 340 can be same as or different from the dopant 324 of the first EML 320.

The CGL 350 is positioned between the first and second emitting parts 310 and 330. Namely, the first and second emitting parts 310 and 330 are connected through the CGL 350. The CGL 350 can be a P-N junction CGL of an N-type CGL 352 and a P-type CGL 354.

The N-type CGL 352 is positioned between the first HBL 318 and the second HTL 332, and the P-type CGL 354 is positioned between the N-type CGL 352 and the second HTL 332.

In the OLED D, since each of the first and second EMLs 320 and 340 includes the host 322 and 342, each of which is an anthracene derivative, and the dopant 324 and 344, each of which is a pyrene derivative, and at least one of an anthracene core of the anthracene derivative and a pyrene core of the pyrene derivative is deuterated. As a result, the OLED D and the organic light emitting display device 100 have advantages in the emitting efficiency and the lifespan.

In addition, since the first and second emitting parts 310 and 330 for emitting blue light are stacked, the organic light emitting display device 100 provides an image having high color temperature.

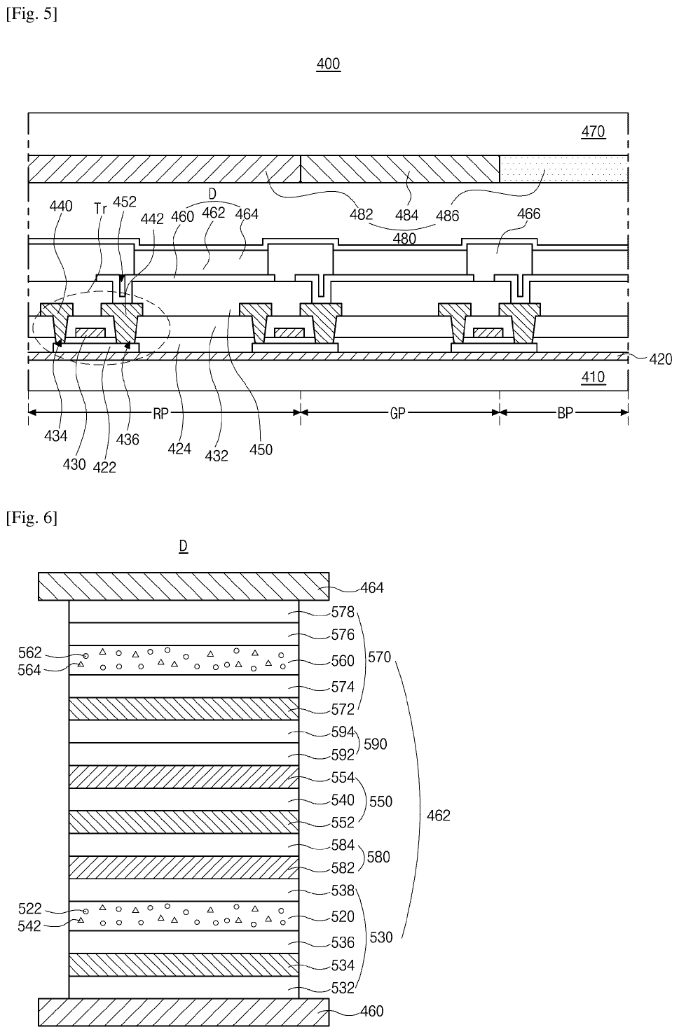

FIG. 5 is a schematic cross-sectional view illustrating an organic light emitting display device according to a second embodiment of the present disclosure, and FIG. 6 is a schematic cross-sectional view illustrating an OLED for the organic light emitting display device according to the second embodiment of the present disclosure.

As shown in FIG. 5, the organic light emitting display device 400 includes a first substrate 410, where a red pixel RP, a green pixel GP and a blue pixel BP are defined, a second substrate 470 facing the first substrate 410, an OLED D, which is positioned between the first and second substrates 410 and 470 and providing white emission, and a color filter layer 480 between the OLED D and the second substrate 470.

Each of the first and second substrates 410 and 470 can be a glass substrate or a plastic substrate. For example, each of the first and second substrates 410 and 470 can be a polyimide substrate.

A buffer layer 420 is formed on the substrate, and the TFT Tr corresponding to each of the red, green and blue pixels RP, GP and BP is formed on the buffer layer 420. The buffer layer 420 can be omitted.

A semiconductor layer 422 is formed on the buffer layer 420. The semiconductor layer 122 can include an oxide semiconductor material or polycrystalline silicon.

A gate insulating layer 424 is formed on the semiconductor layer 422. The gate insulating layer 424 can be formed of an inorganic insulating material such as silicon oxide or silicon nitride.

A gate electrode 430, which is formed of a conductive material, e.g., metal, is formed on the gate insulating layer 424 to correspond to a center of the semiconductor layer 422.

An interlayer insulating layer 432, which is formed of an insulating material, is formed on the gate electrode 430. The interlayer insulating layer 432 can be formed of an inorganic insulating material, e.g., silicon oxide or silicon nitride, or an organic insulating material, e.g., benzocyclobutene or photo-acryl.

The interlayer insulating layer 432 includes first and second contact holes 434 and 436 exposing both sides of the semiconductor layer 422. The first and second contact holes 434 and 436 are positioned at both sides of the gate electrode 430 to be spaced apart from the gate electrode 430.

A source electrode 440 and a drain electrode 442, which are formed of a conductive material, e.g., metal, are formed on the interlayer insulating layer 432.

The source electrode 440 and the drain electrode 442 are spaced apart from each other with respect to the gate electrode 430 and respectively contact both sides of the semiconductor layer 422 through the first and second contact holes 434 and 436.

The semiconductor layer 422, the gate electrode 430, the source electrode 440 and the drain electrode 442 constitute the TFT Tr. The TFT Tr serves as a driving element. Namely, the TFT Tr can correspond to the driving TFT Td (of FIG. 1).

The gate line and the data line cross each other to define the pixel, and the switching TFT is formed to be connected to the gate and data lines. The switching TFT is connected to the TFT Tr as the driving element.

In addition, the power line, which can be formed to be parallel to and spaced apart from one of the gate and data lines, and the storage capacitor for maintaining the voltage of the gate electrode of the TFT Tr in one frame can be further formed.

A passivation layer 450, which includes a drain contact hole 452 exposing the drain electrode 442 of the TFT Tr, is formed to cover the TFT Tr.

A first electrode 460, which is connected to the drain electrode 442 of the TFT Tr through the drain contact hole 452, is separately formed in each pixel. The first electrode 160 can be an anode and can be formed of a conductive material having a relatively high work function. For example, the first electrode 460 can be formed of a transparent conductive material such as indium-tin-oxide (ITO) or indium-zinc-oxide (IZO).

A reflection electrode or a reflection layer can be formed under the first electrode 460. For example, the reflection electrode or the reflection layer can be formed of aluminum-palladium-copper (APC) alloy.

A bank layer 466 is formed on the passivation layer 450 to cover an edge of the first electrode 460. Namely, the bank layer 466 is positioned at a boundary of the pixel and exposes a center of the first electrode 460 in the red, green and blue pixels RP, GP and BP. The bank layer 466 can be omitted.

An organic emitting layer 462 is formed on the first electrode 460.

Referring to FIG. 6, the organic emitting layer 462 includes a first emitting part 530 including a first EML 520, a second emitting part 550 including a second EML 540, a third emitting part 570 including a third EML 560, a first CGL 580 between the first and second emitting parts 530 and 550 and a second CGL 590 between the second and third emitting parts 550 and 570.

The first electrode 460 can be formed of a conductive material having a relatively high work function to serve as an anode for injecting a hole into the organic emitting layer 462. The second electrode 464 can be formed of a conductive material having a relatively low work function to serve as a cathode for injecting an electron into the organic emitting layer 462. The first electrode 460 can be formed of ITO or IZO, and the second electrode 464 can be formed of Al, Mg, Ag, AlMg or MgAg.

The first CGL 580 is positioned between the first and second emitting parts 530 and 550, and the second CGL 590 is positioned between the second and third emitting parts 550 and 570. Namely, the first emitting part 530, the first CGL 580, the second emitting part 550, the second CGL 590 and the third emitting part 570 are sequentially stacked on the first electrode 460. In other words, the first emitting part 530 is positioned between the first electrode 460 and the first CGL 570, the second emitting part 550 is positioned between the first and second CGLs 580 and 590, and the third emitting part 570 is positioned between the second electrode 460 and the second CGL 590.

The first emitting part 530 can include an HIL 532, a first HTL 534, a first EBL 536, the first EML 520 and a first HBL 538 sequentially stacked on the first electrode 460. Namely, the HIL 532, the first HTL 534 and the first EBL 536 are positioned between the first electrode 460 and the first EML 520, and the first HBL 538 is positioned between the first EML 520 and the first CGL 580.

The first EML 520 includes a host 522, which is an anthracene derivative, and a dopant 524, which is a pyrene derivative, and at least one of an anthracene core of the anthracene derivative and a pyrene core of the pyrene derivative is deuterated. The first EML 520 provides a blue emission.

For example, in the first EML 520, when the anthracene core of the host 522 is deuterated (e.g., “core-deuterated anthracene derivative”), the dopant 524 can be non-deuterated (e.g., “non-deuterated pyrene derivative”) or all of the pyrene core and a substituent of the dopant 524 can be deuterated (e.g., “wholly-deuterated pyrene derivative”). Alternatively, the pyrene core of the dopant 524 except the substituent can be deuterated (e.g., “core-deuterated pyrene derivative”), or the substituent of the dopant 524 except the pyrene core can be deuterated (e.g., “substituent-deuterated pyrene derivative”).

Alternatively, in the first EML 520, when the pyrene core of the dopant 524 is deuterated (e.g., “core-deuterated pyrene derivative”), the host 522 can be non-deuterated (e.g., “non-deuterated anthracene derivative”) or all of the anthracene core and a substituent of the host 522 can be deuterated (e.g., “wholly-deuterated anthracene derivative”). Alternatively, the anthracene core of the host 522 except the substituent can be deuterated (e.g., “core-deuterated anthracene derivative”), or the substituent of the host 522 except the anthracene core can be deuterated (e.g., “substituent-deuterated anthracene derivative”).

In the first EML 520, the host 522 can have a weight % of about 70 to 99.9, and the dopant 524 can have a weight % of about 0.1 to 30. To provide sufficient emitting efficiency and lifespan, a weight % of the dopant 524 can be about 0.1 to 10, preferably about 1 to 5.

The second EML 550 can include a second HTL 552, the second EML 540 and an electron transporting layer (ETL) 554. The second HTL 552 is positioned between the first CGL 580 and the second EML 540, and the ETL 554 is positioned between the second EML 540 and the second CGL 590.

The second EML 540 can be a yellow-green EML. For example, the second EML 540 can include a host and a yellow-green dopant. Alternatively, the second EML 540 can include a host, a red dopant and a green dopant. In this instance, the second EML 540 can include a lower layer including the host and the red dopant (or the green dopant) and an upper layer including the host and the green dopant (or the red dopant).

The third emitting part 570 can include a third HTL 572, a second EBL 574, the third EML 560, a second HBL 576 and an EIL 578.

The third EML 560 includes a host 562, which is an anthracene derivative, a dopant 564, which is a pyrene derivative, and at least one of an anthracene core of the anthracene derivative and a pyrene core of the pyrene derivative is deuterated. The third EML 560 provides a blue emission.

For example, in the third EML 560, when the anthracene core of the host 562 is deuterated (e.g., “core-deuterated anthracene derivative”), the dopant 564 can be non-deuterated (e.g., “non-deuterated pyrene derivative”) or all of the pyrene core and a substituent of the dopant 564 can be deuterated (e.g., “wholly-deuterated pyrene derivative”). Alternatively, the pyrene core of the dopant 564 except the substituent can be deuterated (e.g., “core-deuterated pyrene derivative”), or the substituent of the dopant 564 except the pyrene core can be deuterated (e.g., “substituent-deuterated pyrene derivative”).

Alternatively, in the third EML 560, when the pyrene core of the dopant 564 is deuterated (e.g., “core-deuterated pyrene derivative”), the host 562 can be non-deuterated (e.g., “non-deuterated anthracene derivative”) or all of the anthracene core and a substituent of the host 562 can be deuterated (e.g., “wholly-deuterated anthracene derivative”). Alternatively, the anthracene core of the host 562 except the substituent can be deuterated (e.g., “core-deuterated anthracene derivative”), or the substituent of the host 562 except the anthracene core can be deuterated (e.g., “substituent-deuterated anthracene derivative”).

In the third EML 560, the host 562 can have a weight % of about 70 to 99.9, and the dopant 564 can have a weight % of about 0.1 to 30. To provide sufficient emitting efficiency and lifespan, a weight % of the dopant 564 can be about 0.1 to 10, preferably about 1 to 5.

The host 562 of the third EML 560 can be same as or different from the host 522 of the first EML 520, and the dopant 564 of the third EML 560 can be same as or different from the dopant 524 of the first EML 520.

The first CGL 580 is positioned between the first emitting part 530 and the second emitting part 550, and the second CGL 590 is positioned between the second emitting part 550 and the third emitting part 570. Namely, the first and second emitting stacks 530 and 550 are connected through the first CGL 580, and the second and third emitting stacks 550 and 570 are connected through the second CGL 590. The first CGL 580 can be a P-N junction CGL of a first N-type CGL 582 and a first P-type CGL 584, and the second CGL 590 can be a P-N junction CGL of a second N-type CGL 592 and a second P-type CGL 594.

In the first CGL 580, the first N-type CGL 582 is positioned between the first HBL 538 and the second HTL 552, and the first P-type CGL 584 is positioned between the first N-type CGL 582 and the second HTL 552.

In the second CGL 590, the second N-type CGL 592 is positioned between the ETL 554 and the third HTL 572, and the second P-type CGL 594 is positioned between the second N-type CGL 592 and the third HTL 572.

In the OLED D, each of the first and third EMLs 520 and 560 includes the host 522 and 562, each of which is an anthracene derivative, the blue dopant 524 and 564, each of which is a pyrene derivative.

Accordingly, the OLED D including the first and third emitting parts 530 and 570 with the second emitting part 550, which emits yellow-green light or red/green light, can emit white light.

In FIG. 6, the OLED D has a triple-stack structure of the first, second and third emitting parts 530, 550 and 570. Alternatively, the OLED D can have a double-stack structure without the first emitting part 530 or the third emitting part 570.

Referring to FIG. 5 again, a second electrode 464 is formed over the substrate 410 where the organic emitting layer 462 is formed.

In the organic light emitting display device 400, since the light emitted from the organic emitting layer 462 is incident to the color filter layer 480 through the second electrode 464, the second electrode 464 has a thin profile for transmitting the light.

The first electrode 460, the organic emitting layer 462 and the second electrode 464 constitute the OLED D.

The color filter layer 480 is positioned over the OLED D and includes a red color filter 482, a green color filter 484 and a blue color filter 486 respectively corresponding to the red, green and blue pixels RP, GP and BP.

The color filter layer 480 can be attached to the OLED D by using an adhesive layer. Alternatively, the color filter layer 480 can be formed directly on the OLED D.

An encapsulation film can be formed to prevent penetration of moisture into the OLED D. For example, the encapsulation film can include a first inorganic insulating layer, an organic insulating layer and a second inorganic insulating layer sequentially stacked, but it is not limited thereto. The encapsulation film can be omitted.

A polarization plate for reducing an ambient light reflection can be disposed over the top-emission type OLED D. For example, the polarization plate can be a circular polarization plate.

In FIG. 5, the light from the OLED D passes through the second electrode 464, and the color filter layer 480 is disposed on or over the OLED D. Alternatively, when the light from the OLED D passes through the first electrode 460, the color filter layer 480 can be disposed between the OLED D and the first substrate 410.

A color conversion layer can be formed between the OLED D and the color filter layer 480. The color conversion layer can include a red color conversion layer, a green color conversion layer and a blue color conversion layer respectively corresponding to the red, green and blue pixels RP, GP and BP. The white light from the OLED D is converted into the red light, the green light and the blue light by the red, green and blue color conversion layer, respectively.

As described above, the white light from the organic light emitting diode D passes through the red color filter 482, the green color filter 484 and the blue color filter 486 in the red pixel RP, the green pixel GP and the blue pixel BP such that the red light, the green light and the blue light are provided from the red pixel RP, the green pixel GP and the blue pixel BP, respectively.

In FIGS. 5 and 6, the OLED D emitting the white light is used for a display device. Alternatively, the OLED D can be formed on an entire surface of a substrate without at least one of the driving element and the color filter layer to be used for a lightening device. The display device and the lightening device each including the OLED D of the present disclosure can be referred to as an organic light emitting device.

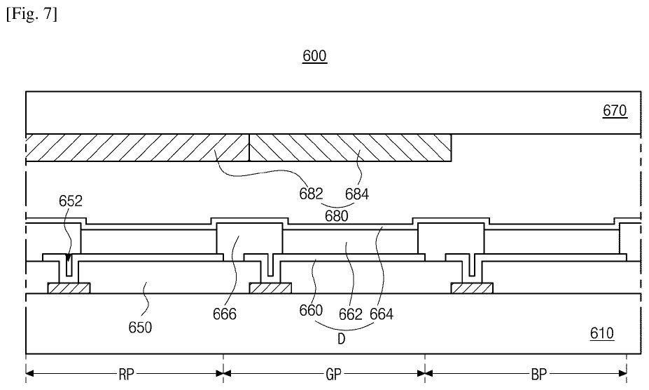

FIG. 7 is a schematic cross-sectional view illustrating an organic light emitting display device according to a third embodiment of the present disclosure.

As shown in FIG. 7, the organic light emitting display device 600 includes a first substrate 610, where a red pixel RP, a green pixel GP and a blue pixel BP are defined, a second substrate 670 facing the first substrate 610, an OLED D, which is positioned between the first and second substrates 610 and 670 and providing white emission, and a color conversion layer 680 between the OLED D and the second substrate 670.

A color filter can be formed between the second substrate 670 and each color conversion layer 680.

A TFT Tr, which corresponding to each of the red, green and blue pixels RP, GP and BP, is formed on the first substrate 610, and a passivation layer 650, which has a drain contact hole 652 exposing an electrode, e.g., a drain electrode, of the TFT Tr is formed to cover the TFT Tr.

The OLED D including a first electrode 660, an organic emitting layer 662 and a second electrode 664 is formed on the passivation layer 650. In this instance, the first electrode 660 can be connected to the drain electrode of the TFT Tr through the drain contact hole 652.

A bank layer 666 covering an edge of the first electrode 660 is formed at a boundary of the red, green and blue pixel regions RP, GP and BP.

The OLED D emits a blue light and can have a structure shown in FIG. 3 or FIG. 4. Namely, the OLED D is formed in each of the red, green and blue pixels RP, GP and BP and provides the blue light.

The color conversion layer 680 includes a first color conversion layer 682 corresponding to the red pixel RP and a second color conversion layer 684 corresponding to the green pixel GP. For example, the color conversion layer 680 can include an inorganic color conversion material such as a quantum dot.

The blue light from the OLED D is converted into the red light by the first color conversion layer 682 in the red pixel RP, and the blue light from the OLED D is converted into the green light by the second color conversion layer 684 in the green pixel GP.

Accordingly, the organic light emitting display device 600 can display a full-color image.

On the other hand, when the light from the OLED D passes through the first substrate 610, the color conversion layer 680 is disposed between the OLED D and the first substrate 610.

While the present disclosure has been described with reference to exemplary embodiments and examples, these embodiments and examples are not intended to limit the scope of the present disclosure. Rather, it will be apparent to those skilled in the art that various modifications and variations can be made in the present disclosure without departing from the spirit or scope of the invention. Thus, it is intended that the present disclosure cover the modifications and variations of the present disclosure provided they come within the scope of the appended claims and their equivalents.

The various embodiments described above can be combined to provide further embodiments. All of patents, patent application publications, patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An organic light emitting diode (OLED), comprising:
a first electrode;
a second electrode facing the first electrode; and
a first emitting material layer including a first host being an anthracene derivative and a first dopant being a pyrene derivative, the first emitting material layer being positioned between the first and second electrodes,
wherein at least one of an anthracene core of the first host and a pyrene core of the first dopant is deuterated.

2. The OLED of claim 1, wherein the first host is represented by Formula 1: 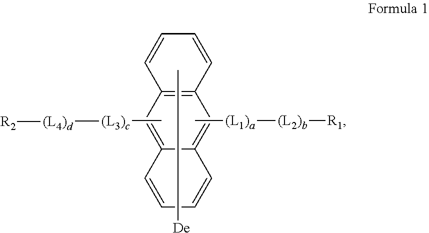
wherein each of R₁ and R₂ is independently C₆˜C₃₀ aryl group or C₅˜C₃₀ heteroaryl group, and each of L₁, L₂, L₃ and L₄ is independently C₆˜C₃₀ arylene group, and
wherein each of a, b, c and d is 0 or 1, and e is an integer of 1 to 8.

3. The OLED of claim 2, wherein the first host is a compound being one of the followings of Formula 2:

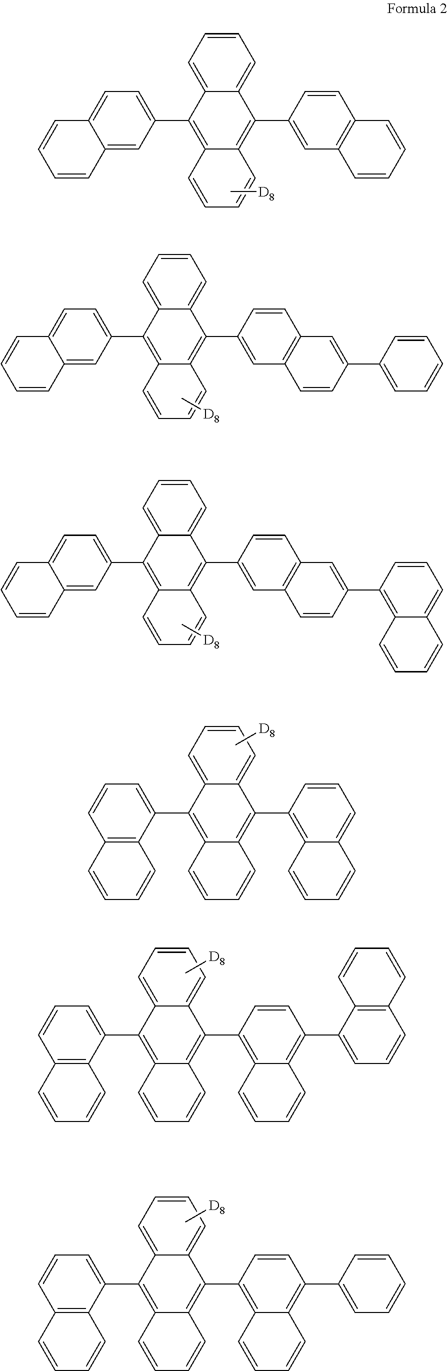

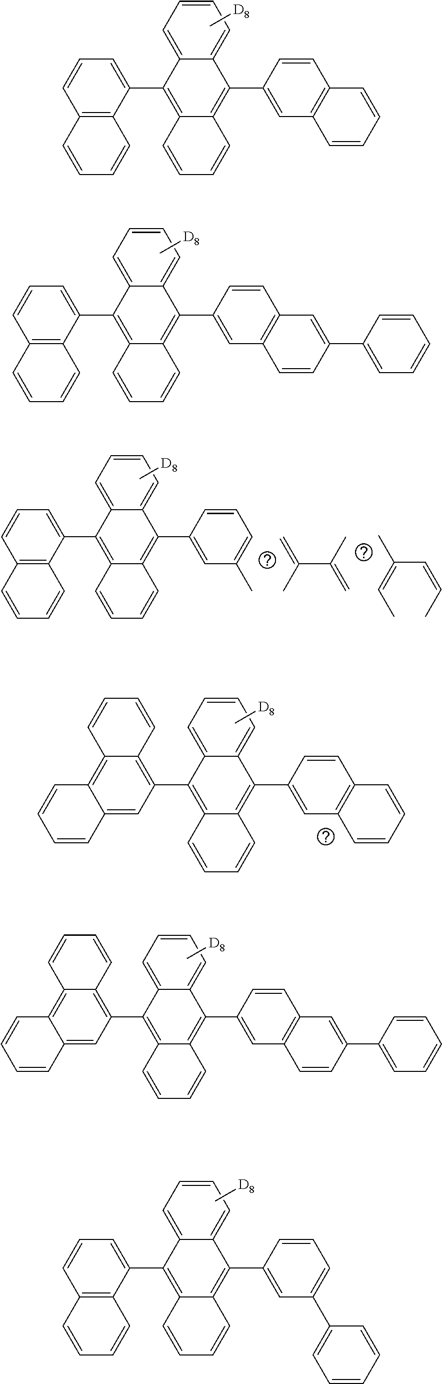

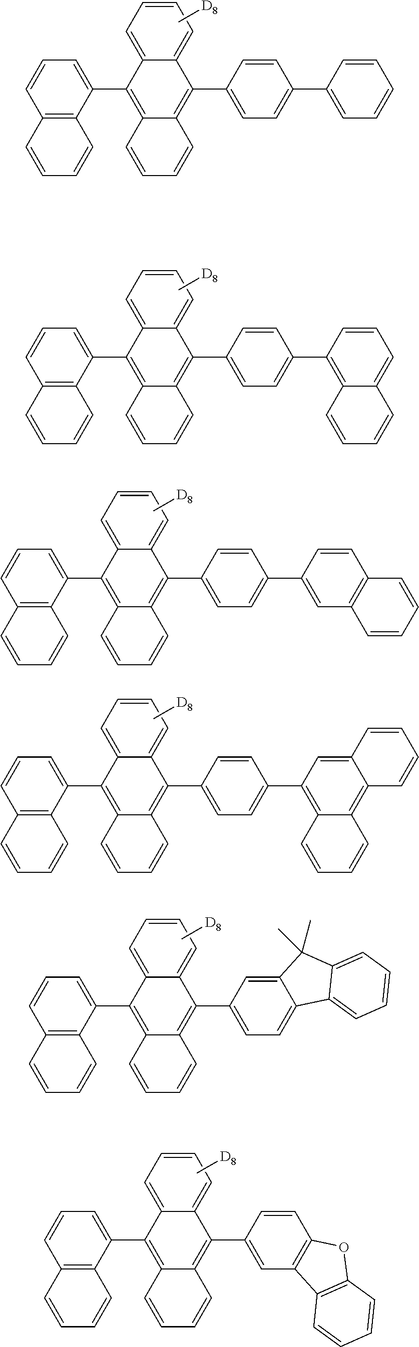

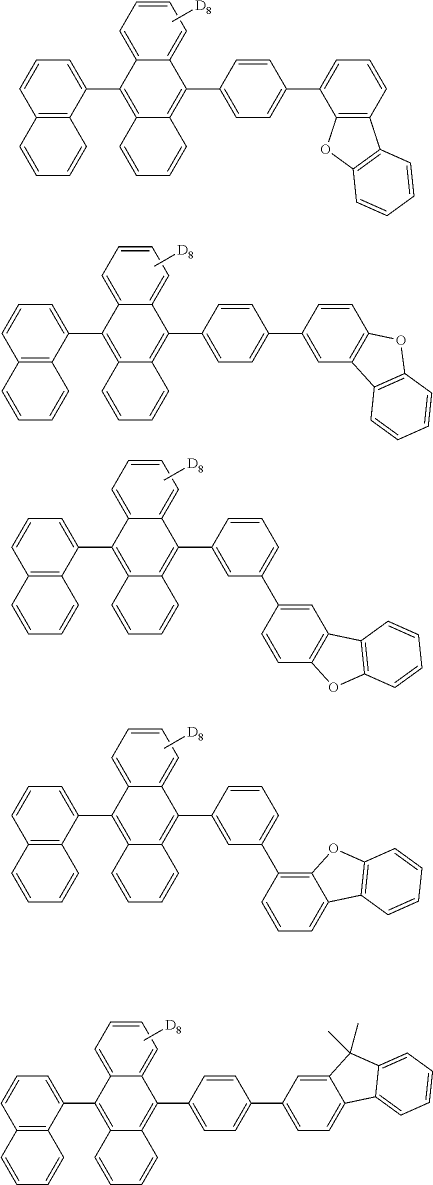

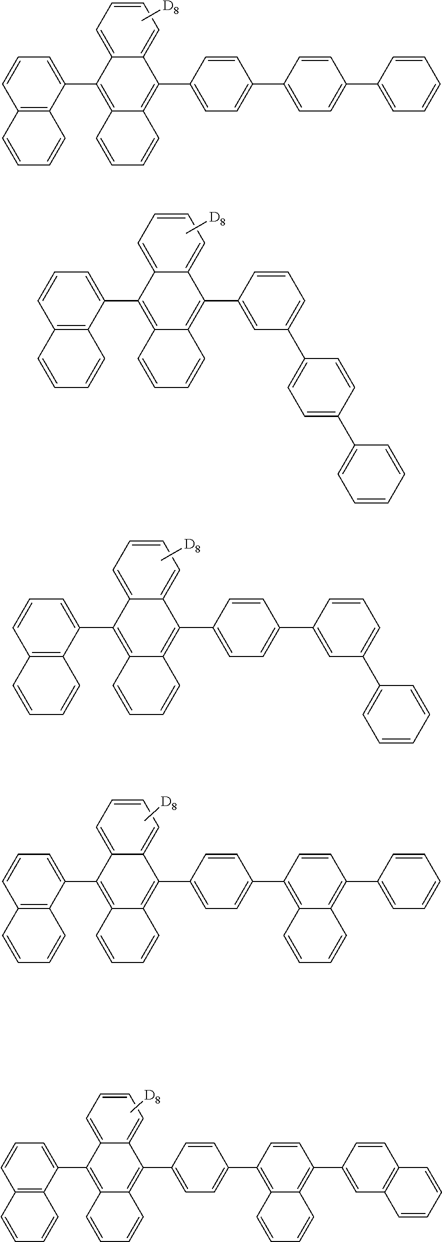

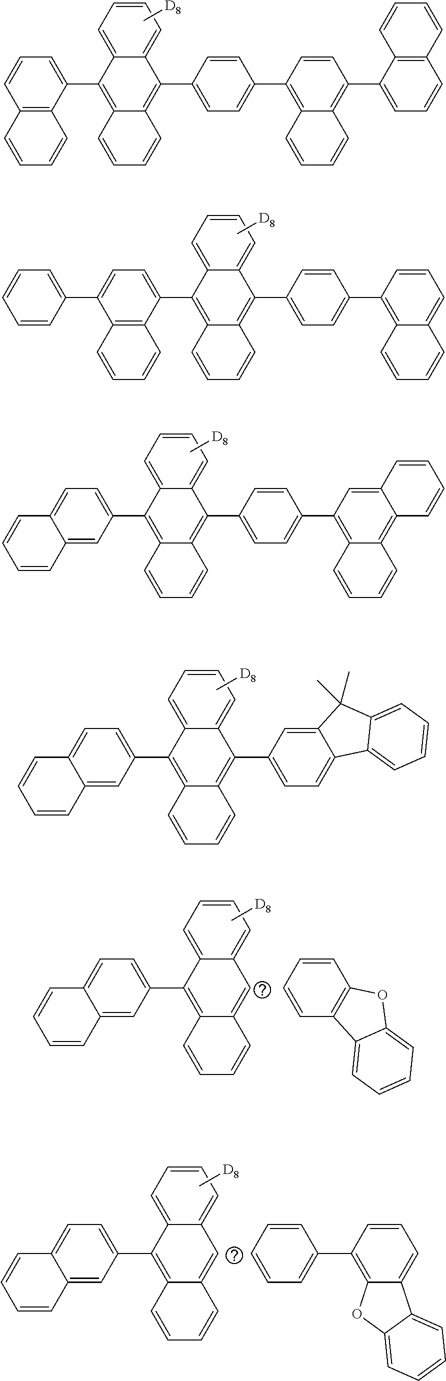

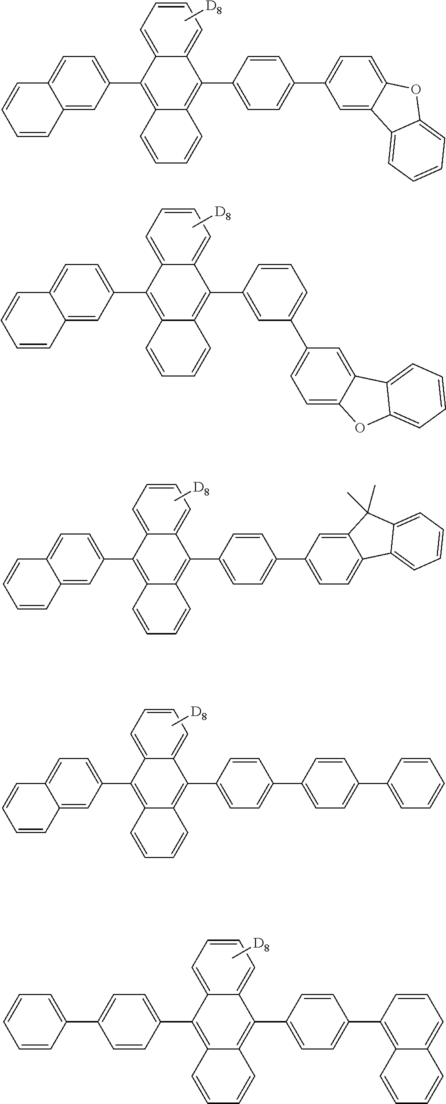

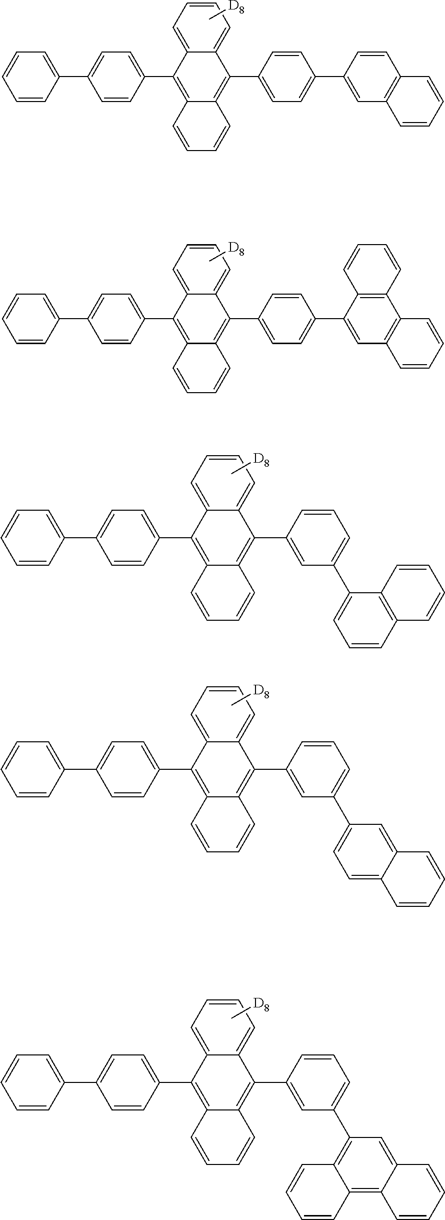

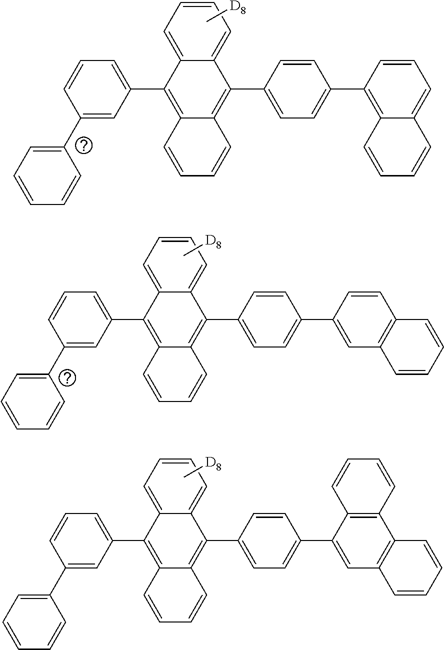

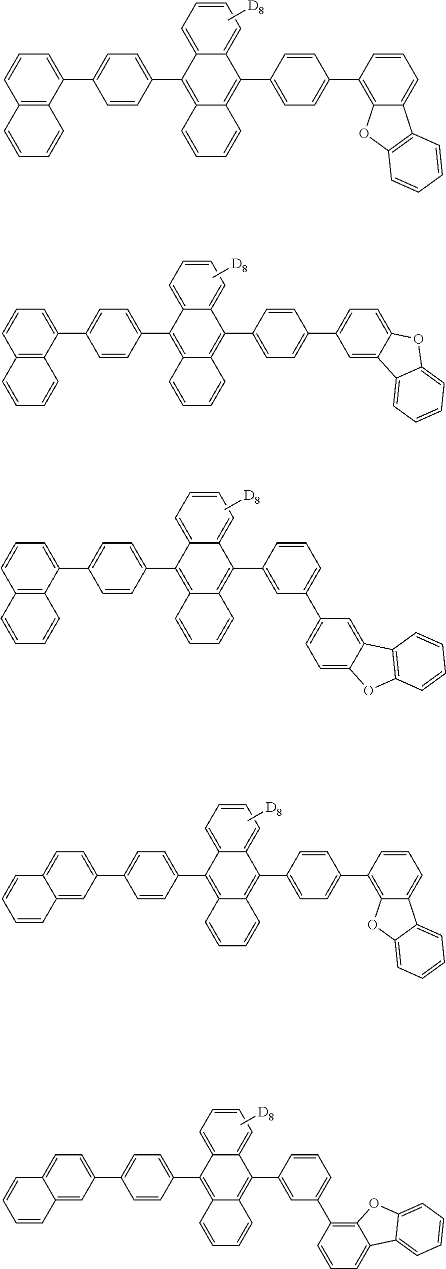

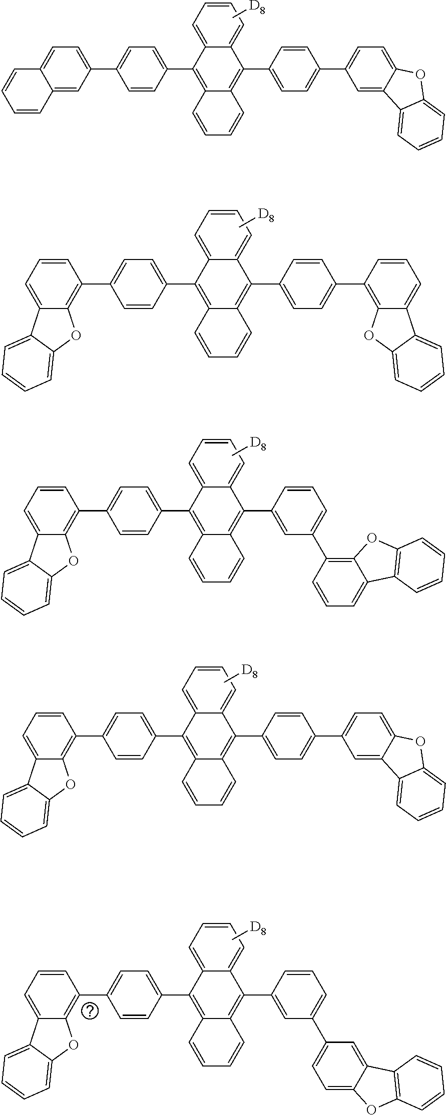

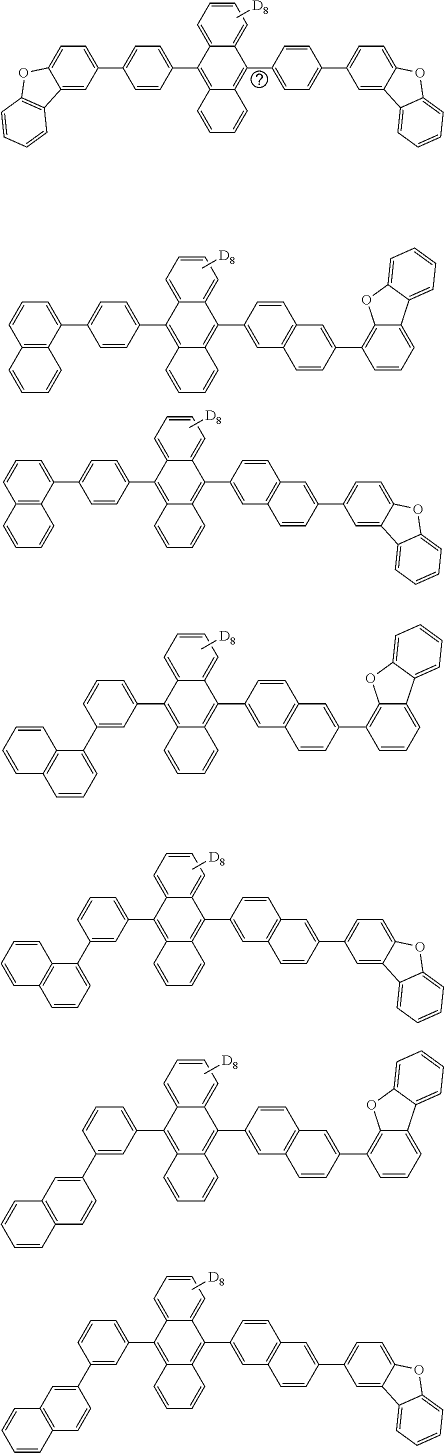

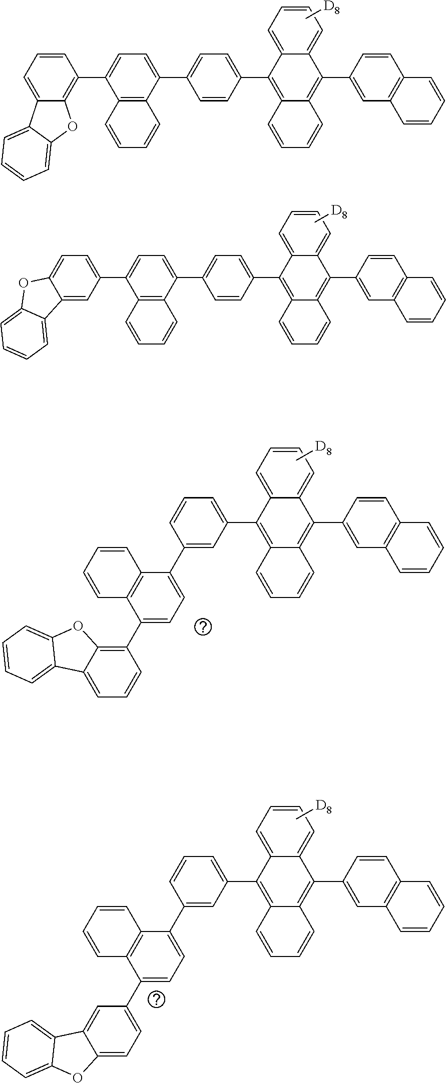

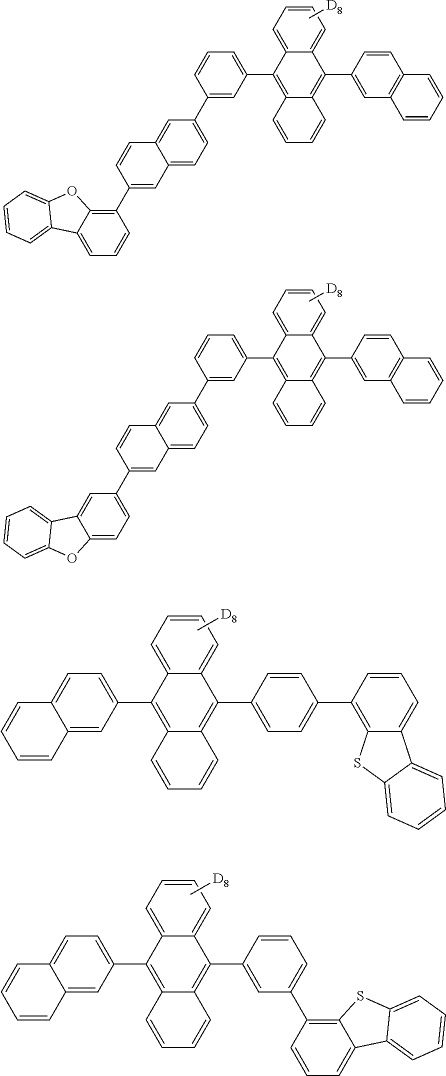

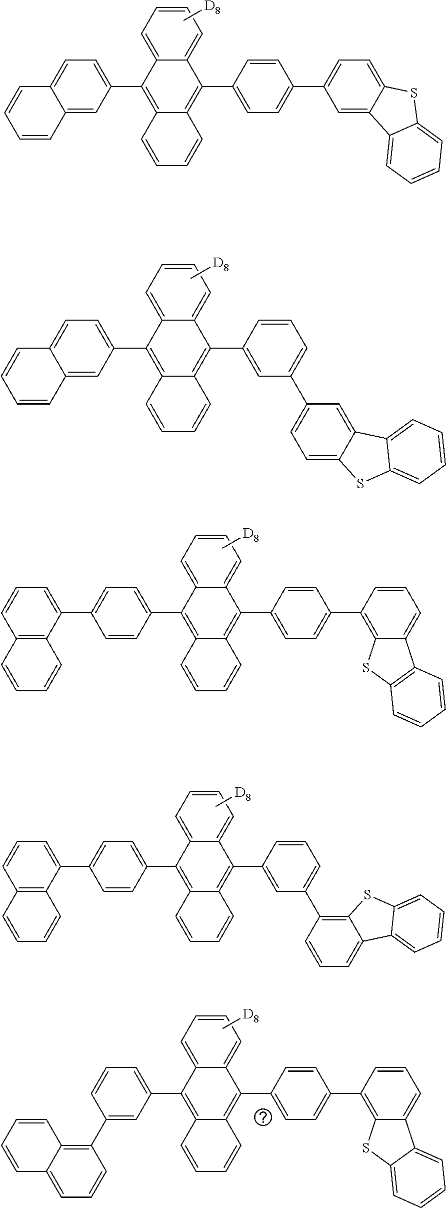

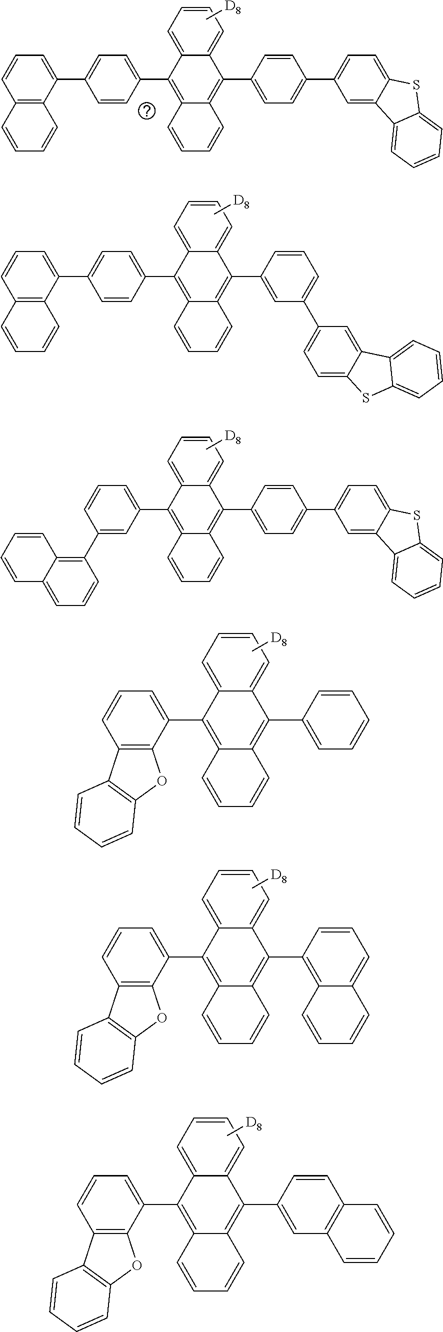

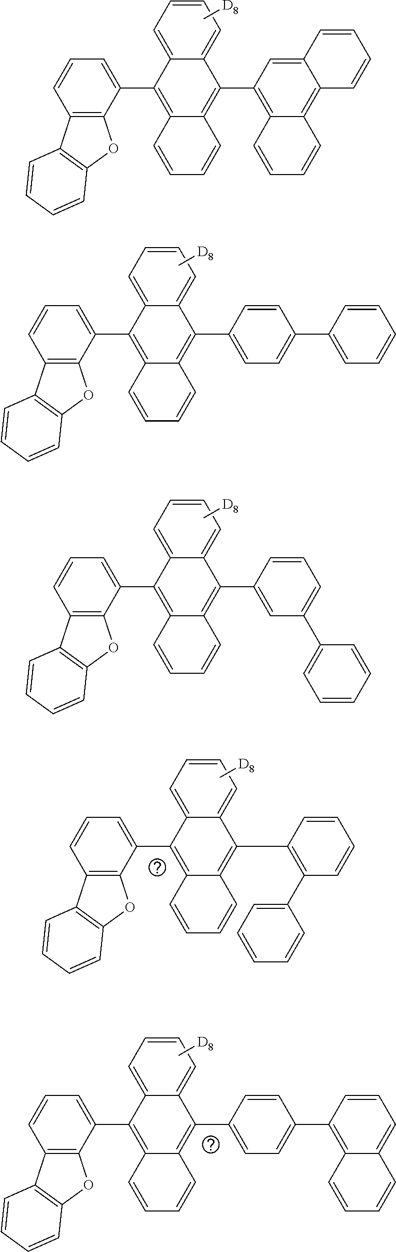

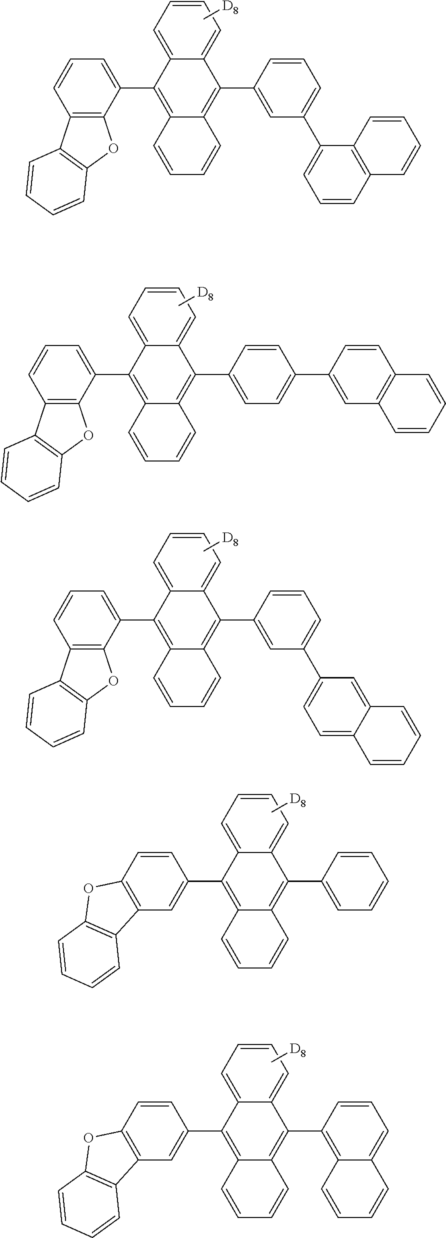

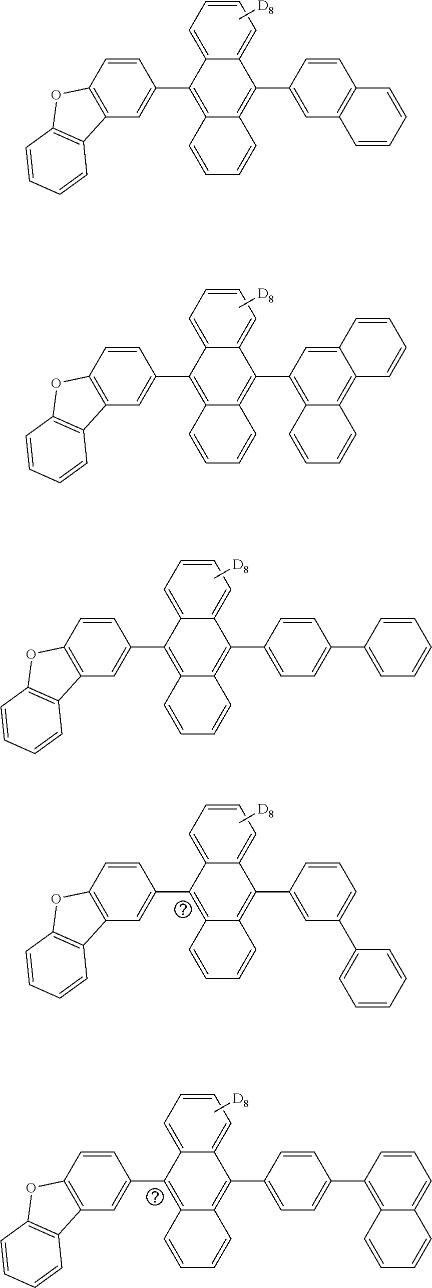

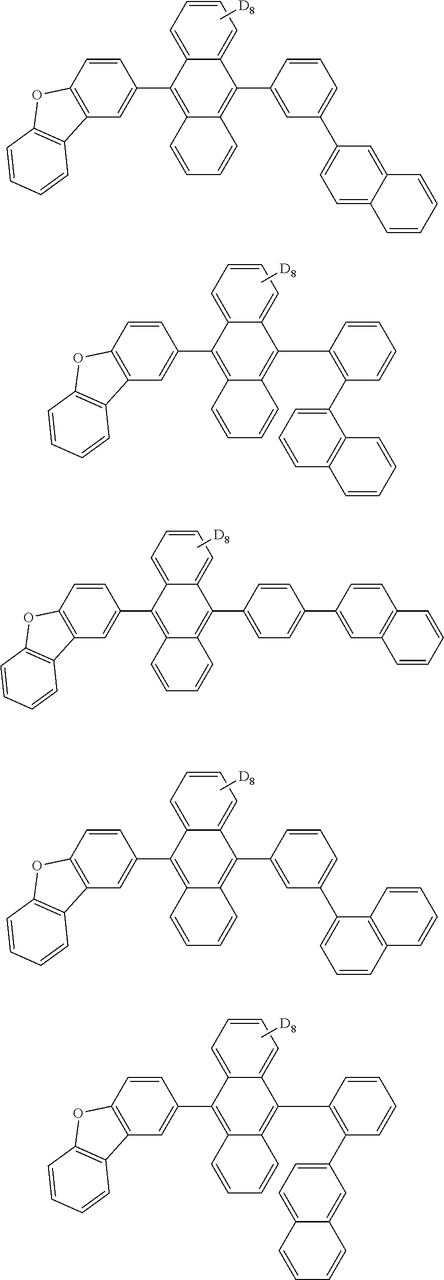

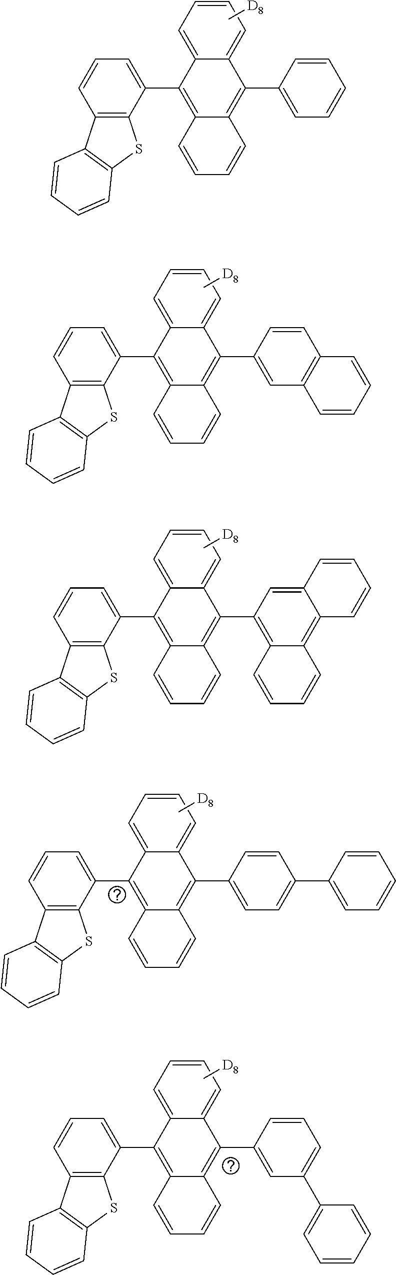

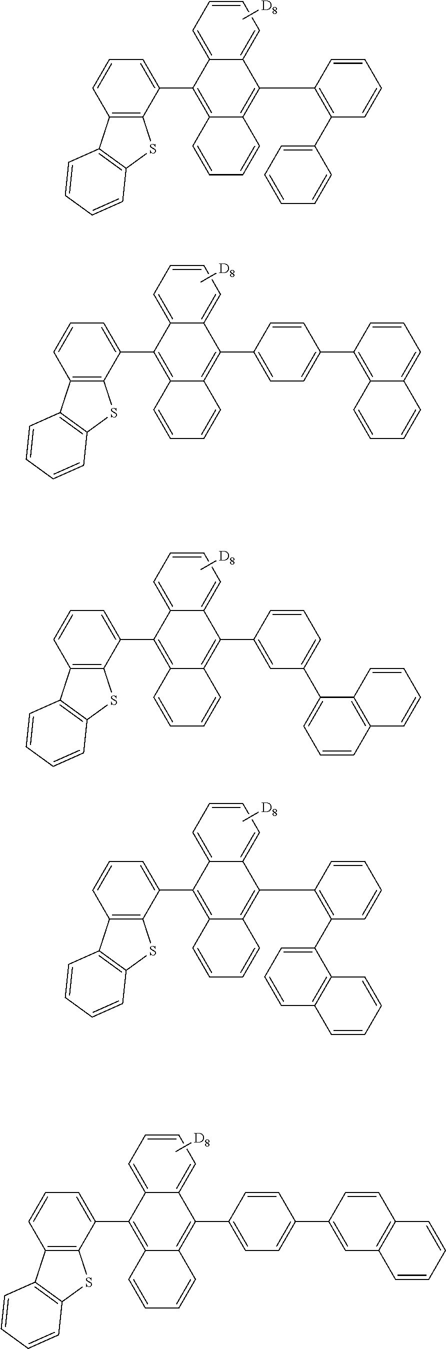

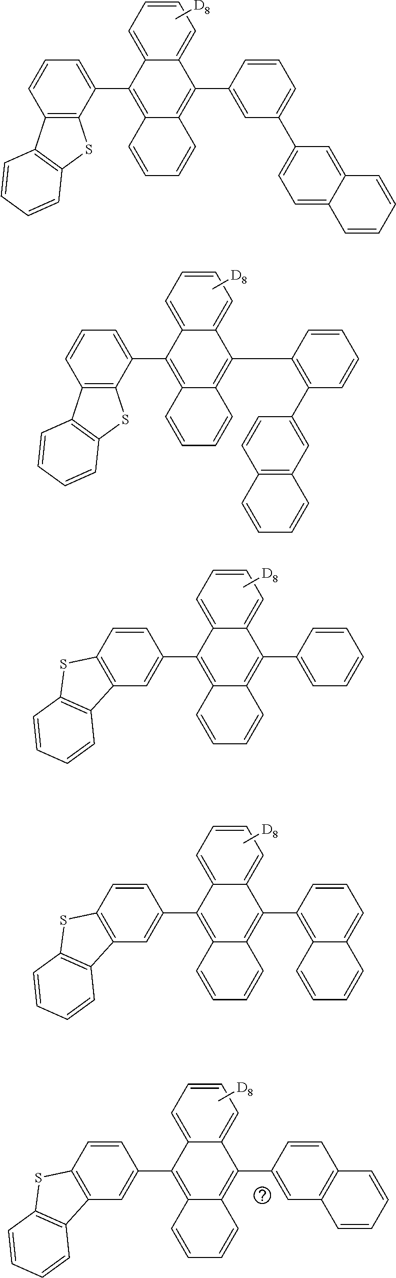

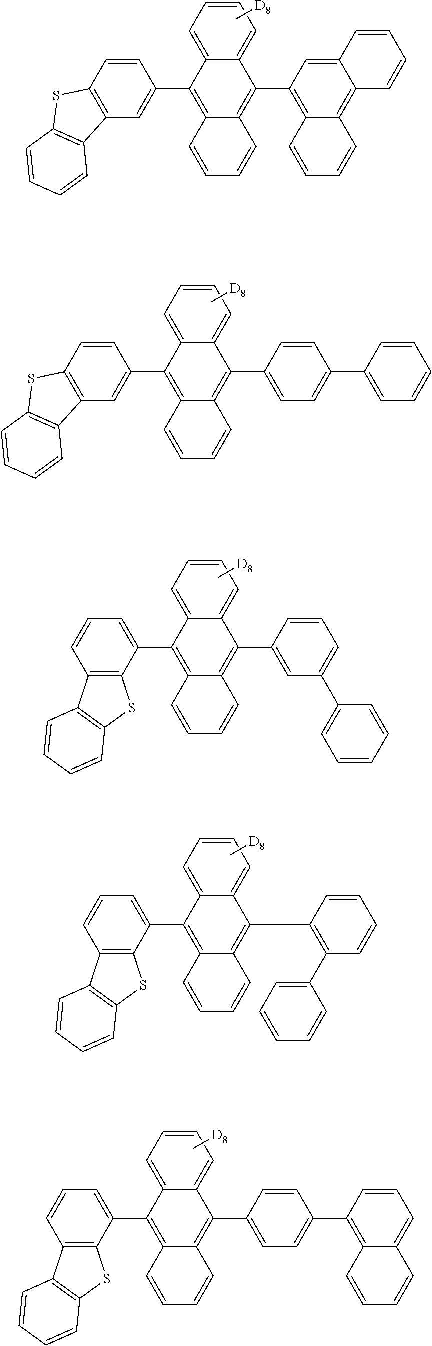

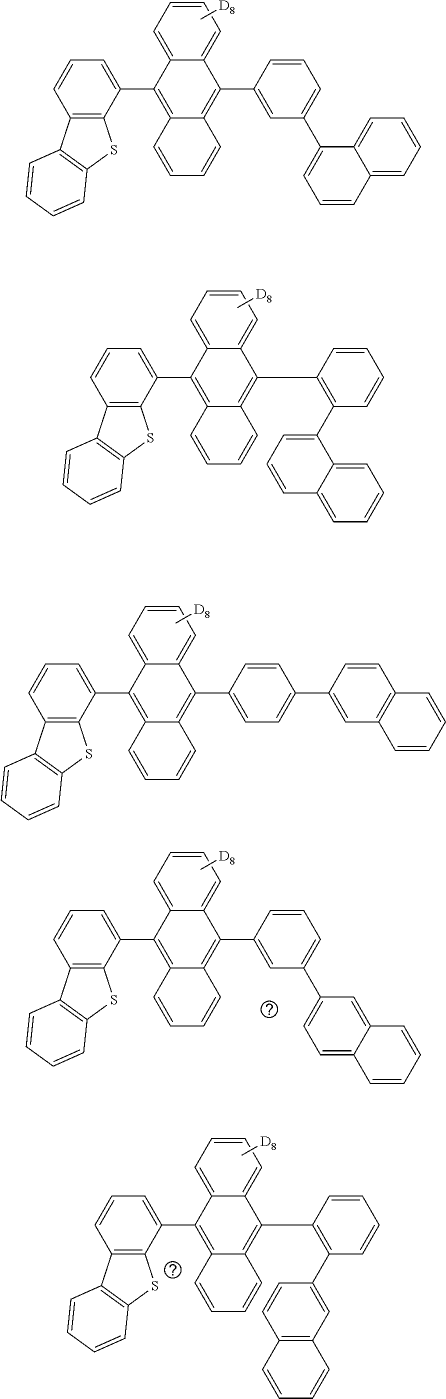

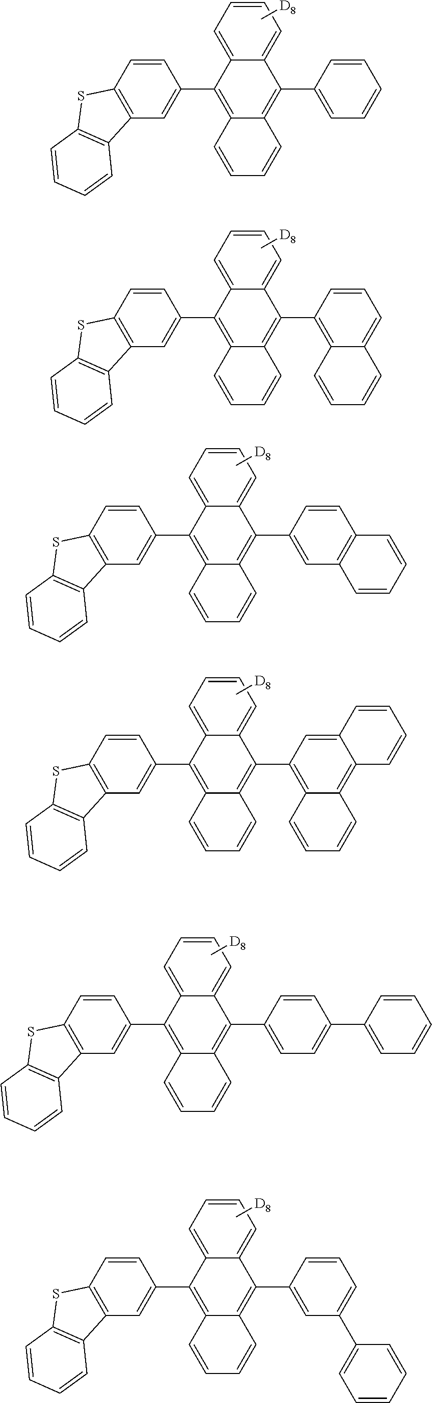

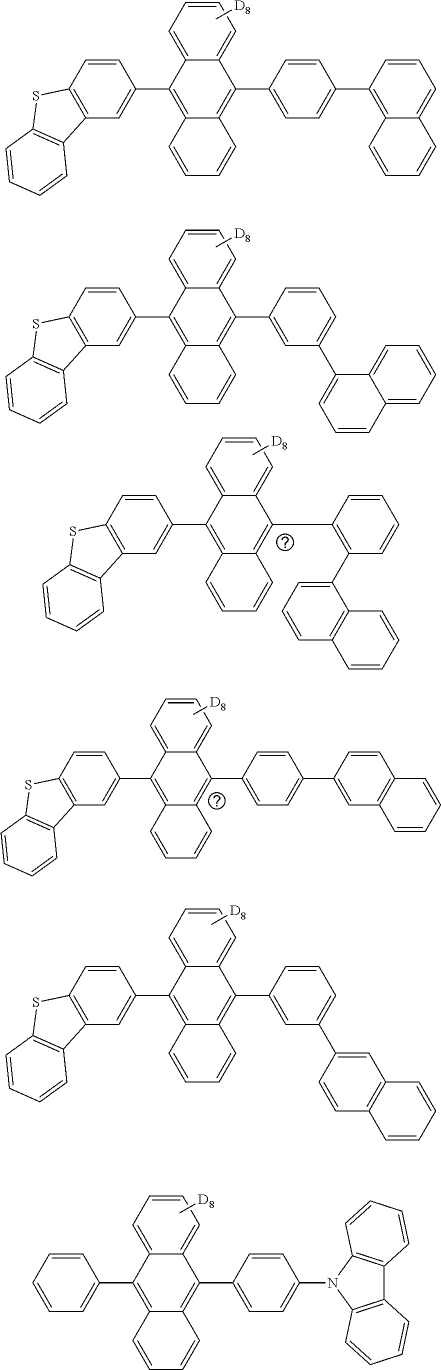

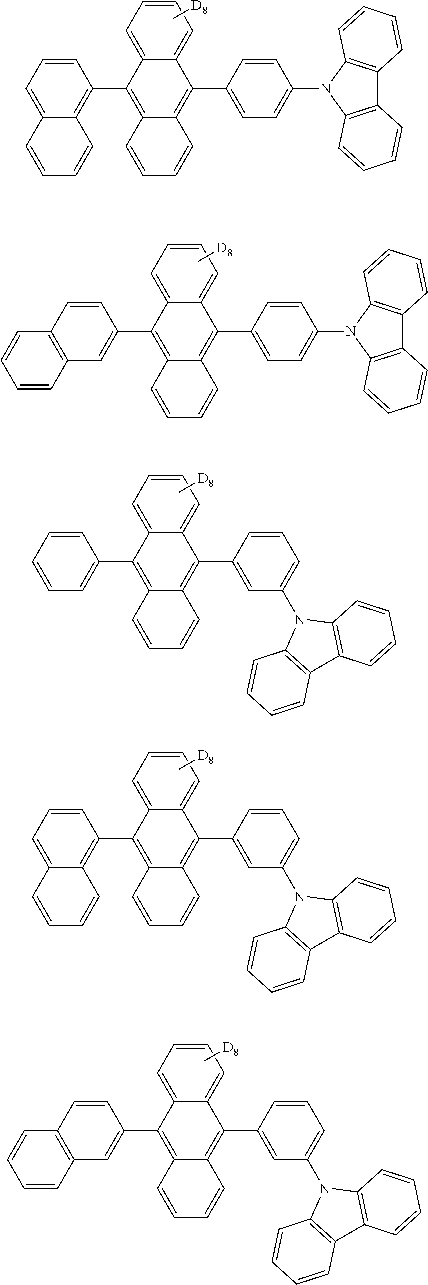

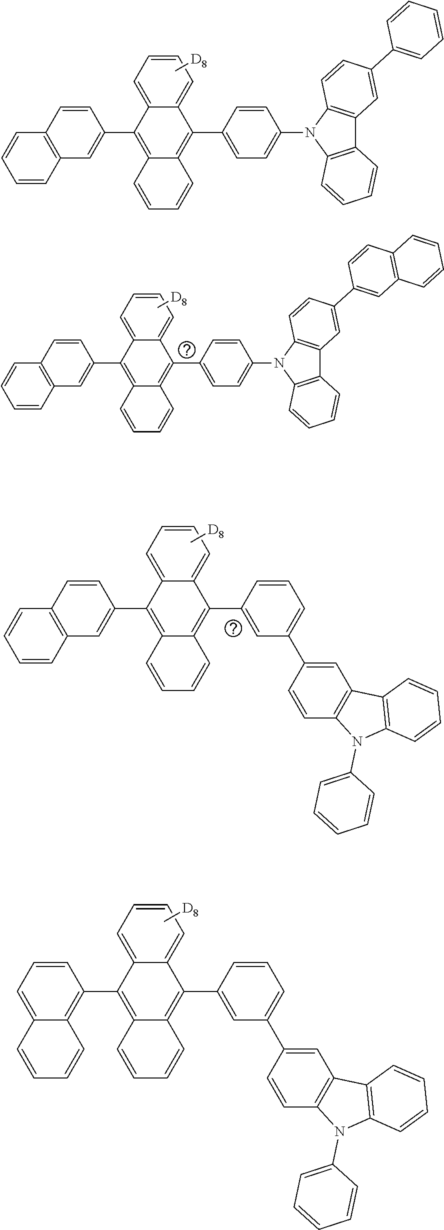

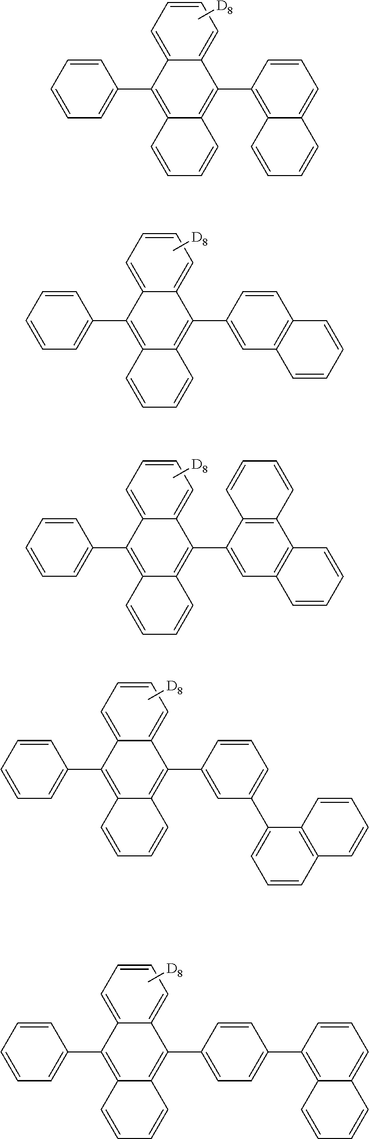

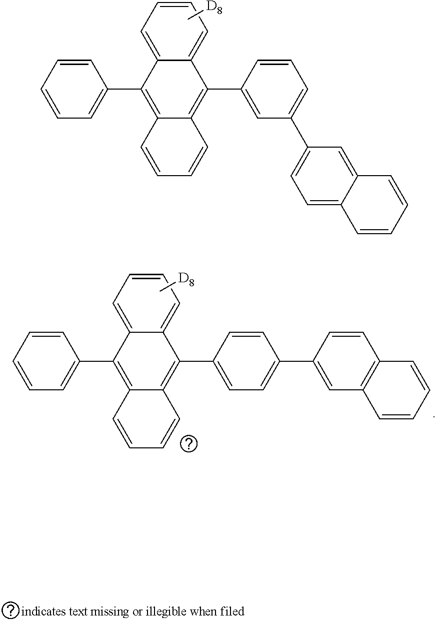

4. The OLED of claim 1, wherein the first dopant is represented by Formula 3: 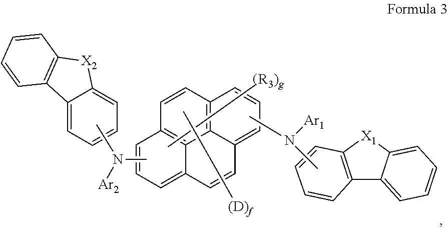
wherein each of X₁ and X₂ is independently O or S, each of Ar₁ and Ar₂ is independently C₆˜C₃₀ aryl group or C₅˜C₃₀ heteroaryl group,
wherein R₃ is C₁˜C₁₀ alkyl group or C₁˜C₁₀ cycloalkyl group, and f is an integer of 1 to 8, and
wherein g is an integer of 0 to 2, and a summation off and g is 8 or less.

5. The OLED of claim 4, wherein the first dopant is a compound being one of the followings of Formula 4:

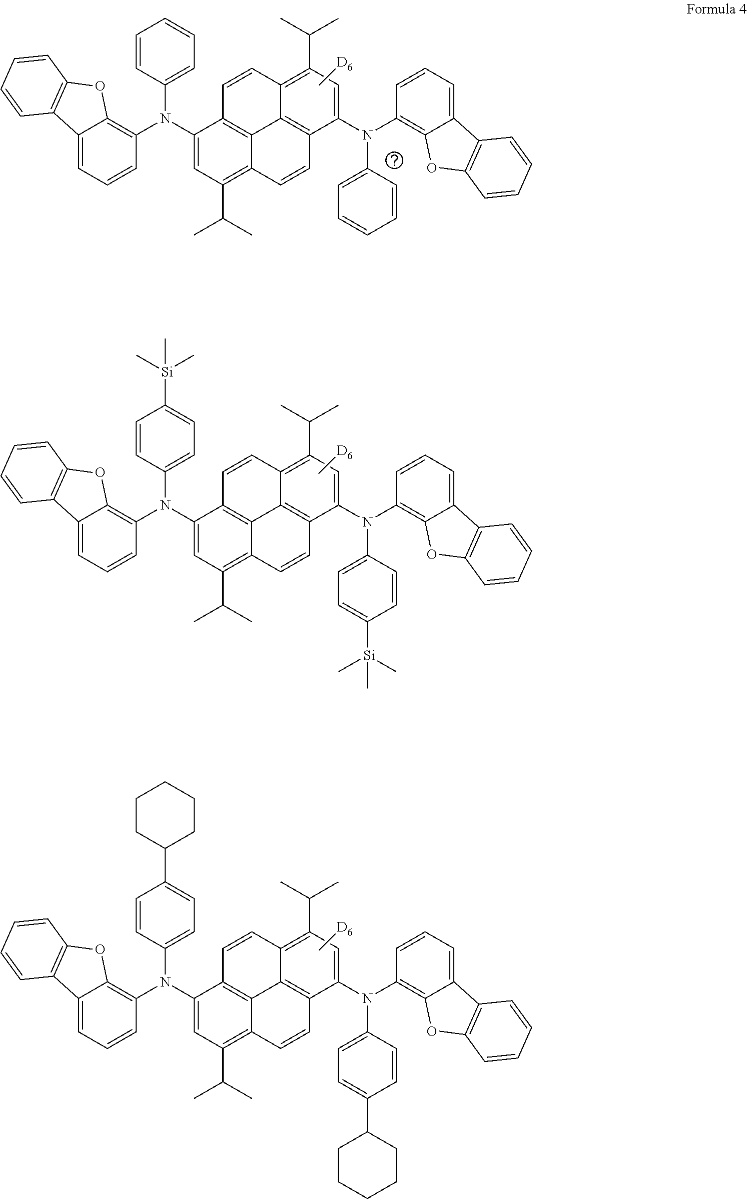

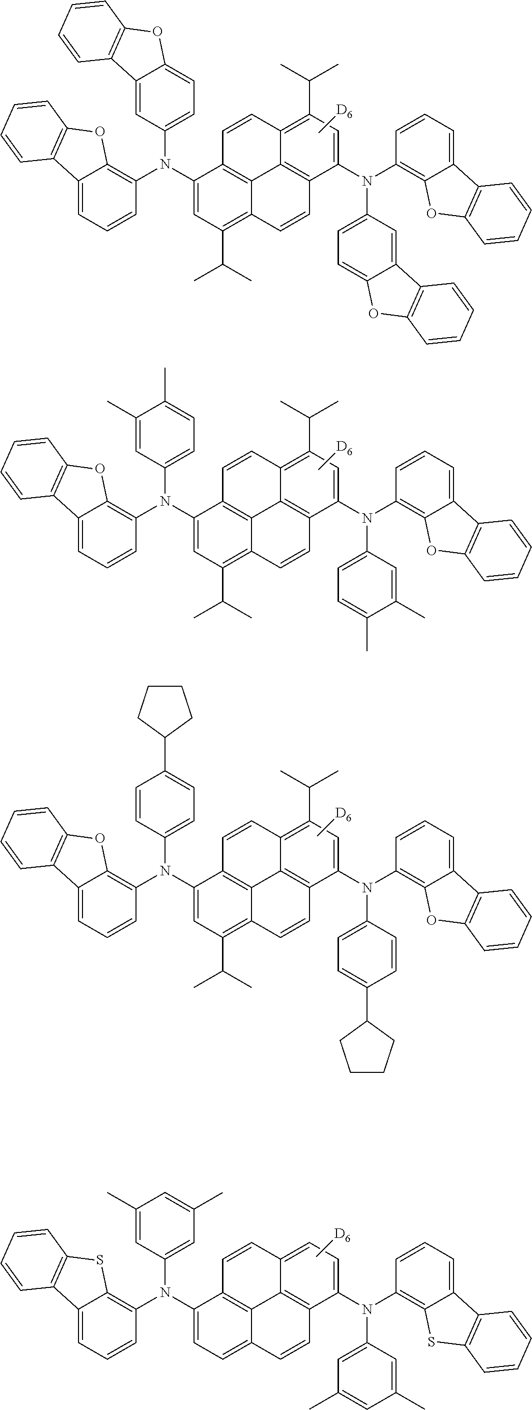

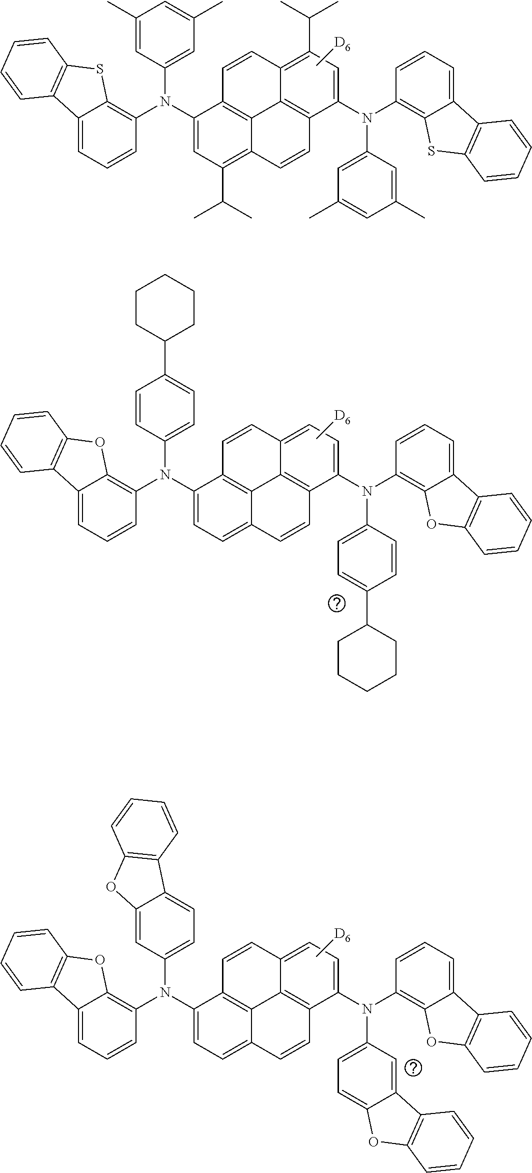

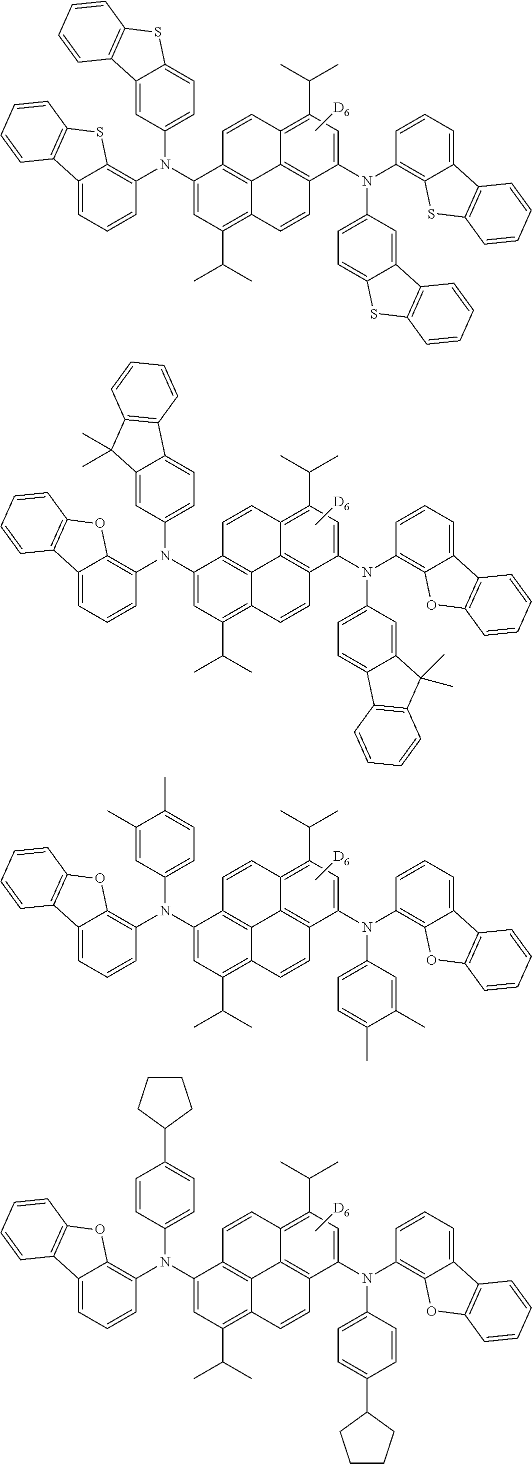

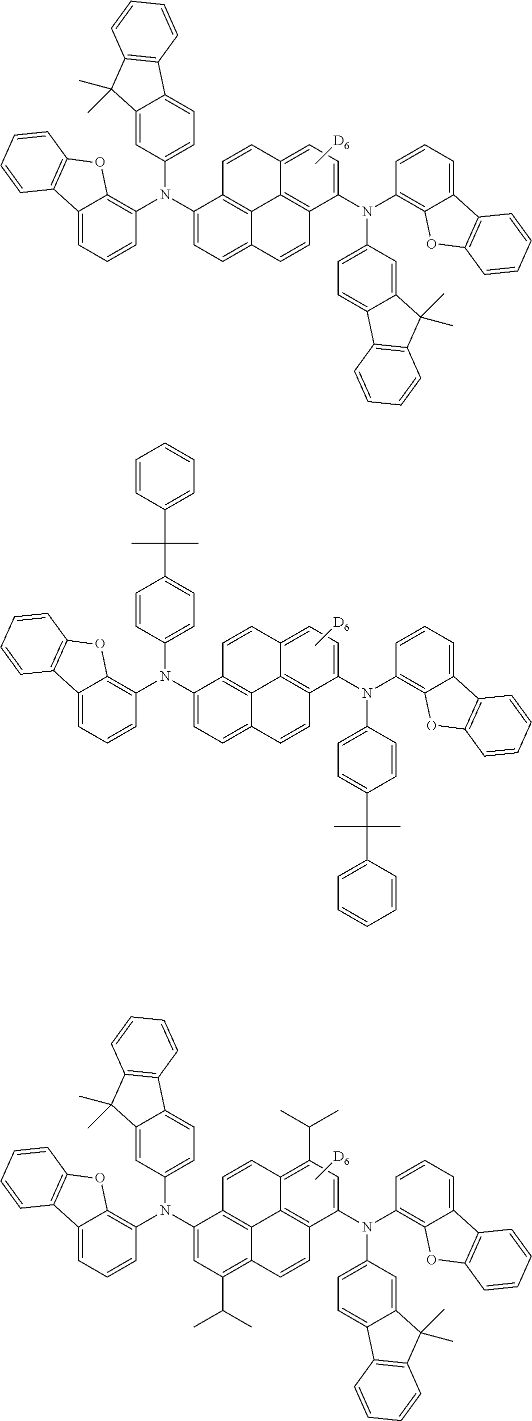

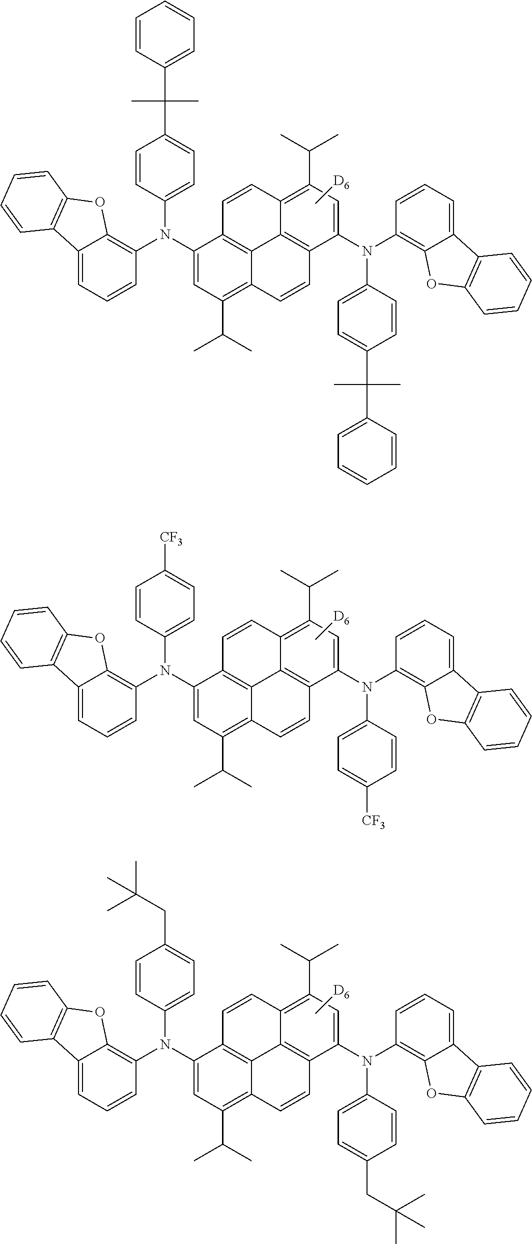

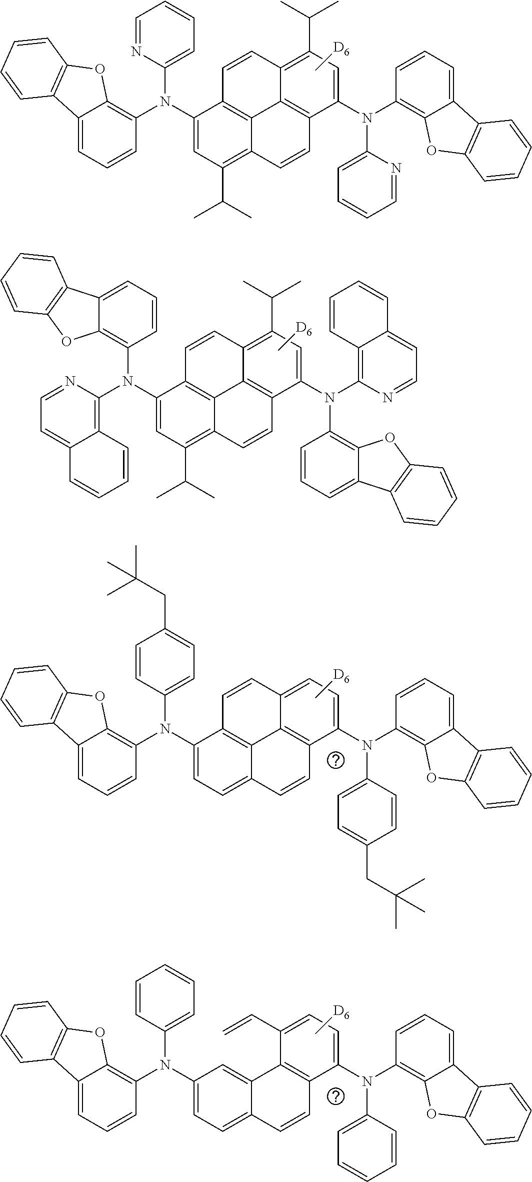

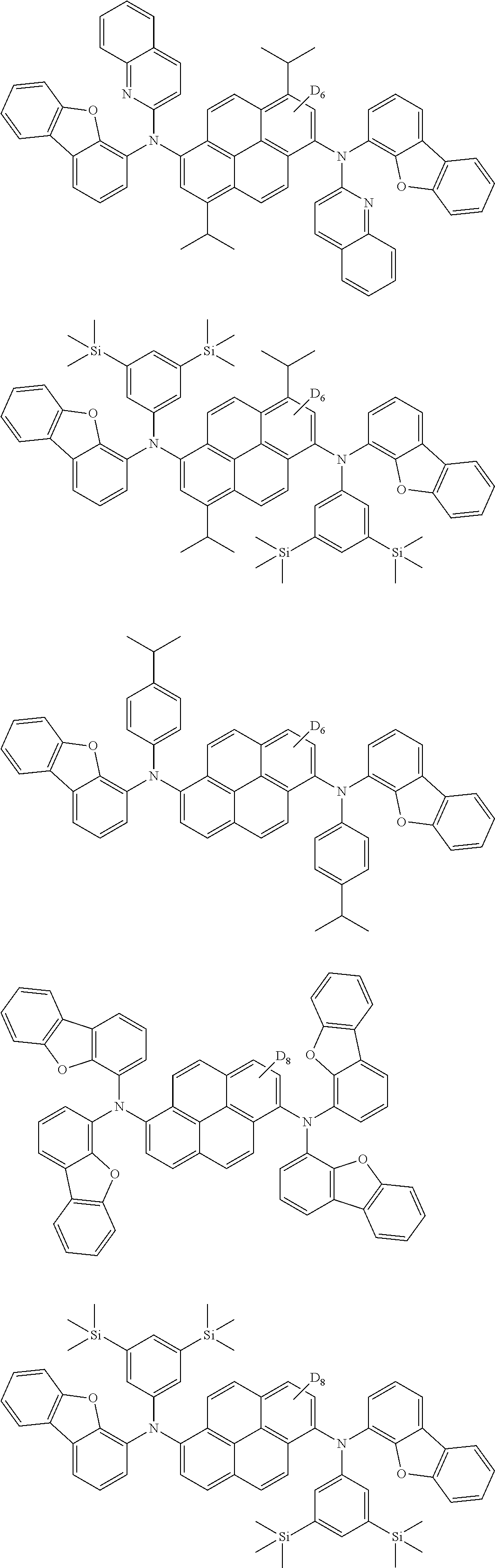

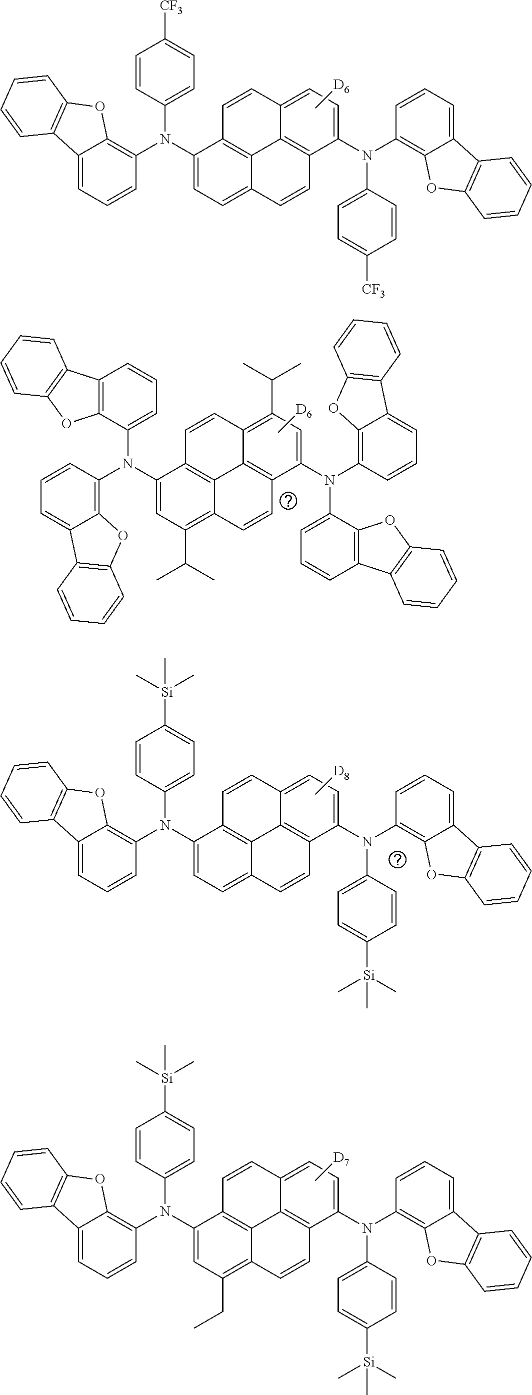

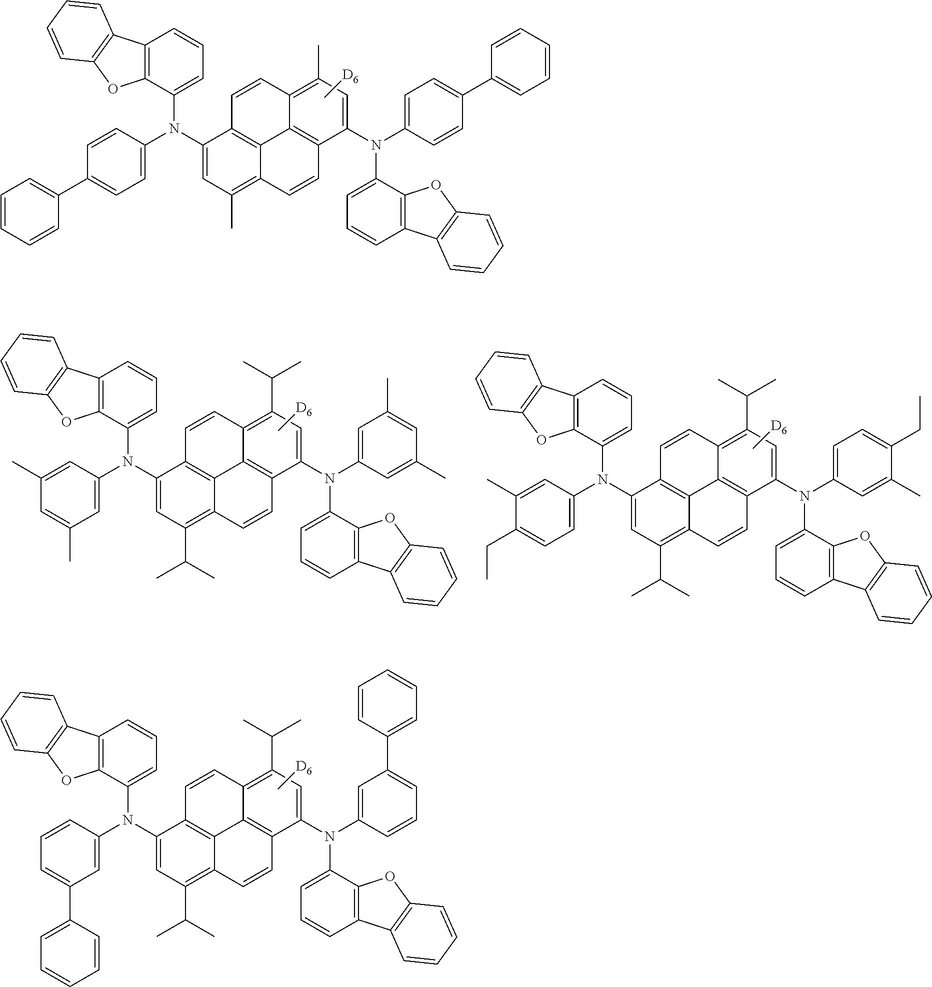

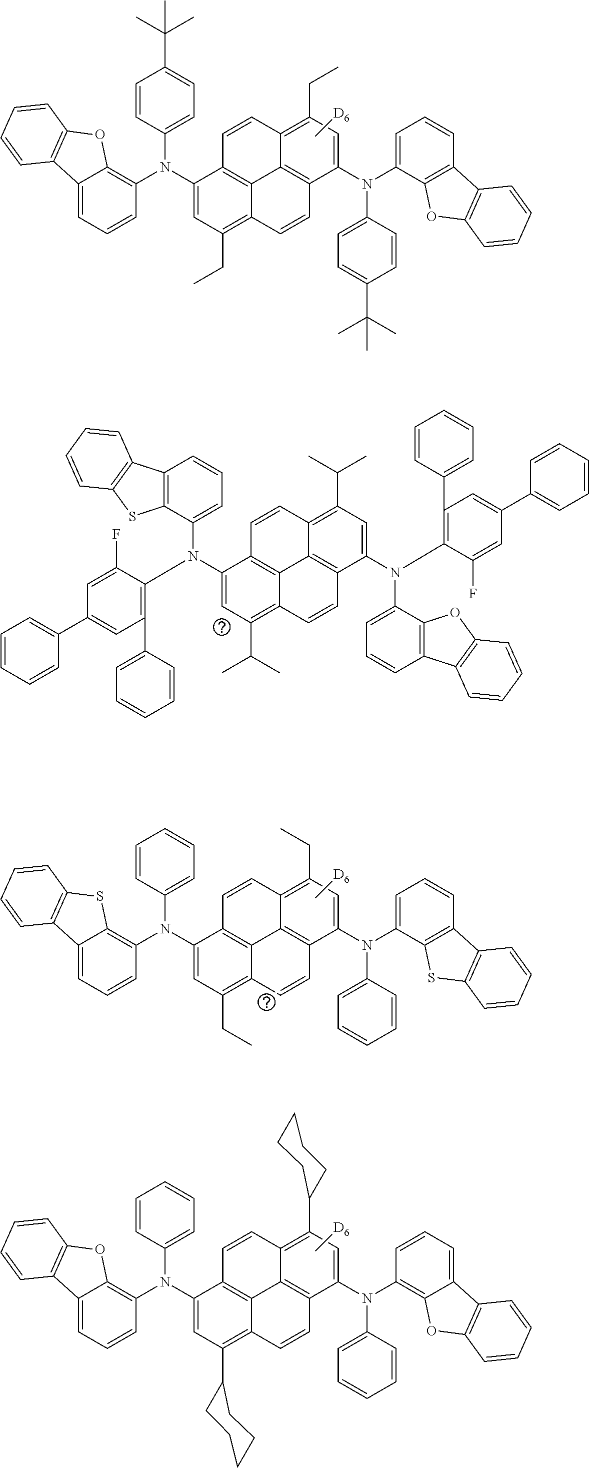

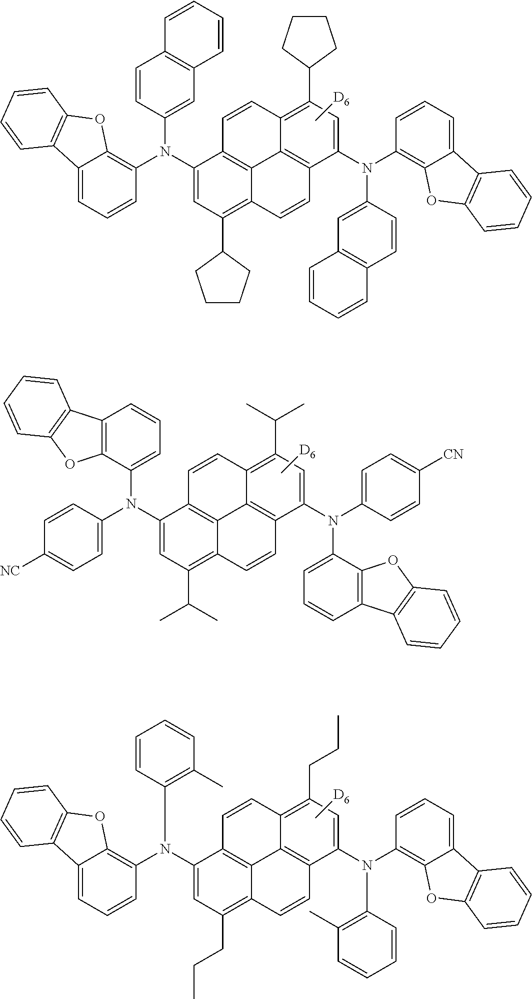

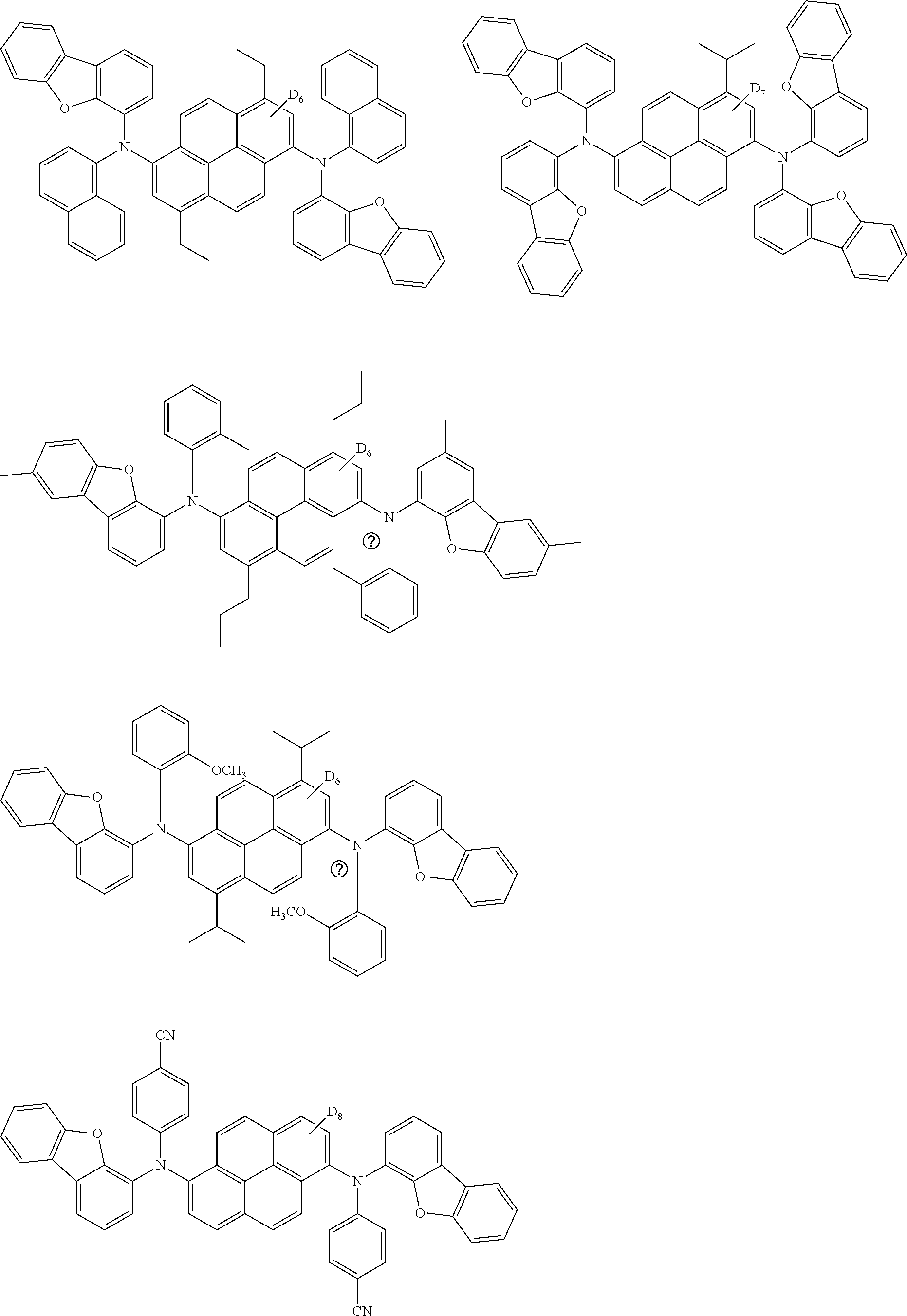

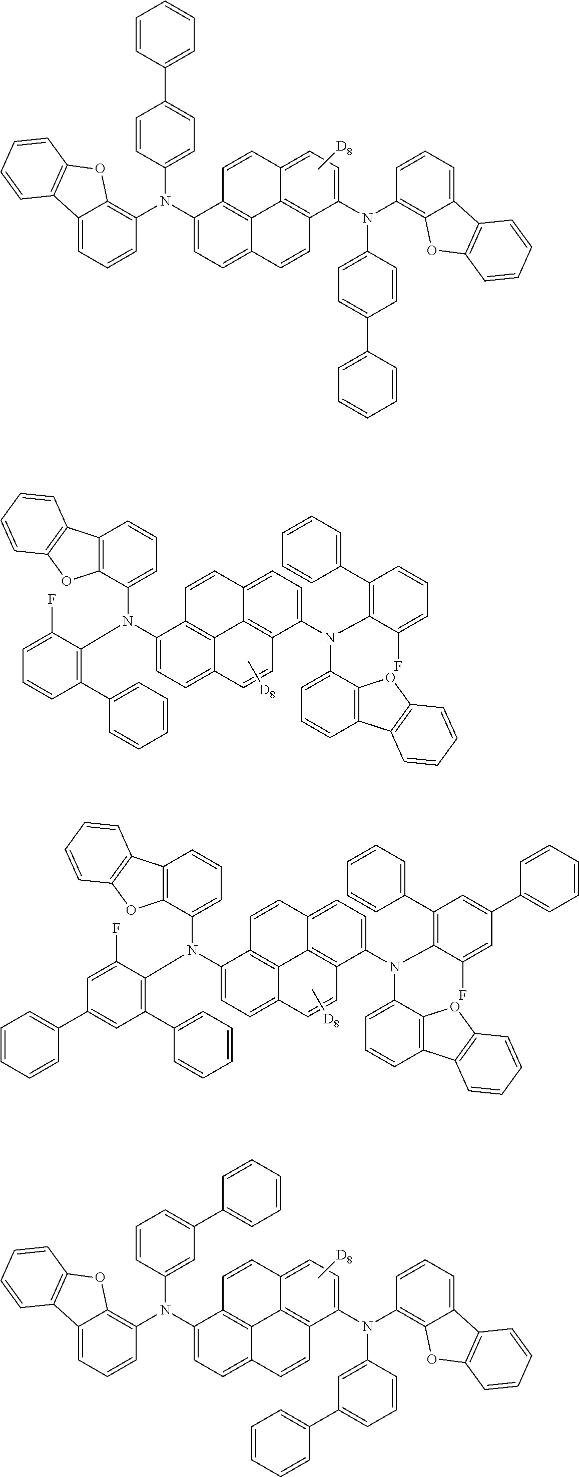

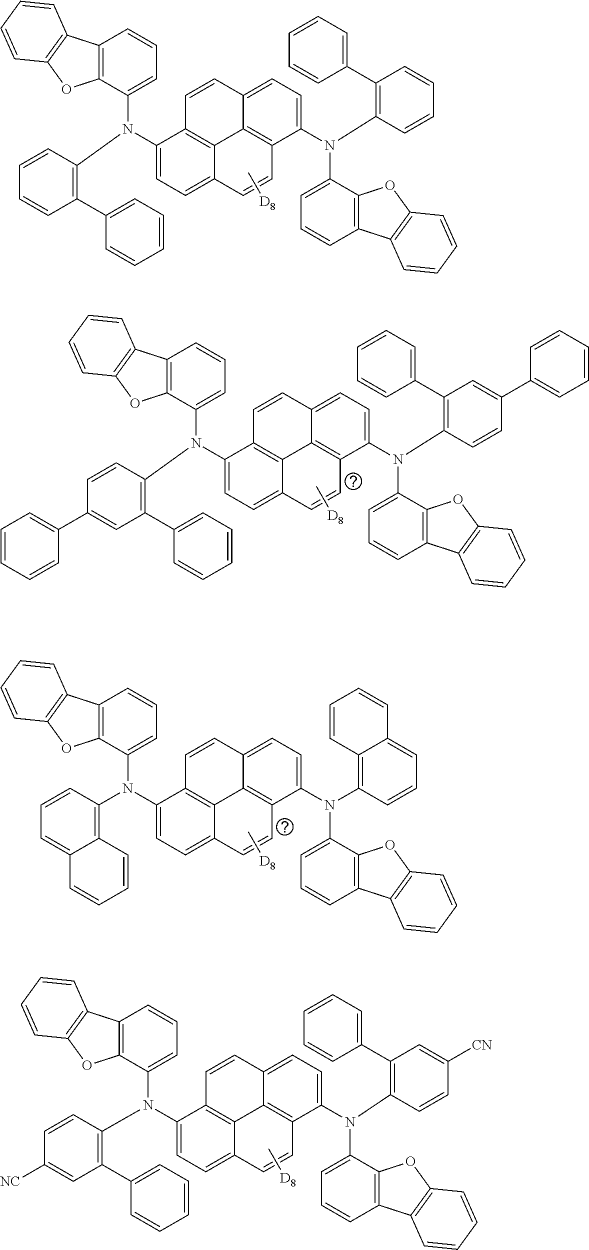

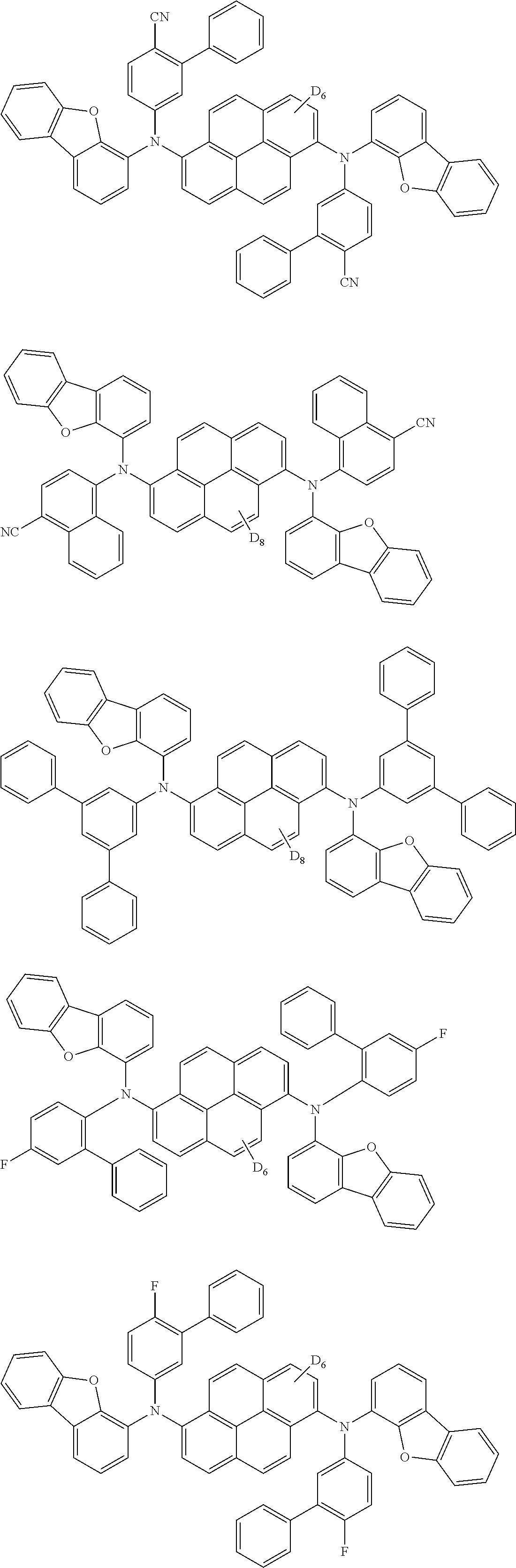

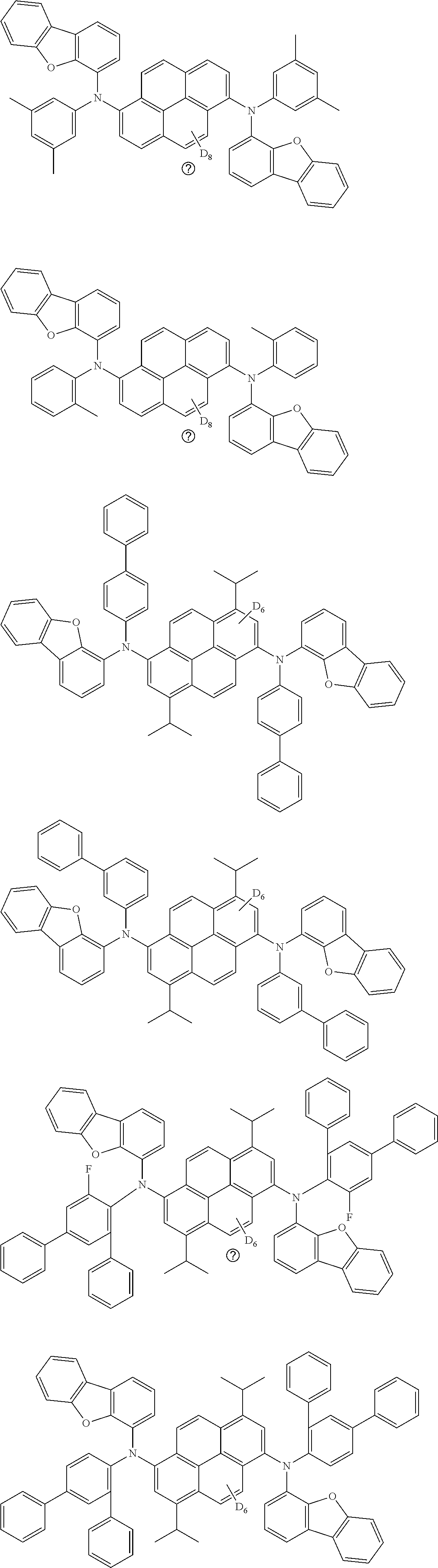

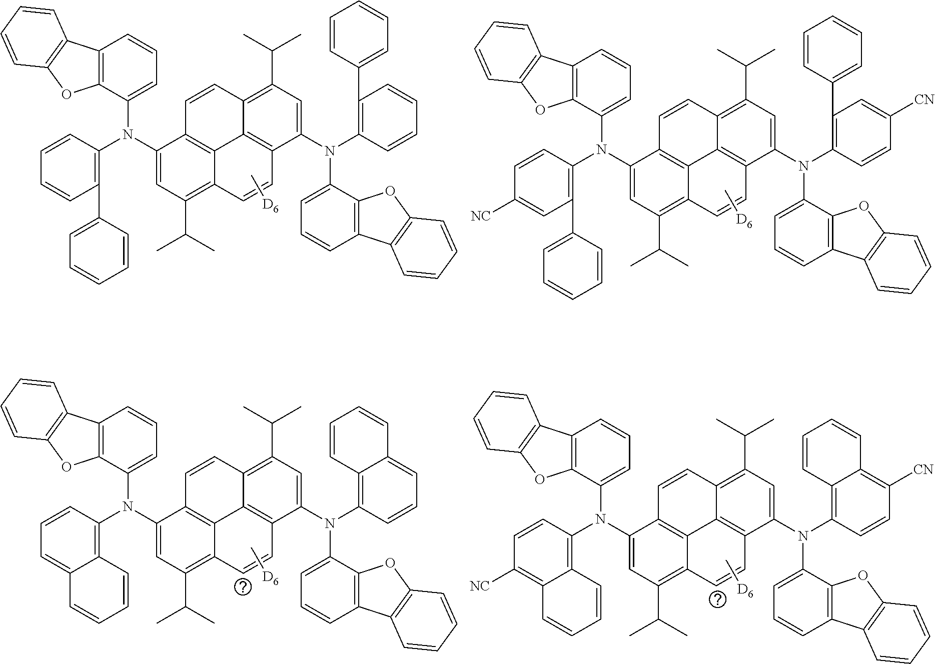

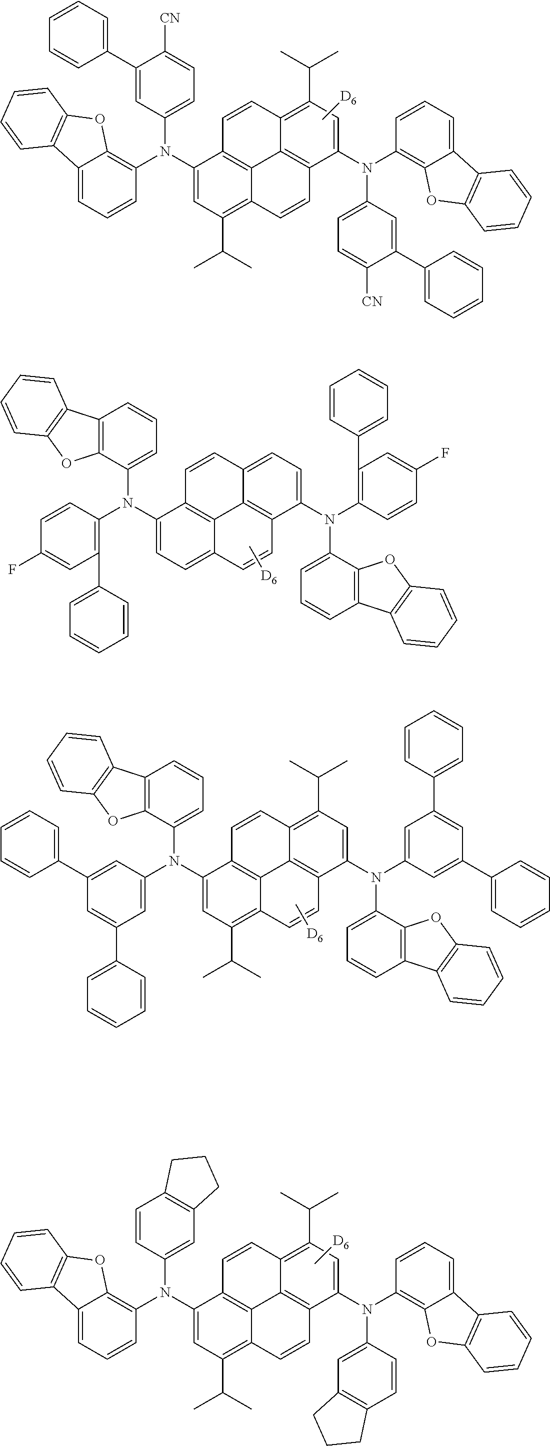

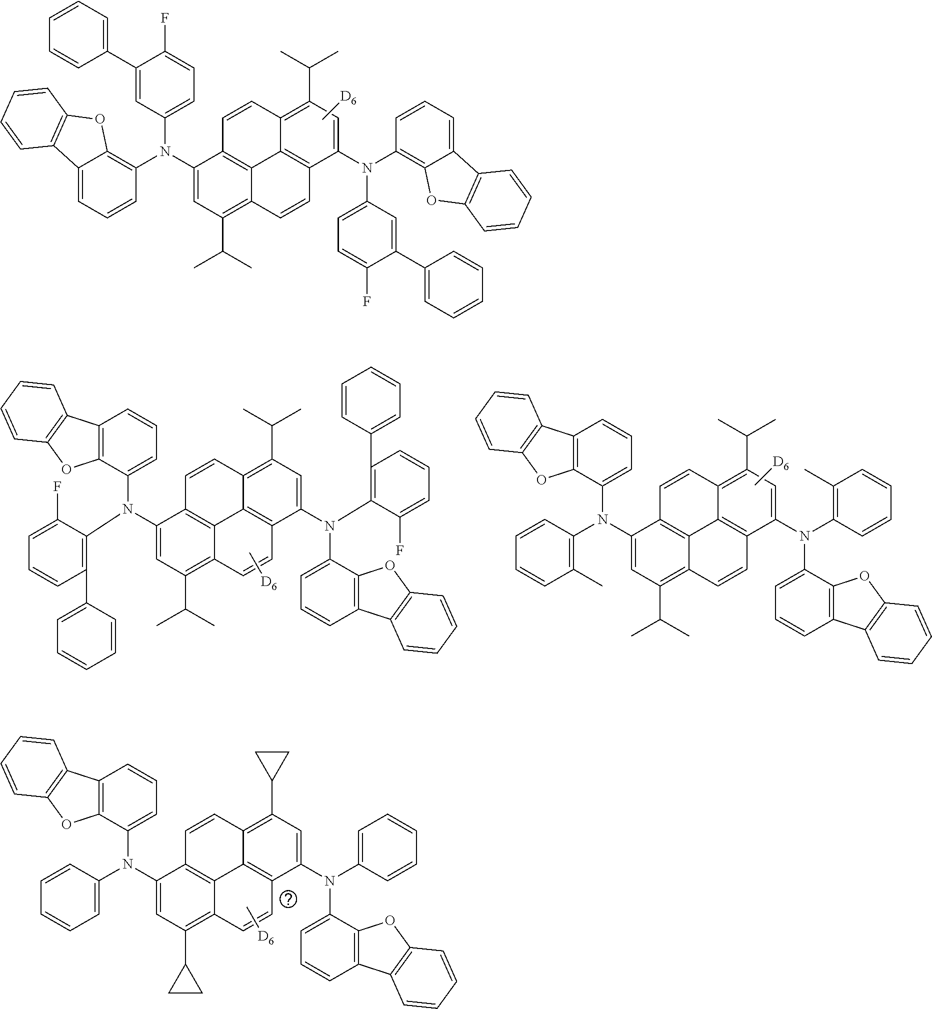

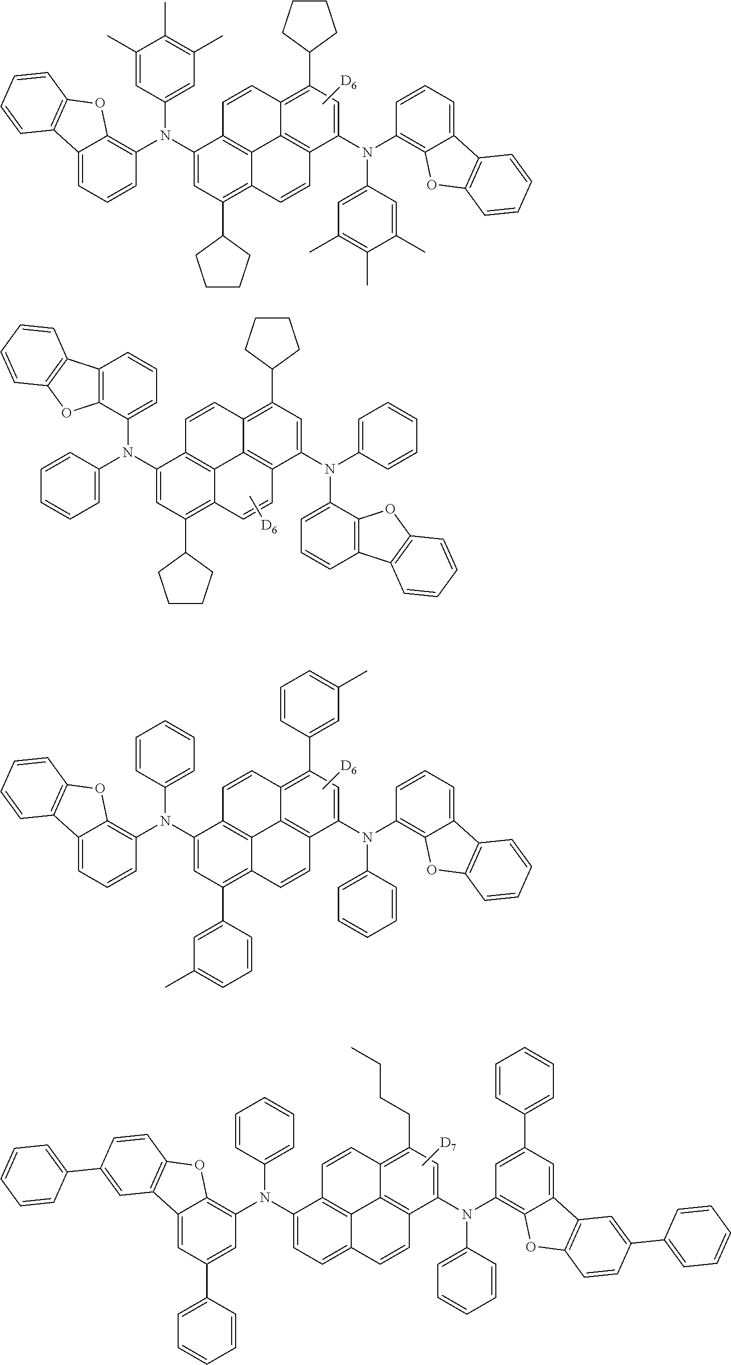

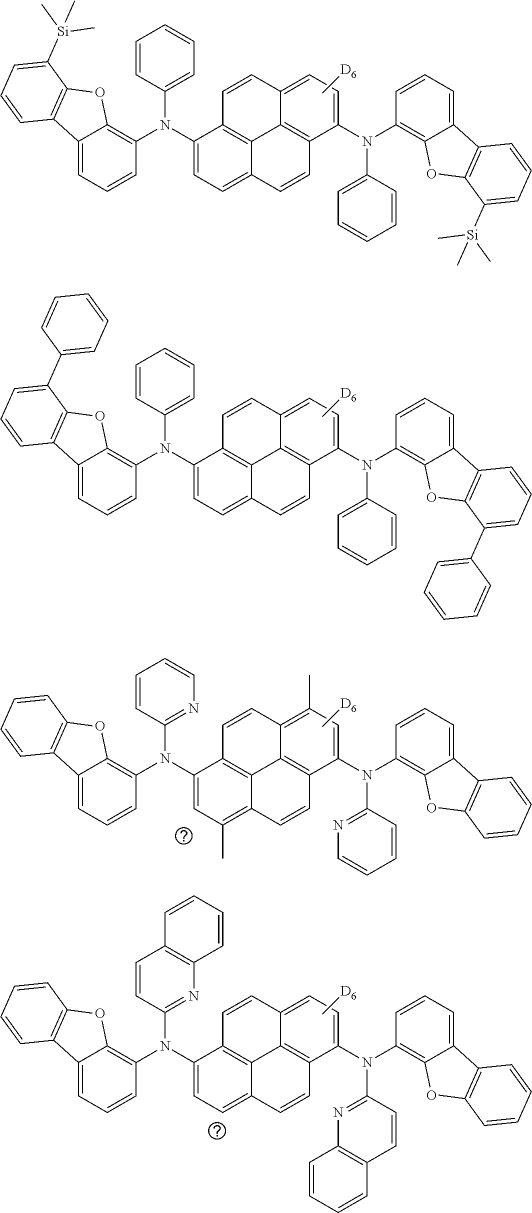

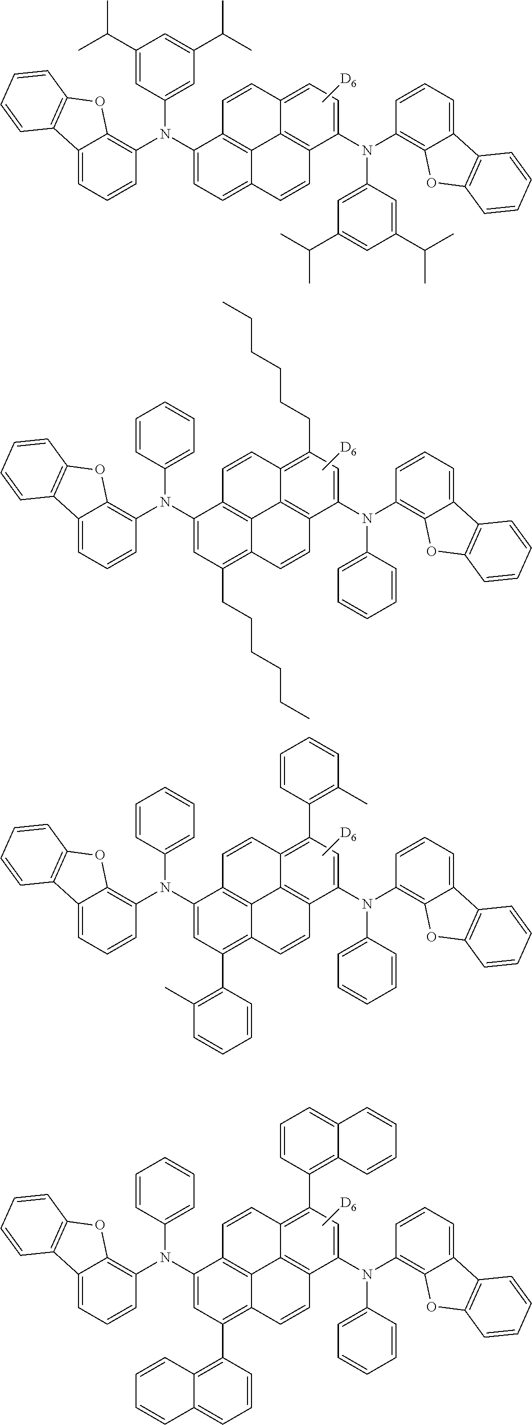

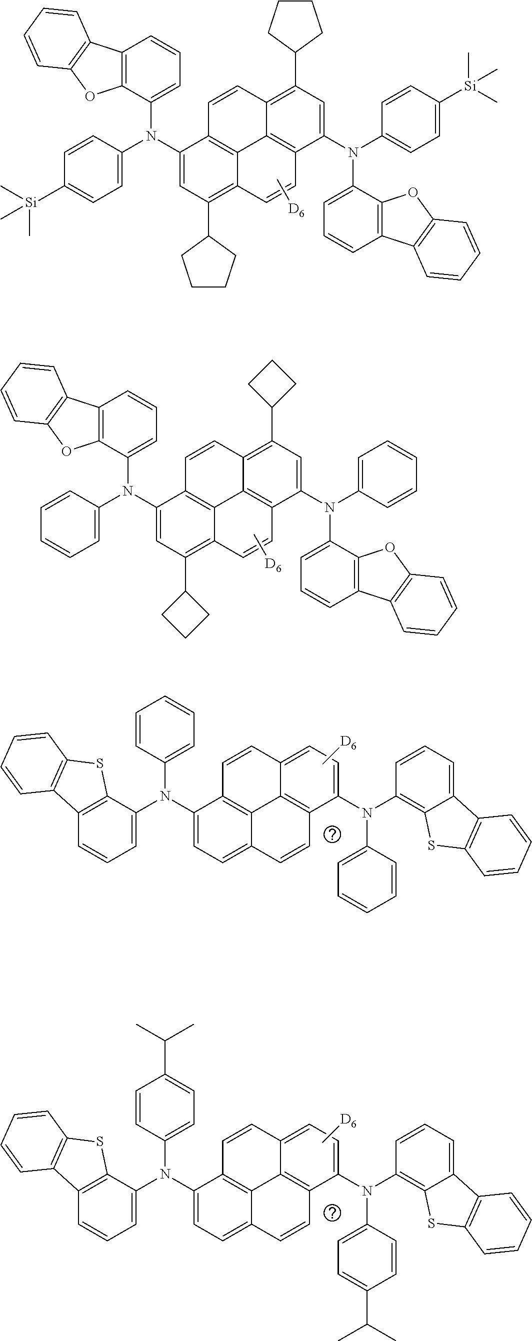

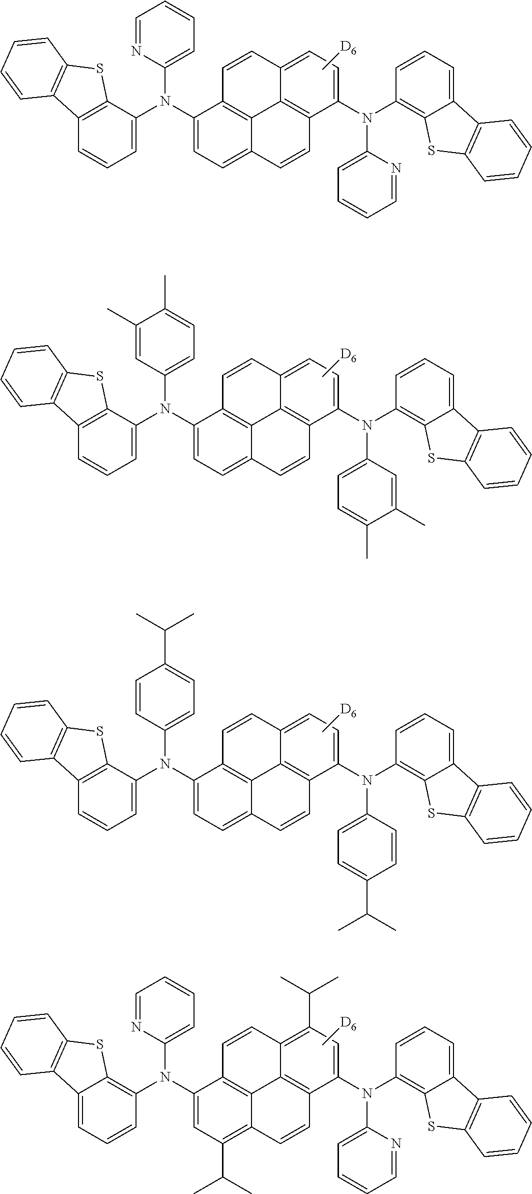

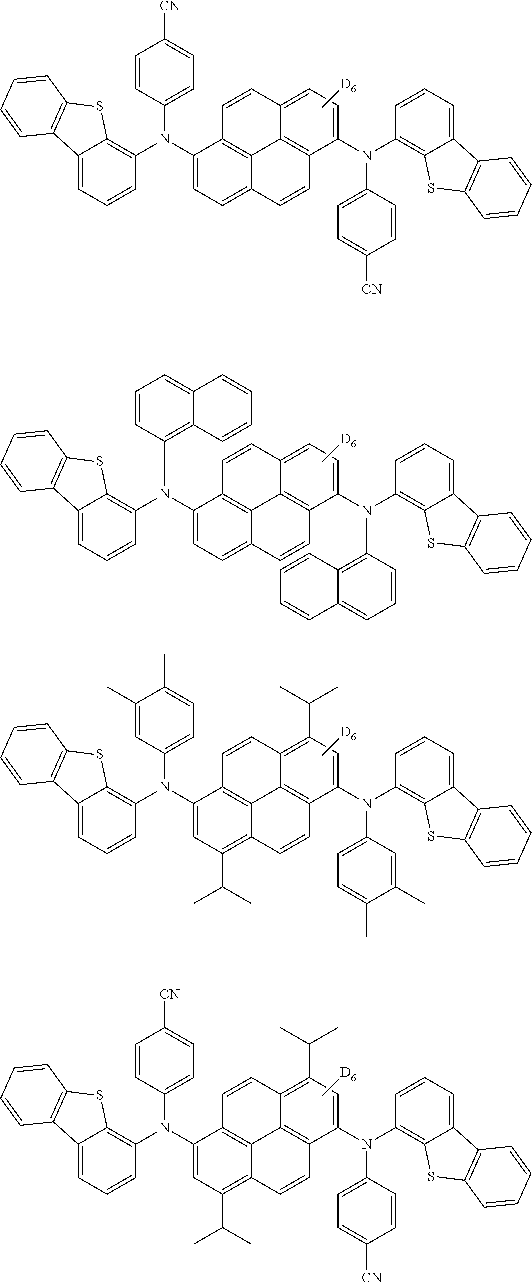

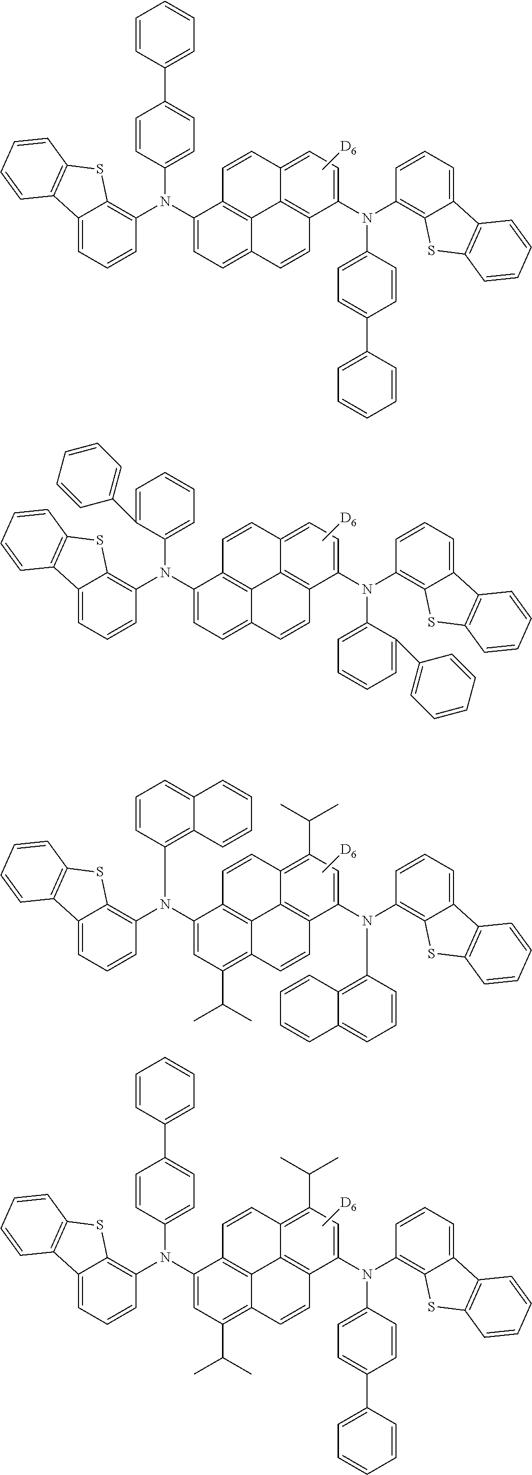

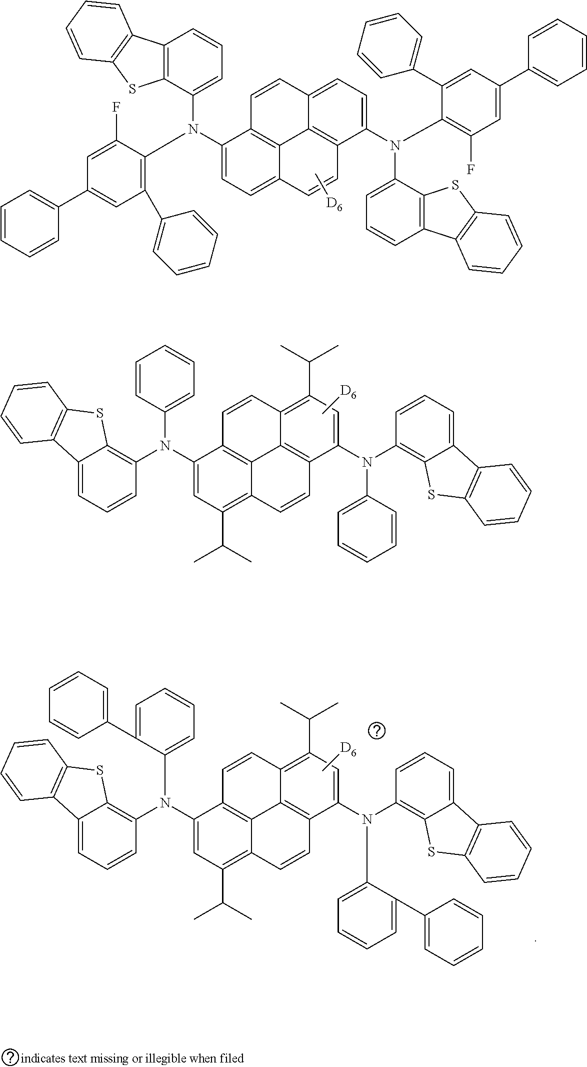

6. The OLED of claim 1, further comprising:
a second emitting material layer including a second host being an anthracene derivative and a second dopant being a pyrene derivative, the second emitting material layer being positioned between the first emitting material layer and the second electrode; and
a first charge generation layer between the first and second emitting material layers,
wherein at least one of an anthracene core of the second host and a pyrene core of the second dopant is deuterated.

7. The OLED of claim 6, further comprising:
a third emitting material layer configured to emit a yellow-green light and positioned between the first charge generation layer and the second emitting material layer; and
a second charge generation layer positioned between the second and third emitting material layers.

8. The OLED of claim 6, further comprising:
a third emitting material layer configured to emit a red light and a green light and positioned between the first charge generation layer and the second emitting material layer; and
a second charge generation layer positioned between the second and third emitting material layers.

9. An organic light emitting device, comprising:
a substrate; and
an organic light emitting diode positioned on the substrate, and including:
a first electrode;
a second electrode facing the first electrode; and
a first emitting material layer including a first host being an anthracene derivative and a first dopant being a pyrene derivative, and positioned between the first and second electrodes,
wherein at least one of an anthracene core of the first host and a pyrene core of the first dopant is deuterated.

10. The organic light emitting device of claim 9, wherein the first host is represented by Formula 1: 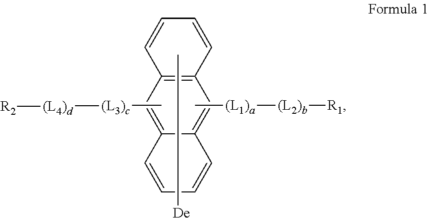
wherein each of R₁ and R₂ is independently C₆˜C₃₀ aryl group or C₅˜C₃₀ heteroaryl group, and each of L₁, L₂, L₃ and L₄ is independently C₆˜C₃₀ arylene group, and
wherein each of a, b, c and d is 0 or 1, and e is an integer of 1 to 8.

11. The organic light emitting device of claim 10, wherein the first host is a compound being one of the followings of Formula 2:

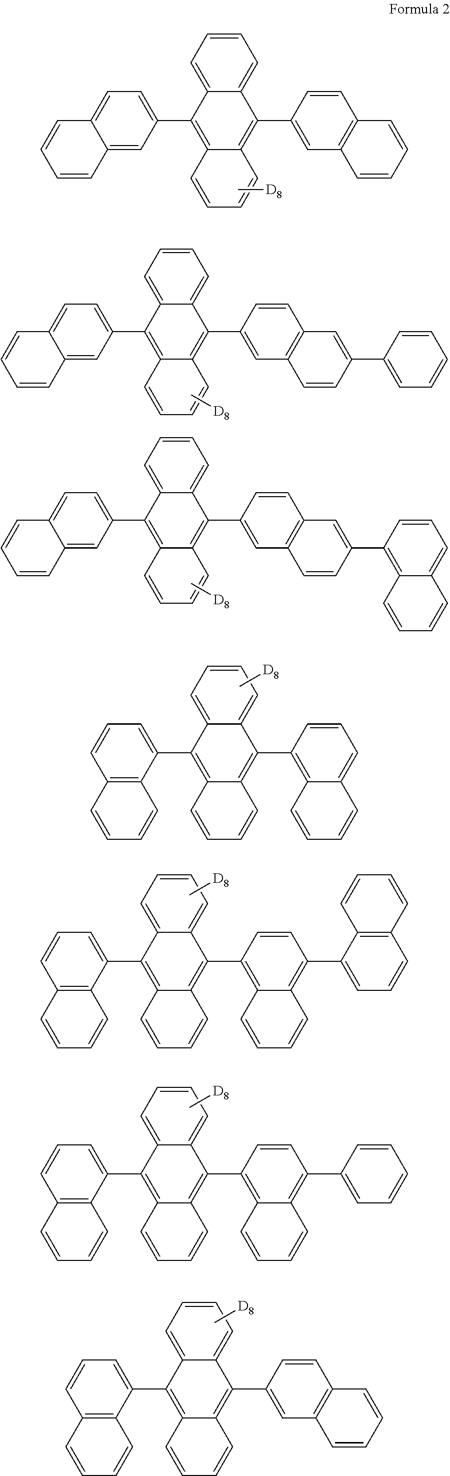

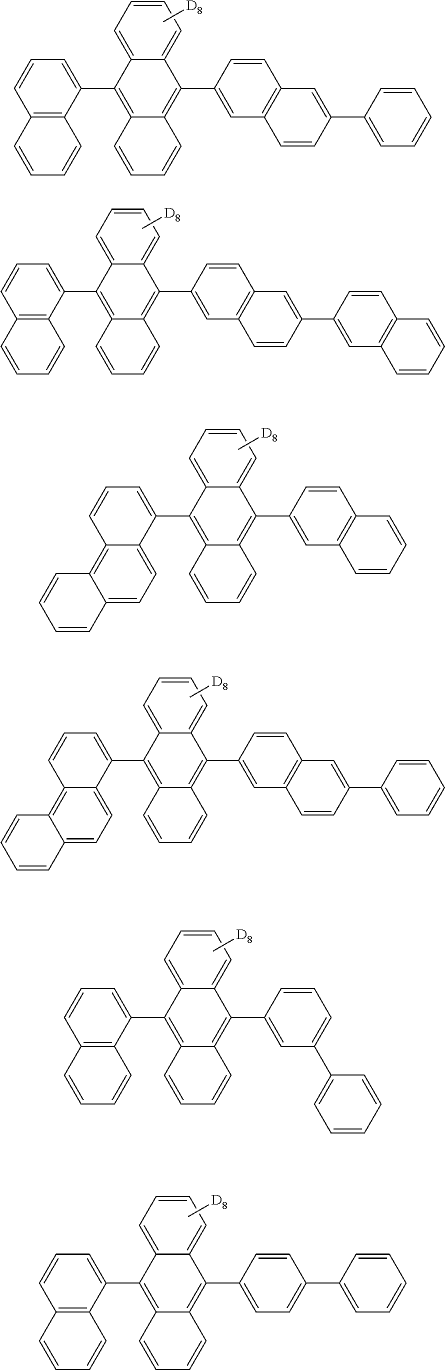

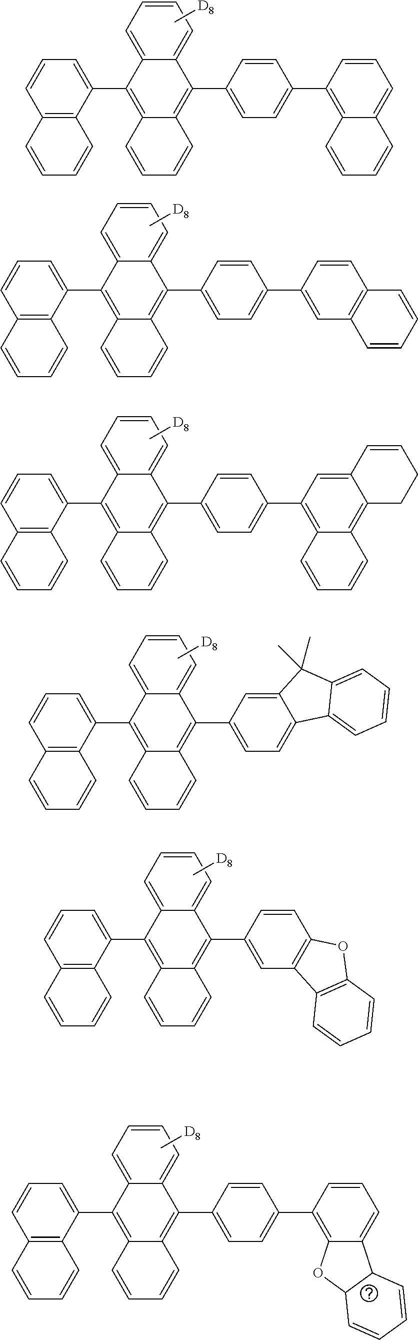

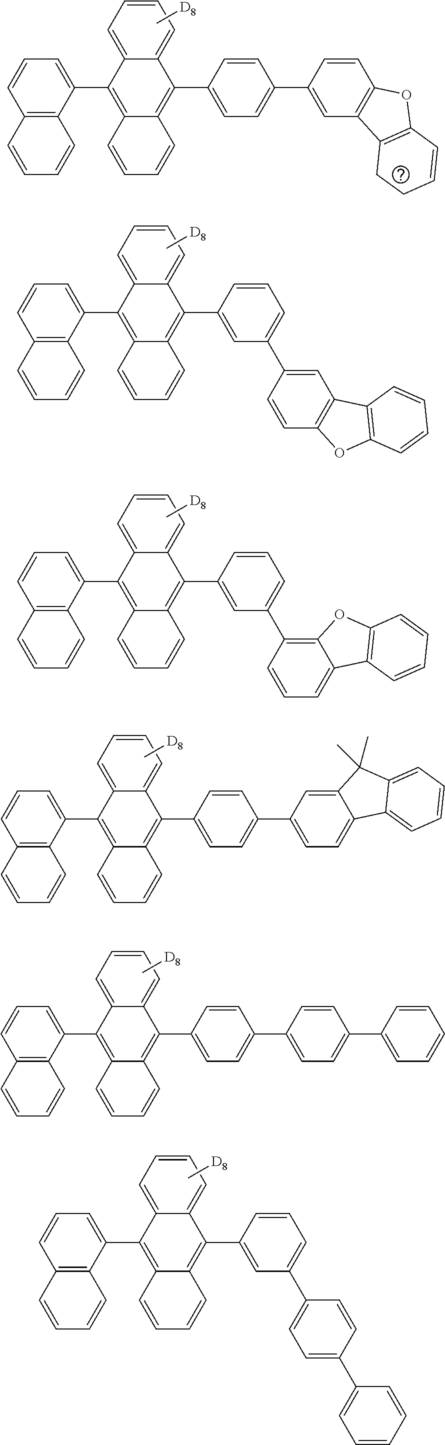

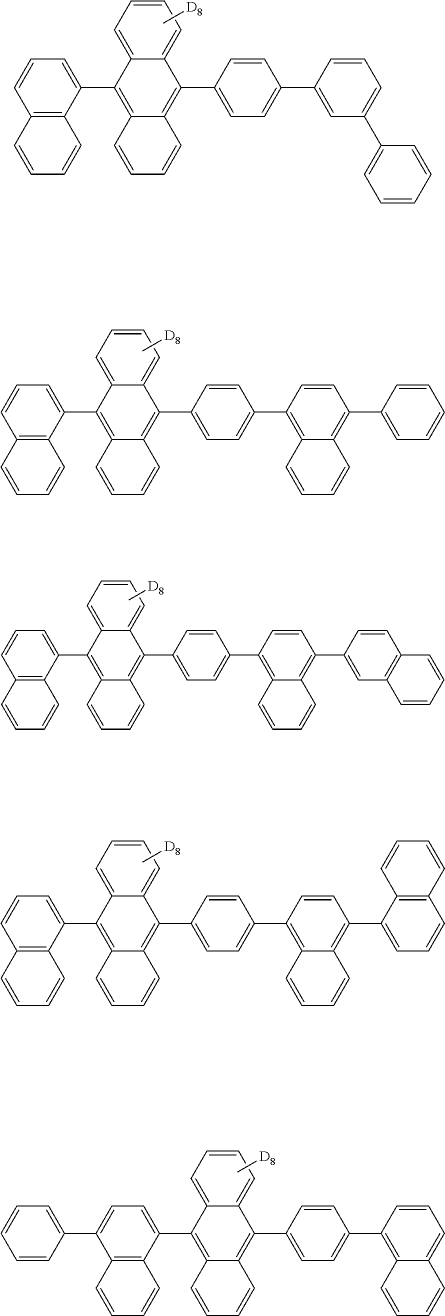

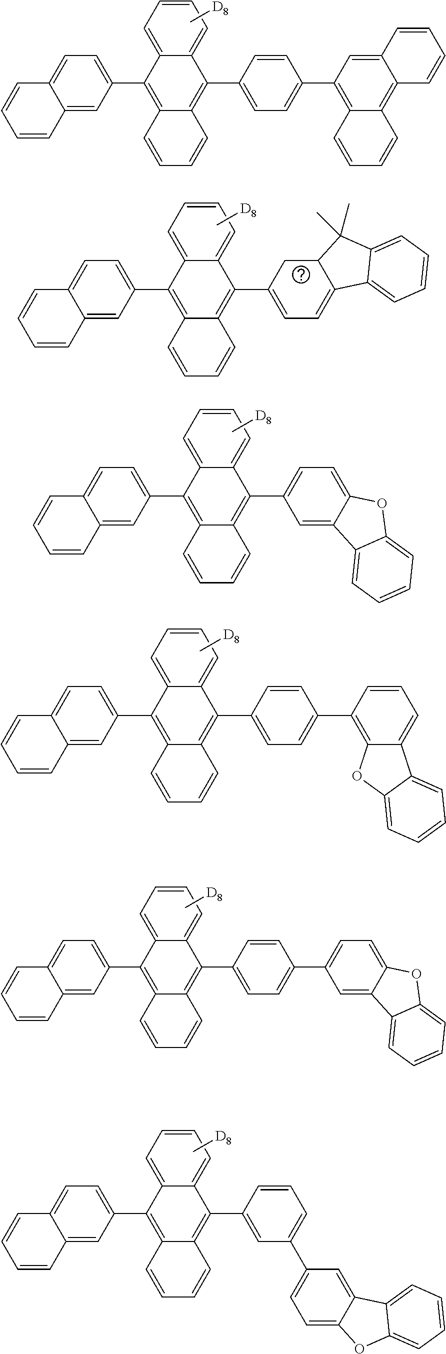

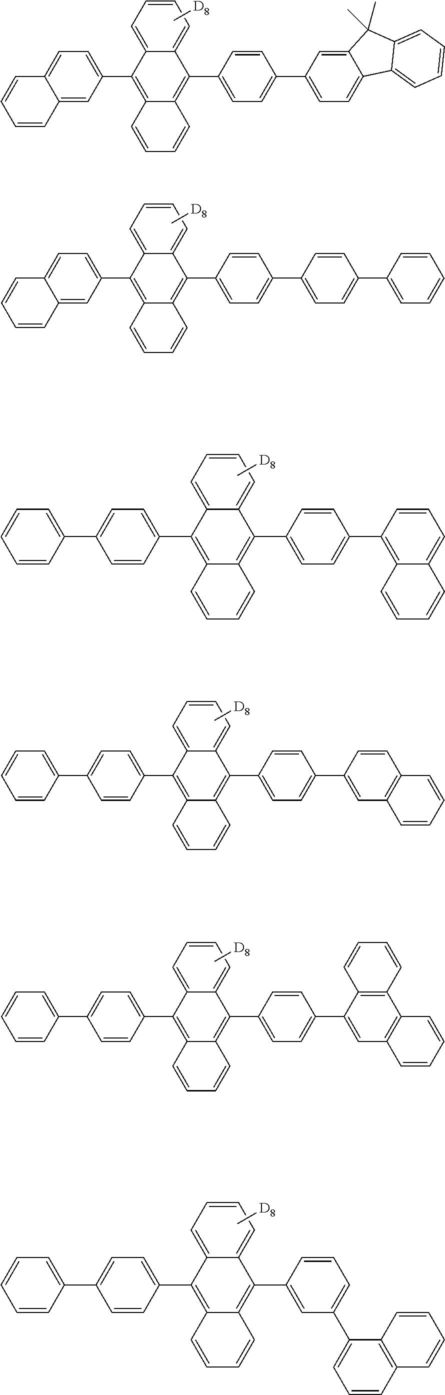

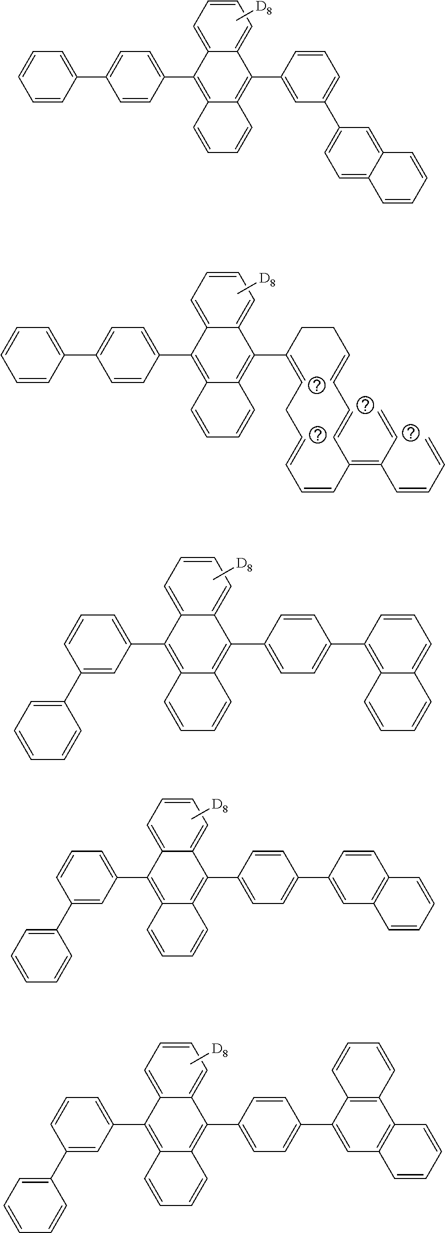

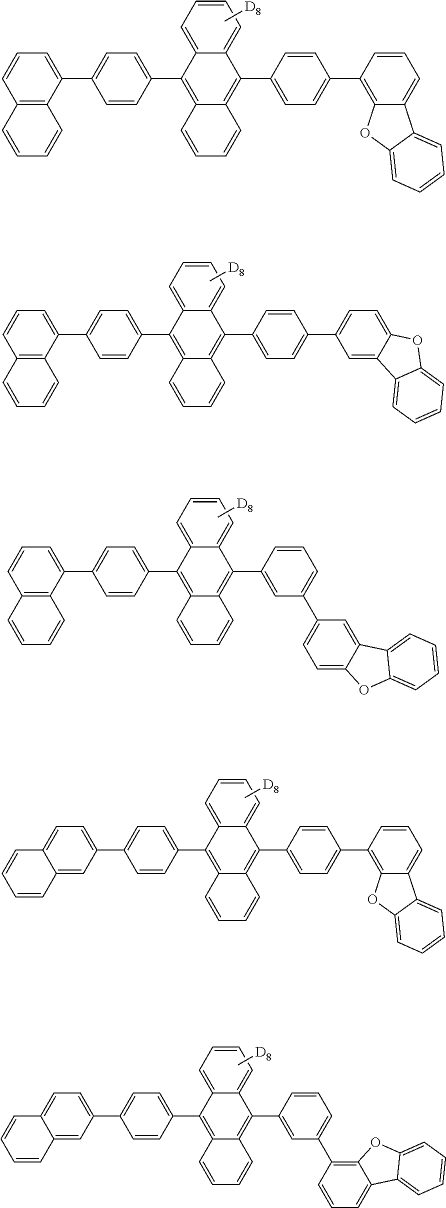

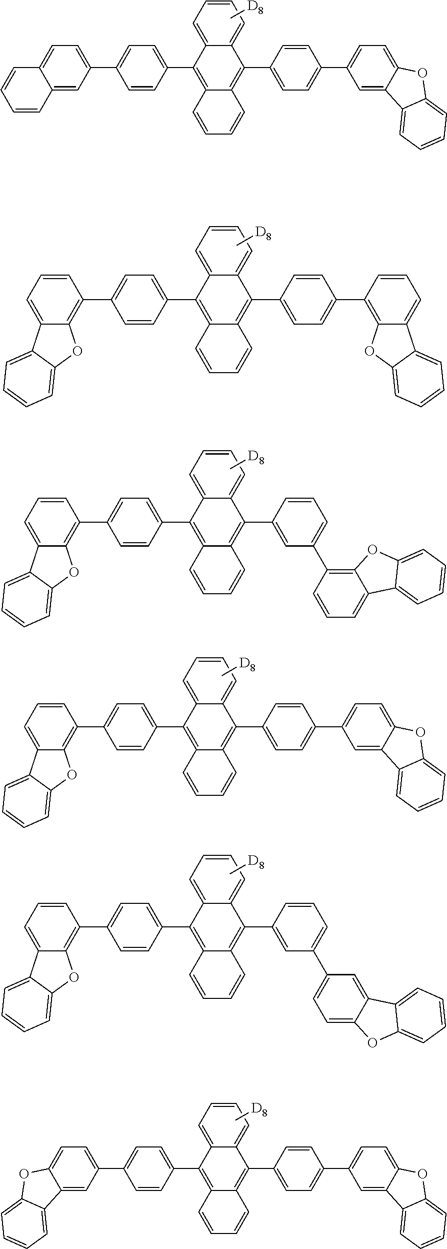

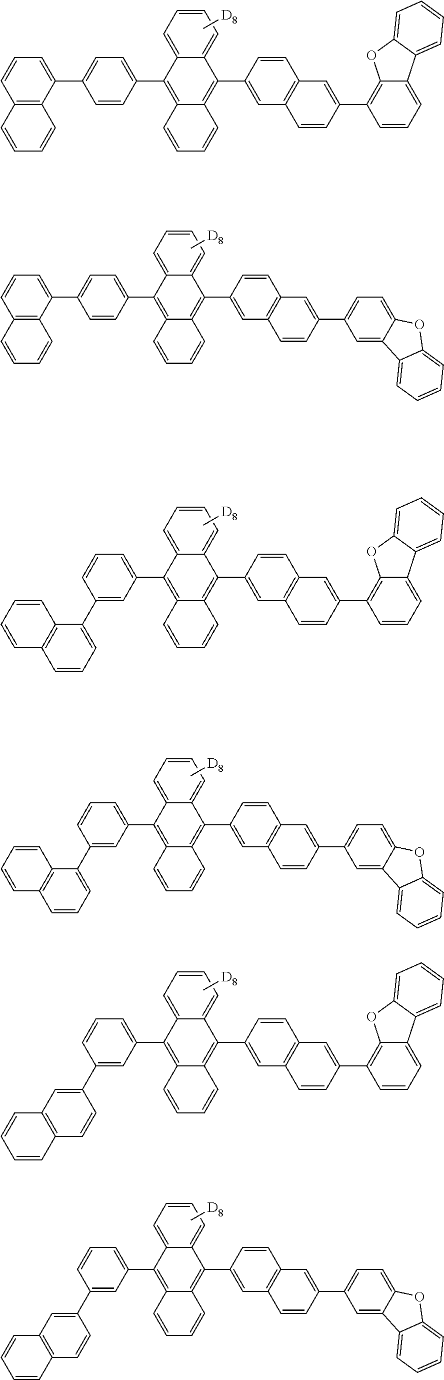

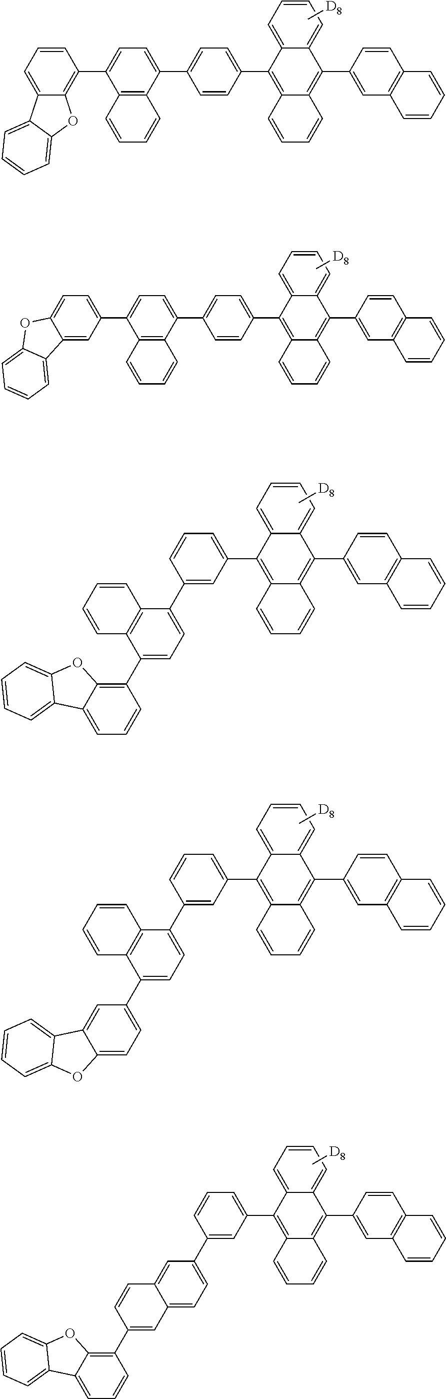

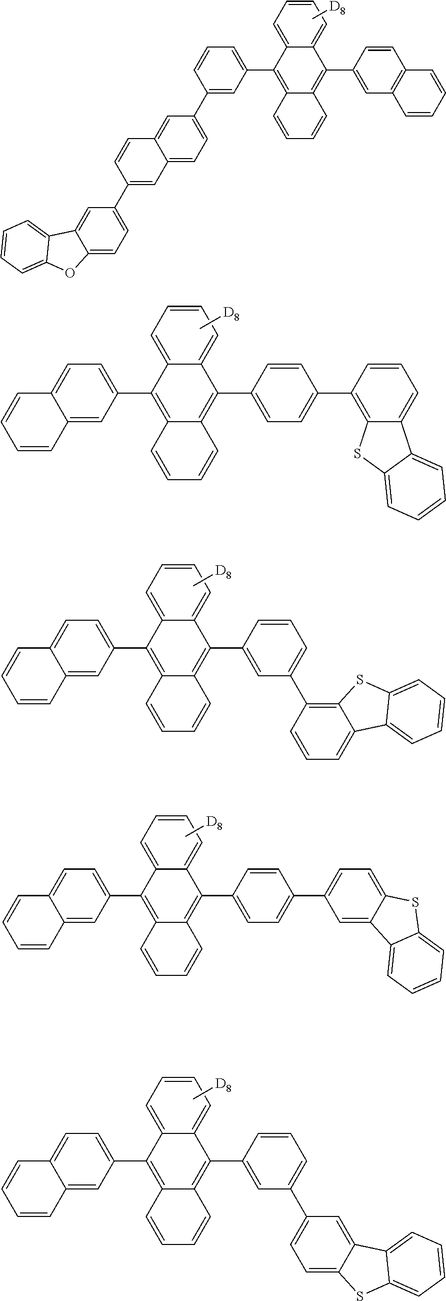

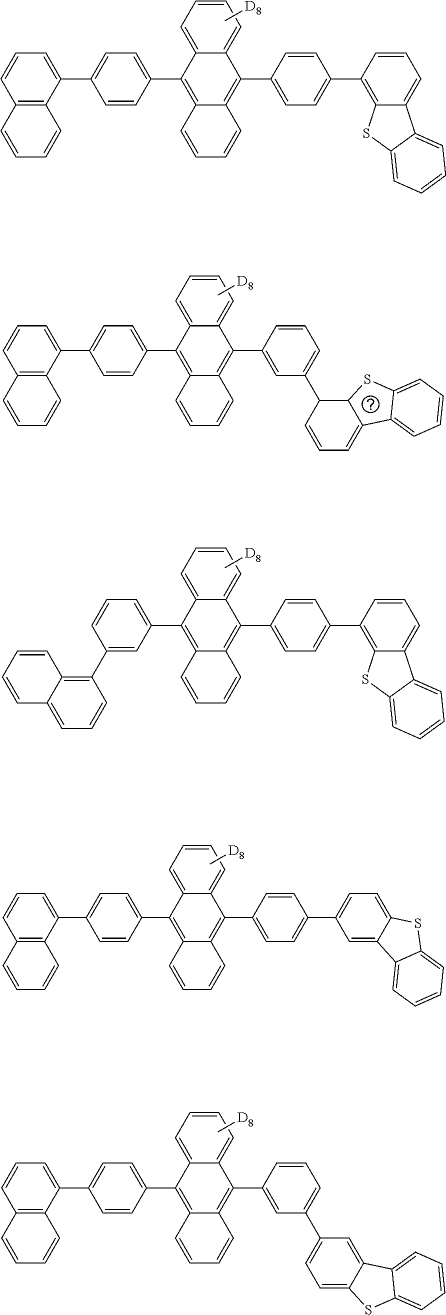

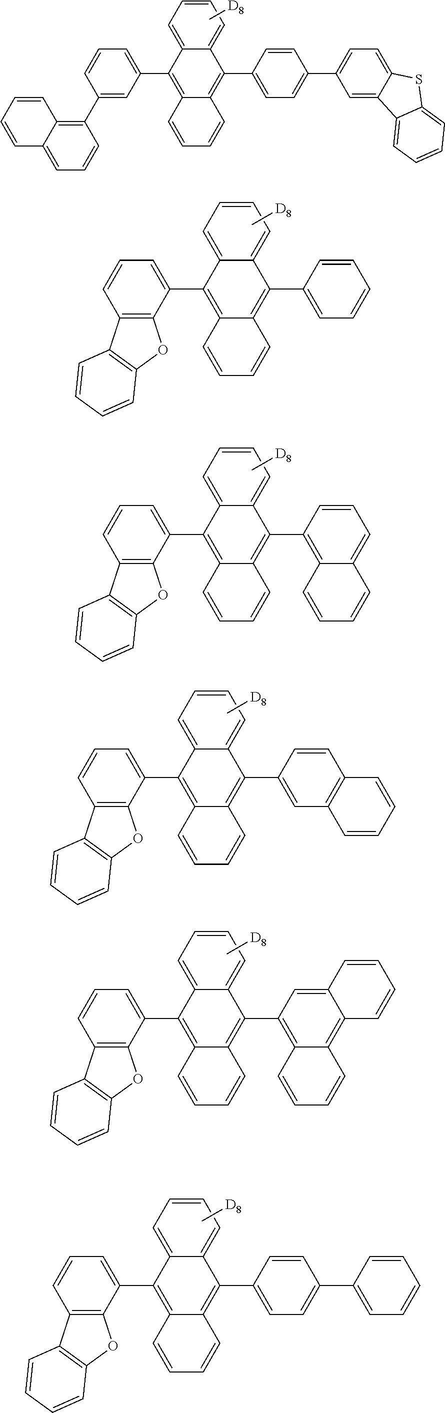

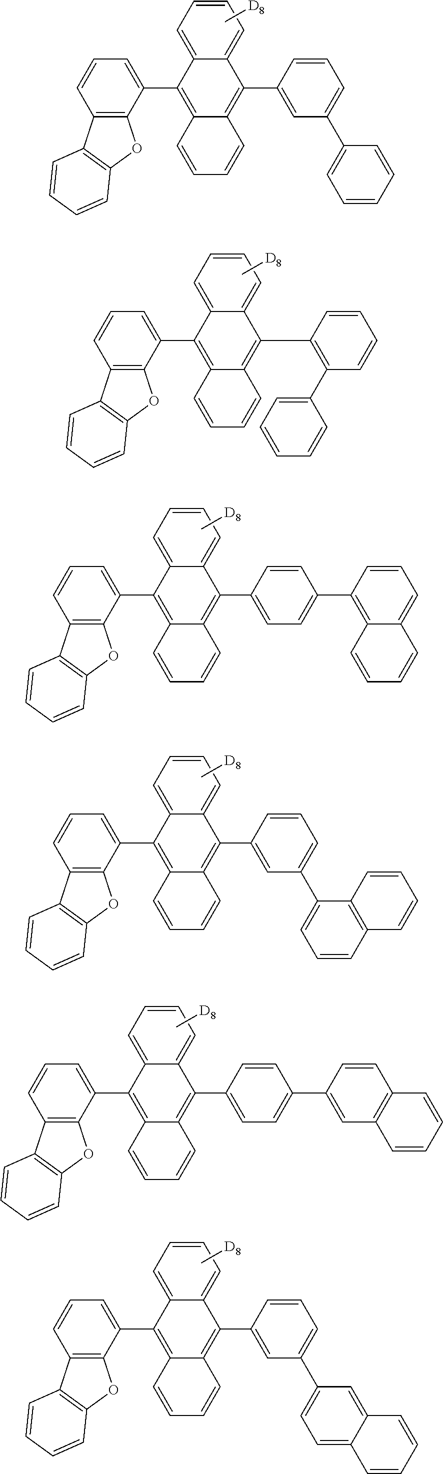

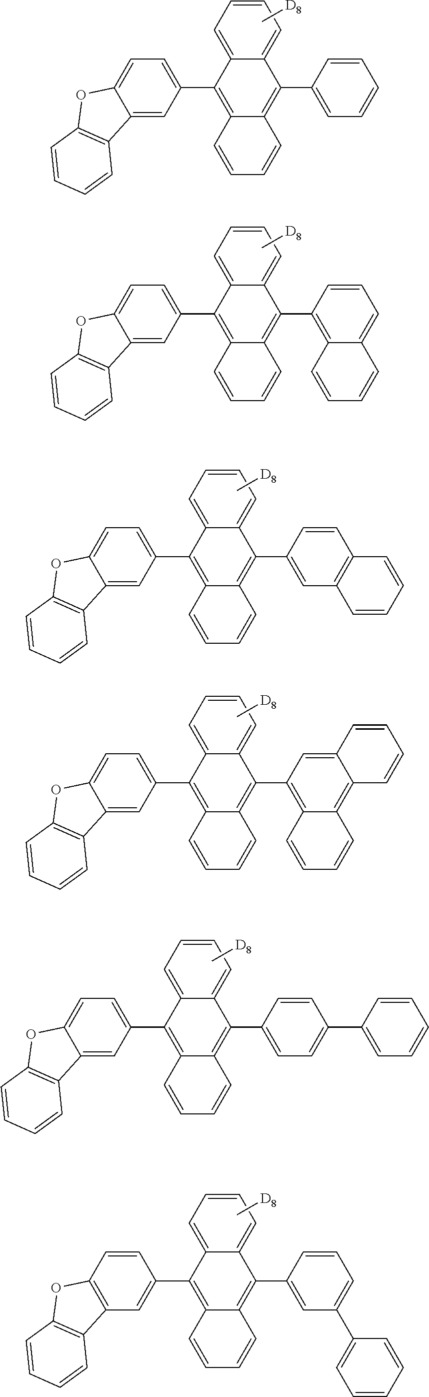

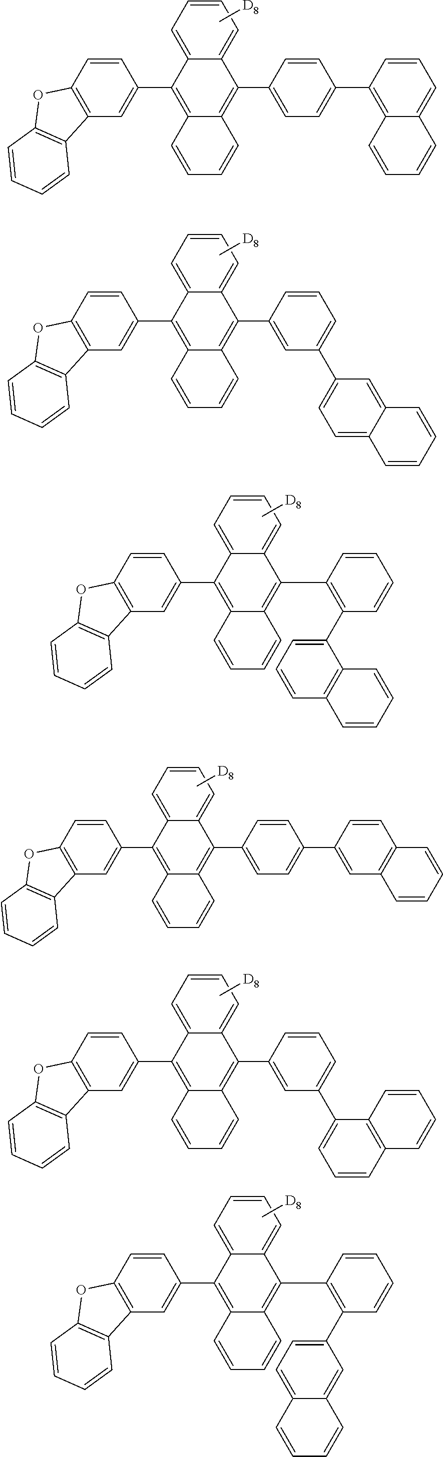

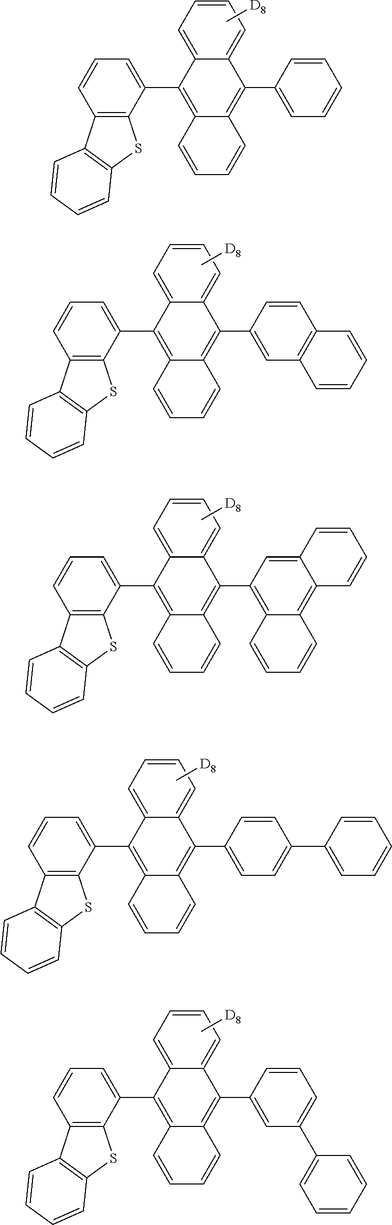

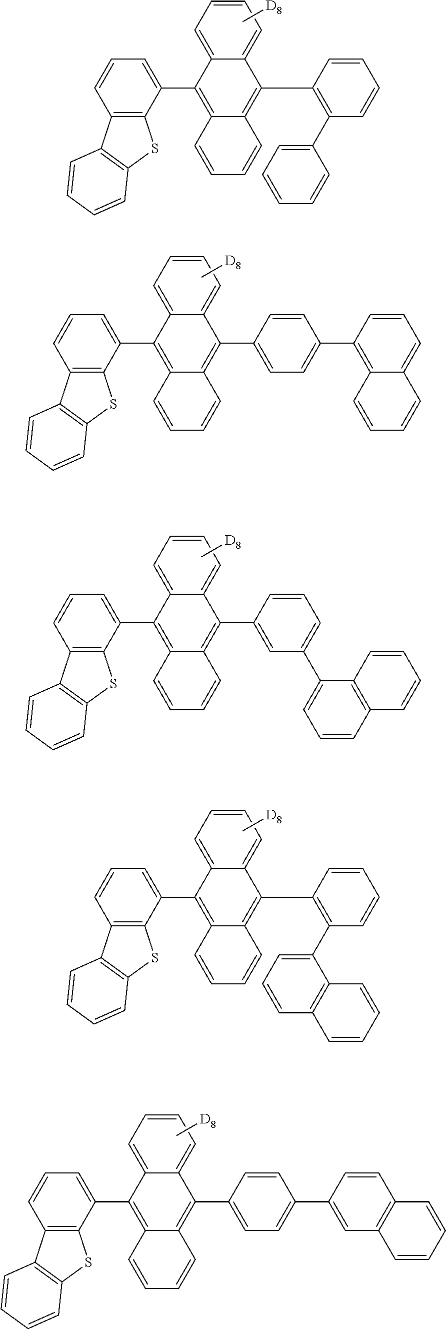

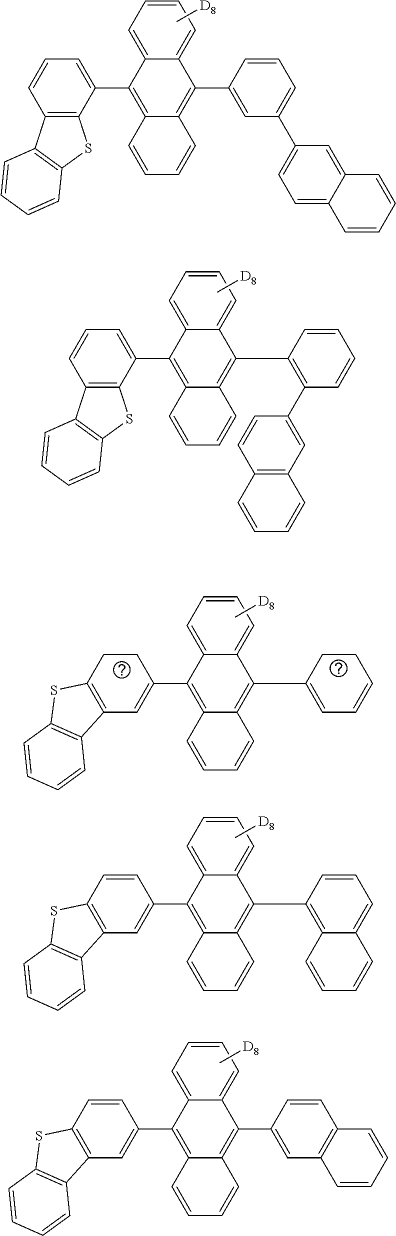

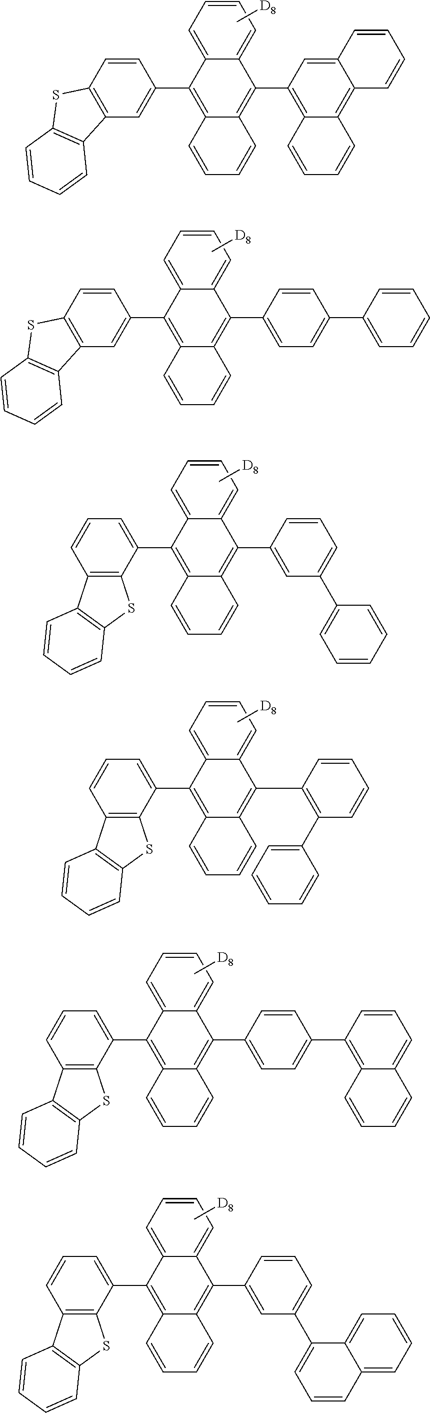

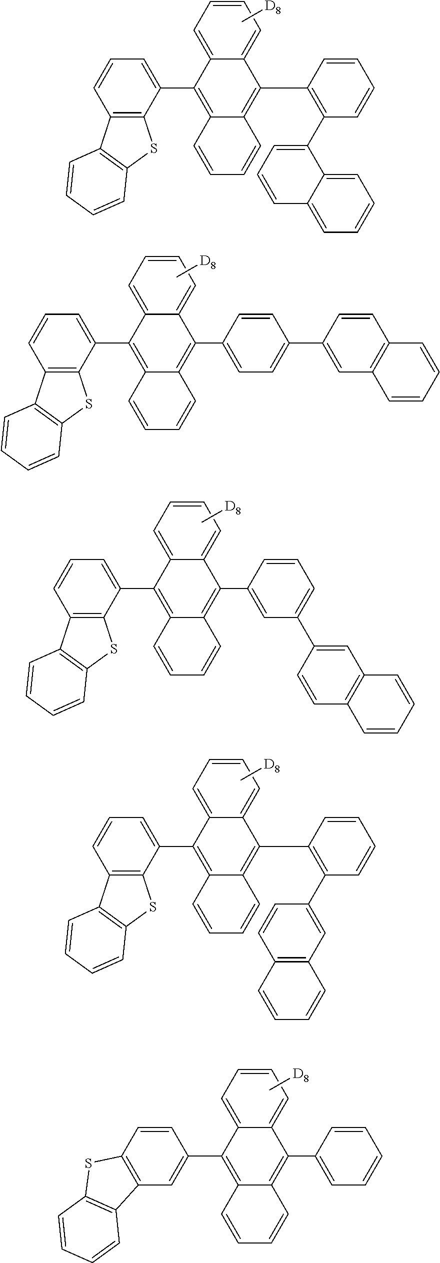

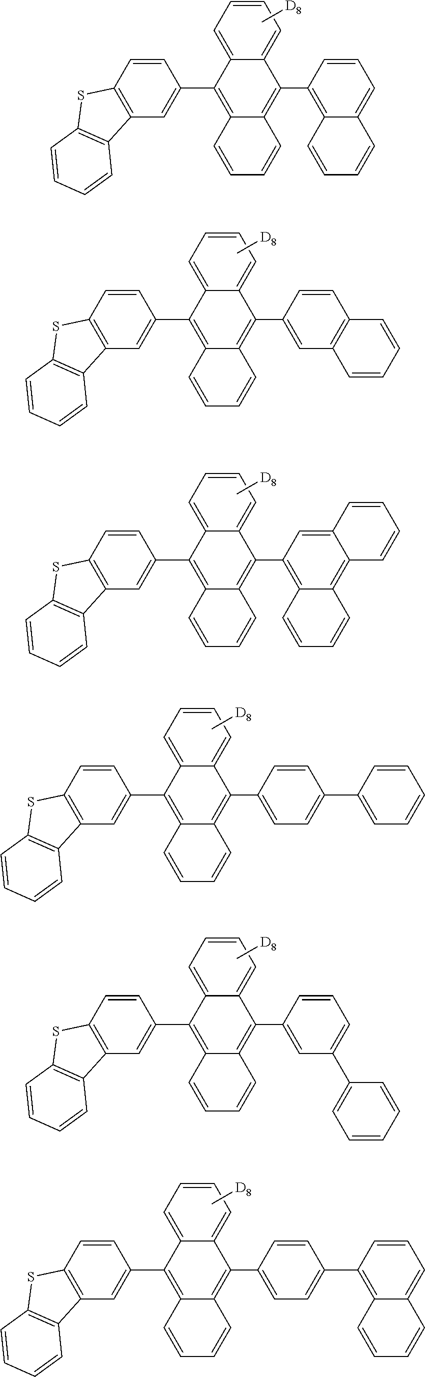

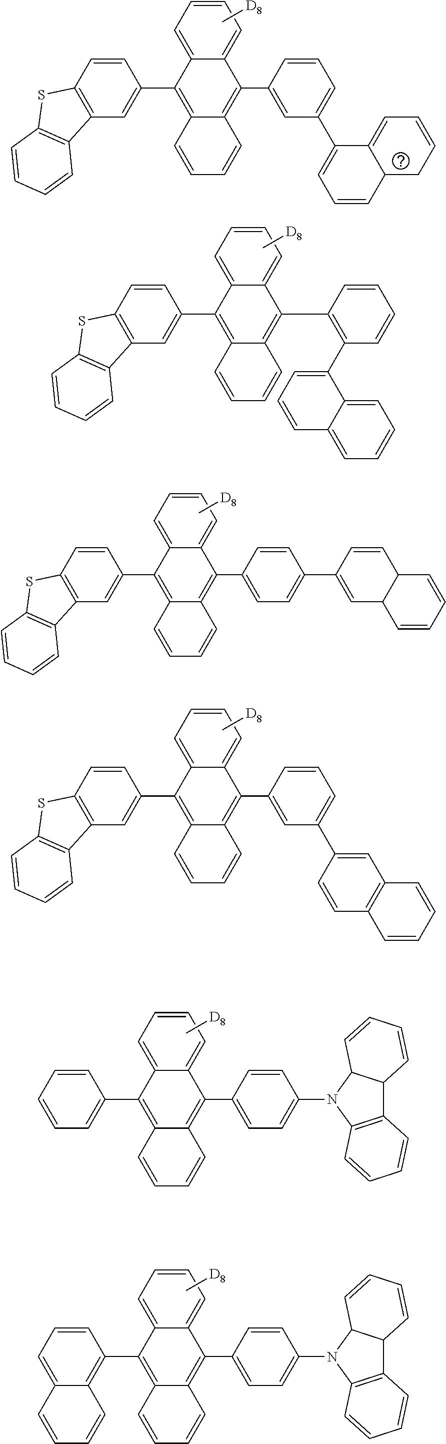

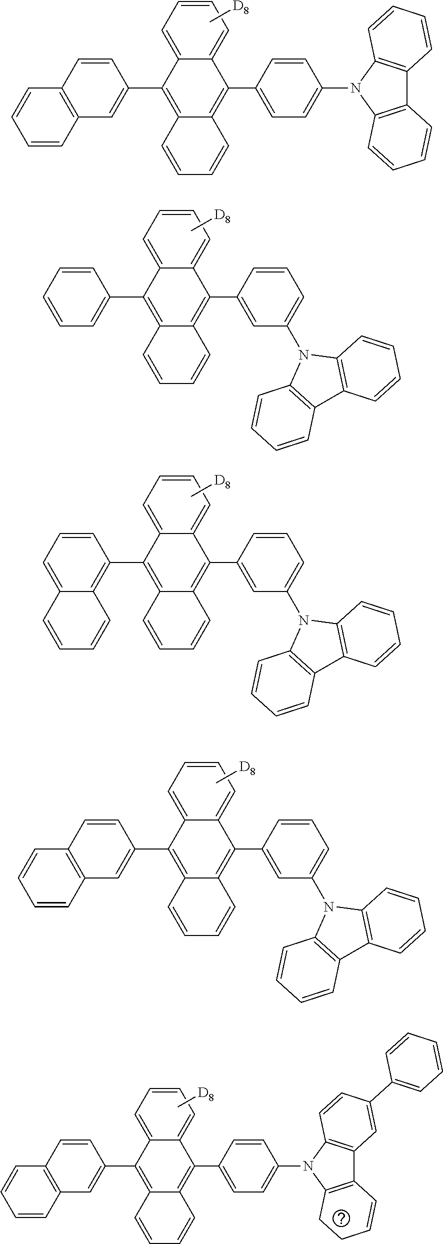

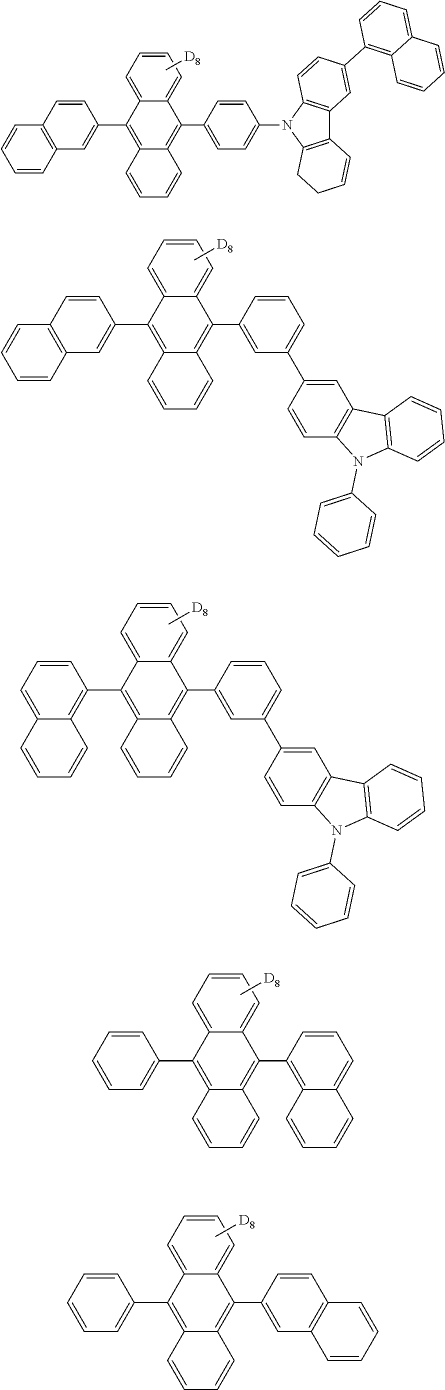

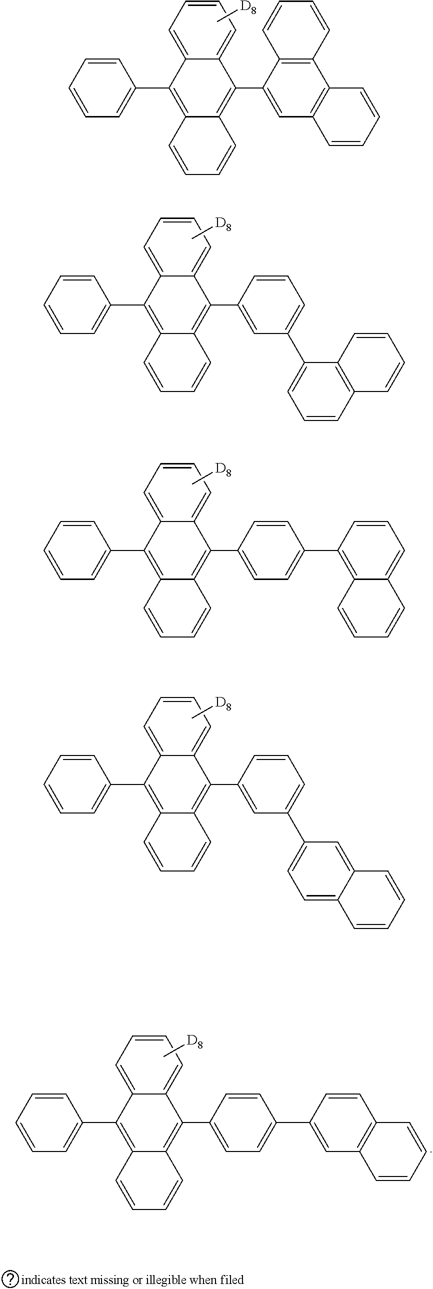

12. The organic light emitting device of claim 9, wherein the first dopant is represented by Formula 3: 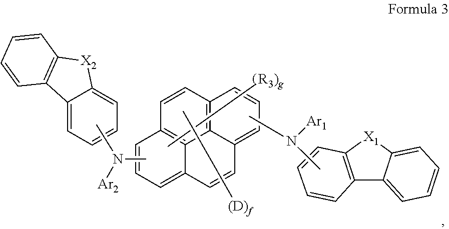
wherein each of X₁ and X₂ is independently O or S, each of Ar₁ and Ar₂ is independently C₆˜C₃₀ aryl group or C₅˜C₃₀ heteroaryl group,
wherein R₃ is C₁˜C₁₀ alkyl group or C₁˜C₁₀ cycloalkyl group, and f is an integer of 1 to 8, and
wherein g is an integer of 0 to 2, and a summation off and g is 8 or less.

13. The organic light emitting device of claim 12, wherein the first dopant is a compound being one of the followings of Formula 4:

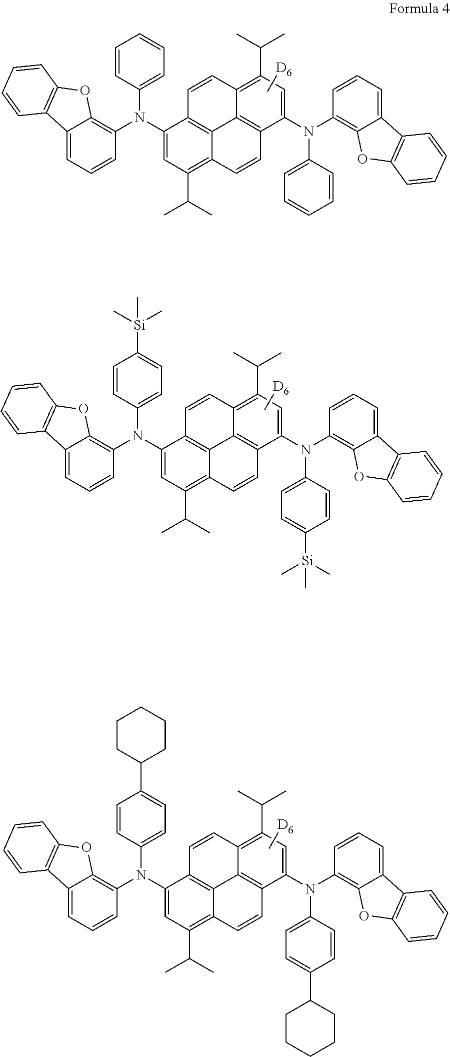

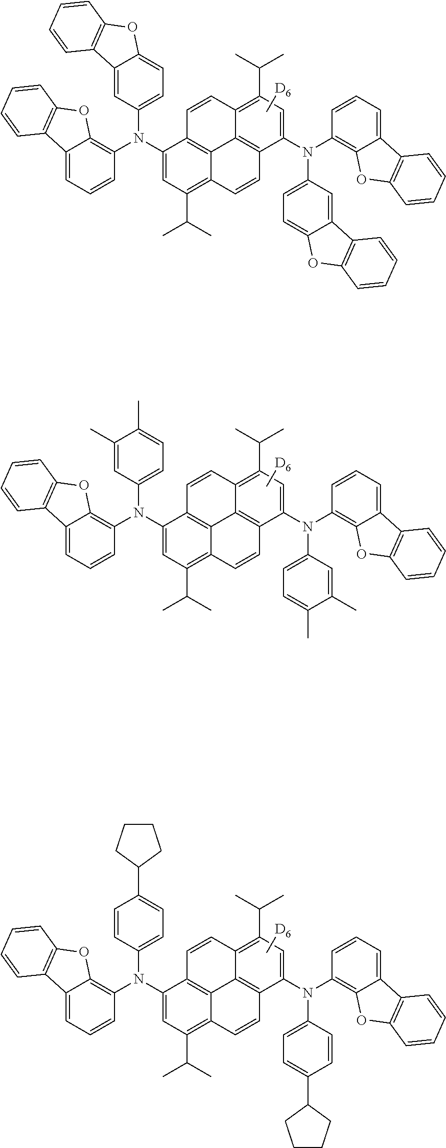

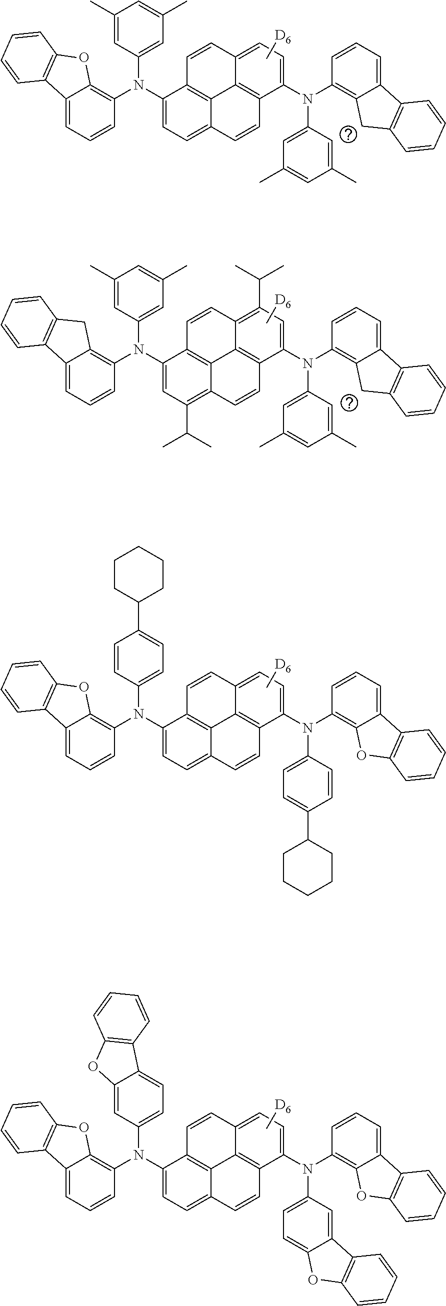

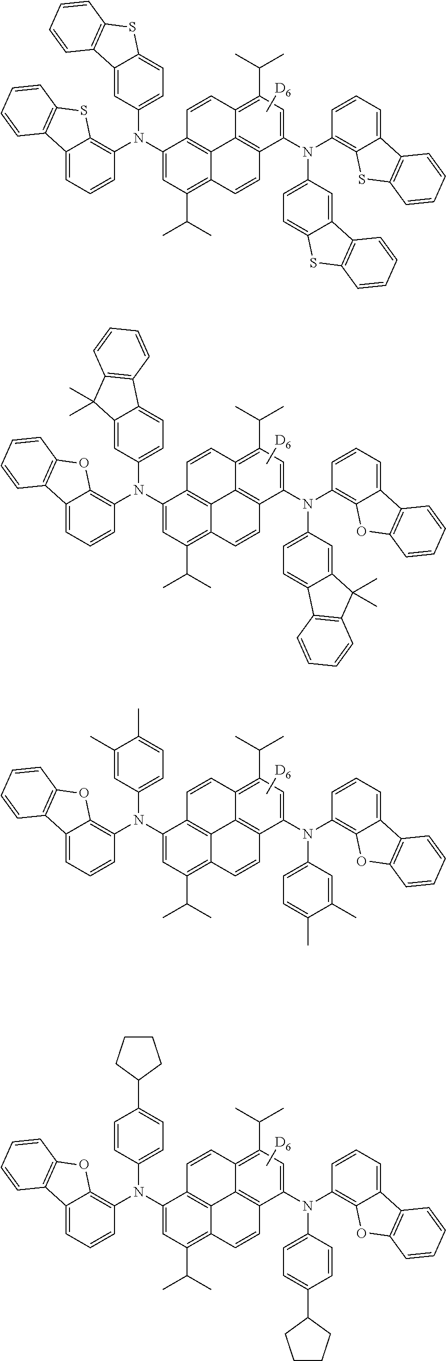

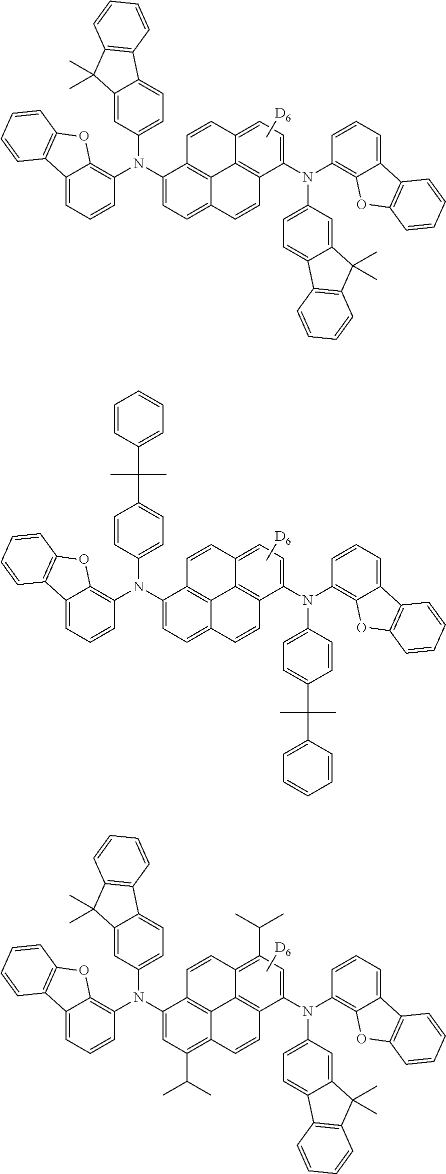

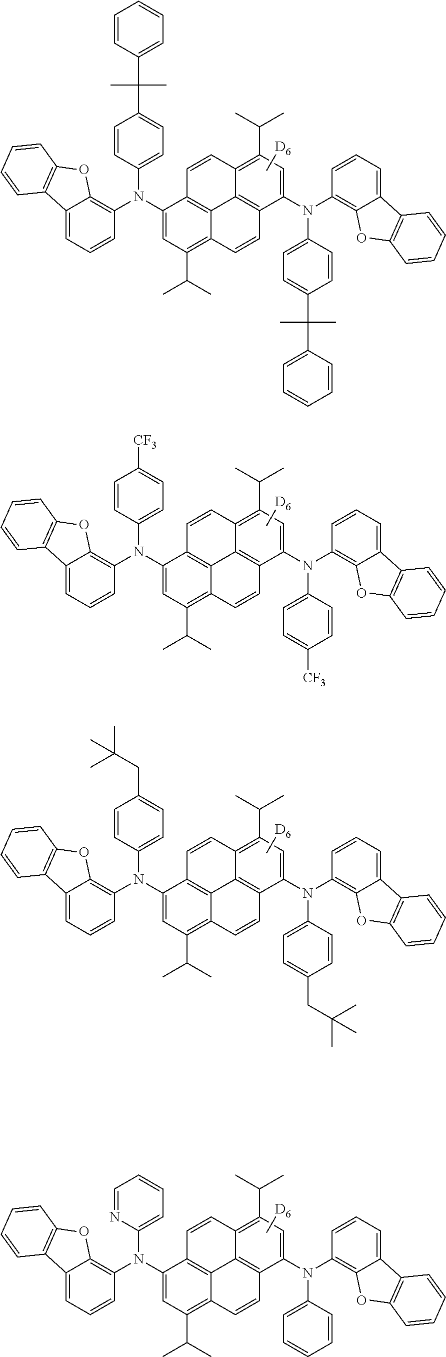

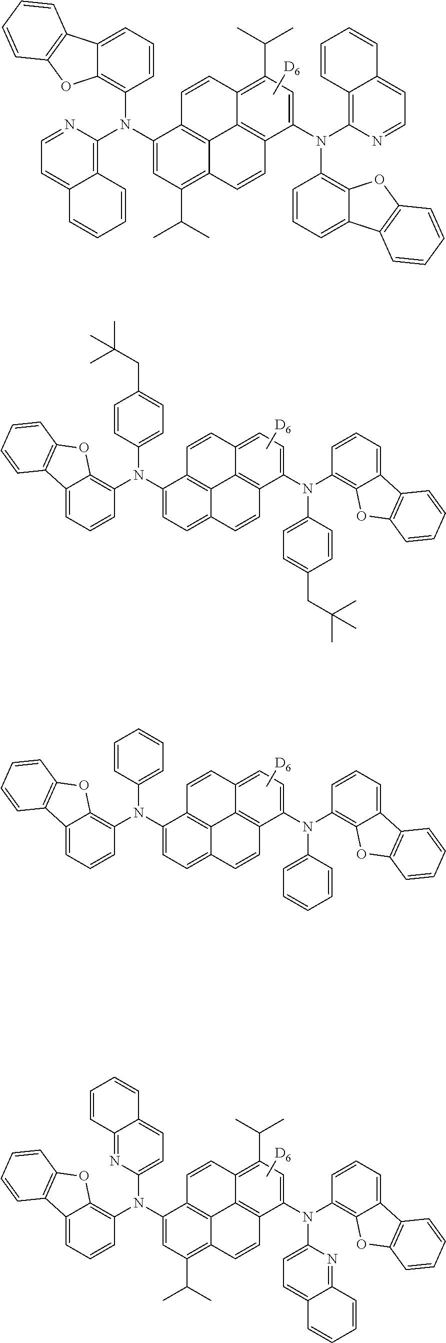

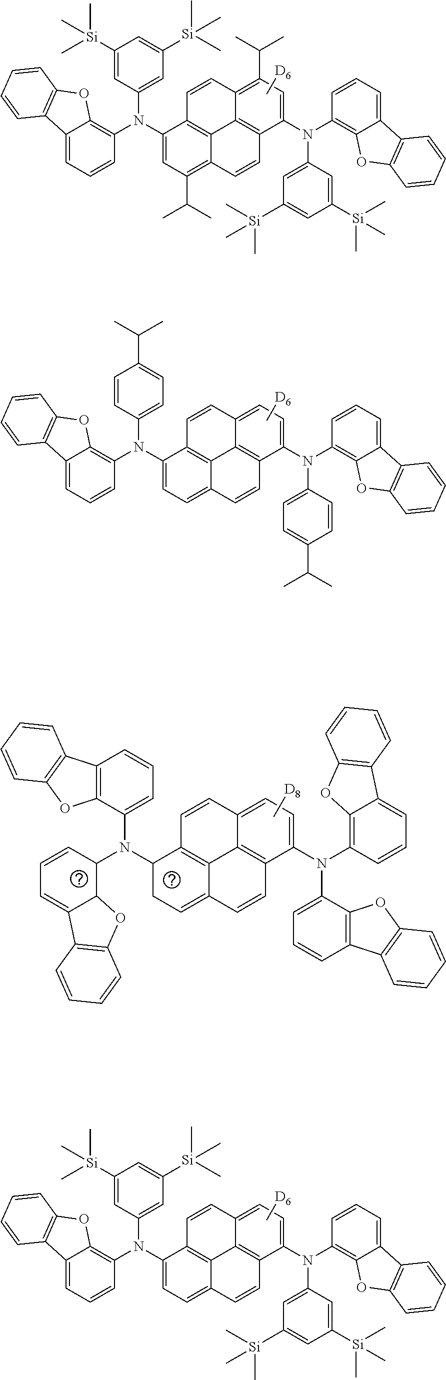

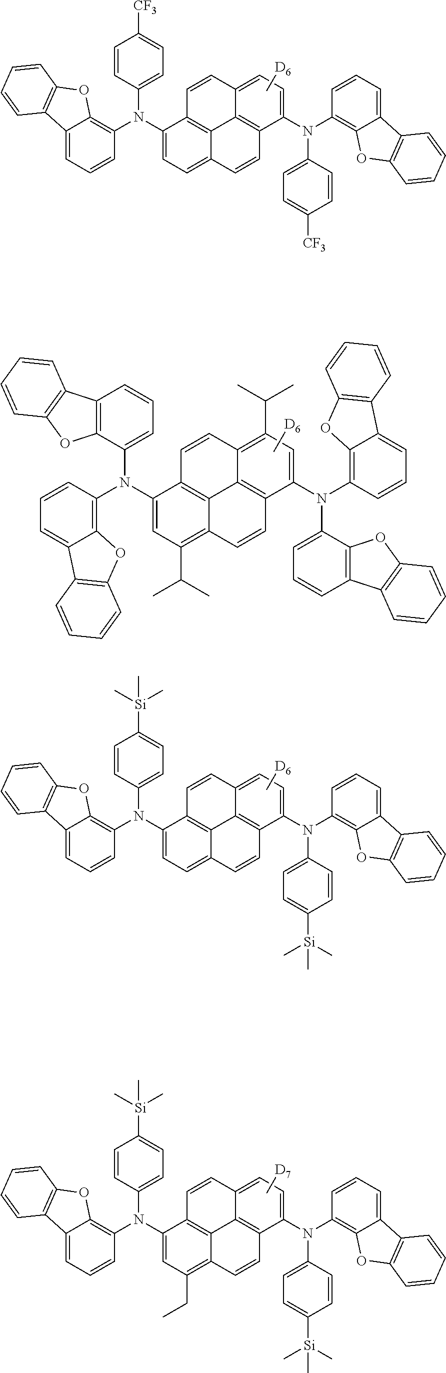

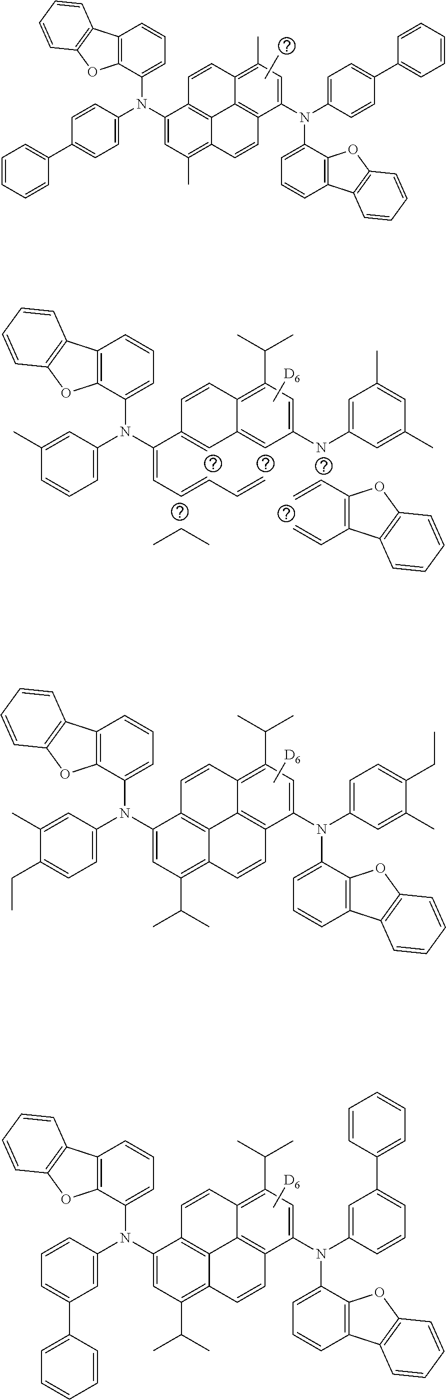

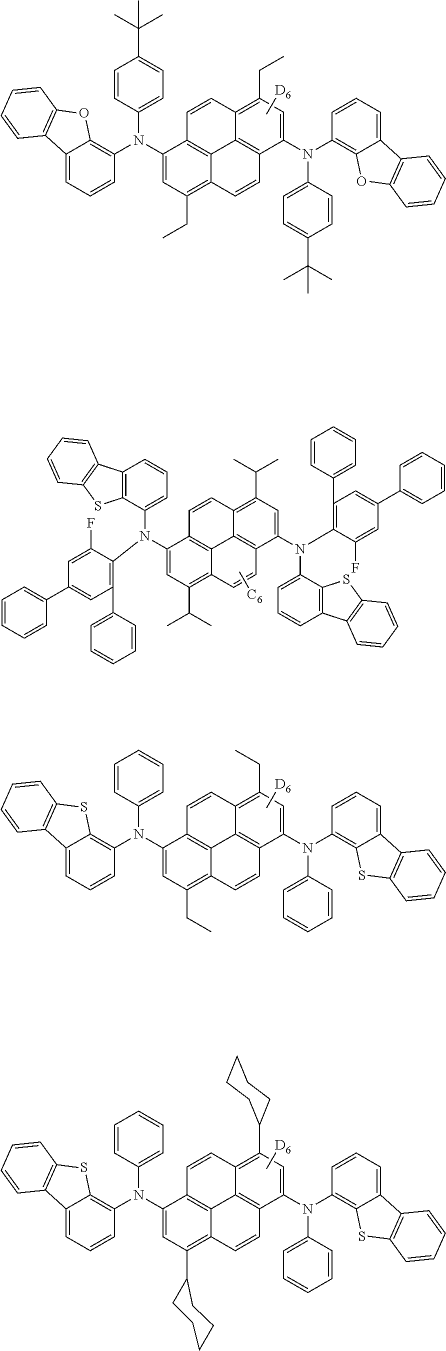

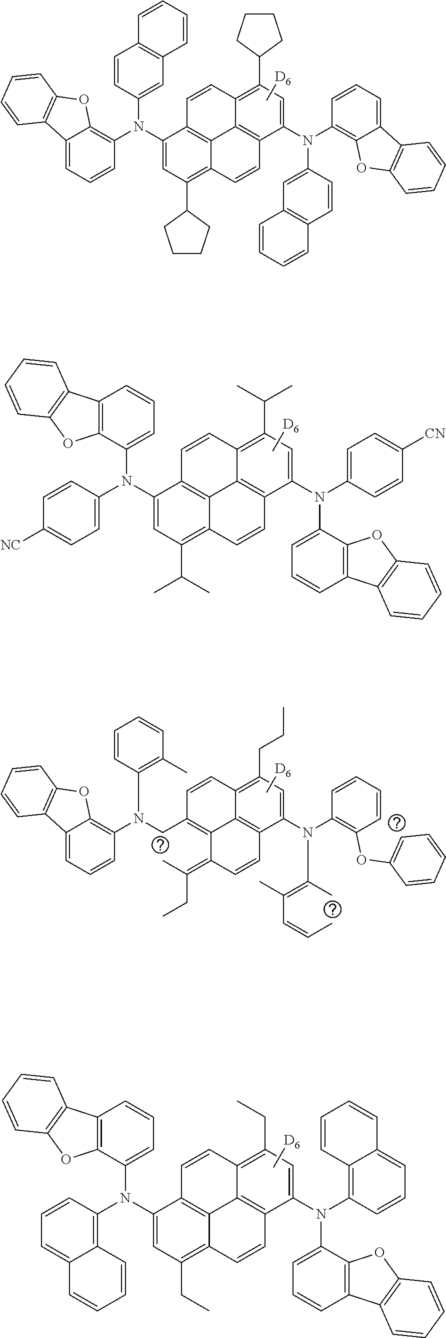

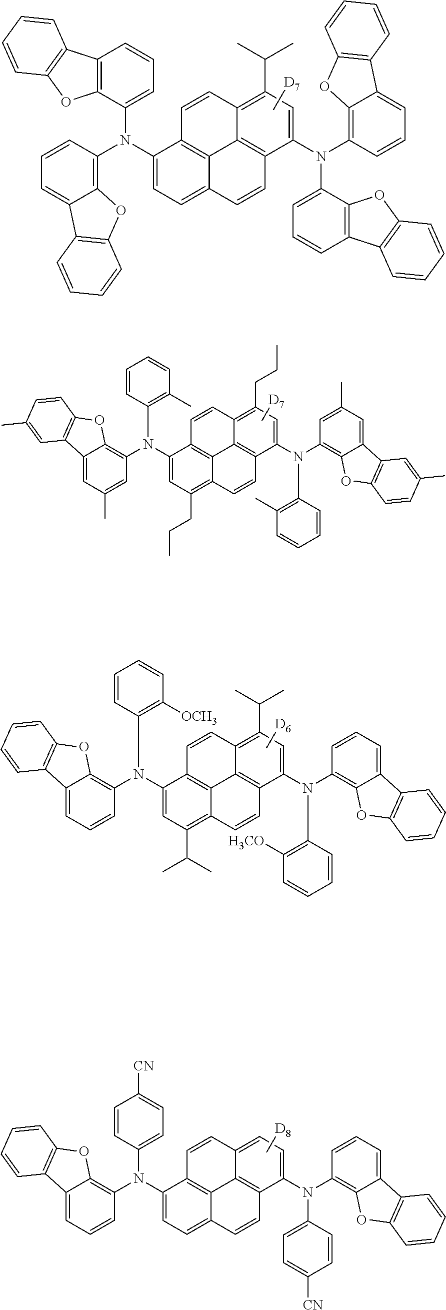

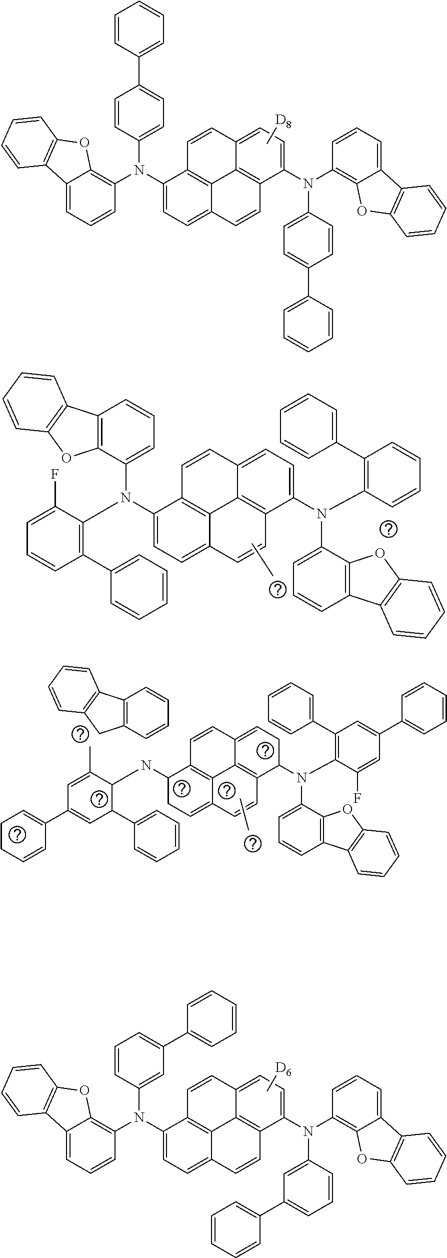

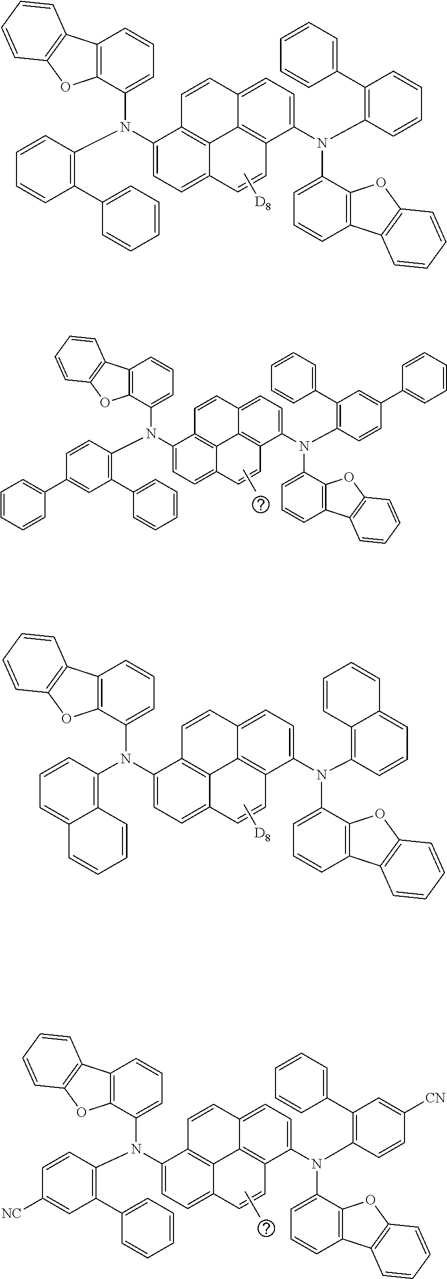

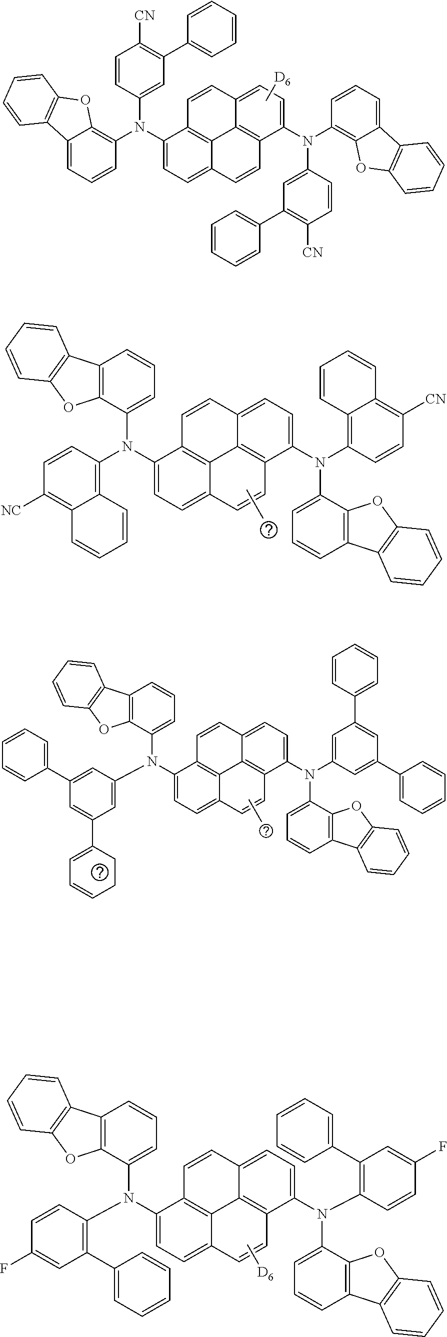

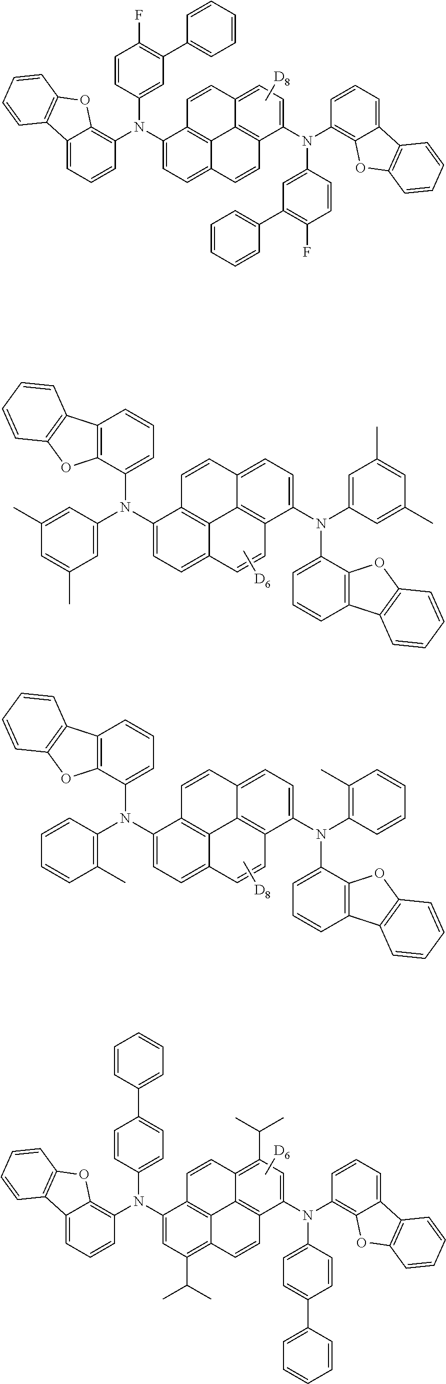

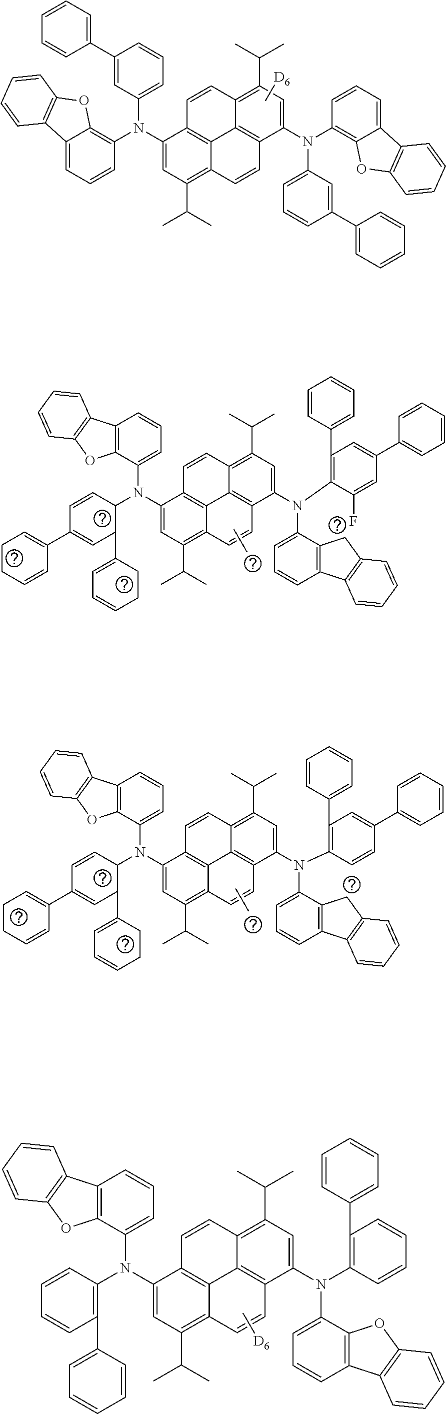

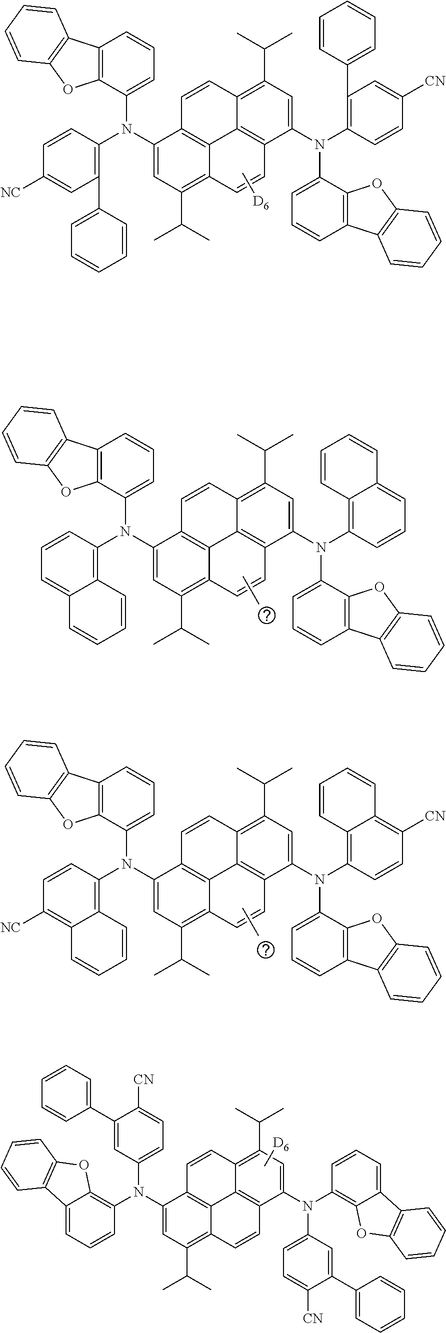

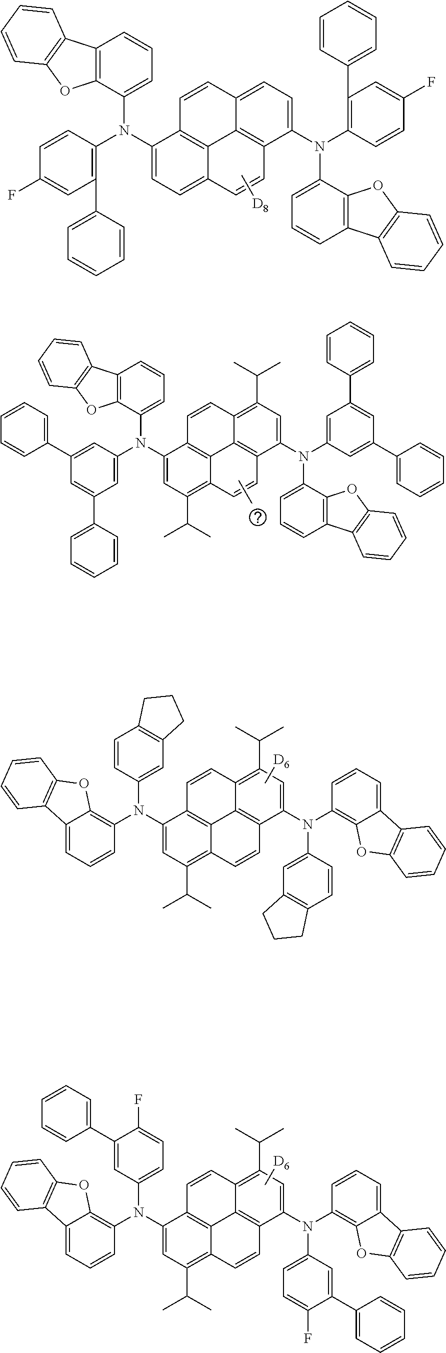

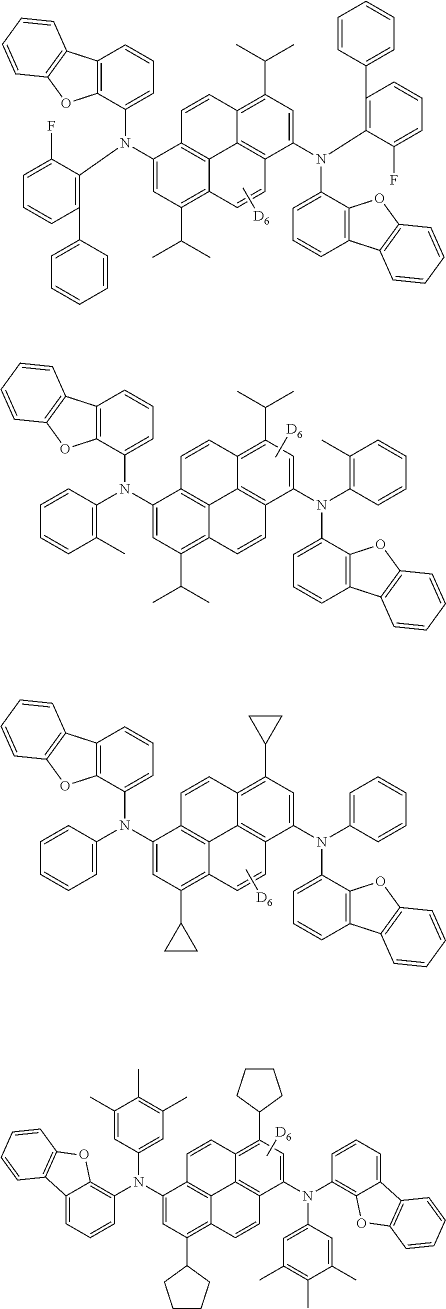

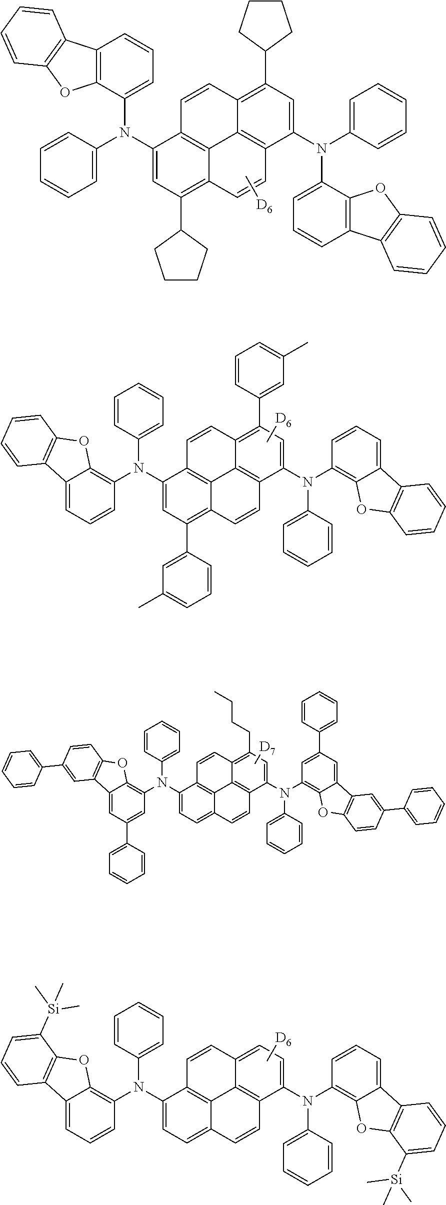

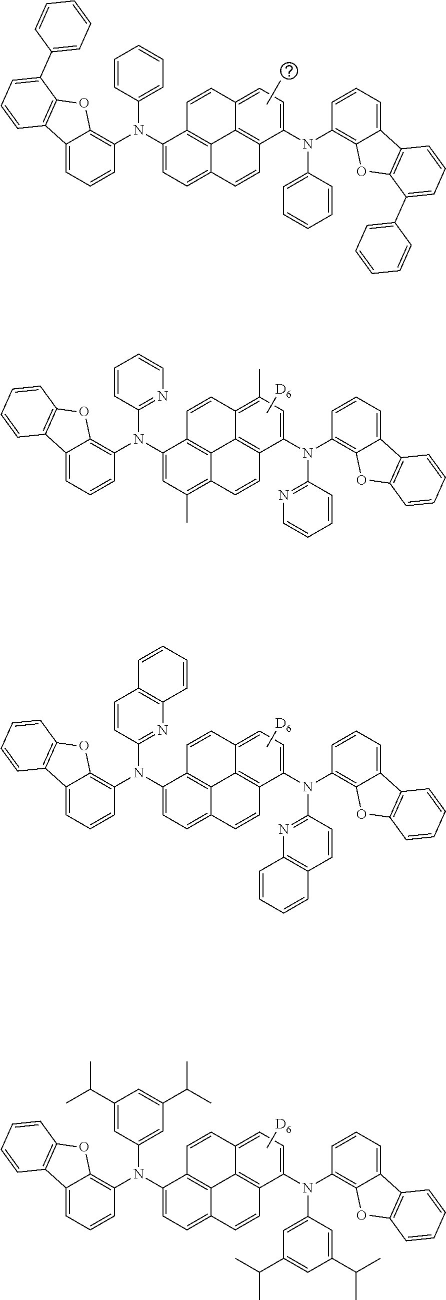

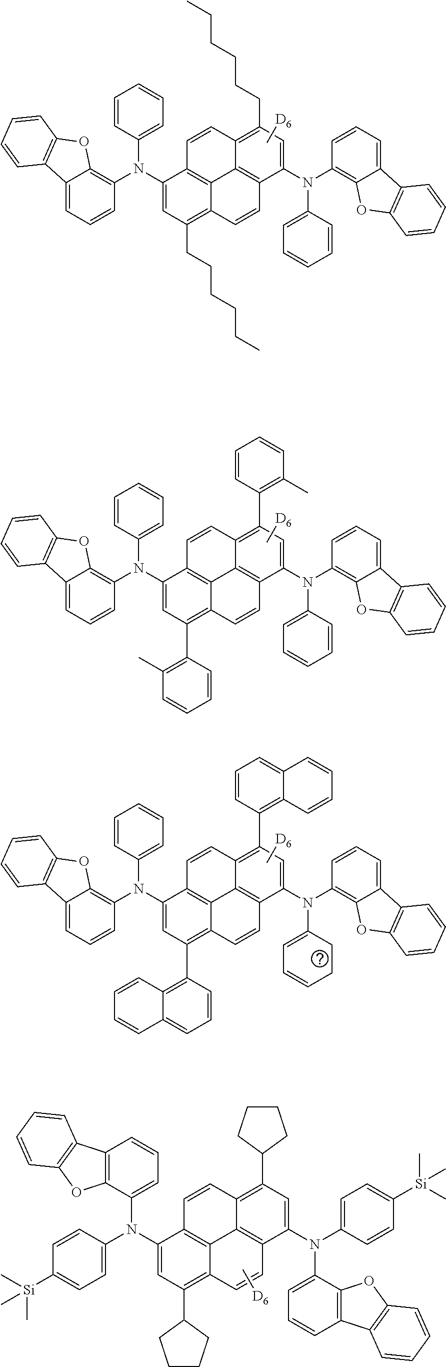

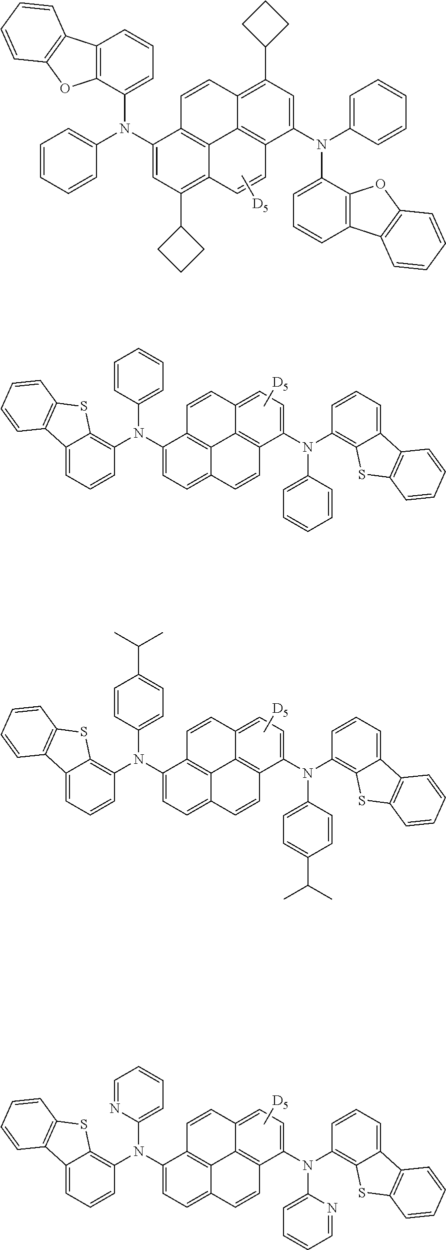

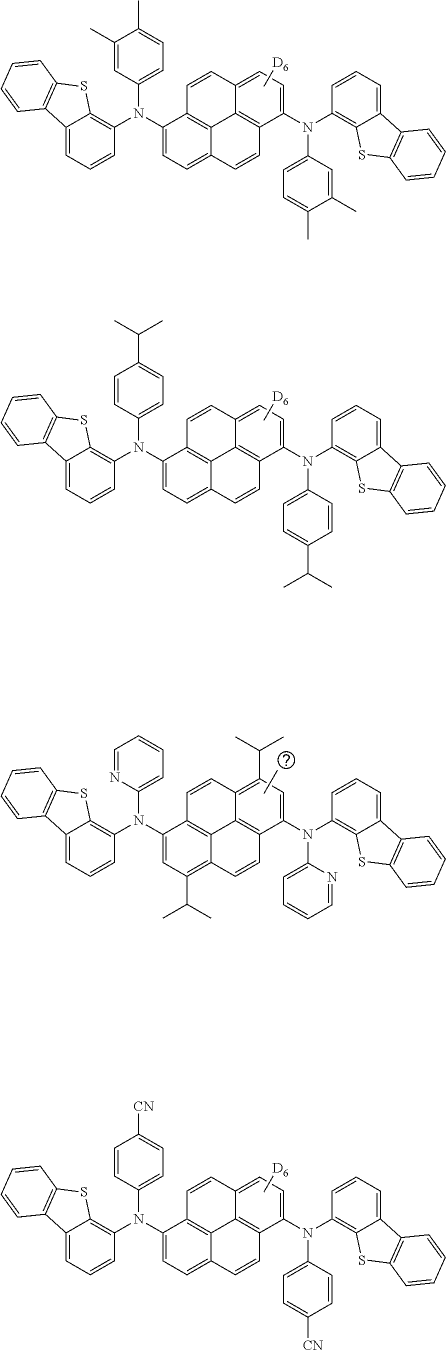

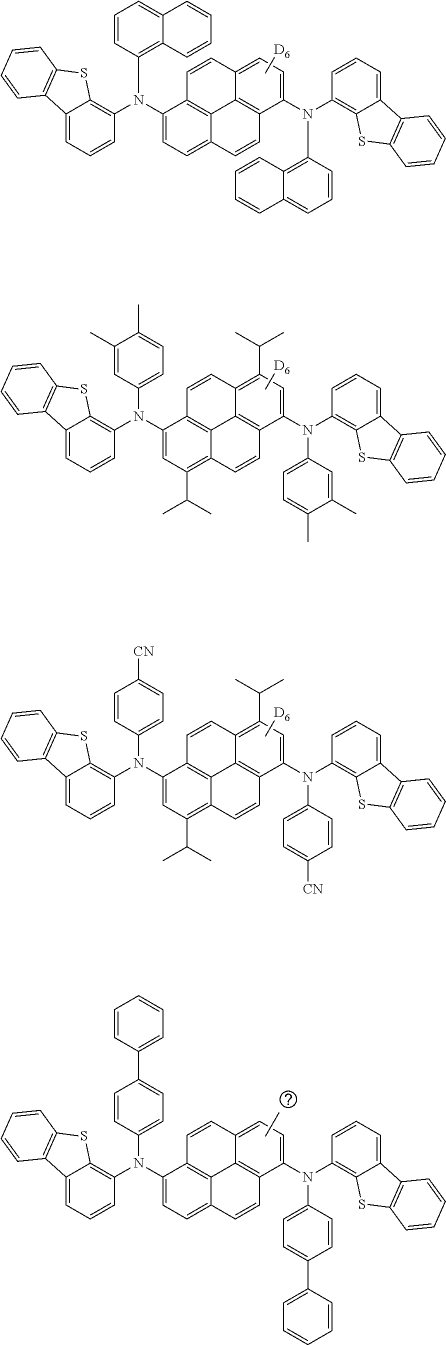

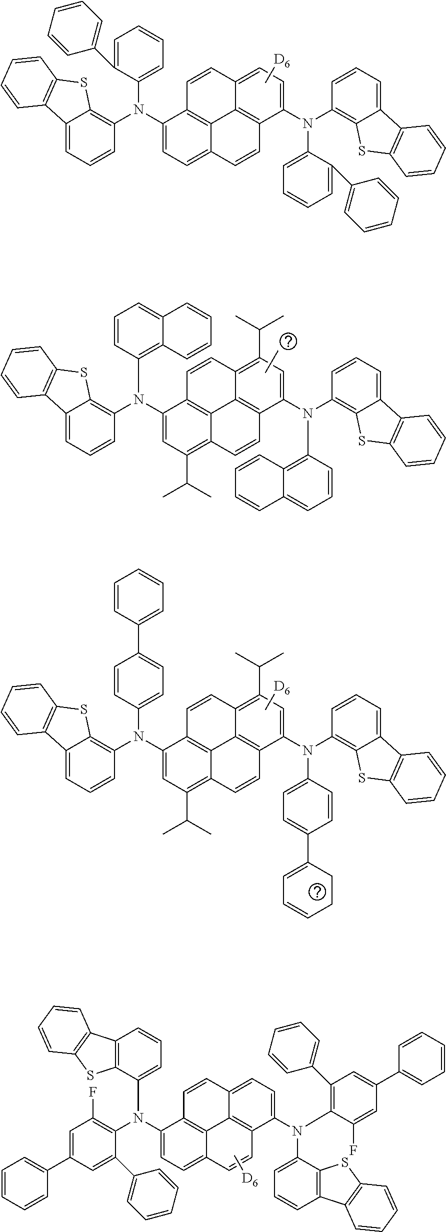

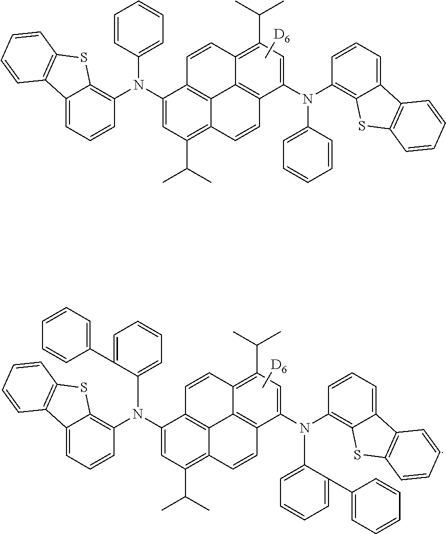

14. The organic light emitting device of claim 9, wherein the organic light emitting diode further includes:
a second emitting material layer including a second host being an anthracene derivative and a second dopant being a pyrene derivative, and positioned between the first emitting material layer and the second electrode; and
a first charge generation layer positioned between the first and second emitting material layers,
wherein at least one of an anthracene core of the second host and a pyrene core of the second dopant is deuterated.

15. The organic light emitting device of claim 9, wherein a red pixel, a green pixel and a blue pixel are defined on the substrate, and the organic light emitting diode corresponds to each of the red, green and blue pixels, and
wherein the organic light emitting device further includes:
a color conversion layer disposed between the substrate and the organic light emitting diode or on the organic light emitting diode and corresponding to the red and green pixels.

16. The organic light emitting device of claim 14, wherein the organic light emitting diode further includes:
a third emitting material layer configured to emit a yellow-green light, and positioned between the first charge generation layer and the second emitting material layer; and
a second charge generation layer positioned between the second and third emitting material layers.

17. The organic light emitting device of claim 14, wherein the organic light emitting diode further includes:
a third emitting material layer configured to emit a red light and a green light, and positioned between the first charge generation layer and the second emitting material layer; and
a second charge generation layer positioned between the second and third emitting material layers.

18. The organic light emitting device of claim 28, wherein a red pixel, a green pixel and a blue pixel are defined on the substrate, and the organic light emitting diode corresponds to each of the red, green and blue pixels, and
wherein the organic light emitting device further includes:
a color filter layer disposed between the substrate and the organic light emitting diode or on the organic light emitting diode and corresponding to the red, green and blue pixels.
